(19) United States
(12) Patent Application Publication
(10) Pub. No.: US 2023/0248834 A1
(43) Pub. Date: Aug. 10, 2023

(54) DEGRADATION OF (EGFR) BY CONJUGATION OF EGFR INHIBITORS WITH E3 LIGASE LIGAND AND METHODS OF USE

(71) Applicant: BEIGENE, LTD., Camana Bay, Grand Cayman (KY)

(72) Inventors: Bailin LEI, Beijing (CN); Huaqing LIU, Beijing (CN); Songzhe HAN, Beijing (CN); Zhiwei WANG, Beijing (CN)

(21) Appl. No.: 18/015,561

(22) PCT Filed: Jul. 15, 2021
(86) PCT No.: PCT/CN2021/106485

§ 371 Date: Jan. 11, 2023

(30) Foreign Application Priority Data
Jul. 16, 2020 (WO) PCT/CN2020/102501
Sep. 3, 2020 (WO) PCT/CN2020/113237
Jan. 8, 2021 (WO) PCT/CN2021/070896
Jun. 21, 2021 (WO) PCT/CN2021/101279

(51) Int. Cl. A61K 47/55; A61K 47/54

(52) U.S. Cl. CPC A61K 47/55; A61K 47/545

(57) ABSTRACT

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9; Sakamoto K. M. et al., Methods Enzymol. 2005; 399:833-847). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4A^{CRBN} E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) In the recent years, PROTACs as useful modulators promote the selective degradation of a wide range of target proteins have been reported in antitumor studies. (Lu J. et al., Chem Biol. 2015; 22(6):755-763; Ottis P. et al., Chem Biol. 2017; 12(4):892-898; Crews C. M. et al., J Med Chem. 2018; 61(2):403-404; Neklesa T. K. et al., Pharmacol Ther 2017, 174:138-144; Cermakova K. et al., Molecules, 2018.23(8); An S. et al., E Bio Medicine, 2018; Lebraud H. et al., Essays Biochem. 2017; 61(5): 517-527; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808(2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, and WO2018071606.

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137). Homo- or heterodimerization of EGFR and other ErbB family members activates cytoplasmic tyrosine kinase domains to initiate intracellular signaling. Overexpression or activating mutations of EGFR are associated the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611). The first-generation EGFR tyrosine kinase inhibitors (EGFR-TKIs) gefitinib and erlotinib have approved for NSCLC patients with EGFR activation mutations (M. Maemondo, N. Engl. J. Med. 362 (2010) 2380-2388). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562). There is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562 and US20190106417.

Although, a number of EGFR-targeting PROTACs which were designed to degrade EGFR mutant proteins have been published (Zhang X., et al. Eur. J. Med. Chem. 2020, 192, 112199; Zhang H, et al. Eur. J. Med. Chem. 2020, 189, 112061; Lu X, Med. Res. Rev. 2018, 38(5):1550-1581. He K., et al. Bioorg. Med. Chem. Lett. 2020, 15, 127167). Most of the published molecules are based on first, second, and third generation of EGFR inhibitors. However, there were no data which showed those EGFR-Targeting PROTACs degrading all the main EGFR mutations, Such as Del19, L858R, Del19/T790M, L858R/T790M, Del19/T790M/C797S, L858R/T790M/C797S.

The present application provides novel bifunctional compounds and compositions for the treatment of serious diseases.

### SUMMARY OF THE INVENTION

One objective of the present invention is to provide compounds and derivatives formed by conjugating EGFR inhibitor moieties with E3 ligase Ligand moieties, which function to recruit targeted proteins to E3 ubiquitin ligase for degradation, and methods of preparation and uses thereof.

Aspect 1: A compound of Formula (I):

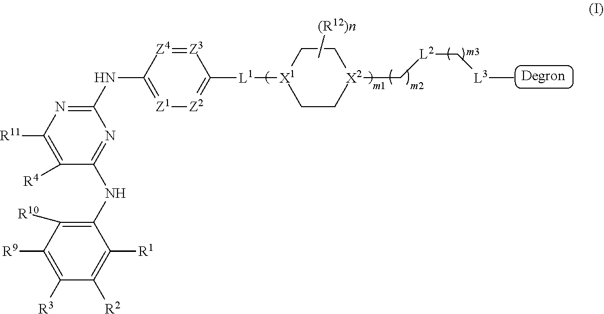

or a N-oxide thereof, or a pharmaceutically acceptable salt thereof, or a stereoisomer thereof, or a deuterated analog thereof, or a prodrug thereof,

wherein:

R¹ is selected from —P(O)R^{1a}R^{1b};

R^{1a} and R^{1b} are each independently selected from hydrogen, —C₁-C₈alkyl or C₃-C₈cycloalkyl, said —C₁-C₈alkyl or C₃-C₈cycloalkyl is optionally substituted with at least one halogen;

R² and R³ are each independently selected from hydrogen, halogen, —C₁-C₈alkyl, —C₃-C₈cycloalkyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, or —NR^{2a}SO₂R^{2b}; each of —C₁- C₈alkyl, C₃-C₈cycloalkyl, 3- to 8-membered heterocyclyl, C₆-C₁₂aryl or 5- to 12-membered heteroaryl is optionally substituted with at least one substituent R^{2d}, or

R² and R³ together with the carbon atoms to which they are attached, form a 5 or 6-membered unsaturated or saturated ring, said ring comprising 0-3 heteroatoms independently selected from nitrogen, oxygen or sulfur; said ring is optionally substituted with at least one substituent R^{2e};

R^{2e}, at each occurrence, is independently hydrogen, halogen, —C₁-C₈alkyl, —C₁-C₈alkoxy, —C₃-C₈cycloalkyl, 3- to 8-membered heterocyclyl, C₆-C₁₂aryl, 5- to 12-membered heteroaryl, oxo (═O), —OR^{2a}, thioxo (═S), —SR^{2a}, —CN, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b} or —NR^{2a}SO₂R^{2b}; each of —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent R^{2d};

R^{2a} and R^{2b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;

R^{2d}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;

R⁴ is selected from hydrogen, halogen, —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl, 5- to 12-membered heteroaryl, —CN, —SO₂R^{4a}, —SO₂NR^{4a}R^{4b}, —COR^{4a}, —CO₂R^{4a}, —CONR^{4a}R^{4b}, —NR^{4a}R^{4b}, —NR^{4a}COR^{4b}, —NR^{4a}CO₂R^{4b} or —NR^{4a}SO₂R^{4b}; each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{4c}, —SO₂R^{4c}, —SO₂NR^{4c}R^{4d}, —COR^{4c}, —CO₂R^{4c}, —CONR^{4c}R^{4d}, —NR^{4c}R^{4d}, —NR^{4c}COR^{4d}, —NR^{4c}CO₂R^{4d} or —NR^{4c}SO₂R^{4d};

R^{4a}, R^{4b}, R^{4c} and R^{4d} are each independently hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;

R⁹, R¹⁰, R¹¹ and R¹² are each independently selected from hydrogen, halogen, —C₁-C₈alkyl, —NR^{9a}R^{9b}, —OR^{9a}, —C₃-C₈cycloalkyl, 3- to 8-membered heterocyclyl, —C₆-C₁₂aryl, 5- to 12-membered heteroaryl, oxo (═O) or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{9c}; or

two R¹² together with the carbon atoms to which they are attached, form a 3- to 12-membered ring, said ring comprising 0-3 heteroatoms independently selected from nitrogen, oxygen or sulfur; said ring is optionally substituted with at least one substituent R^{9c};

R^{9a} and R^{9b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{9d}; or R^{9c} and R^{9d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;

Z¹, Z², Z³ and Z⁴ are each independently selected from —CR^{Z}, or N;

R^{Z}, at each occurrence, is independently selected from hydrogen, halogen, —C₁-C₈alkyl, —NR^{Za}R^{Zb}, —OR^{Za}, —SR^{Za}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc};

R^{Za} and R^{Zb} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Zd};

R^{Zc} and R^{Zd} are each independently halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;

L¹ is selected from a single bond, —O—, —SO₂—, —C(O)—, —NR^{L1a}—, —C₃-C₈cycloalkylene-, *^{L1}—O—C₁-C₈alkylene-**^{L1}, *^{L1}—C₁-C₈alkylene-O—**^{L1}, *^{L1}—SO₂—C₁-C₈alkylene-**^{L1}, *^{L1}—C₁-C₈alkylene-SO₂—**^{L1}, *^{L1}—CO—C₁-C₈alkylene-**^{L1}, *^{L1}—C₁-C₈alkylene-CO—**^{L1}, *^{L1}—NR^{L1a}C₁-C₈alkylene-**^{L1}, *^{L1}—C₁-C₈alkylene-NR^{L1a}—**^{L1}, *^{L1}—NR^{L1a}C(O)—**^{L1}, *^{L1}—C(O)NR^{L1a}—**^{L1}, —C₁-C₈alkylene-, —C₂-C₈alkenylene-, —C₂-C₈alkynylene-, —[O(CR^{L1a}R^{L1b})ₘ₄]ₘ₅—,

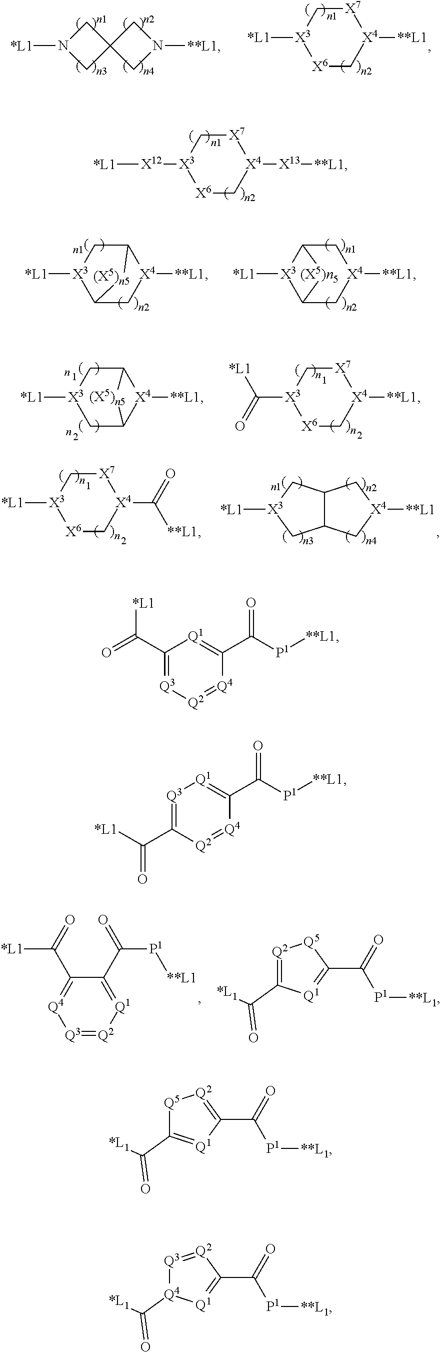

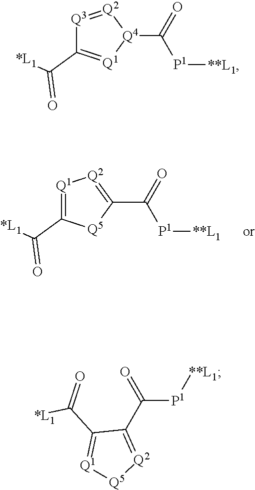

wherein each of said —C₃-C₈cycloalkylene-, *^{L1}—O—C₁-C₈alkylene-**^{L1}, *^{L1}—C₁-C₈alkylene-O—**^{L1}, *^{L1}—SO₂—C₁-C₈alkylene-**^{L1}, *^{L1}—C₁-C₈alkylene-SO₂—**^{L1}, *^{L1}—CO—C₁-C₈alkylene-**^{L1}, *^{L1}—C₁-C₈alkylene- CO—**^{L1}, *^{L1}—NR^{L1a}—C₁-C₈alkylene-**^{L1}, *^{L1}—C₁-C₈alkylene-NR^{L1a}—**^{L1}, —C₁-C₈alkylene-, —C₂-C₈alkenylene-, —C₂-C₈alkynylene-,

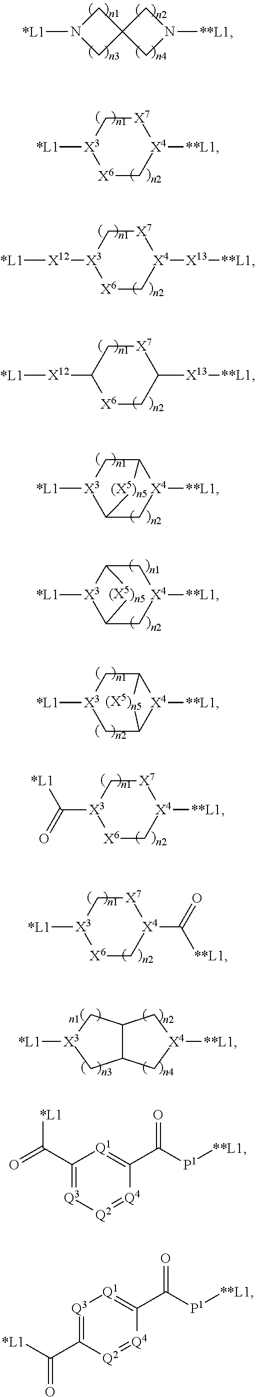

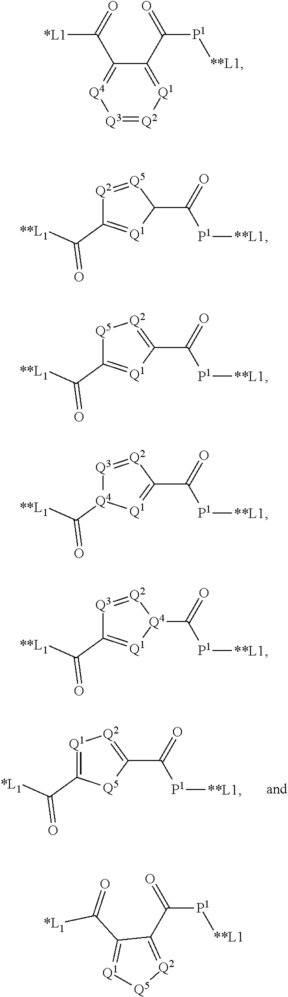

is optionally substituted with at least one R^{L1c};

wherein *^{L1} refers to the position attached to the

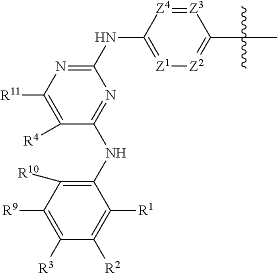

moiety, and **^{L1} refers to the position attached to the

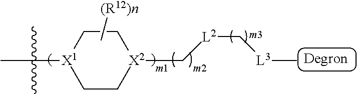

moiety;

R^{L1a} and R^{L1b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L1d}; each of said R^{L1c} and R^{L1d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or

two R^{L1c} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

L² is selected from a single bond, —O—, —SO₂—, —CO—, —NR^{L2a}—, —C₃-C₈cycloalkylene-, *^{L2}—O—C₁-C₈alkylene-**^{L2}, *^{L2}—C₁-C₈alkylene-O—**^{L2}, *^{L2}—SO₂—C₁-C₈alkylene-**^{L2}, *^{L2}—C₁-C₈alkylene-SO₂—**^{L2}, *^{L2}—CO—C₁-C₈alkylene-**^{L2}, *^{L2}—C₁-C₈alkylene-CO—**^{L2}, *^{L2}—NR^{L2a}—C₁-C₈alkylene-**^{L2}, *^{L2}—C₁-C₈alkylene-NR^{L2a}—**^{L2}, *_{L2}—NR^{L2a}C(O)—**^{L2}, *^{L2}—C(O)NR^{L2a}—**^{L2}—C₁-C₈alkylene-, —C₂-C₈alkenylene-, —C₂-C₈alkynylene-, —[O(CR^{L2a}R^{L2b})ₘ₄]ₘ₅—,

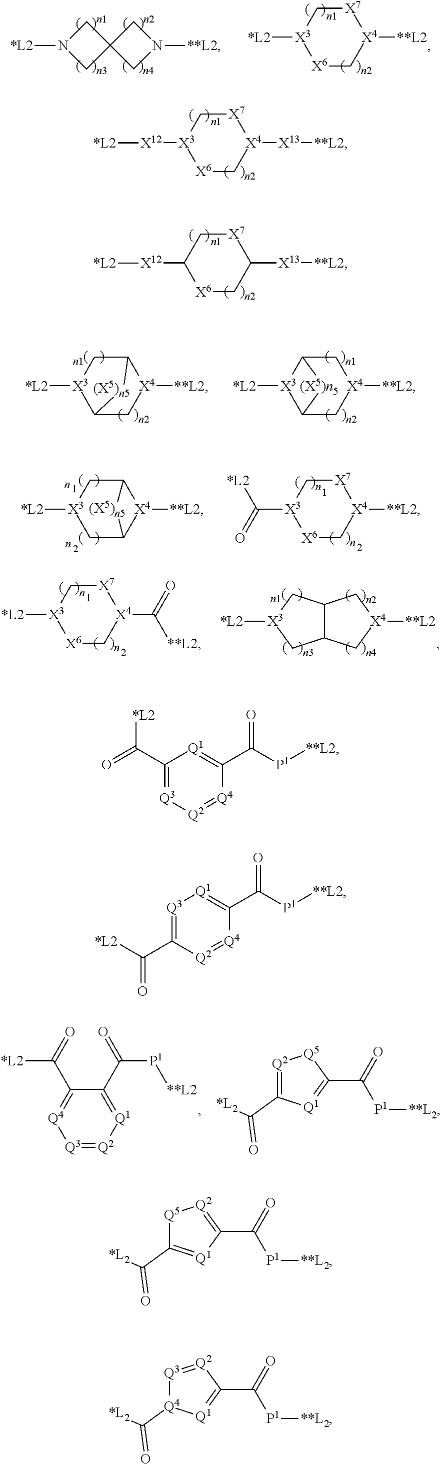

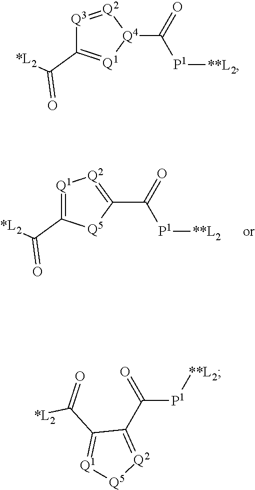

wherein each of said —C₃-C₈cycloalkylene-, *^{L2}—O—C₁-C₈alkylene-**^{L2}, *^{L2}—C₁-C₈alkylene-O—**^{L2}, *^{L2}—SO₂—C₁-C₈alkylene-**^{L2}, *^{L2}—C₁-C₈alkylene-SO₂—**^{L2}, *^{L2}—CO—C₁-C₈alkylene-**^{L2}, *^{L2}—C₁-C₈alkylene- CO—**^{L2}, *^{L2}—NR^{L2a}C₁-C₈alkylene-**^{L2}, *^{L2}—C₁-C₈alkylene-NR^{2a}—**^{L2}, —C₁-C₈alkylene-, —C₂-C₈alkenylene-, —C₂-C₈alkynylene-,

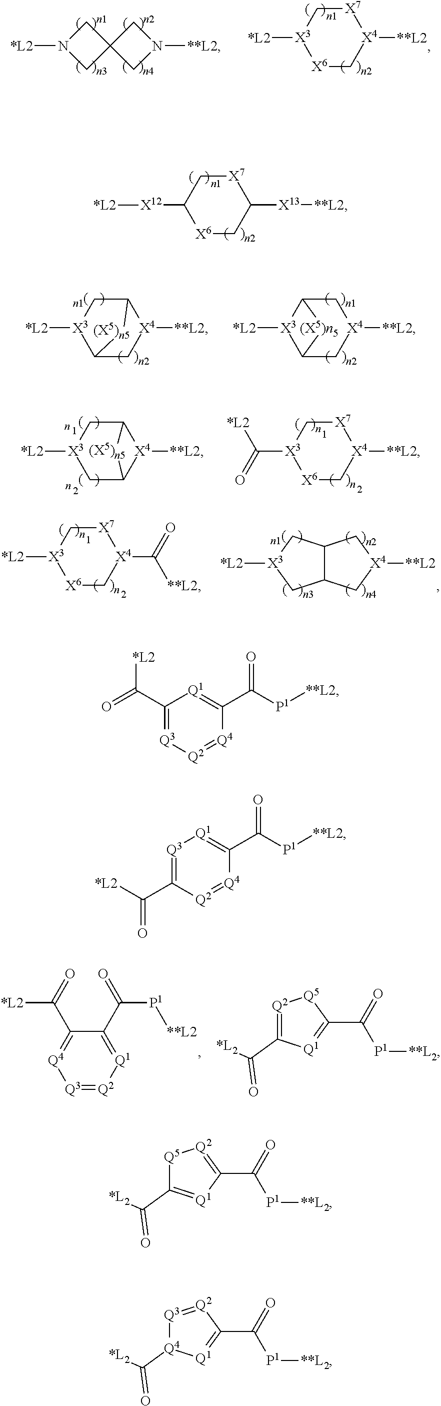

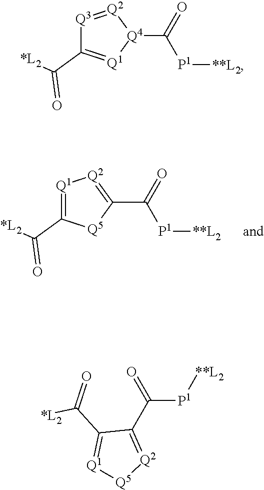

is optionally substituted with at least one substituent R^{L2c};

wherein *^{L2} refers to the position attached to

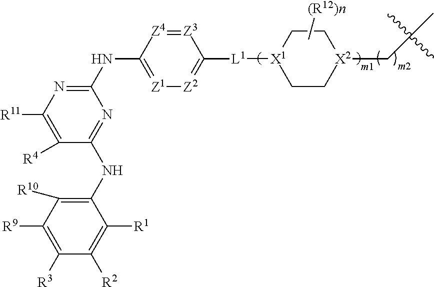

moiety, and **^{L2} refers to the position attached to the

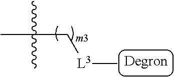

moiety;

R^{L2a} and R^{L2b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L2d};

each of said R^{L2c} and R^{L2d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₂aryl or 5- to 12-membered heteroaryl; or

two R^{L2c} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

L³ is selected from a single bond, —O—, —SO₂—, —CO—, —NR^{L3a}—, —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂—**^{L3}, *^{L3}—CO—C₁-C₈alkylene-**^{L3}, *^{L3}—C₁-C₈alkylene-CO—**^{L3}, *^{L3}—NR^{L3a}—C₁-C₈alkylene-**^{L3}, *^{L3}—C₁-C₈alkylene- NR^{L3a}—**^{L3}, *^{L3}—NR^{L3a}C(O)—**^{L3}, *^{L3}—C(O)NR^{L3a}—**^{L3}, —C₁-C₈alkylene-, —C₂-C₈alkenylene-, —C₂-C₈alkynylene-, —[O(CR^{L3a}R^{L3b})ₘ₄]ₘ₅—,

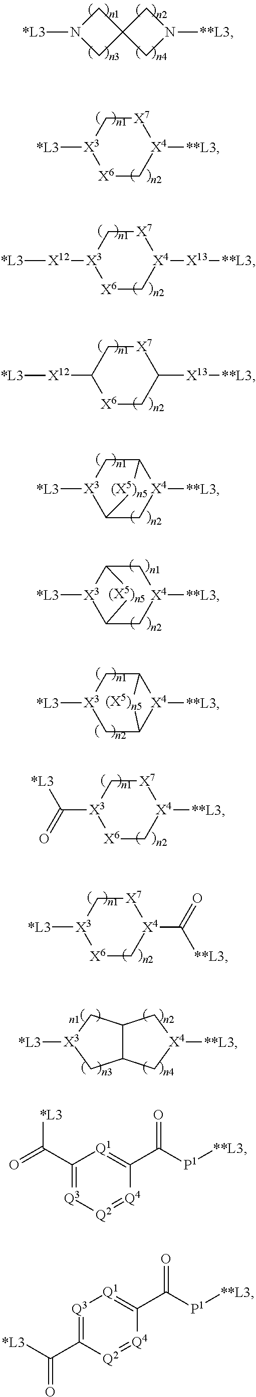

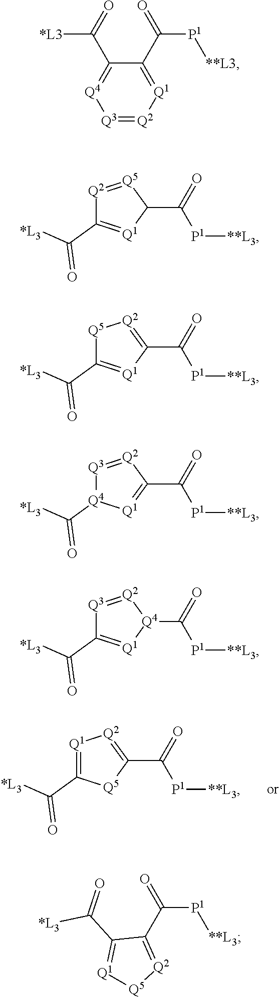

wherein each of said —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂—**^{L3}, *^{L3}—CO—C₁-C₈alkylene-**^{L3}, *^{L3}—C₁-C₈alkylene- CO—**^{L3}, *^{L3}—NR^{L3a}—C₁-C₈alkylene-**^{L3}, *^{L3}—C₁-C₈alkylene-NR^{L3a}—**^{L3}, —C₁-C₈alkylene, —C₁-C₈alkenylene, —C₂-C₈alkynylene-,

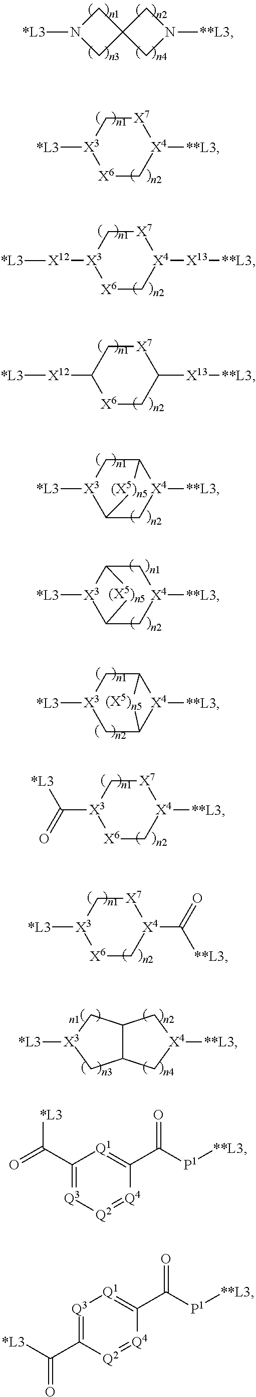

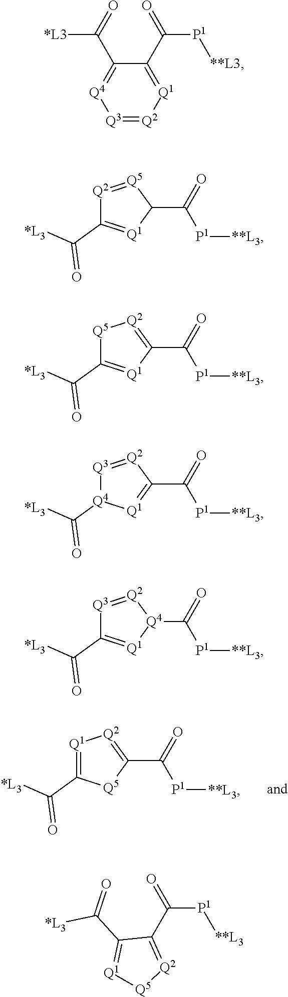

is optionally substituted with at least one substituent R^{L3c};

wherein *^{L3} refers to the position attached to

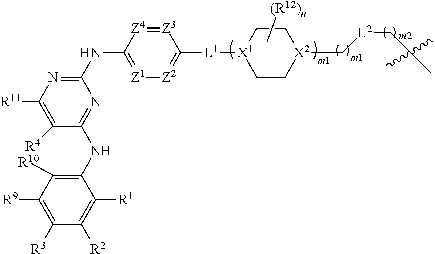

moiety, and **^{L3} refers to the position attached to the

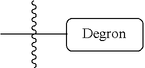

moiety;

R^{L3a} and R^{L3b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L3d}; each of said R^{L3c} and R^{L3d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or

two R^{L3c} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;

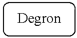

is selected from

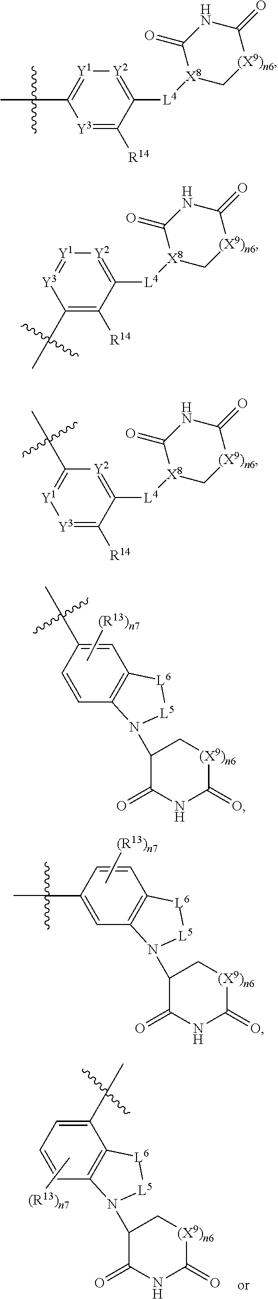

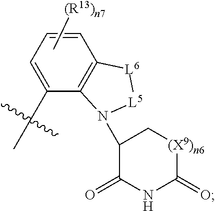

R¹³ and R¹⁴ are each independently selected from hydrogen, halogen, CN, —C₁-C₈alkyl, —C₁-C₈alkoxy, C₃-C₈cycloalkyl, 3- to 8-membered heterocyclyl, C₆-C₁₂aryl or 5- to 12-membered heteroaryl; said each —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent halogen, —C₁-C₈alkyl, C₁-C₈alkoxy-C₁-C₈alkyl-, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;

X¹, X², X³, X⁴ and X⁸ are each independently selected from —CR^{a}, or N;

X⁵, X⁶, X⁷ and X⁹ are each independently selected from —NR^{a}—, —O—, —S— and —CR^{a}R^{b}—;

X¹² and X¹³ are each independently selected from —C(O)—, —NR^{a}— and —O—;

L⁴, L⁵ and L⁶ are each independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;

Q¹, Q², Q³, Q⁴, Y¹, Y², Y³ are each independently selected from CR^{a} or N;

Q⁵ is each independently selected from —O—, —NR^{a}—, —CR^{a}R^{b}—, —S— or —C(O)—;

P¹ is a single bond, —O—, —NR^{a}—, —CR^{a}R^{b}—, —S—, —SO— or —SO₂—;

at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₂aryl or 5- to 12-membered heteroaryl; or

R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₂aryl or 5- to 12-membered heteroaryl;

m₁ is 0, 1 or 2;

m₂ and m₃ are each independently 0, 1, 2, 3, 4, 5, 6, 7 or 8;

m₄ and m₅ are each independently 0, 1, 2 or 3;

n, n₁, n₂, n₃, n₄ and n₅ are each independently 0, 1, 2 or 3; and

n₆, n₇, n₈ and n₉ are each independently 0, 1, 2, 3 or 4.

Aspect 2. The compound of Aspect 1, wherein the compound is selected from formula (II), (III)

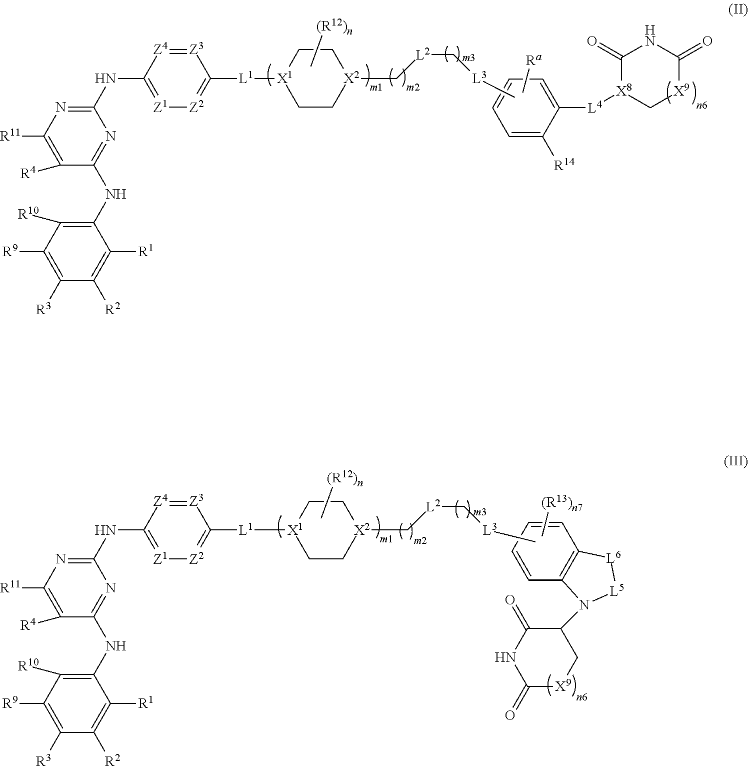

R¹, R², R³, R⁴, R⁹, R¹⁰, R¹¹, R¹², R¹³, R¹⁴, R^{a}, Z¹, Z², Z³, Z⁴, L¹, L², L³, L⁴, L⁵, L⁶, X¹, X², X⁸, X⁹, n, n6, n7, m1, m2 and m3 are each independently defined as Aspect 1.

Aspect 3. The compound of Aspects 1-2, wherein R¹ is selected from —P(O)R^{1a}R^{1b}, wherein R^{1a} and R^{1b} are each independently selected from hydrogen, —C₁-C₈alkyl (preferably —CH₃, —C₂H₅, —C₃H₇, —C₄H₉ or —C₅H₁₁; more preferably —CH₃, —CH₂CH₃, —CH₂CH₂CH₃, -iso-C₃H₇, —CH₂CH₂CH₂CH₃, -iso-C₄H₉, -sec-C₄H₉ or -tert-C₄H₉) or C₃-C₈cycloalkyl (preferably cyclopropyl, cyclobutyl or cyclopentyl).

Aspect 4. The compound of any one of Aspects 1-3, wherein R₁ is selected from —P(O)(CH₃)₂.

Aspect 5. The compound of any one of Aspects 1-4, wherein R² and R³ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, or —NR^{2a}SO₂R²¹; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl is optionally substituted with at least one substituent R^{2d}, or

R² and R³ together with the carbon atoms to which they are attached, form a 5 or 6-membered unsaturated or saturated ring, said ring comprising 0, 1, 2 or 3 heteroatoms independently selected from nitrogen, oxygen or sulfur; said ring is optionally substituted with at least one substituent R^{2e};

R^{2e}, at each occurrence, is independently —H, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl, oxo (═O), —CN, CF₃, CHF₂, CH₂F, thioxo (═S), —SCF₃, —SCHF₂, —SCH₂F, —SCH₂CF₃, —SCF₂CH₃, —SCF₂CF₃, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b} or —NR^{2a}SO₂R²¹; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl is optionally substituted with at least one substituent R^{2d};

R^{2a} and R^{2b} are each independently selected from hydrogen, methyl, ethyl, propyl, butyl, pentyl, hexyl, heptyl, octyl, methoxy, ethoxy, propoxy, butoxy, pentoxy, hexoxy, heptyloxy, octyloxy, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;

R^{2d}, at each occurrence, is independently halogen, —OH, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 6. The compound of any one of Aspects 1-5, wherein R² and R³ together with the carbon atoms to which they are attached, form a 5 or 6-membered unsaturated (preferred aromatic) or saturated ring, said ring comprising 1 or 2 nitrogen heteroatoms; said ring is optionally substituted with at least one substituent —H, —F, —Cl, —Br, —I, methyl, ethyl, propyl (n- or iso-), butyl, pentyl, methoxy, ethoxy, propoxy, butoxy, pentoxy, cyclopropyl, cyclobutyl, cyclopentyl, cyclohexyl, cycloheptyl, cyclooctyl, —CH₂OH, —SCH₃, —SC₂H₅, oxo (═O), thioxo (═S), —CF₃, —CHF₂, —CH₂F, —SCF₃, —OMe, —OC₂H₅, —CN, —C(O)CH₃,

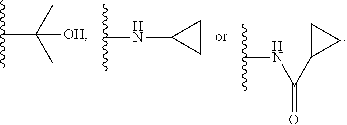

Aspect 7. The compound of any one of Aspects 1-6, wherein R² and R³ together with the carbon atoms to which they are attached, form a 6-membered unsaturated (preferred aromatic), said ring comprising 1 or 2 nitrogen heteroatoms; said ring is optionally substituted with one substituent —H, —F, —Cl, —Br, —I, methyl, ethyl or cyclopropyl.

Aspect 8. The compound of any one of Aspects 1-7, wherein R⁴ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl, —C₂-C₈alkynyl or —C₁-C₈alkoxy; each of methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl or —C₂-C₈alkynyl is optionally substituted with —F, —Cl, —Br, —I, oxo (═O), or —CN.

Aspect 9. The compound of any one of Aspects 1-8, wherein R⁴ is hydrogen, —F, —Cl, —Br, —I, —CH₃, —CF₃, —CH₂F, or —CHF₂.

Aspect 10. The compound of any one of Aspects 1-9, wherein R⁴ is hydrogen, —F, —Cl, —Br or —I.

Aspect 11. The compound of any one of Aspects 1-10, wherein R⁹, R¹⁰, R¹¹ and R¹² are each independently selected from hydrogen, —F, —Cl, —Br, —I, methyl, ethyl, propyl, butyl, pentyl, hexyl, heptyl, octyl, —NR^{9a}R^{9b}, —OR^{9a}, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), or —CN; each of—methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9c};

R^{9a} and R^{9b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9d};

R^{9c} and R^{9d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 12. The compound of any one of Aspects 1-11, wherein R⁹, R¹⁰, R¹¹ and R¹² are each independently selected from hydrogen, —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, cyclohexyl, —CH₂OH, —CH₂OMe, oxo (═O), or —CN.

Aspect 13. The compound of any one of Aspects 1-12, wherein R⁹, R¹⁰, R¹¹ and R¹² are each independently selected from hydrogen, —CH₃, —F, —Cl, —Br or —I.

Aspect 14. The compound of any one of Aspects 1-10, wherein two R¹² together with the carbon atoms to which they are attached, form a 3, 4, 5, 6, 7 or 8-membered ring, said ring comprising 0, 1, 2 or 3 heteroatoms independently selected from nitrogen, oxygen or sulfur; said ring is optionally substituted with at least one substituent R^{9c};

R^{9c} is independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 15. The compound of any one of Aspects 1-10, wherein two R¹² together with the carbon atoms to which they are attached, form a 3, 4, 5, 6, 7 or 8-membered ring, preferably form a 3, 4, 5 or 6-membered ring, said ring comprising 0, 1, 2 or 3 heteroatoms independently selected from nitrogen, oxygen or sulfur; said ring is optionally substituted with at least one substituent —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, or cyclohexyl.

Aspect 16. The compound of any one of Aspects 1-4, wherein the

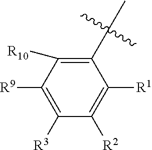

moiety is

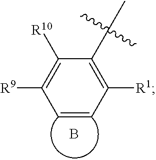

Ring B is a 5 or 6-membered unsaturated or saturated ring, said ring comprising 0, 1, 2 or 3 heteroatoms; said heteroatoms are independently selected from N, NR^{2e}, O or S;

said ring is optionally substituted with at least one substituent R^{2e}.

Aspect 17. The compound of any one of Aspects 1-16, wherein the

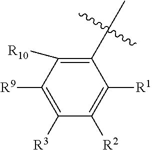

moiety is selected from

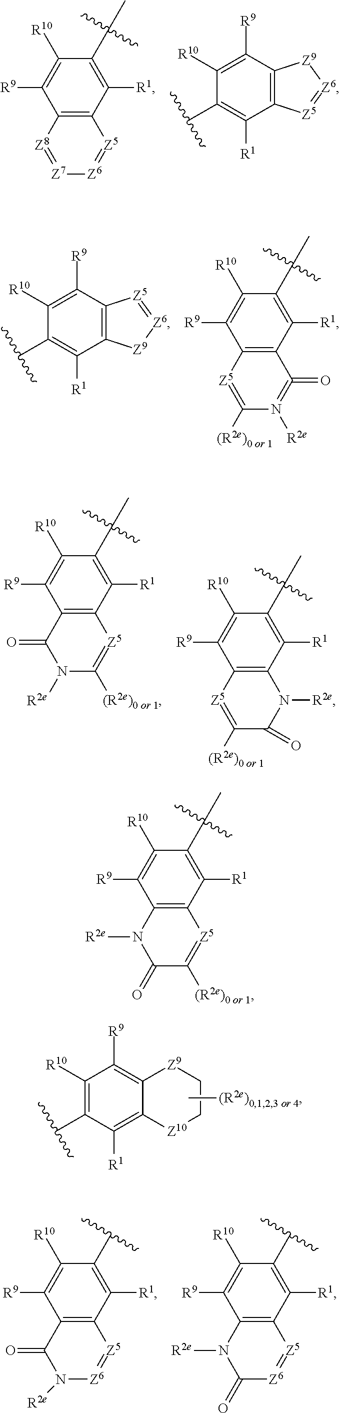

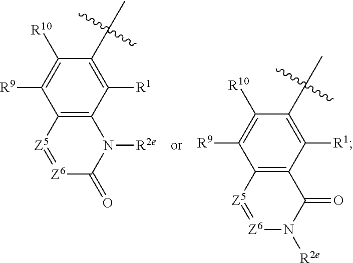

Z⁵, Z⁶, Z⁷ and Z⁸ are each independently selected from N, CH or CR^{2e};

Z⁹ and Z⁰ are each independently selected from O, S, NH or NR^{2e}.

Aspect 18. The compound of any one of Aspects 1-17, wherein the

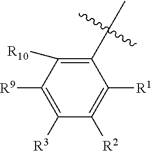

moiety is selected from

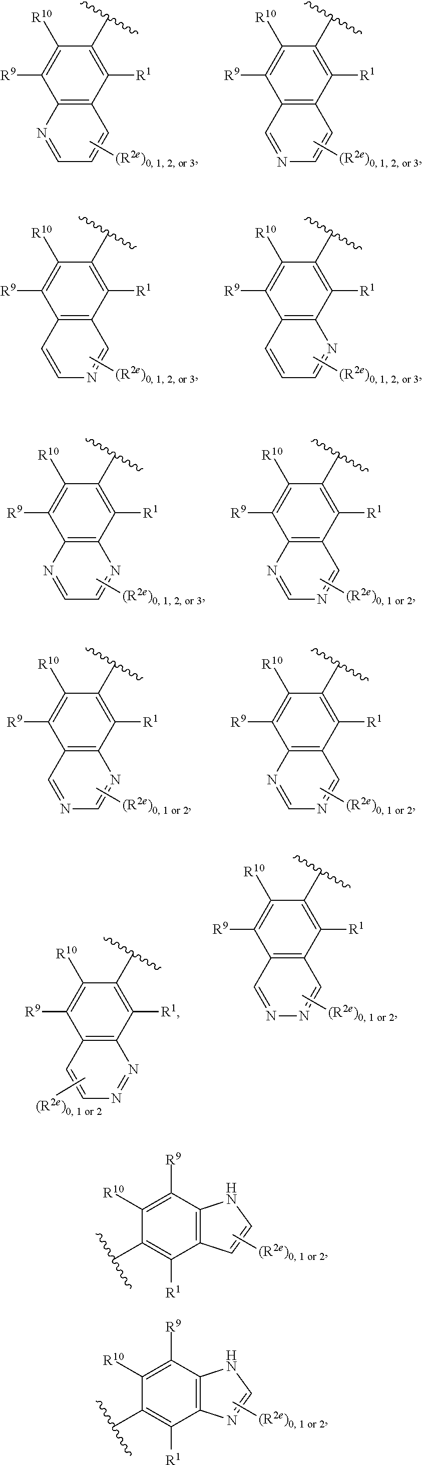

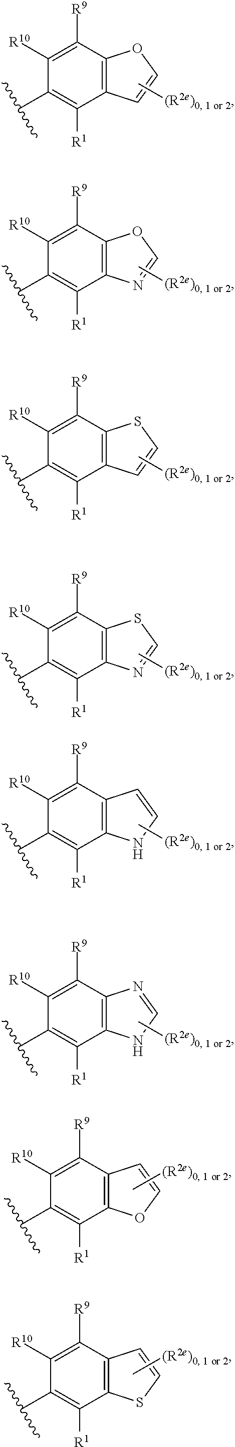

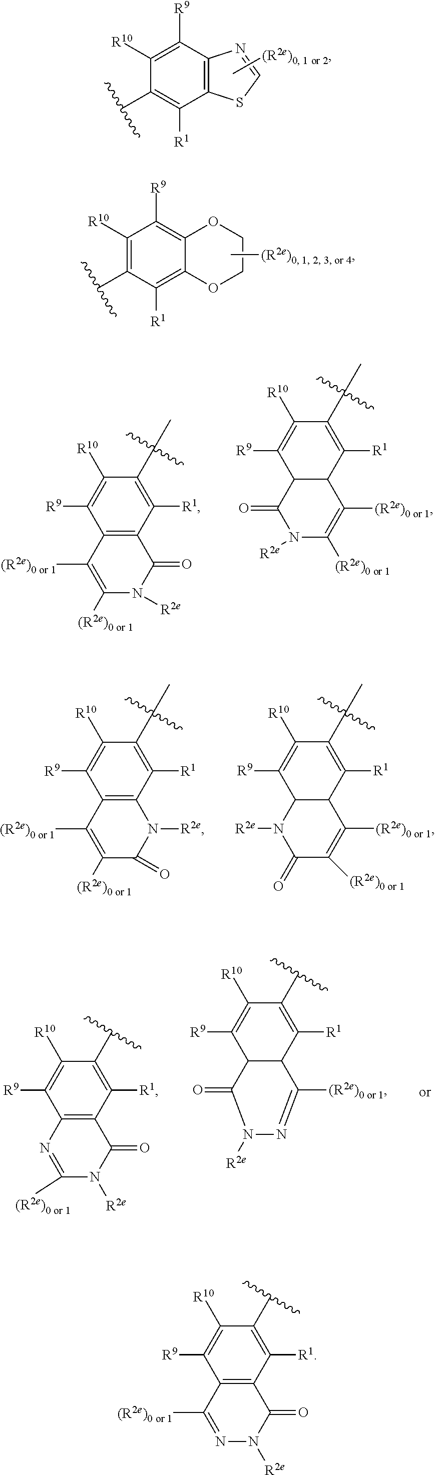

Aspect 19. The compound of any one of Aspects 1-18, wherein the

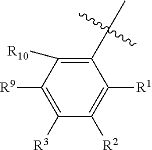

moiety is selected from

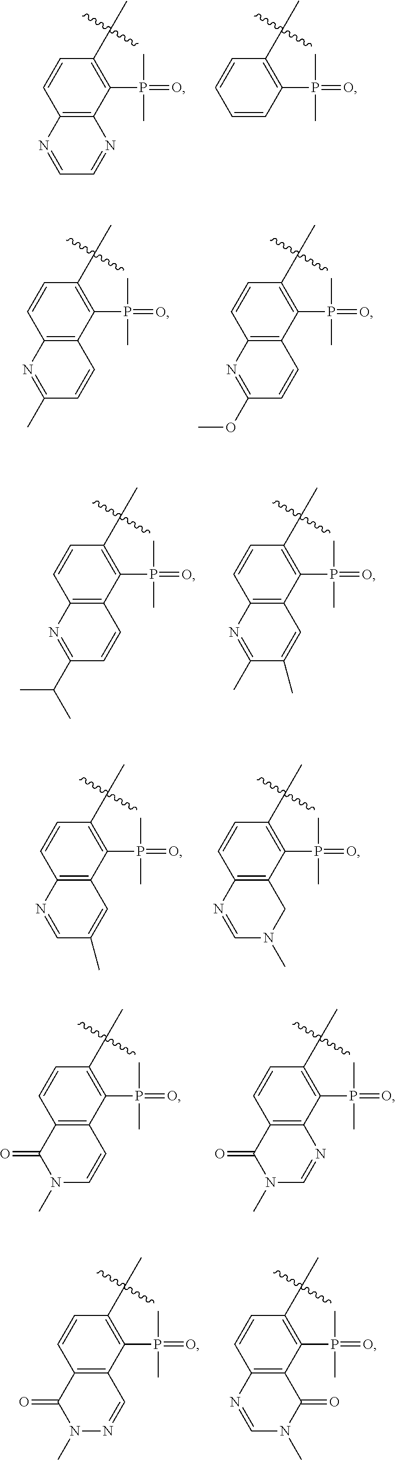

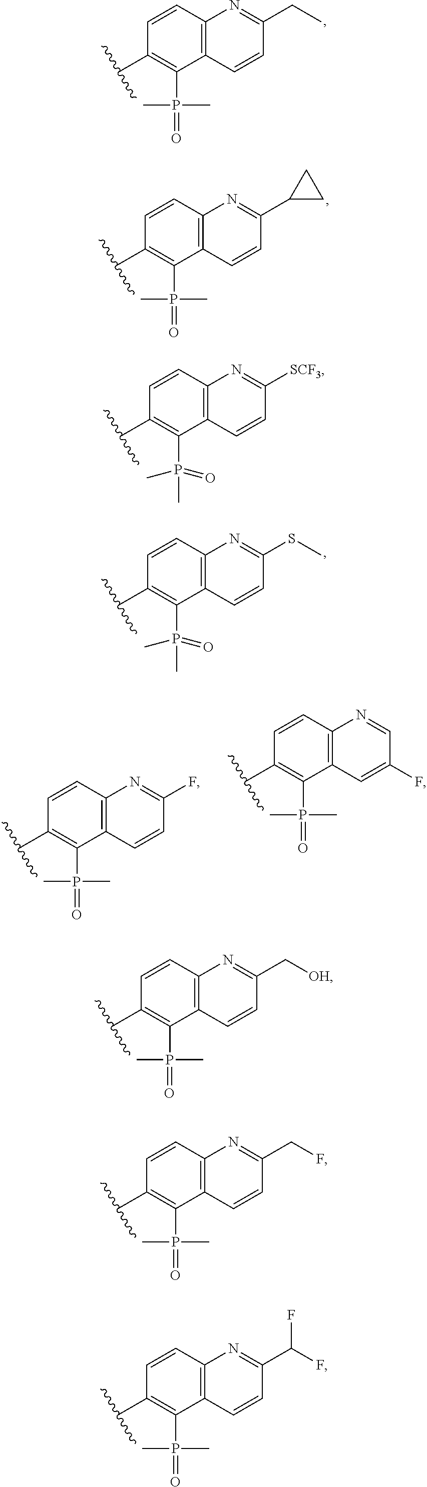

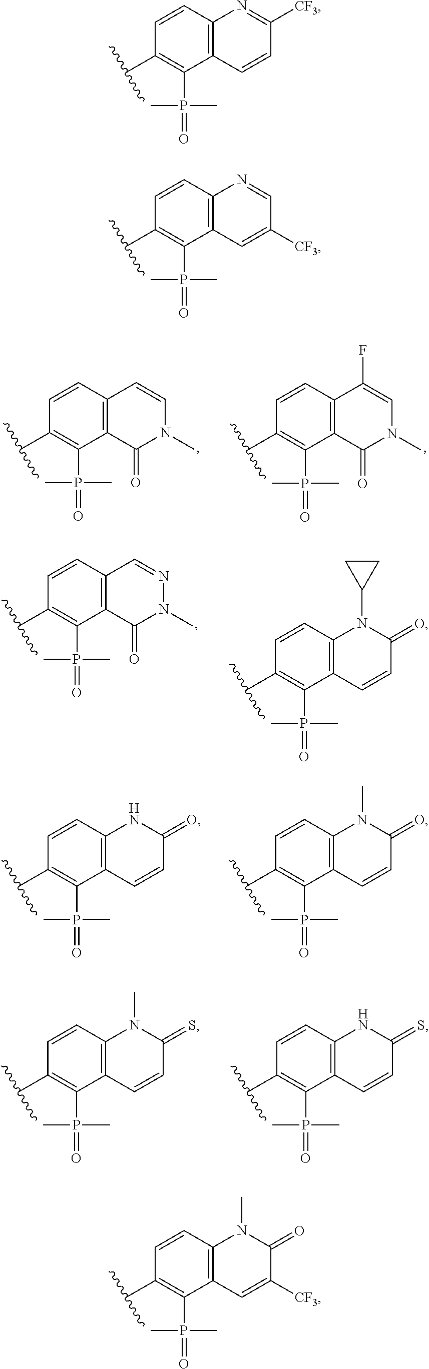

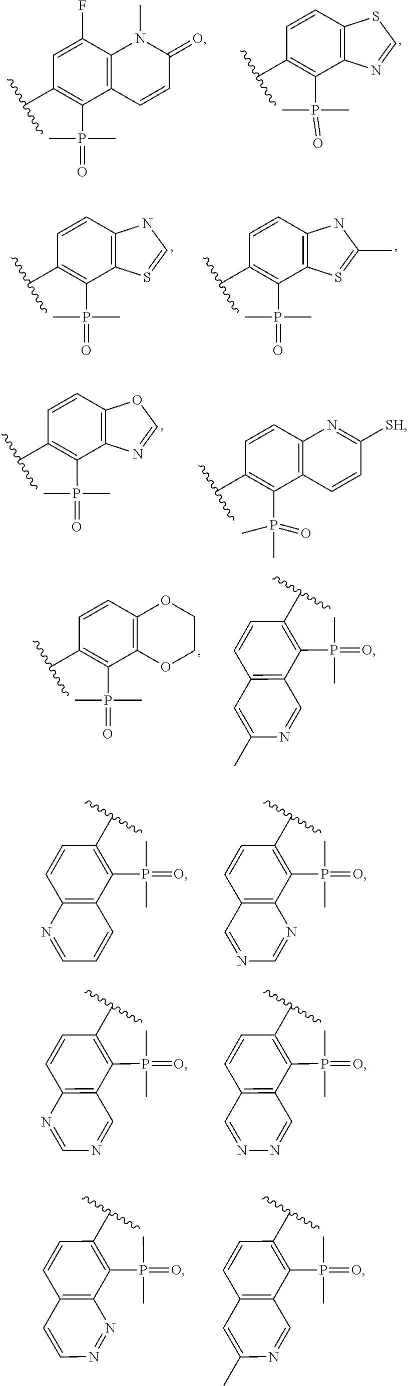

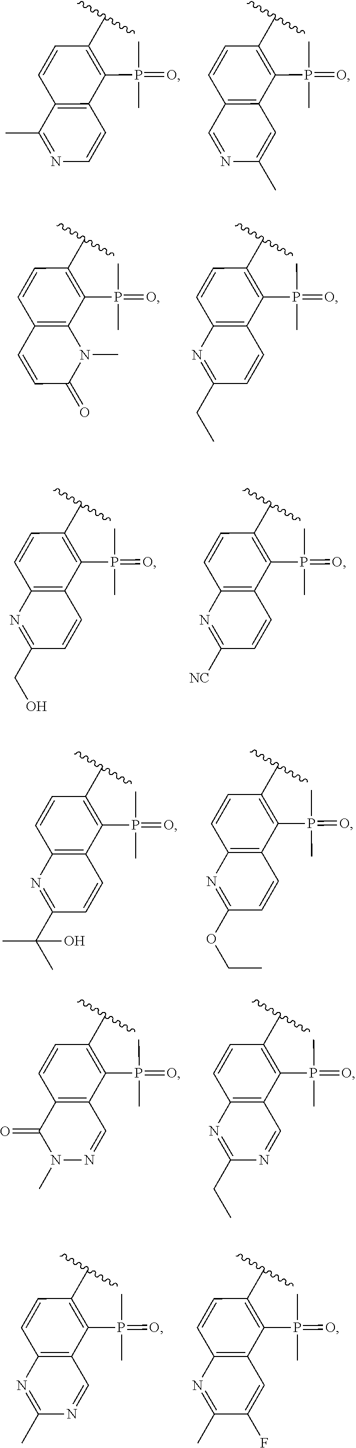

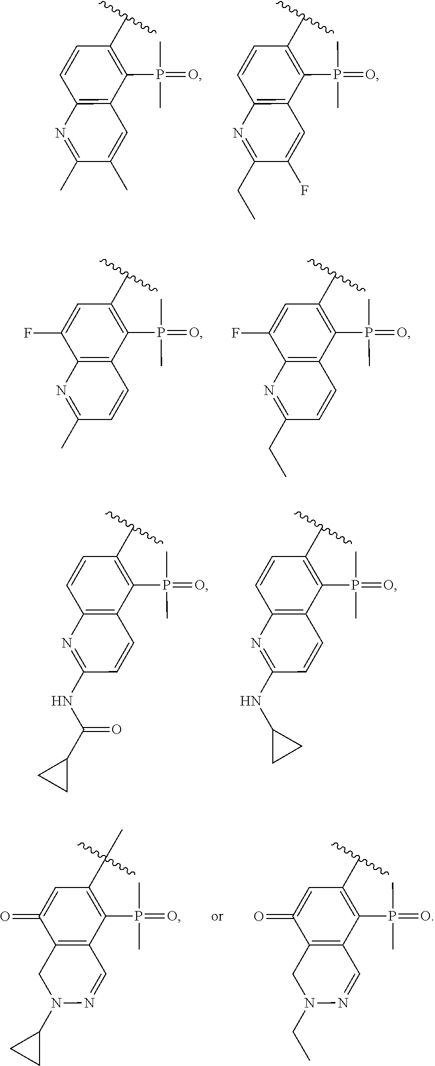

Aspect 20. The compound of any one of Aspects 1-19, wherein L¹ is selected from a single bond, —C₁-C₈alkylene- (preferably —CH₂—, —C₂H₄—, —C₃H₆—), —C(O)—C₁-C₈alkylene- (preferably —C(O)—CH₂—, —C(O)—C₂H₄—, —C(O)—C₃H₆—), —C₁-C₈alkylene-C(O)— (preferably —CH₂—C(O)—, —C₂H₄—C(O)—, —C₃H₆—C(O)—), —CO—, —O—, —N(CH₃)—, —NH—,

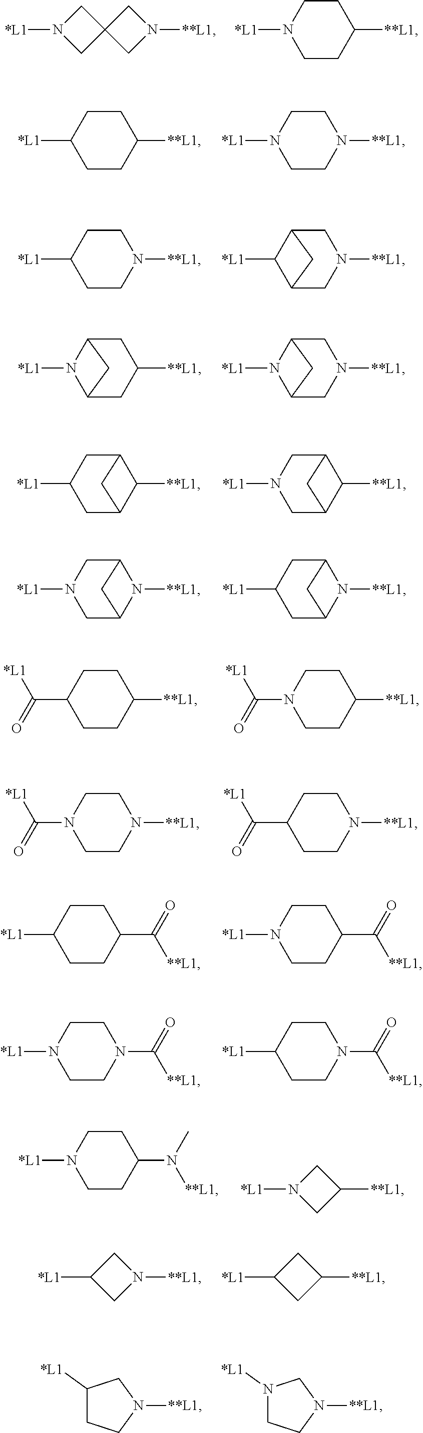

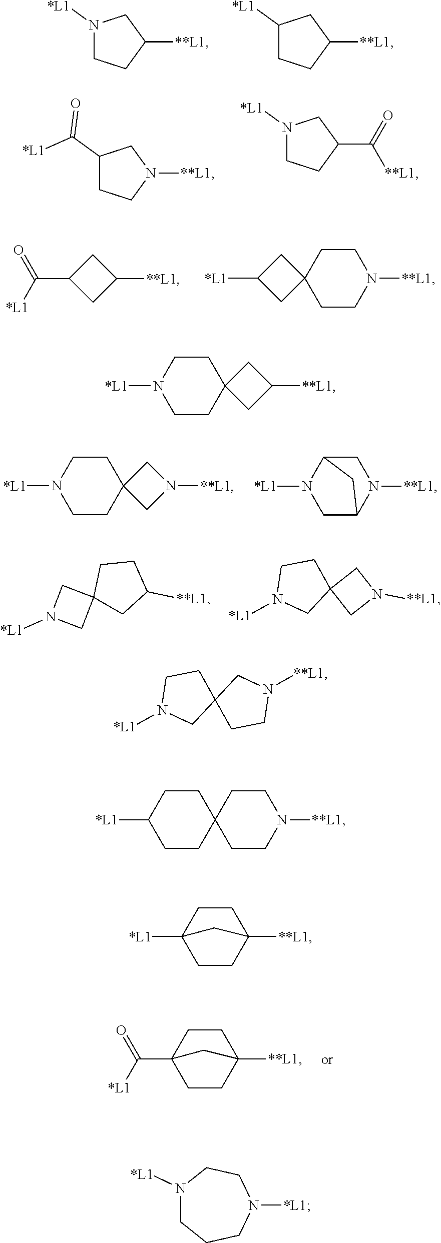

wherein each of said C₁-C₈alkylene- (preferably —CH₂—, —C₂H₄—, —C₃H₆—), *^{L1}—C(O)—C₁-C₈alkylene-*^{L1} (preferably *^{L1}—C(O)—CH₂—**^{L1}, *^{L1}—C(O)—C₂H₄—**^{L1}, *^{L1}—C(O)—C₃H₆—**^{L1}), *^{L1}—C₁-C₈alkylene-C(O)—**_{L1} (preferably *^{L1}—CH₂—C(O)—**^{L1}, *^{L1}—C₂H₄—C(O)—**^{L1}, *^{L1}—C₃H₆—C(O)—**^{L1}), —N(CH₃)—, —NH—,

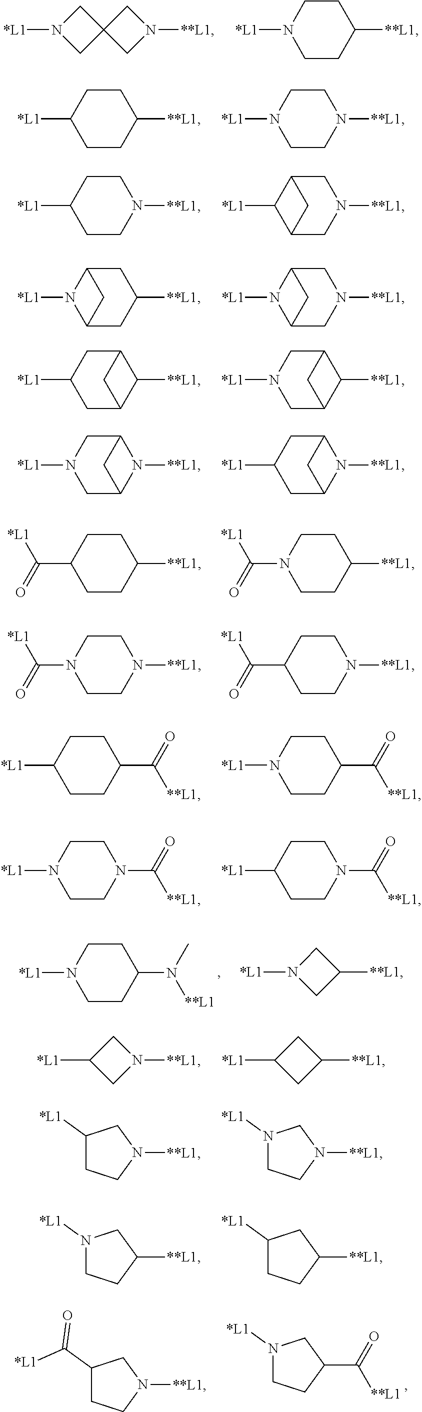

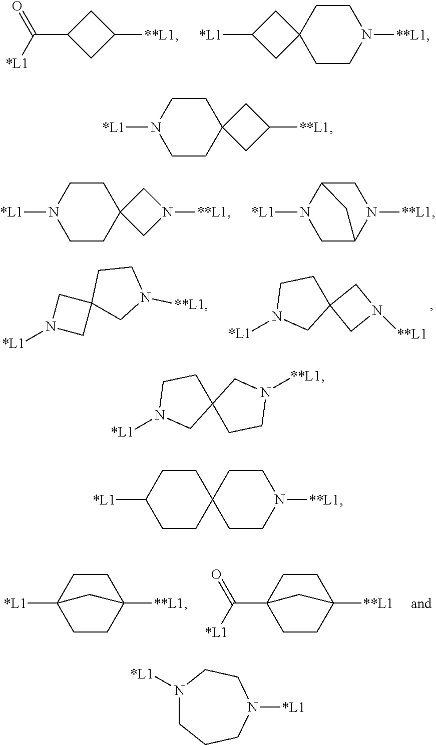

Aspect 21. The compound of any one of Aspects 1-20, wherein L¹ is selected from a single bond, —C₁-C₈alkylene- preferably —CH₂—, —C₂H₄—, —C₃H₆—), —CO—, —O—, —NH—,

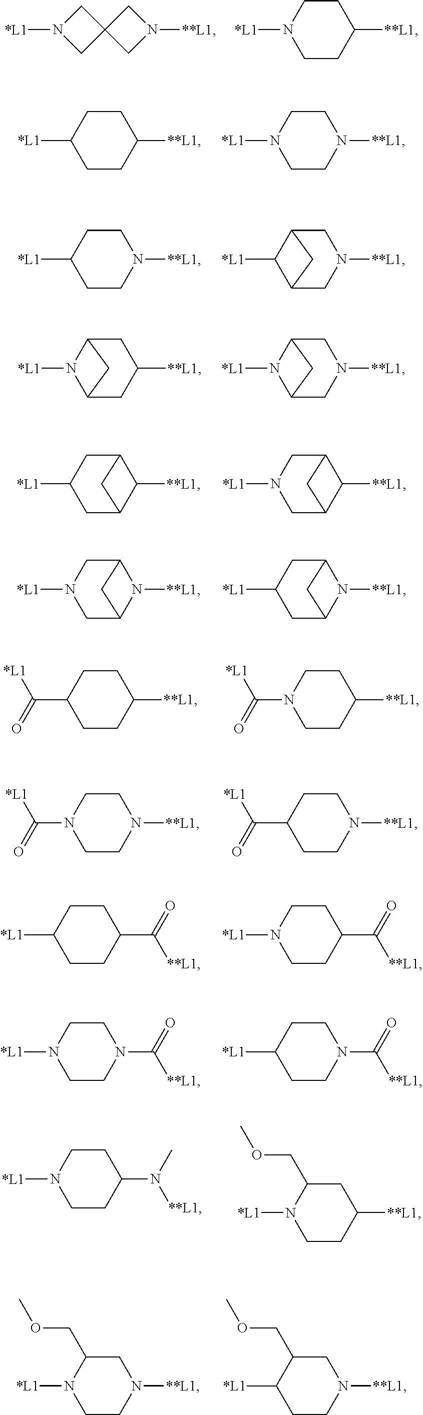

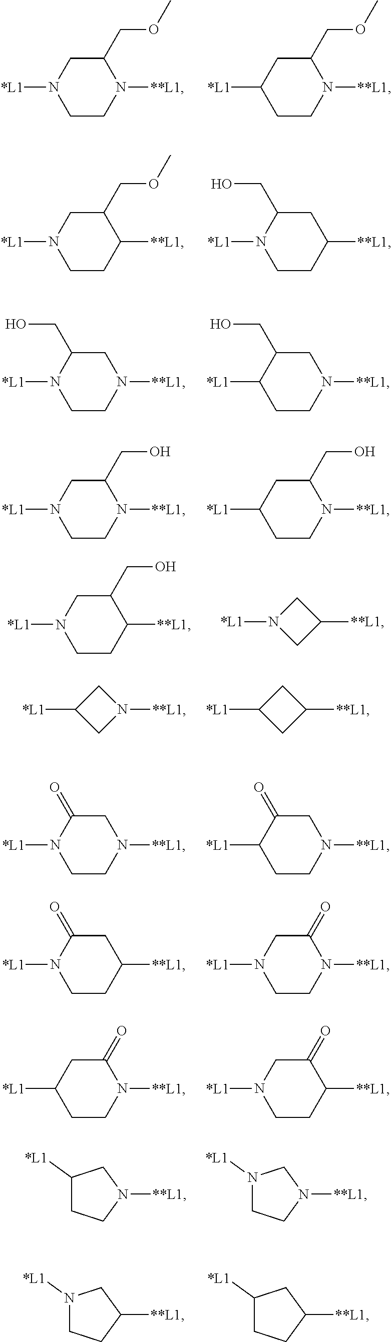

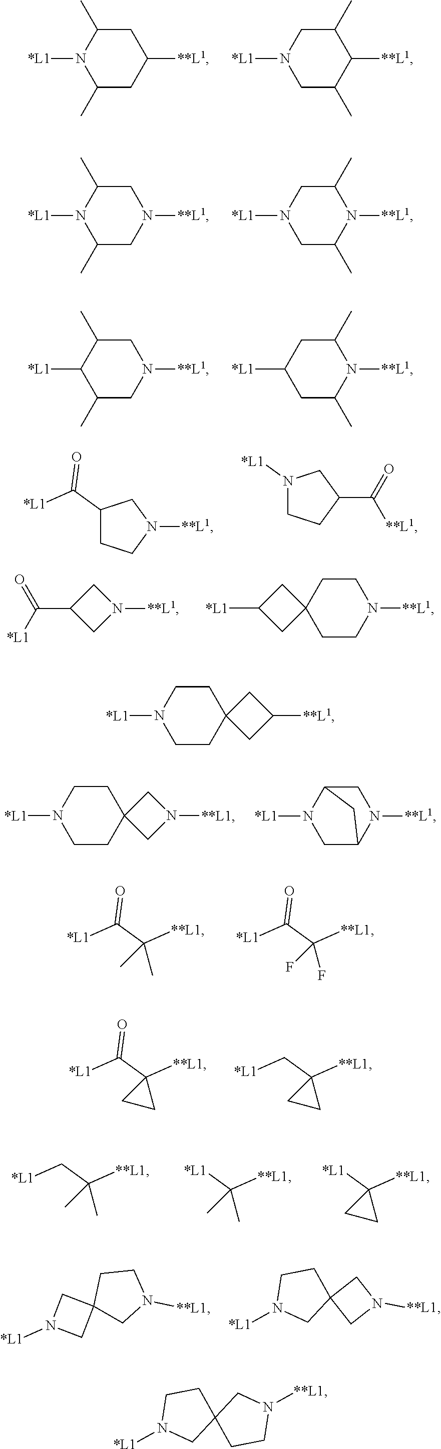

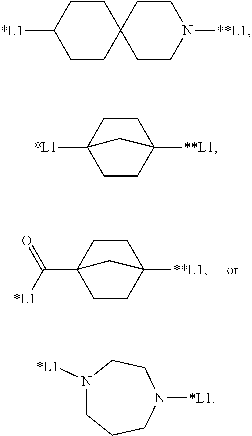

Aspect 22. The compound of any one of Aspects 1-21, wherein X¹ and X² are each independently selected from —CR^{a} or N;

R^{a} is selected from hydrogen, —F, —Cl, —Br, —I, CN, methyl, ethyl, methoxy, ethoxy, cyclopropyl, each of said methyl, ethyl, methoxy, ethoxy, cyclopropyl, is optionally substituted with at least one substituent —F, —Cl, —Br, —I, hydroxy, methyl, ethyl, (preferably, X¹ and X² are each independently selected from CH, C(F), C(CH₃) or N);

m1=1 or 0;

R¹² is hydrogen, oxo (═O), methoxymethyl, hydroxymethyl, —CN or —CH₃.

Aspect 23. The compound of any one of Aspects 1-22, wherein m1 is 1; preferably,

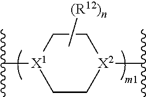

moiety is

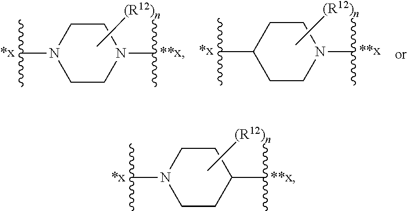

wherein *^{X} refers to the position attached to

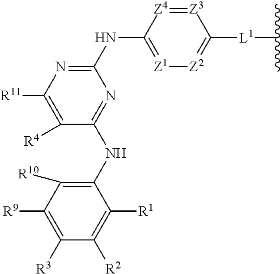

moiety, and **^{X} refers to the position attached to the

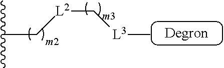

moiety.

Aspect 24. The compound of any one of Aspects 1-23, wherein m1 is 1; preferably,

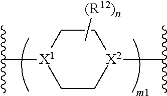

moiety is

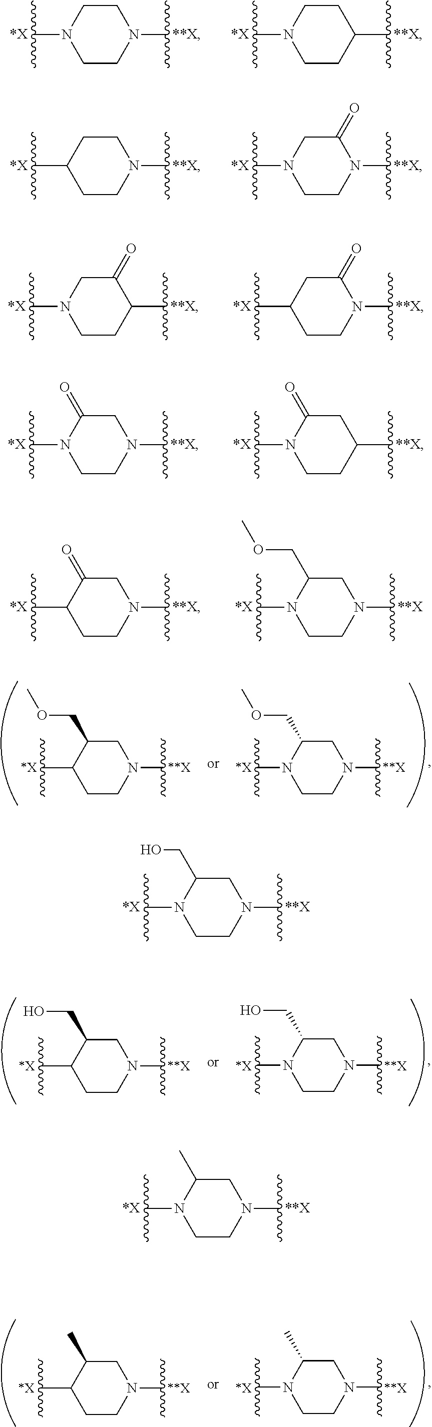

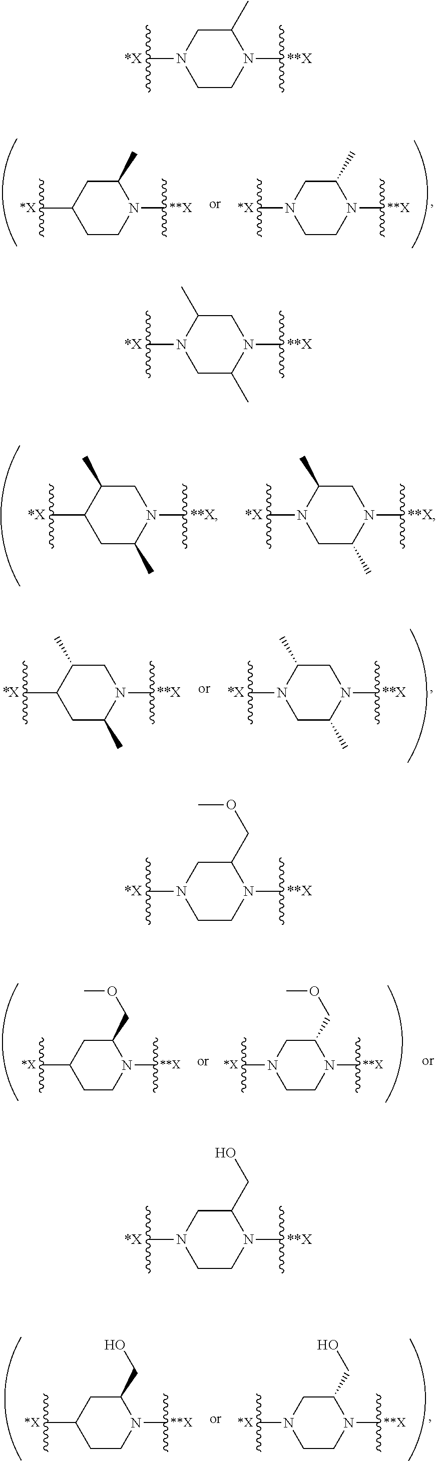

wherein *^{X} refers to the position attached to

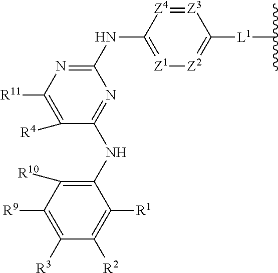

moiety, and **^{X} refers to the position attached to the

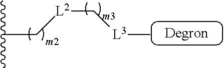

moiety.

Aspect 25. The compound of any one of Aspects 1-24, wherein m1 is 1,

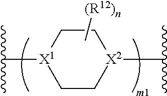

moiety is

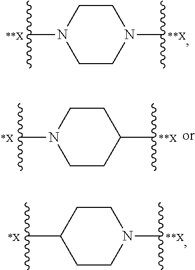

wherein *^{X} refers to the position attached to

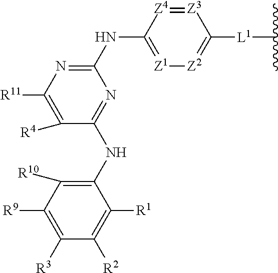

moiety, and **^{X} refers to the position attached to the

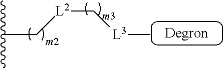

moiety.

Aspect 26. The compound of any one of Aspects 1-25, wherein L² is selected from a single bond, —C₁-C₈alkylene-(preferably —CH₂—, —C₂H₄—, —C₃H₆—), *^{L2}—CO—C₁-C₈alkylene-**^{L2} (preferably *^{L2}—CO—CH₂—**^{L2}, *^{L2}—CO—C₂H₄—**^{L2}, *^{L2}—CO—C₃H₆—**^{L2}), *^{L2}—C₁-C₈alkylene-CO—**^{L2} (preferably *^{L2}—CH₂—CO—**^{L2}, *^{L2}—C₂H₄—CO—**^{L2}, *^{L2}—C₃H₆—CO—**^{L2}), —CO—, —O—, —N(CH₃)—, —NH—,

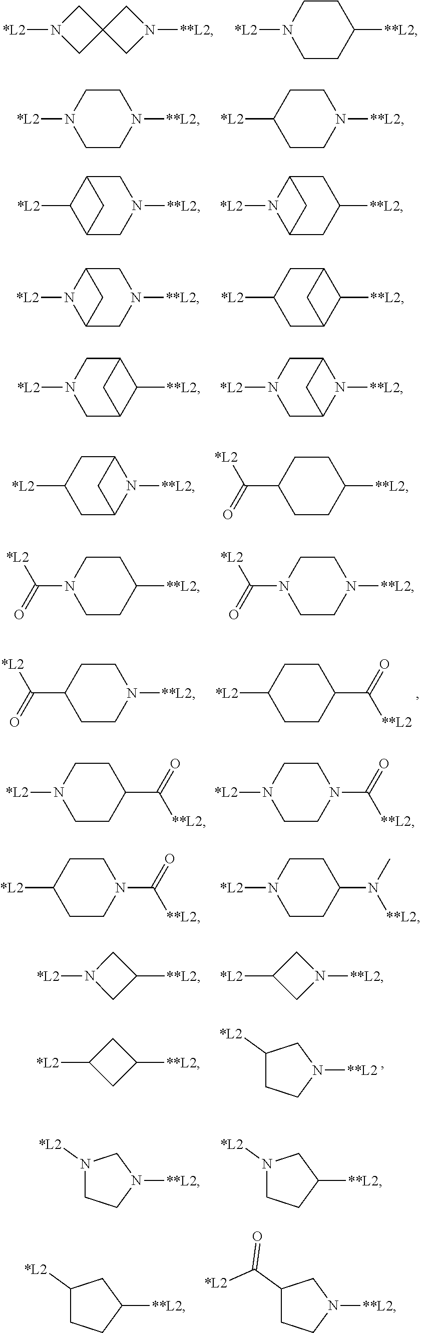

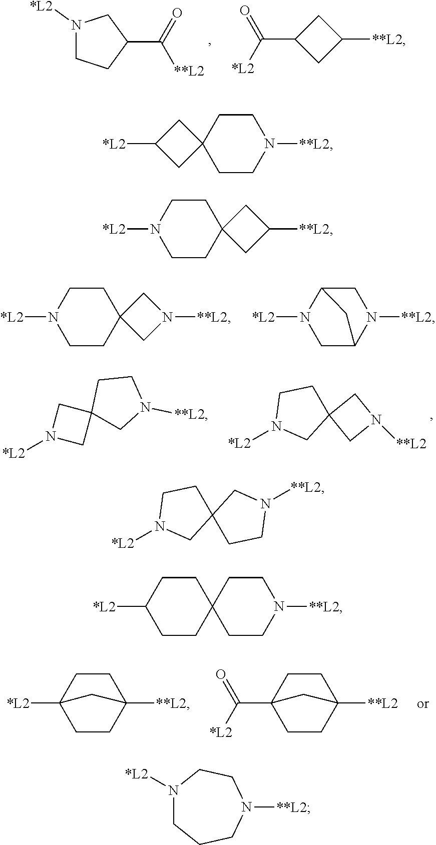

wherein each of said —C₁-C₈alkylene-(preferably —CH₂—, —C₂H₄—, —C₃H₆—), *^{L2}—CO—C₁-C₈alkylene-**^{L2} (preferably *^{L2}—CO—CH₂—**^{L2}, *^{L2}—CO—C₂H₄—**^{L2}, *^{L2}—CO—C₃H₆—**^{L2}), *^{L2}—C₁-C₈alkylene-CO—**^{L2} (preferably *^{L2}—CH₂—CO—**^{L2}, *^{L2}—C₂H₄—CO—**^{L2}, *^{L2}—C₃H₆—CO—**^{L2}), —N(CH₃)—, —NH—,

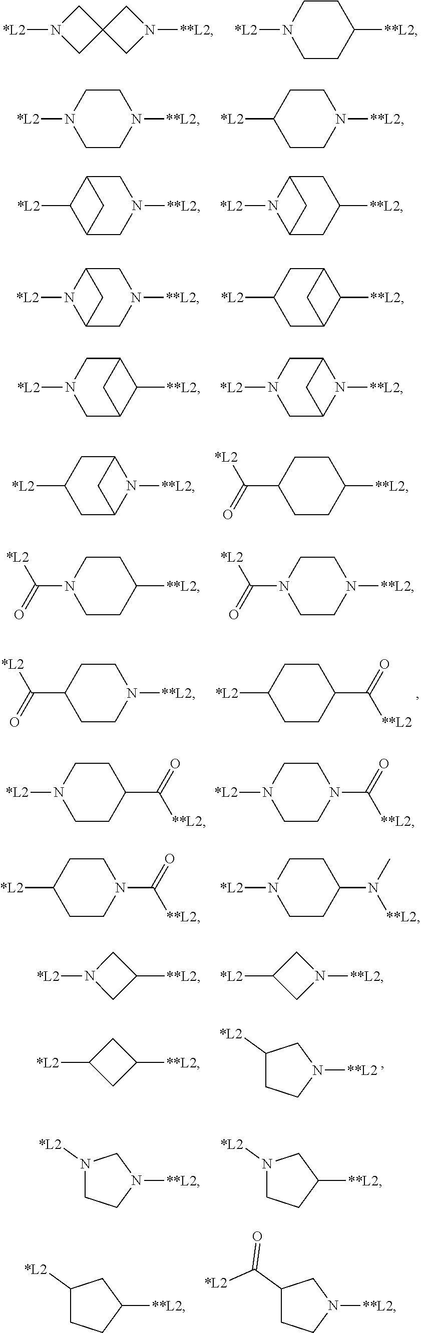

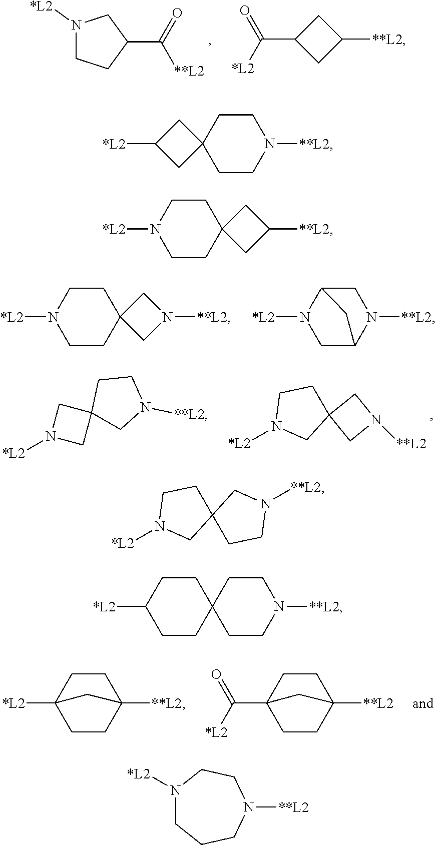

is optionally substituted with at least one R^{L2c};

each of said R^{L2}° is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or

two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

Aspect 27. The compound of any one of Aspects 1-26, wherein L² is selected from a single bond, —C₁-C₈alkylene-(preferably —CH₂—, —C₂H₄—, —C₃H₆—), —CO—, —O—, —N(CH₃)—, —NH—,

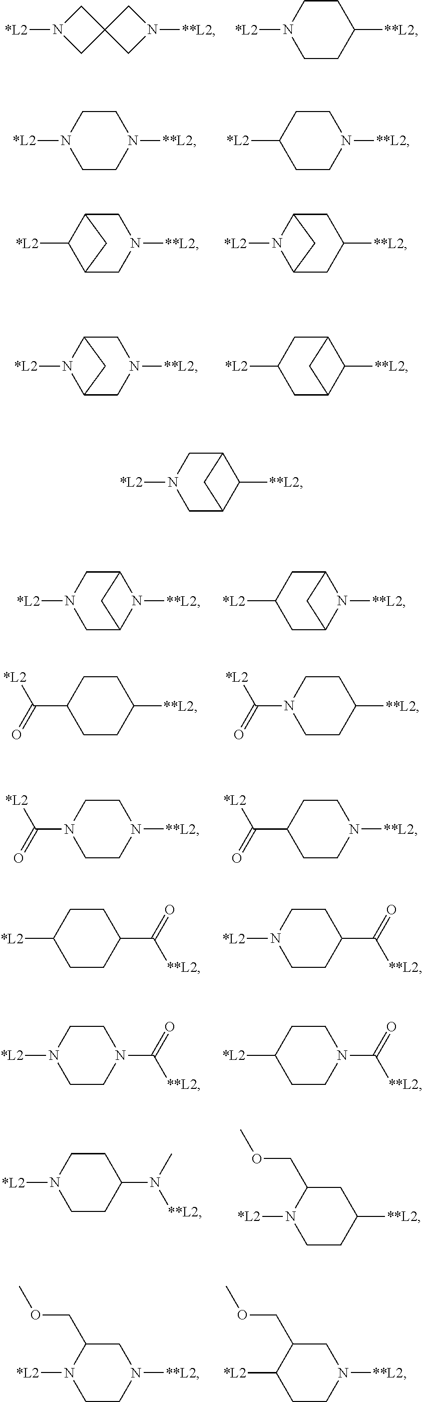

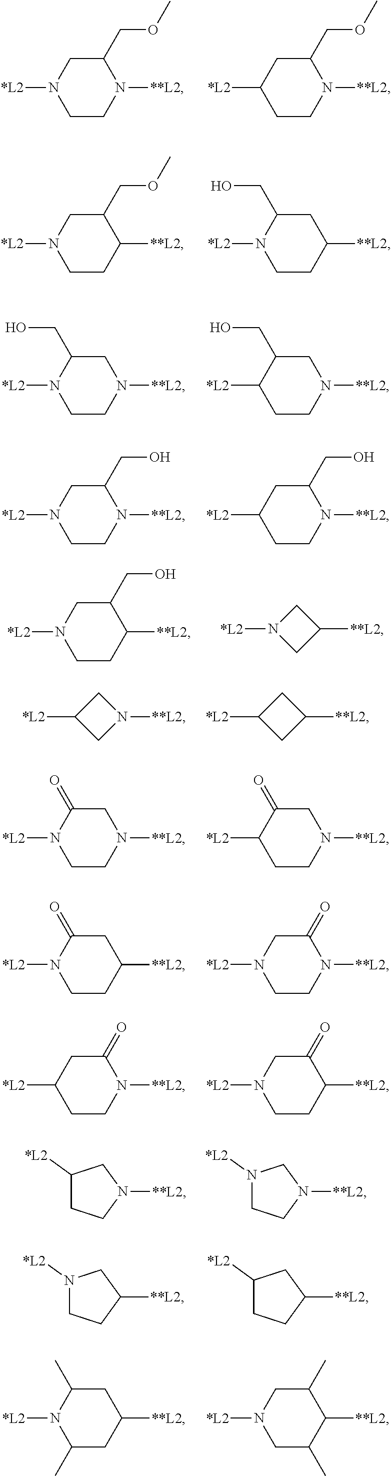

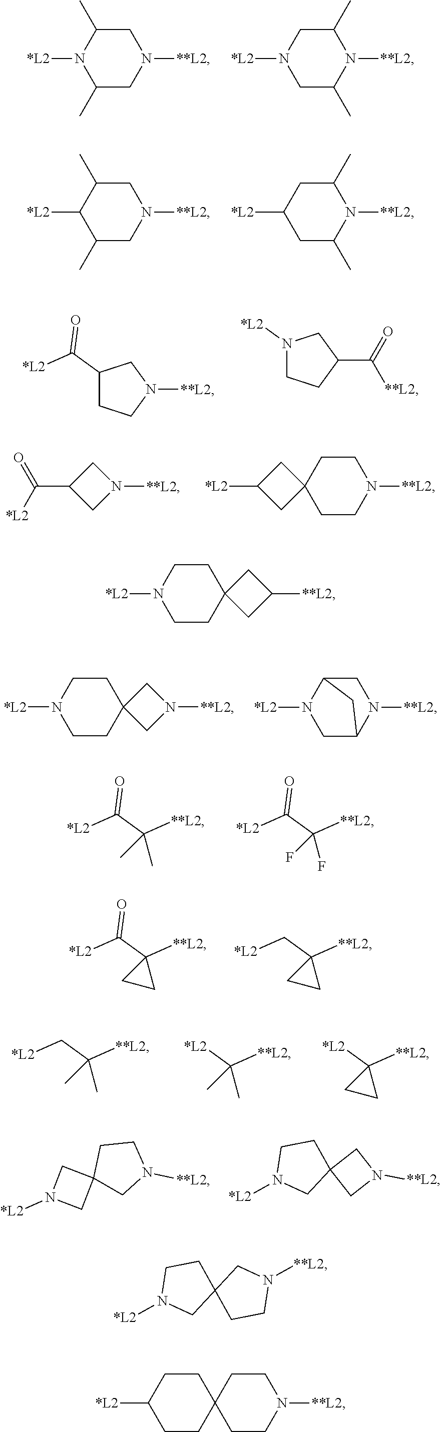

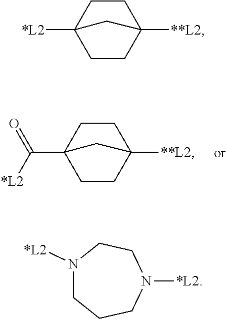

Aspect 28. The compound of any one of Aspects 1-27, wherein L³ is selected from single bond, —C₁-C₈alkylene-(preferably —CH₂—, —C₂H₄—, —C₃H₆—), *^{L3}—CO—C₁-C₈alkylene-**^{L3} (preferably *^{L3}—CO—CH₂—**^{L3}, *^{L3}—CO—C₂H₄—**^{L3}, *^{L3}—CO—C₃H₆—**^{L3}), *^{L3}—C₁-C₈alkylene-CO—**^{L3} (preferably *^{L3}—CH₂CO—**^{L3}, *^{L3}—C₂H₄—CO—**^{L3}, *^{L3}—C₃H₆—CO—**^{L3}), —CO—, —O—, —N(CH₃)—, —NH—,

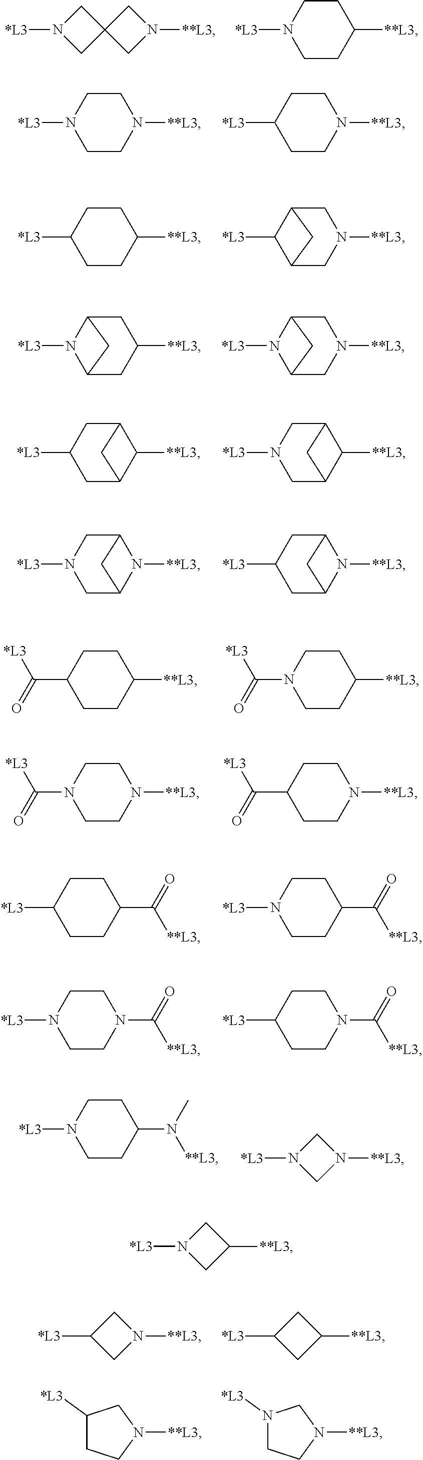

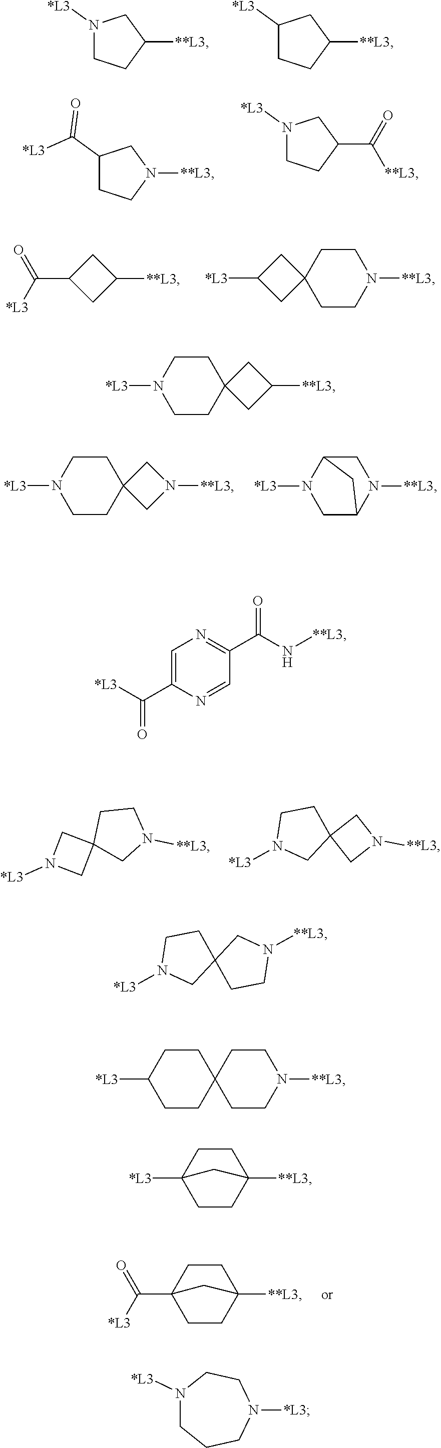

wherein each of said —C₁-C₈alkylene-(preferably —CH₂—, —C₂H₄—, —C₃H₆—), *^{L3}—CO—C₁-C₈alkylene-**^{L3} (preferably *^{L3}—CO—CH₂—**^{L3}, *^{L3}—CO—C₂H₄—**^{L3}, *^{L3}—CO—C₃H₆—**^{L3}), *^{L3}—C₁-C₈alkylene-CO—**^{L3} (preferably *^{L3}—CH₂—CO—**^{L3}, *^{L3}—C₂H₄—CO**^{L3}, *^{L3} C₃H₆—CO—**^{L3}—, —NH—,

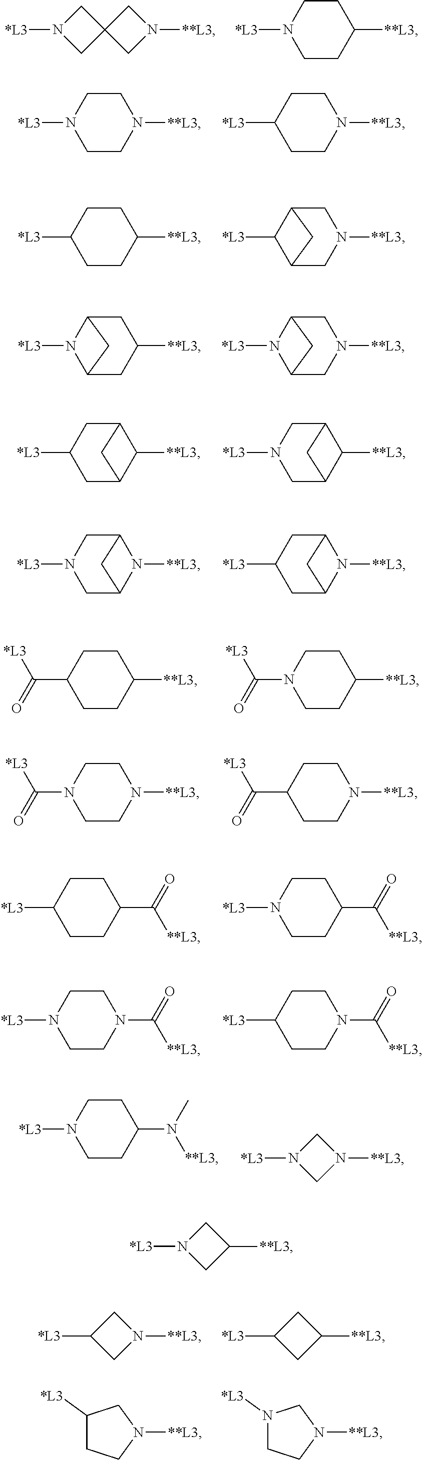

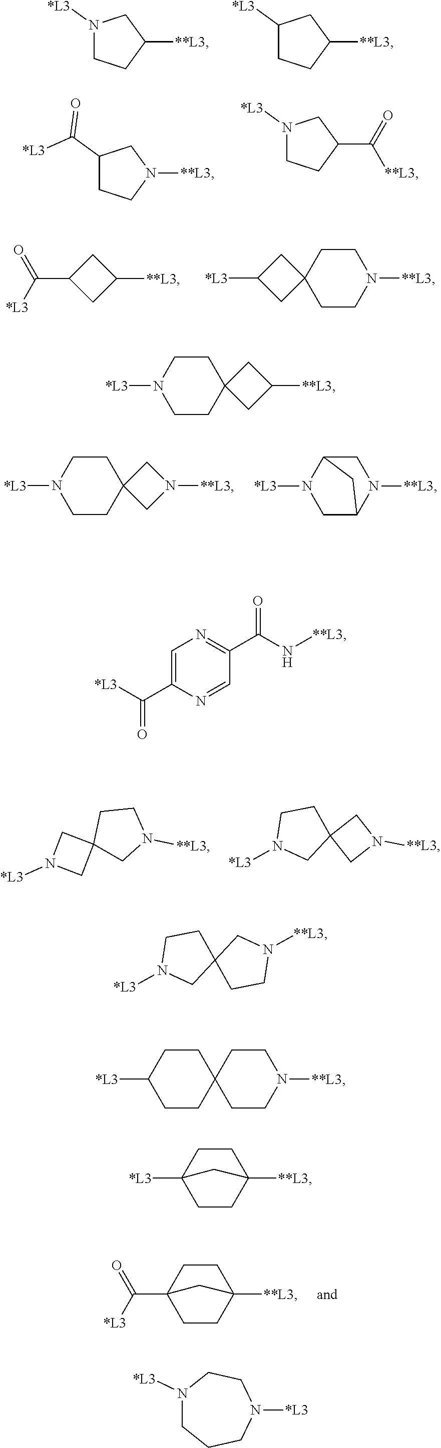

is optionally substituted with at least one R^{L3c};

each of said R^{L3e} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or

two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

Aspect 29. The compound of any one of Aspects 1-28, wherein L³ is selected from a single bond, —C₁-C₈alkylene-(preferably —CH₂—, —C₂H₄—, —C₃H₆—), —CO—, —O—, —N(CH₃)—, —NH—,

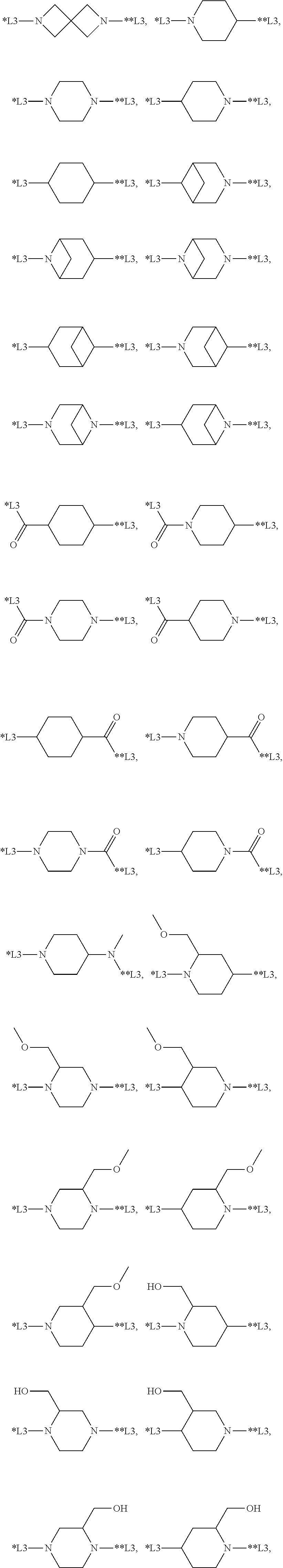

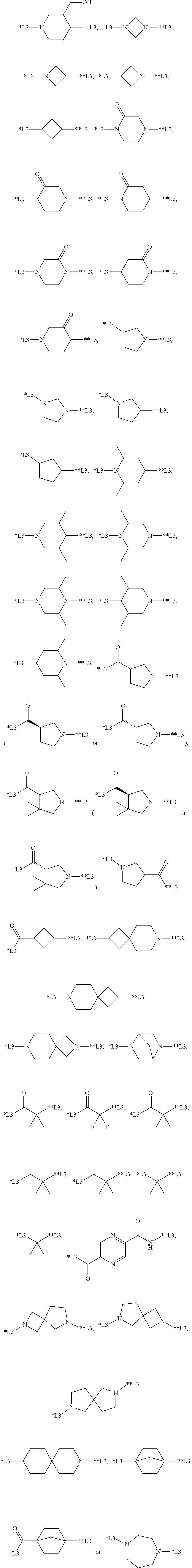

Aspect 30. The compound of any one of Aspects 1-29, wherein L² is a single bond, L³ is a single bond, or L² and L3 are both single bond.

Aspect 31. The compound of any one of Aspects 1_-30, wherein

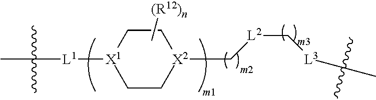

is selected from

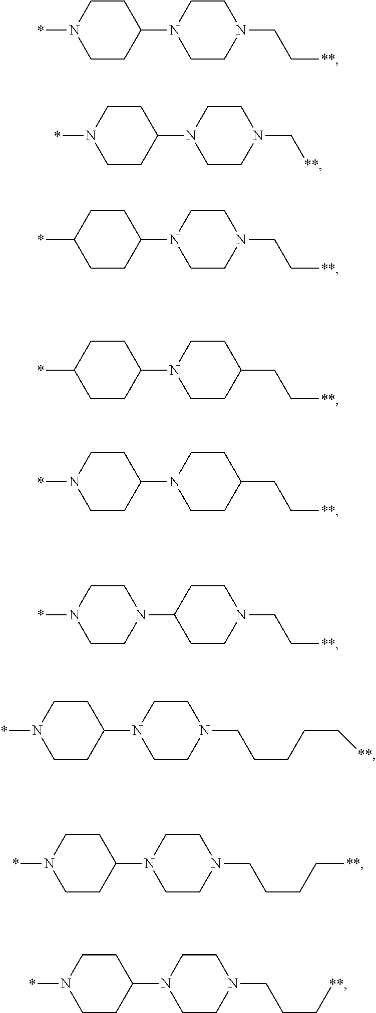

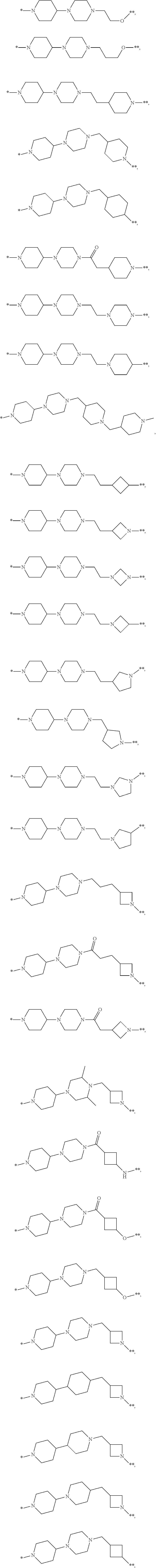

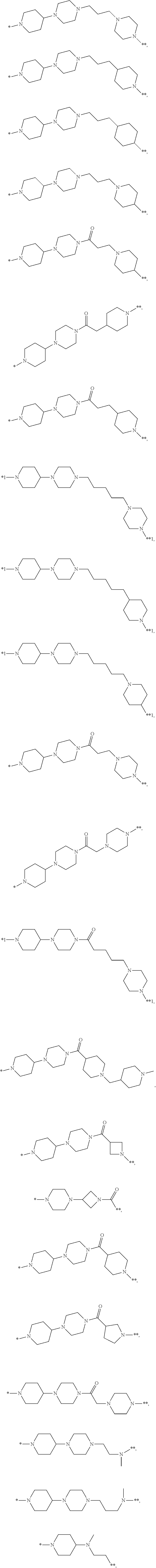

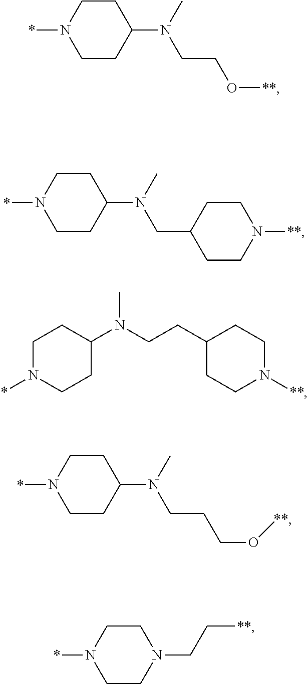

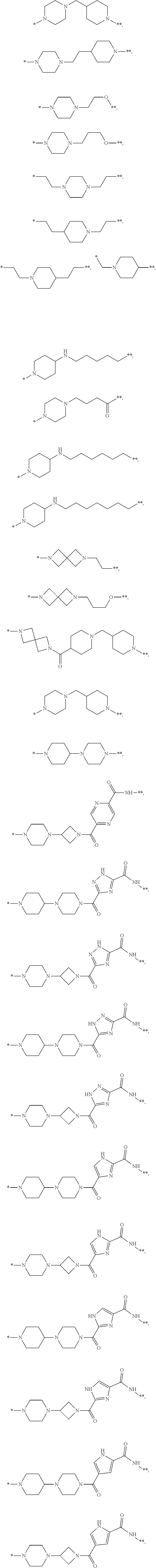

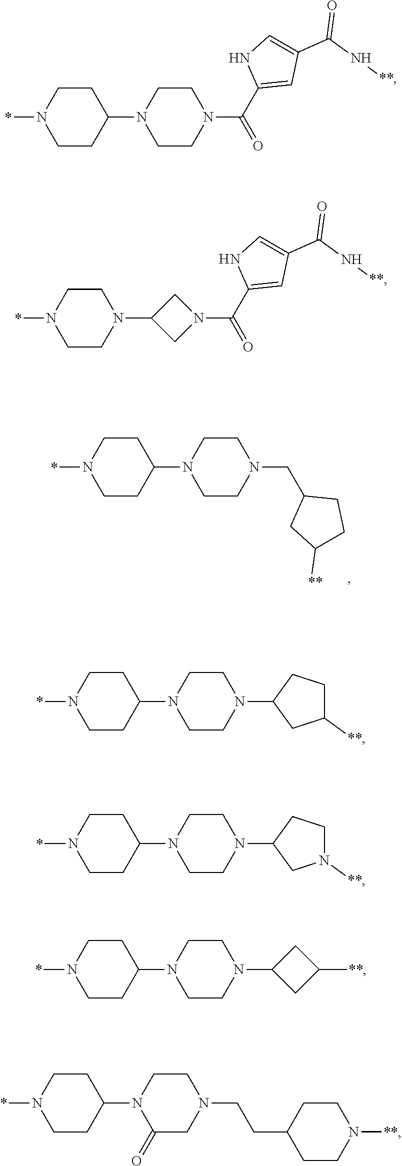

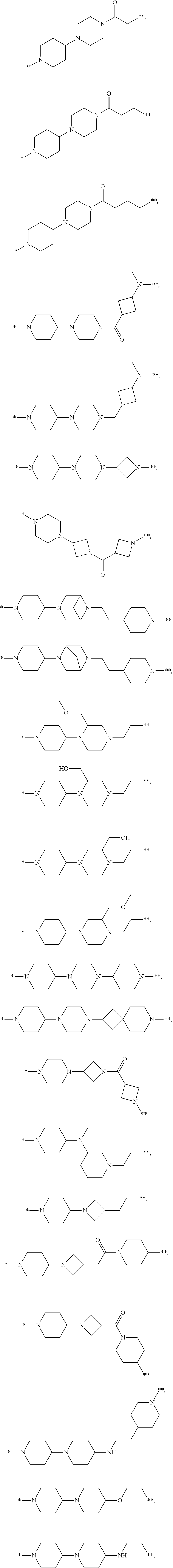

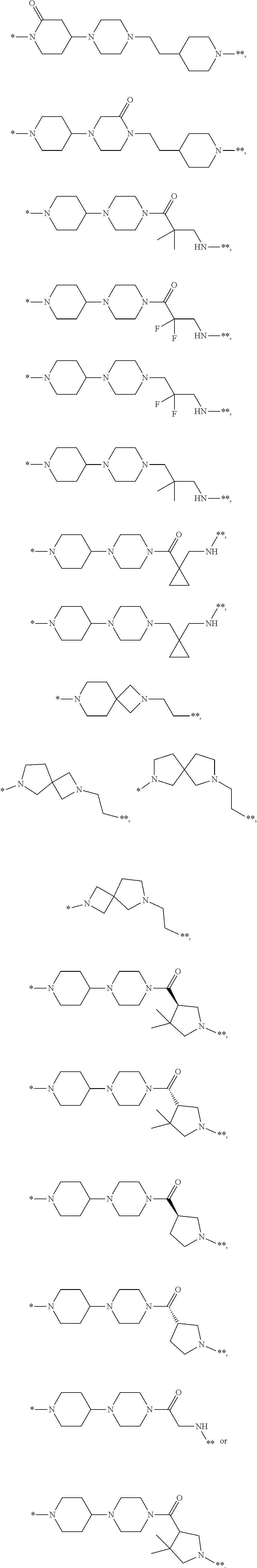

Aspect 32. The compound of any one of Aspects 1-30, wherein R¹³, R¹⁴, R¹⁵, R¹⁶ and R¹⁷ are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl; said each methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, methyl, ethyl, propyl, butyl, pentyl, hexyl, heptyl, octyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 33. The compound of any one of Aspects 1-32, wherein at each occurrence, R^{a} and R^{b} are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or

Ra and R^{b} together with the carbon atoms to which they are attached, form a 3, 4, 5, 6, 7 or 8-membered ring, said ring comprising 0-3 heteroatoms independently selected from nitrogen, oxygen or sulfur; said ring is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₂aryl or 5- to 12-membered heteroaryl.

Aspect 34. The compound of any one of Aspects 1-33, wherein

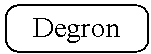

is selected from

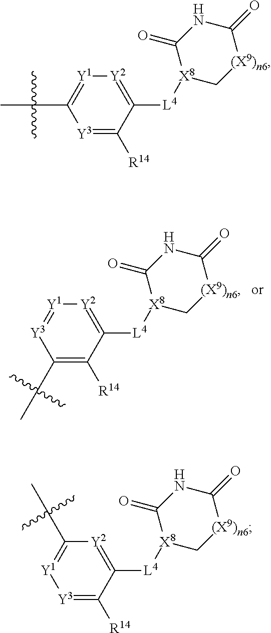

R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen or —C₁-C₈alkyl; preferably R¹⁴ is independently selected from H, F, Cl, Br, I, CH₃, —OCH₃, CH₂F, CN, CHF₂, or CF₃;

X⁸ is independently selected from CH, CD, C(CH₃), C(C₂H₅), C(C₃H₇), C(F) or N;

L⁴ is independently selected from a single bond, —O—, —NH—, —CH₂—, —CHF—, or —CF₂—;

Y¹, Y², and Y³ are each independently selected from CR^{a} or N;

X⁹ is CH₂;

R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen, hydroxy, —C₁-C₈alkyl, or —C₁-C₈alkoxy; and

n6 is independently 0, 1 or 2.

Aspect 35. The compound of any one of Aspects 1-34, wherein

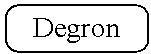

is selected from

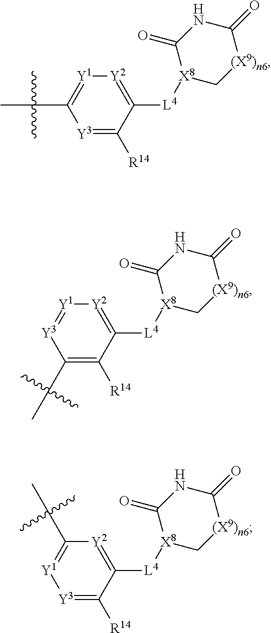

R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, CH₂F, CN, CHF₂, or CF₃; X⁸ is independently selected from CH, CD, C(CH₃), C(C₂H₅), C(C₃H₇), C(F) or N;

L⁴ is a single bond;

Y¹, Y², and Y³ are each independently selected from CR^{a} or N;

X⁹ is CH₂;

R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen; and n6 is 1.

Aspect 36. The compound of any one of Aspects 1-35, wherein

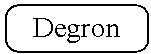

is selected from

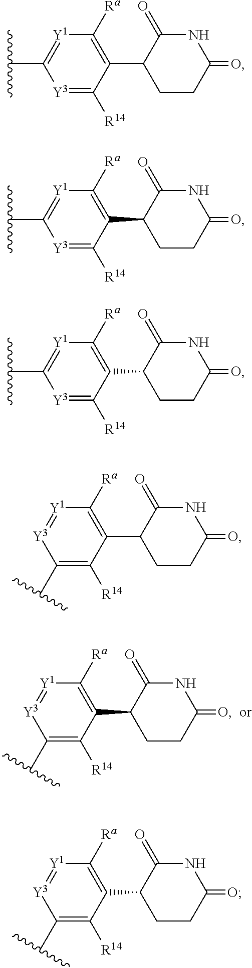

R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, CH₂F, CN, CHF₂, or CF₃;

Y¹ and Y³ are each independently selected from CH or N;

R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen.

Aspect 37. The compound of any one of Aspects 1-36, wherein

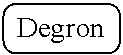

is selected from

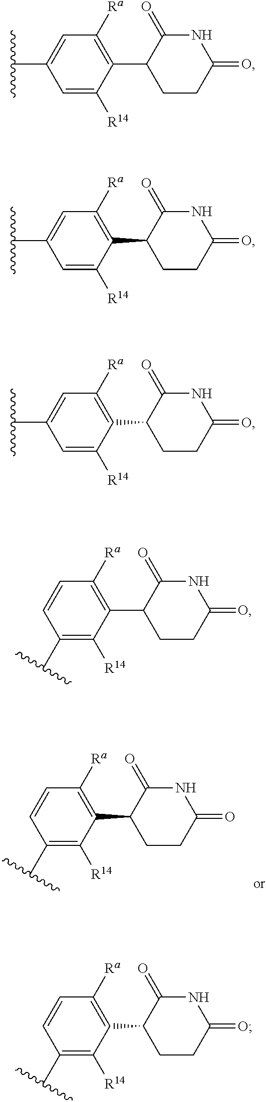

R¹⁴ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, or CN; said each methyl, ethyl, propyl, butyl, pentyl, hexyl, heptyl, octyl, methoxy, ethoxy, propoxy, butoxy, pentoxy, hexoxy, heptyloxy, octyloxy is optionally substituted by one or more F, Cl, Br, I; preferably R¹⁴ is independently selected from H, F, Cl, Br, I, CH₃, —OCH₃, CH₂F, CN, CHF₂, or CF₃;

R^{a} is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, each of said methyl, ethyl, propyl, butyl, pentyl, hexyl, heptyl, octyl, methoxy, ethoxy, propoxy, butoxy, pentoxy, hexoxy, heptyloxy, octyloxy is optionally substituted with at least one or more F, Cl, Br, I.

Aspect 38. The compound of any one of Aspects 1-37, wherein

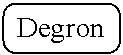

is selected from

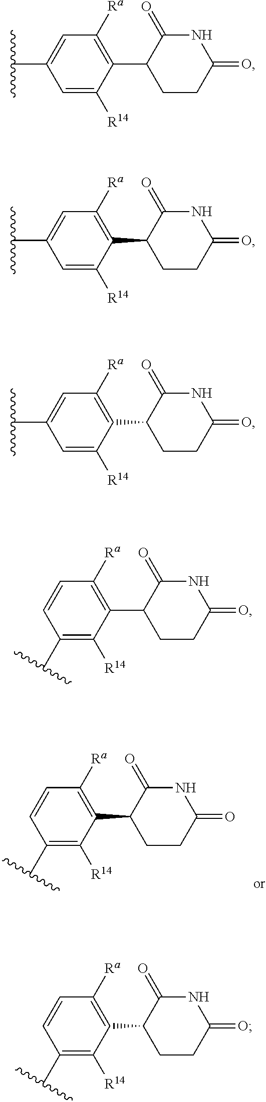

R¹⁴ is independently selected from F, Cl, Br, I, —CH₃, —OCH₃, CH₂F, CN, CHF₂, or CF₃;

R^{a} is each independently selected from F, Cl, Br, I, —CH₃, —OCH₃, CH₂F, CN, CHF₂, or CF₃.

Aspect 39. The compound of any one of Aspects 1-38, wherein

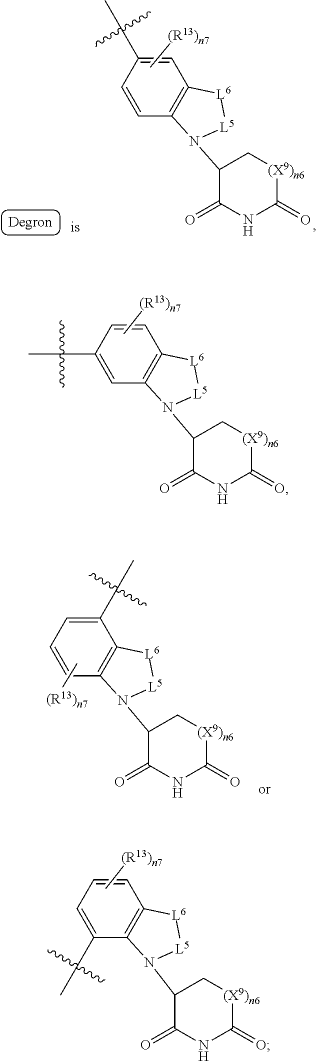

Wherein L⁵ and L⁶ are independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;

X⁹ is —CR^{a}R^{b}—;

R^{a} and R^{b} are each independently selected from hydrogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl and 5- to 12-membered heteroaryl is optionally substituted with at least one substituent halogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or

R^{a} and R^{b} together with the carbon atoms to which they are attached, form a 3-, 4-, 5-, 6-, 7-, 8-membered ring, said ring comprising 0-3 heteroatoms independently selected from nitrogen, oxygen or sulfur; said ring is optionally substituted with at least one substituent halogen, hydroxy, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;

each R¹³ is independently selected from hydrogen, halogen, CN, —C₁-C₈alkyl, or —C₁-C₈alkoxy;

n₆ is 0 or 1; and

n₇ is 0, 1 or 2.

Aspect 40. The compound of any one of Aspects 1-39, wherein

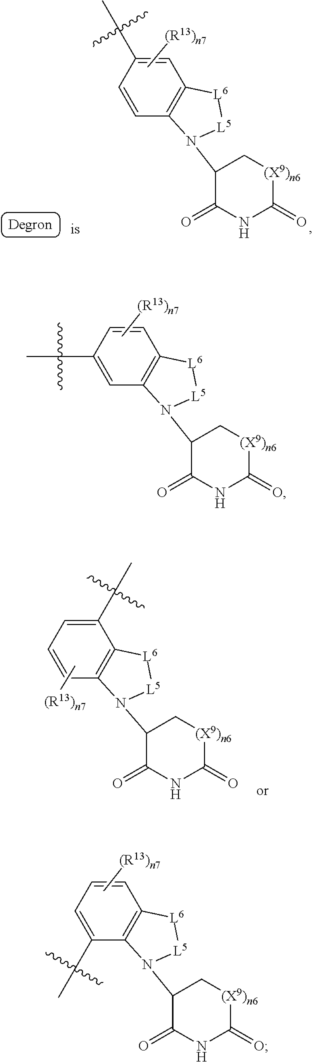

Wherein L⁵ and L⁶ is independently selected from a single bond,

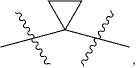 [SMILES: *C1(*)CC1]

—O—, —NH—, —NMe-, —N(CH₂CH₃)—, —CH₂—, —CHF—, —CF₂—, —C(CH₃)₂— or —CO— (preferably L⁵ is —CO— or —CH₂—, and L⁶ is

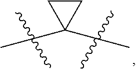 [SMILES: *C1(*)CC1]

—O—, —NH—, —NMe-, —N(CH₂CH₃)—, —CH₂—, —CHF—, —CF₂—, —C(CH₃)₂— or —CO—);

X⁹ is CH₂;

each R¹³ is independently selected from hydrogen, halogen, CN, —C₁-C₈alkyl, or —C₁-C₈alkoxy;

n₆ is 0 or 1; and

n₇ is 0, 1 or 2.

Aspect 41. The compound of any one of Aspects 1-40, wherein

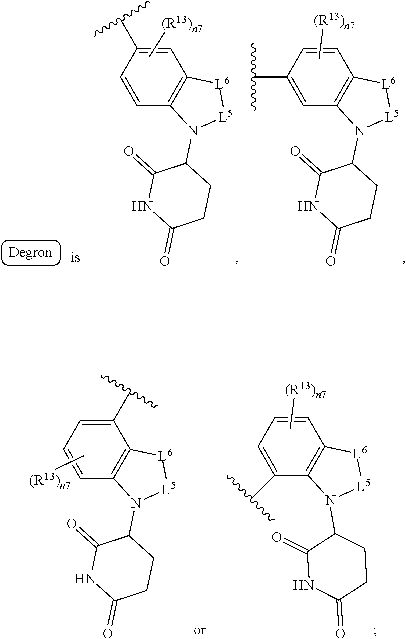

Wherein L⁵ and L⁶ are each independently selected from

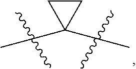 [SMILES: *C1(*)CC1]

—O—, —NH—, —NMe-, —N(CH₂CH₃)—,

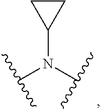 [SMILES: *N(*)C1CC1]

—CH₂—, —CHF—, —CF₂—, —C(CH₃)₂— or —CO—;

each R¹³ is independently selected from hydrogen, F, Cl, Br, I, CN, -Me, -Et, —C₃H₇, —C₄H₉, —OMe, —OEt, —OC₃H₇ or —OC₄H₉;

n₇ is 0, 1 or 2.

Aspect 42. The compound of any one of Aspects 1-41, wherein

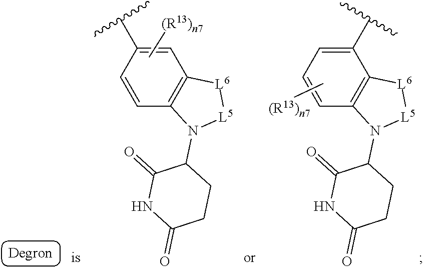

Wherein L⁶ is selected from

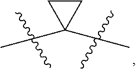 [SMILES: *C1(*)CC1]

—O—, —NMe-, —N(CH₂CH₃)—, —CH₂—, —CHF—, —CF₂— or —C(CH₃)₂—;

Wherein L⁵ is —CO—;

each R¹³ is independently selected from hydrogen, F, Cl, Br, I, CN, —C₁-C₈alkyl, or —C₁-C₈alkoxy;

n₇ is 0, 1 or 2.

Aspect 43. The compound of any one of Aspects 1-42, wherein

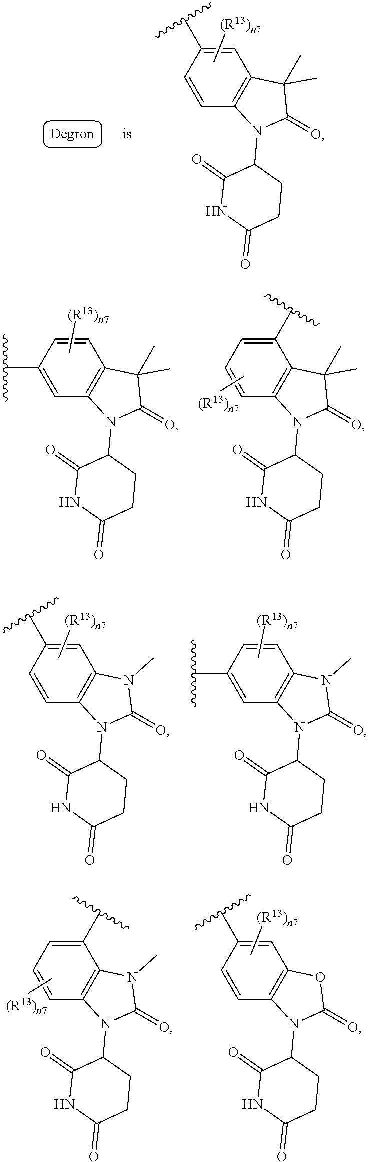

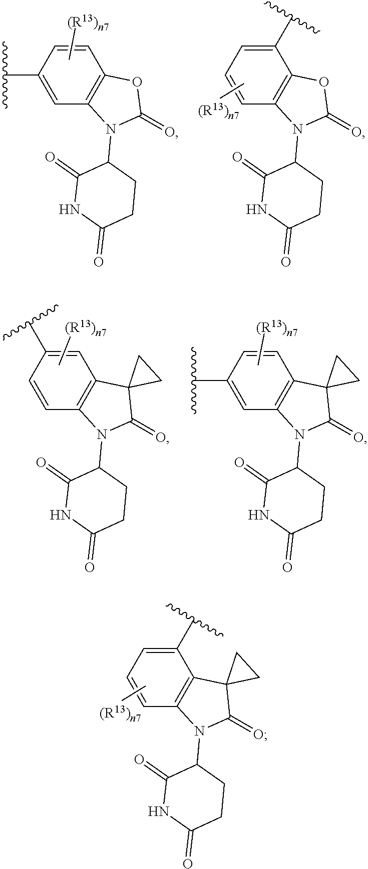

each R¹³ is independently selected from hydrogen, F, Cl, Br, I, CN, -Me, -Et, —C₃H₇, —C₄H₉, —OMe, —OEt, —OC₃H₇ or —OC₄H₉;

n₇ is 0, 1 or 2.

Aspect 44. The compound of any one of Aspects 1-43, wherein

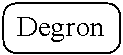

is selected from

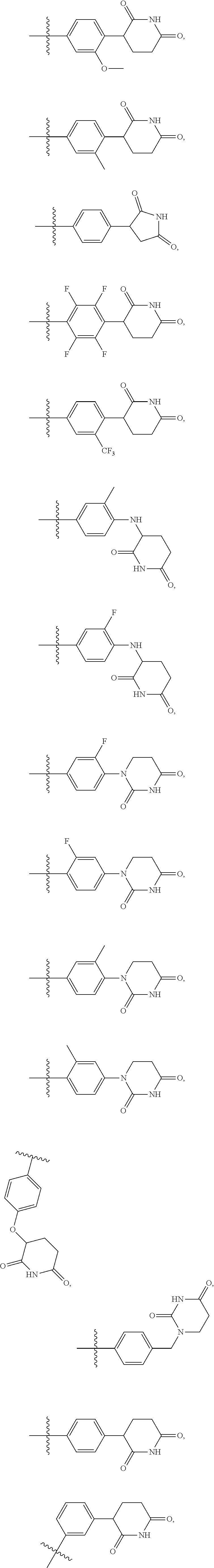

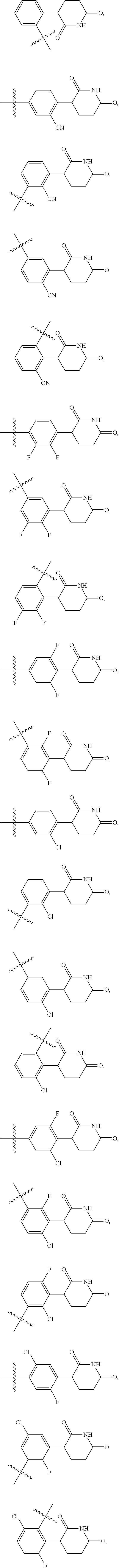

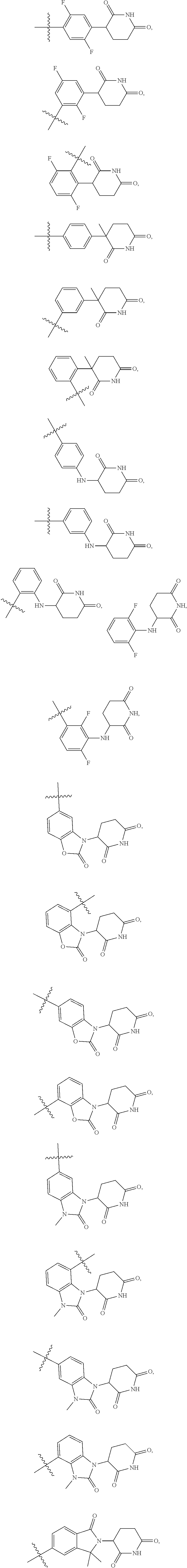

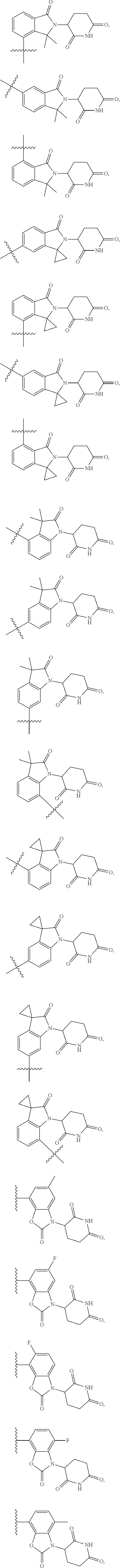

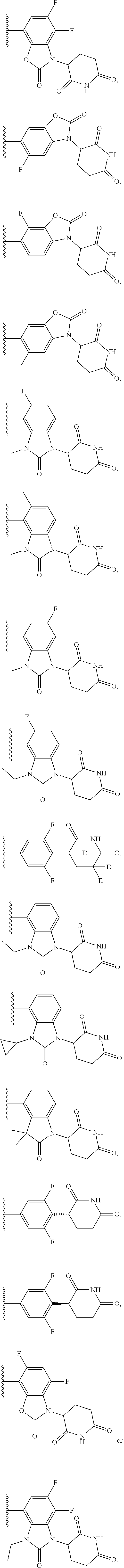

Aspect 45. The compound of any one of Aspects 1-44, wherein Z¹, Z², Z³ and Z⁴ are each independently —CR^{z};

R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{Zc}.

R^{Za} and R^{Zb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};

R^{Zc} and R^{Zd} are each independently —F, —Cl, —Br, —I, hydroxy, methyl, ethyl, propyl, butyl, pentyl, hexyl, heptyl, octyl, —C₁-C₈alkoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 46. The compound of any one of Aspects 1-45, wherein R^{z} is selected from H, —CH₃, —C₂H₅, F, —CH₂F, —CHF₄, —CF₃a —OCH₃, —OC₂H₅, —C₃H₇, —OCH₂F, —OCHF₂, —OCH₂CF₃, —OCF₃, —SCF₃, —CF₃, —CH(OH)CH₃,

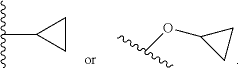

Aspect 47. The compound of any one of Aspects 1-46 selected from example 23, 318, 413, 459, 460, 462, 473, 483, 484, 485, 486, 487, 488, 489, 491, 492, 493, 499, 501, 502, 503, 504, 505, 541, 549, 551, 558, 559, 563, 566, 570, 571, 572, 576, 577, 584, 589, 643, 711, 725, 727, 728, 729, 730, 731, 732, 733, 736, 737, 738, 739, 740, 741, 742, 744, 745, 746, 747, 748, 749, 750, 751, 752, 753, 754, 755 or 756.

On the other aspect, the compound is selected from

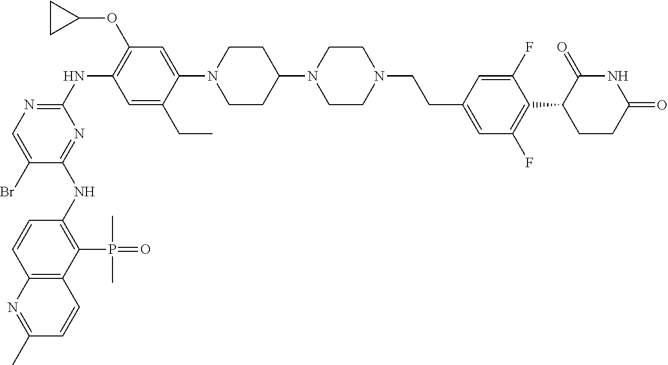 [SMILES: CCc1cc(Nc2ncc(Br)c(Nc3ccc4nc(C)ccc4c3P(C)(C)=O)n2)c(OC2CC2)cc1N1CCC(N2CCN(CCc3cc(F)c([C@H]4CCC(=O)NC4=O)c(F)c3)CC2)CC1]

Example 459
(R)-3-(4-(2-(4-(1-(4-((5-bromo-4-((5-
(dimethylphosphoryl)-2-methylquinolin-6-
yl)amino)pyrimidin-2-yl)amino)-5-
cyclopropoxy-2-ethylphenyl)piperidin-4-
yl)piperazin-1-yl)ethyl)-2,6-
difluorophenyl)piperidine-2,6-dione

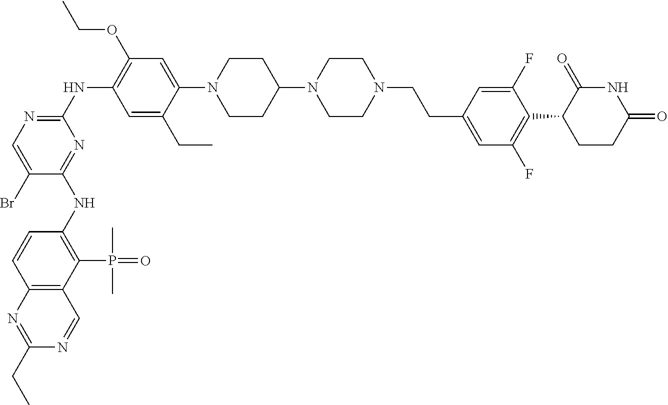 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ncc3c2P(C)(C)=O)n1]

Example 460
(R)-3-(4-(2-(4-(1-(4-((5-bromo-4-((5-
(dimethylphosphoryl)-2-ethylquinazolin-6-
yl)amino)pyrimidin-2-yl)amino)-5-ethoxy-2-
ethylphenyl)piperidin-4-yl)piperazin-1-yl)ethyl)-
2,6-difluorophenyl)piperidine-2,6-dione

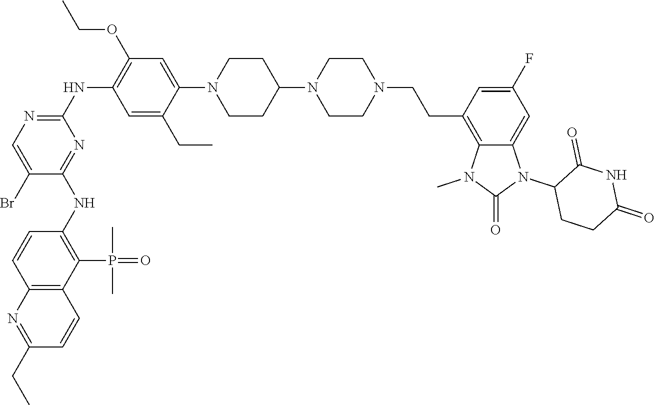 [SMILES: CCOc1cc(N2CCC(N3CCN(CCc4cc(F)cc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

Example 462
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
6-fluoro-3-methyl-2-oxo-2,3-dihydro-1H-
benzo[d]imidazol-1-yl)piperidine-2,6-dione

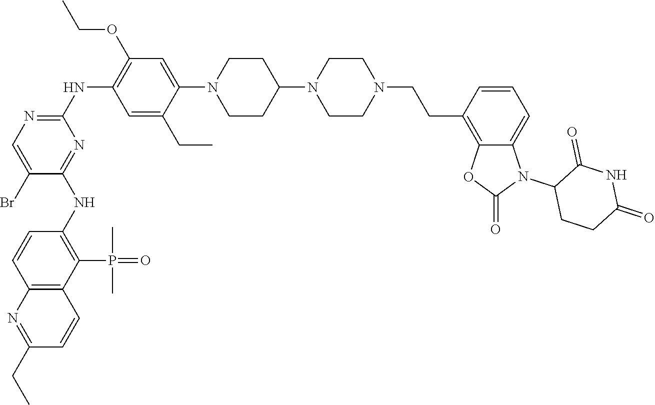 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

Example 473
3-(7-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
2-oxobenzo[d]oxazol-3(2H)-yl)piperidine-2,6-
dione

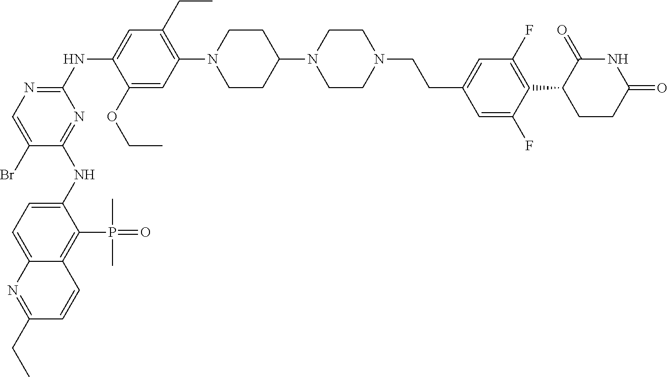 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ccc3c2P(C)(C)=O)n1]

Example 483
(R)-3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
2,6-difluorophenyl)piperidine-2,6-dione

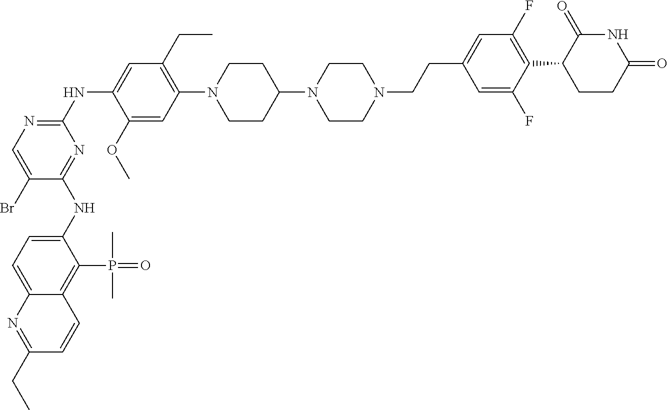 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Br)ccc2n1]

Example 484
(R)-3-(4-(2-(4-(1-(4-((5-bromo-4-((5-
(dimethylphosphory])-2-ethylquinolin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

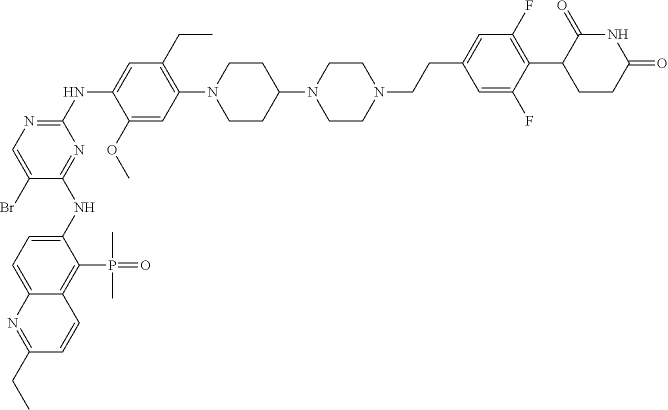 [SMILES: CCc1ccc2c(P(C)(C)=O)c(Nc3nc(Nc4cc(CC)c(N5CCC(N6CCN(CCc7cc(F)c(C8CCC(=O)NC8=O)c(F)c7)CC6)CC5)cc4OC)ncc3Br)ccc2n1]

Example 485
3-(4-(2-(4-(1-(4-((5-bromo-4-((5-
(dimethylphosphoryl)-2-ethylquinolin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

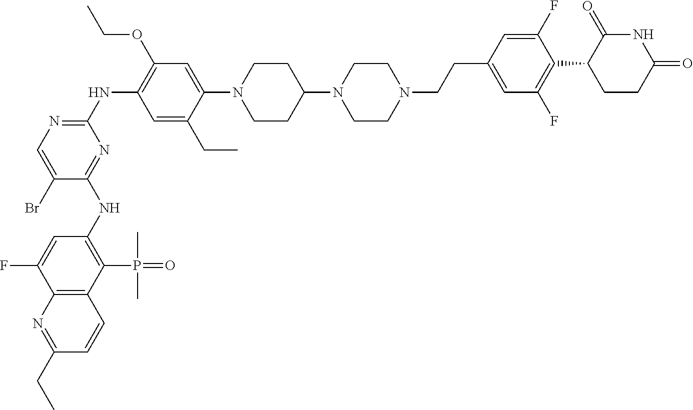 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3nc(CC)ccc3c2P(C)(C)=O)n1]

Example 486
(R)-3-(4-(2-(4-(1-(4-((5-bromo-4-((5-
(dimethylphosphoryl)-2-ethyl-8-fluoroquinolin-
6-yl)amino)pyrimidin-2-yl)amino)-5-ethoxy-2-
ethylphenyl)piperidin-4-yl)piperazin-1-yl)ethyl)-
2,6-difluorophenyl)piperidine-2,6-dione

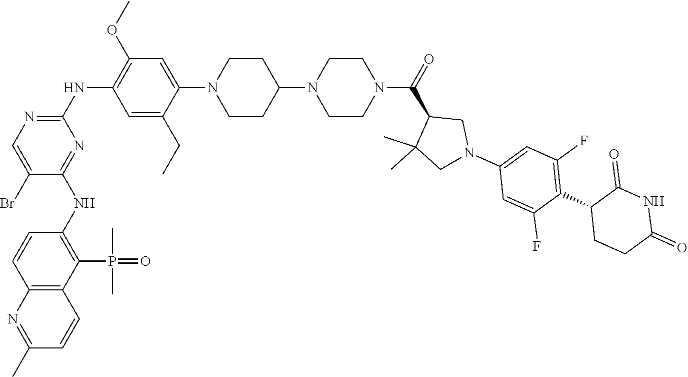 [SMILES: CCc1cc(Nc2ncc(Br)c(Nc3ccc4nc(C)ccc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

Example 487
(R)-3-(4-((R)-4-(4-(1-(4-((5-bromo-4-((5-
(dimethylphosphoryl)-2-methylquinolin-6-
yl)amino)pyrimidin-2-yl)amino)-2-ethyl-5-
methoxyphenyl)piperidin-4-yl)piperazine-1-
carbonyl)-3,3-dimethylpyrrolidin-1-yl)-2,6-
difluorophenyl)piperidine-2,6-dione

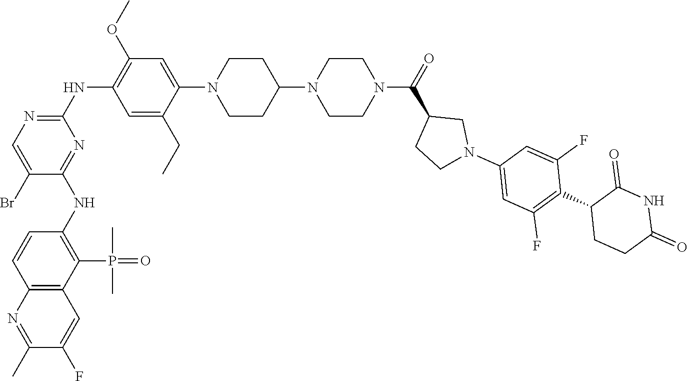 [SMILES: CCc1cc(Nc2ncc(Br)c(Nc3ccc4nc(C)c(F)cc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

Example 488
(R)-3-(4-((R)-3-(4-(1-(4-((5-bromo-4-((5-
(dimethylphosphoryl)-3-fluoro-2-
methylquinolin-6-yl)amino)pyrimidin-2-
yl)amino)-2-ethyl-5-methoxyphenyl)piperidin-4-
yl)piperazine-1-carbonyl)pyrrolidin-1-yl)-2,6-
difluorophenyl)piperidine-2,6-dione

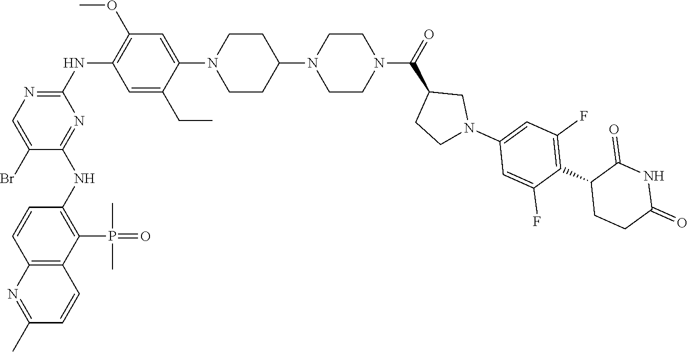 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

Example 489
(R)-3-(4-((R)-3-(4-(1-(4-((5-bromo-4-((5-
(dimethylphosphoryl)-2-ethylquinolin-6-
yl)amino)pyrimidin-2-yl)amino)-2-ethyl-5-
methoxyphenyl)piperidin-4-yl)piperazine-1-
carbonyl)pyrrolidin-1-yl)-2,6-
difluorophenyl)piperidine-2,6-dione

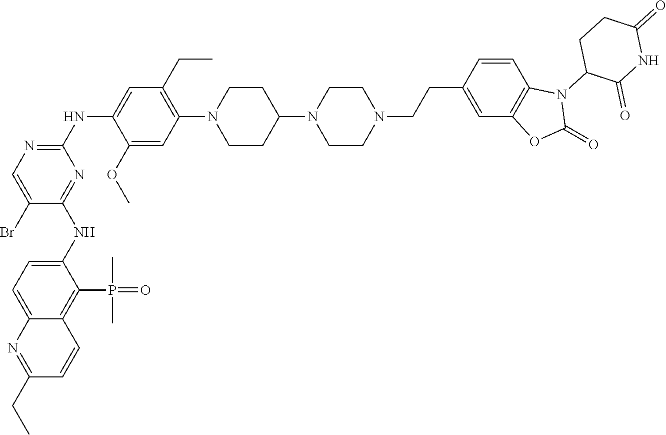 [SMILES: CCc1ccc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1]

Example 491
3-(6-(2-(4-(1-(4-((5-bromo-4-((5-
(dimethylphosphoryl)-2-ethylquinolin-6-
yl)amino)pyrimidin-2-yl)amino)-2-ethyl-5-
methoxyphenyl)piperidin-4-yl)piperazin-1-
yl)ethyl)-2-oxobenzo[d]oxazol-3(2H)-
yl)piperidine-2,6-dione

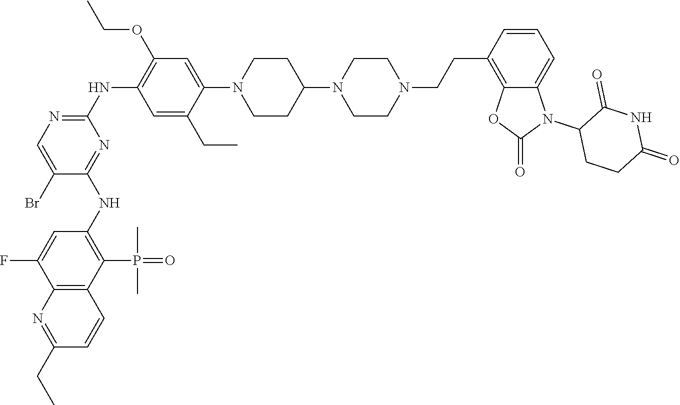 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

Example 493
3-(7-(2-(4-(1-(4-((5-bromo-4-((5-
(dimethylphosphoryl)-2-ethyl-8-fluoroquinolin-
6-yl)amino)pyrimidin-2-yl)amino)-5-ethoxy-2-
ethylphenyl)piperidin-4-yl)piperazin-1-yl)ethyl)-
2-oxobenzo[d]oxazol-3(2H)-yl)piperidine-2,6-
dione

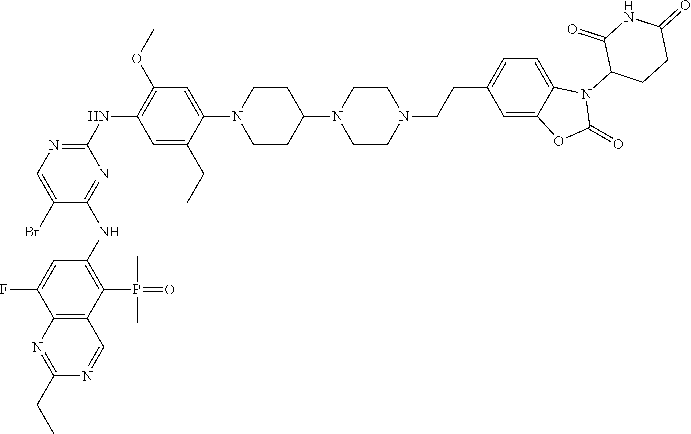 [SMILES: CCc1ncc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)cc(F)c2n1]

Example 499
3-(6-(2-(4-(1-(4-((5-bromo-4-((5-
(dimethylphosphoryl)-2-ethylquinazolin-6-
yl)amino)pyrimidin-2-yl)amino)-2-ethyl-5-
methoxyphenyl)piperidin-4-yl)piperazin-1-
yl)ethyl)-2-oxobenzo[d]oxazol-3(2H)-
yl)piperidine-2,6-dione

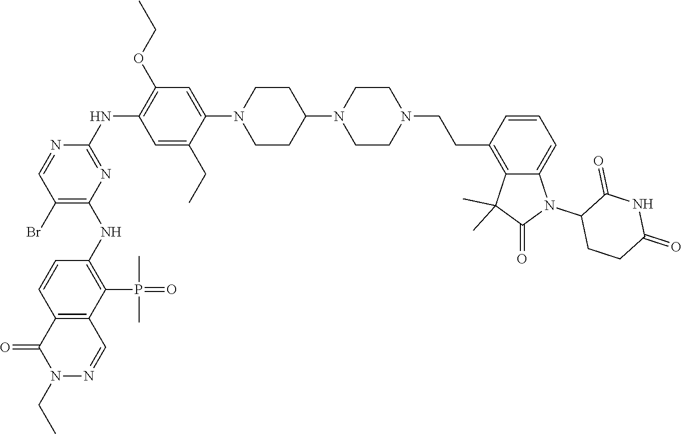 [SMILES: CCOc1cc(N2CCC(N3CCN(CCc4cccc5c4C(C)(C)C(=O)N5C4CCC(=O)NC4=O)CC3)CC2)c(CC)cc1Nc1ncc(Br)c(Nc2ccc3c(=O)n(CC)ncc3c2P(C)(C)=O)n1]

Example 501
3-(4-(2-(4-(1-(4-((5-bromo-4-((5-
(dimethylphosphoryl)-2-ethyl-1-oxo-1,2-
dihydrophthalazin-6-yl)amino)pyrimidin-2-
yl)amino)-5-ethoxy-2-ethylphenyl)piperidin-4-
yl)piperazin-1-yl)ethyl)-3,3-dimethyl-2-
oxoindolin-1-yl)piperidine-2,6-dione

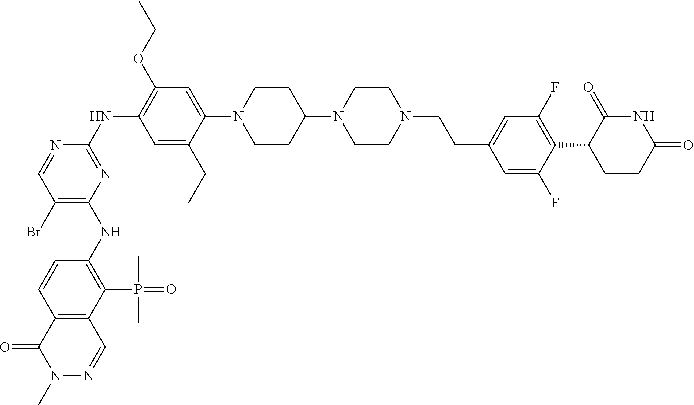 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3c(=O)n(C)ncc3c2P(C)(C)=O)n1]

Example 502
(R)-3-(4-(2-(4-(1-(4-((5-bromo-4-((5-
(dimethylphosphoryl)-2-methyl-1-oxo-1,2-
dihydrophthalazin-6-yl)amino)pyrimidin-2-
yl)amino)-5-ethoxy-2-ethylphenyl)piperidin-4-
yl)piperazin-1-yl)ethyl)-2,6-
difluorophenyl)piperidine-2,6-dione

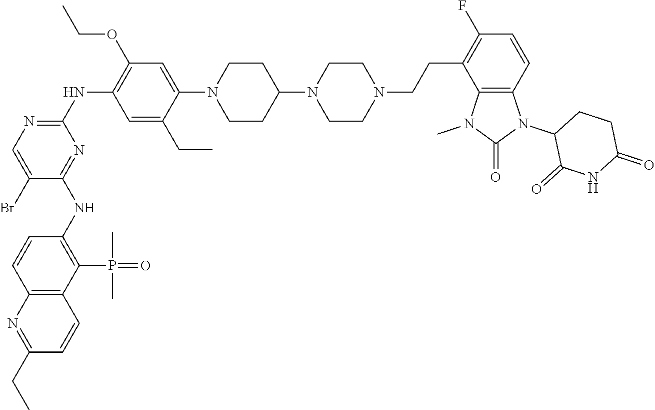 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

Example 503
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
5-fluoro-3-methyl-2-oxo-2,3-dihydro-1H-
benzo[d]imidazol-1-yl)piperidine-2,6-dione

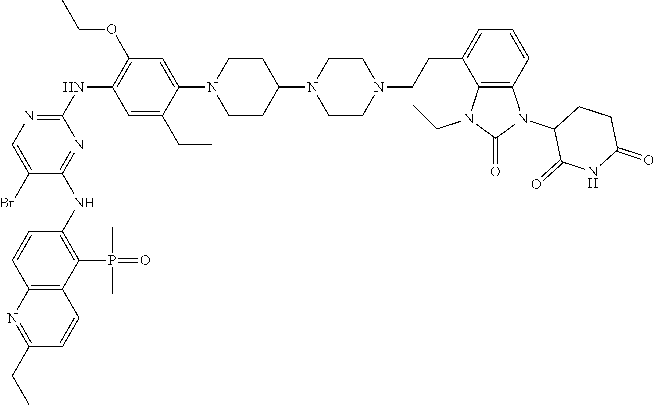 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

Example 504
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
3-ethyl-2-oxo-2,3-dihydro-1H-
benzo[d]imidazol-1-yl)piperidine-2,6-dione

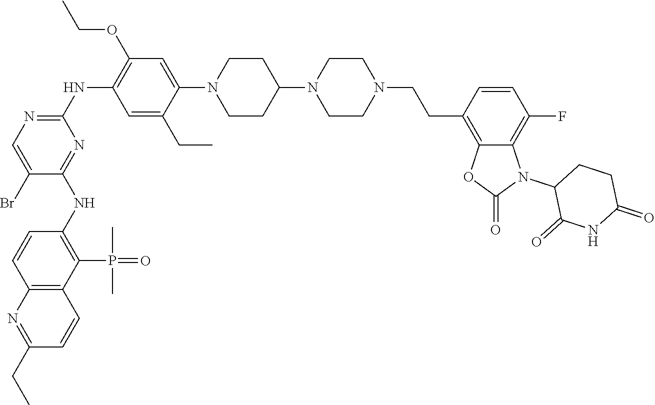 [SMILES: CCOc1cc(N2CCC(N3CCN(CCc4ccc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

Example 505
3-(7-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
4-fluoro-2-oxobenzo[d]oxazol-3(2H)-
yl)piperidine-2,6-dione

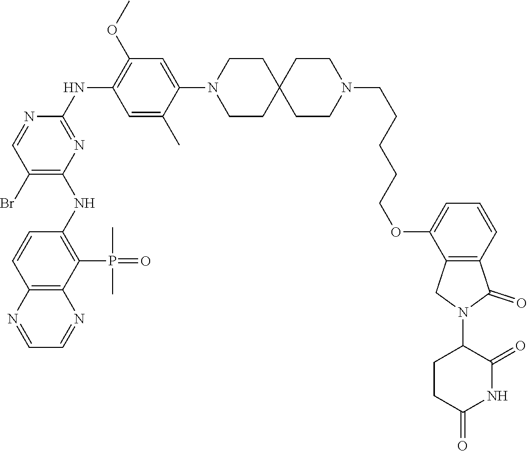 [SMILES: COc1cc(N2CCC3(CCN(CCCCCOc4cccc5c4CN(C4CCC(=O)NC4=O)C5=O)CC3)CC2)c(C)cc1Nc1ncc(Br)c(Nc2ccc3nccnc3c2P(C)(C)=O)n1]

Example 542
3-(4-((5-(9-(4-((5-bromo-4-((5-
(dimethylphosphoryl)quinoxalin-6-
yl)amino)pyrimidin-2-yl)amino)-5-methoxy-2-
methylphenyl)-3,9-diazaspiro[5.5]undecan-3-
yl)pentyl)oxy)-1-oxoisoindolin-2-yl)piperidine-
2,6-dione

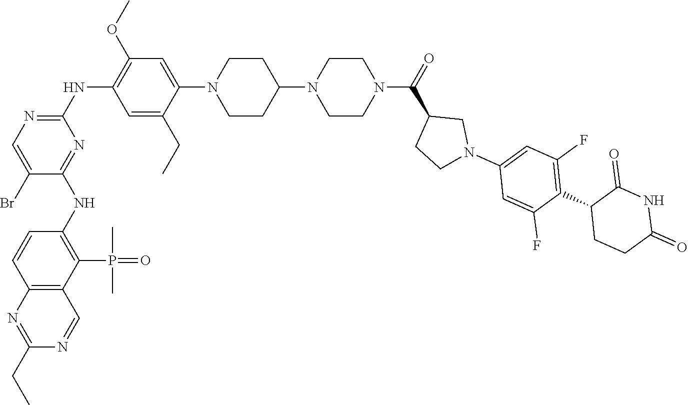 [SMILES: CCc1ncc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

Example 549
(R)-3-(4-((R)-3-(4-(1-(4-((5-bromo-4-((5-
(dimethylphosphoryl)-2-ethylquinazolin-6-
yl)amino)pyrimidin-2-yl)amino)-2-ethyl-5-
methoxyphenyl)piperidin-4-yl)piperazine-1-
carbonyl)pyrrolidin-1-yl)-2,6-
difluorophenyl)piperidine-2,6-dione

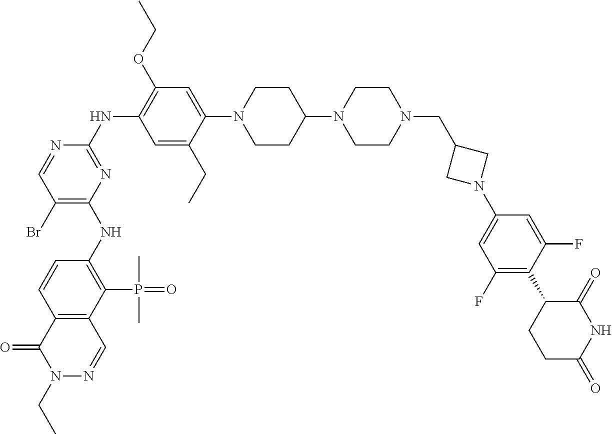 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3c(=O)n(CC)ncc3c2P(C)(C)=O)n1]

Example 551
(R)-3-(4-(3-((4-(1-(4-((5-bromo-4-((5-
(dimethylphosphoryl)-2-ethyl-1-oxo-1,2-
dihydrophthalazin-6-yl)amino)pyrimidin-2-
yl)amino)-5-ethoxy-2-ethylphenyl)piperidin-4-
yl)piperazin-1-yl)methyl)azetidin-1-yl)-2,6-
difluorophenyl)piperidine-2,6-dione

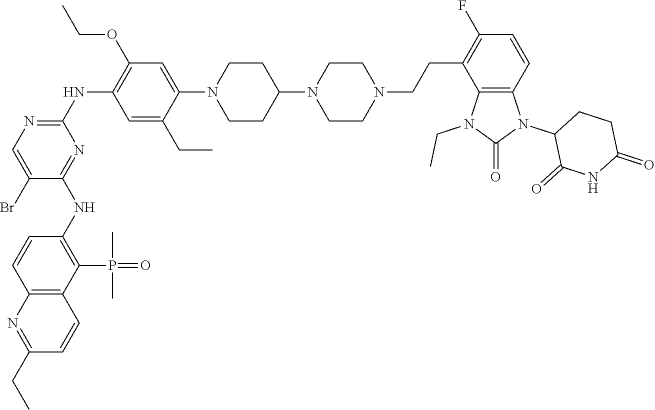 [SMILES: CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

Example 558
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
3-ethyl-5-fluoro-2-oxo-2,3-dihydro-1H-
benzo[d]imidazol-1-yl)piperidine-2,6-dione

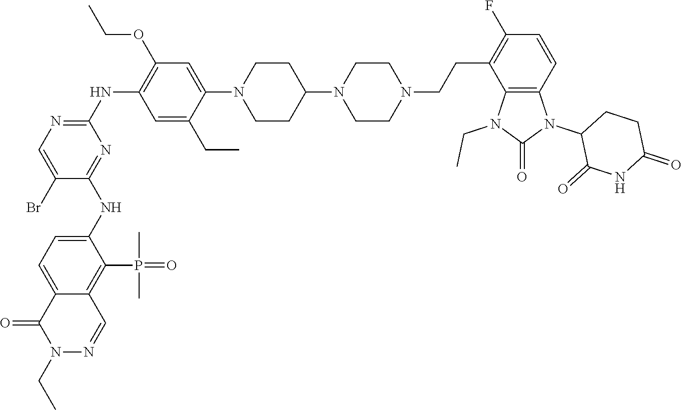 [SMILES: CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1]

Example 559
3-(4-(2-(4-(1-(4-((5-bromo-4-((5-
(dimethylphosphoryl)-2-ethyl-1-oxo-1,2-
dihydrophthalazin-6-yl)amino)pyrimidin-2-
yl)amino)-5-ethoxy-2-ethylphenyl)piperidin-4-
yl)piperazin-1-yl)ethyl)-3-ethyl-5-fluoro-2-oxo-
2,3-dihydro-1H-benzo[d]imidazol-1-
yl)piperidine-2,6-dione

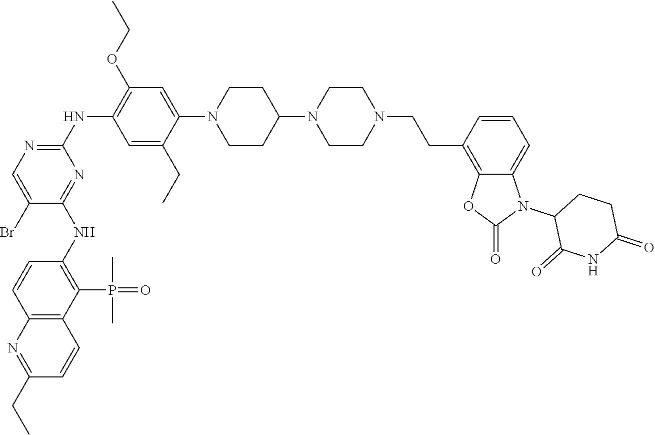 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

Example 563
3-(7-(2-(4-(1-(4-((5-bromo-4-((5-
(dimethylphosphoryl)-2-ethylquinazolin-6-
yl)amino)pyrimidin-2-yl)amino)-5-ethoxy-2-
ethylphenyl)piperidin-4-yl)piperazin-1-yl)ethyl)-
2-oxobenzo[d]oxazol-3(2H)-yl)piperidine-2,6-
dione

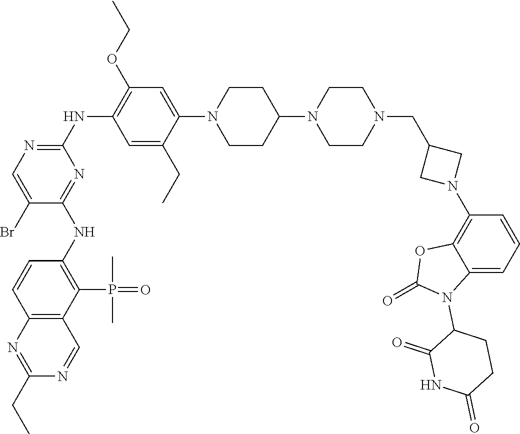 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ncc3c2P(C)(C)=O)n1]

Example 566
3-(7-(3-((4-(1-(4-((5-bromo-4-((5-
(dimethylphosphoryl)-2-ethylquinazolin-6-
yl)amino)pyrimidin-2-yl)amino)-5-ethoxy-2-
ethylphenyl)piperidin-4-yl)piperazin-1-
yl)methyl)azetidin-1-yl)-2-oxobenzo[d]oxazol-
3(2H)-yl)piperidine-2,6-dione

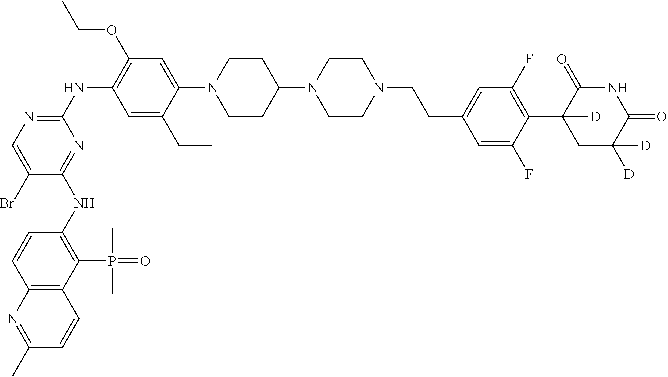

Example 570
3-(4-(2-(4-(1-(4-((5-bromo-4-((5-
(dimethylphosphoryl)-2-methylquinolin-6-
yl)amino)pyrimidin-2-yl)amino)-5-ethoxy-2-
ethylphenyl)piperidin-4-yl)piperazin-1-yl)ethyl)-
2,6-difluorophenyl)piperidine-2,6-dione-3,5,5-d3

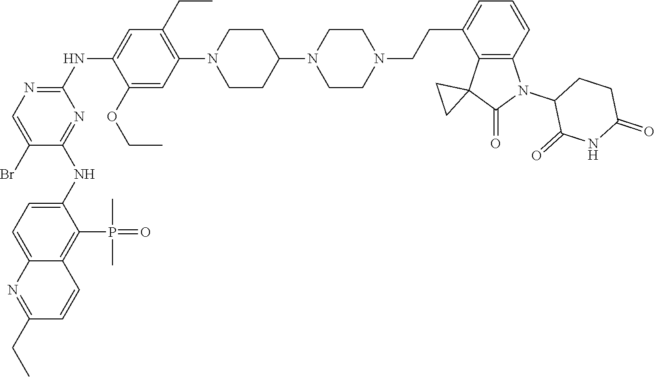 [SMILES: CCOc1cc(N2CCC(N3CCN(CCc4cccc5c4C4(CC4)C(=O)N5C4CCC(=O)NC4=O)CC3)CC2)c(CC)cc1Nc1ncc(Br)c(Nc2ccc3nc(CC)ccc3c2P(C)(C)=O)n1]

Example 571
3-(4′-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
2′-oxospiro[cyclopropane-1,3′-indolin]-1′-
yl)piperidine-2,6-dione

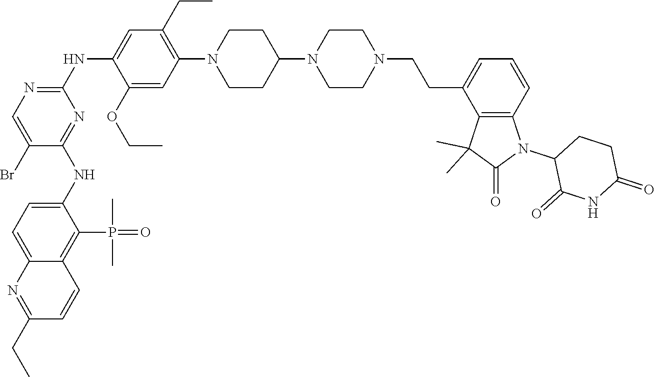 [SMILES: CCOc1cc(N2CCC(N3CCN(CCc4cccc5c4C(C)(C)C(=O)N5C4CCC(=O)NC4=O)CC3)CC2)c(CC)cc1Nc1ncc(Br)c(Nc2ccc3nc(CC)ccc3c2P(C)(C)=O)n1]

Example 572
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
3,3-dimethyl-2-oxoindolin-1-yl)piperidine-2,6-
dione

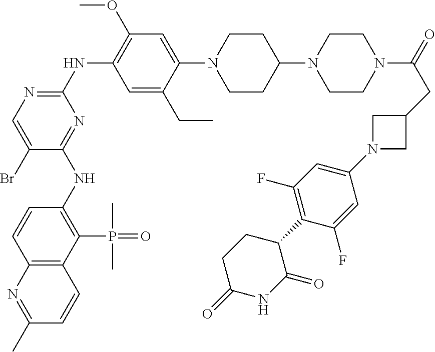 [SMILES: CCc1cc(Nc2ncc(Br)c(Nc3ccc4nc(C)ccc4c3P(C)(C)=O)n2)c(OC)cc1N1CCC(N2CCN(C(=O)CC3CN(c4cc(F)c([C@H]5CCC(=O)NC5=O)c(F)c4)C3)CC2)CC1]

Example 573
(R)-3-(4-(3-(2-(4-(1-(4-((5-bromo-4-((5-
(dimethylphosphoryl)-2-methylquinolin-6-
yl)amino)pyrimidin-2-yl)amino)-2-ethyl-5-
methoxyphenyl)piperidin-4-yl)piperazin-1-yl)-2-
oxoethyl)azetidin-1-yl)-2,6-
difluorophenyl)piperidine-2,6-dione

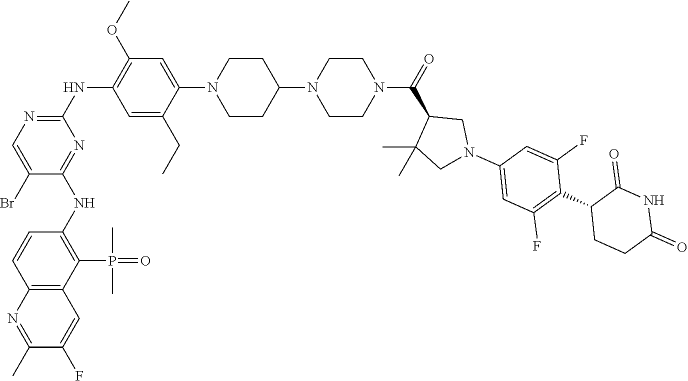 [SMILES: CCc1cc(Nc2ncc(Br)c(Nc3ccc4nc(C)c(F)cc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

Example 576
(R)-3-(4-((R)-4-(4-(1-(4-((5-bromo-4-((5-
(dimethylphosphoryl)-3-fluoro-2-
methylquinolin-6-yl)amino)pyrimidin-2-
yl)amino)-2-ethyl-5-methoxyphenyl)piperidin-4-
yl)piperazine-1-carbonyl)-3,3-
dimethylpyrrolidin-1-yl)-2,6-
difluorophenyl)piperidine-2,6-dione

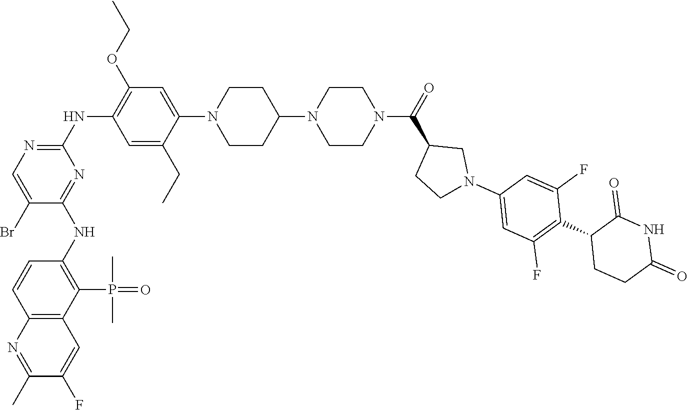 [SMILES: CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3nc(C)c(F)cc3c2P(C)(C)=O)n1]

Example 577
(R)-3-(4-((R)-3-(4-(1-(4-((5-bromo-4-((5-
(dimethylphosphoryl)-3-fluoro-2-
methylquinolin-6-yl)amino)pyrimidin-2-
yl)amino)-5-ethoxy-2-ethylphenyl)piperidin-4-
yl)piperazine-1-carbonyl)pyrrolidin-1-yl)-2,6-
difluorophenyl)piperidine-2,6-dione

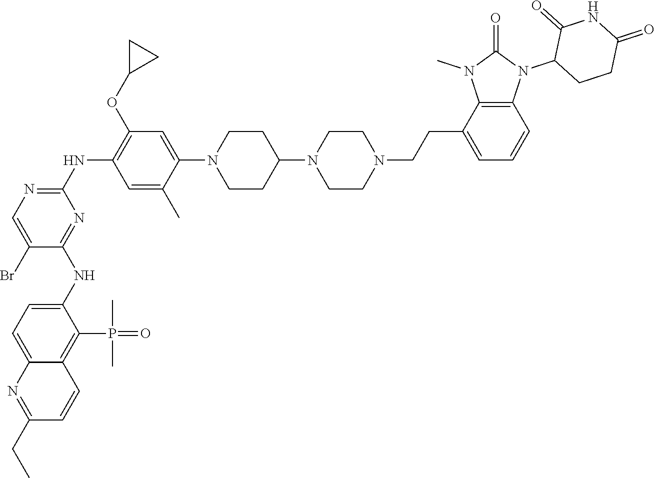 [SMILES: CCc1ccc2c(P(C)(C)=O)c(Nc3nc(Nc4cc(C)c(N5CCC(N6CCN(CCc7cccc8c7n(C)c(=O)n8C7CCC(=O)NC7=O)CC6)CC5)cc4OC4CC4)ncc3Br)ccc2n1]

Example 584
3-(4-(2-(4-(1-(4-((5-bromo-4-((5-
(dimethylphosphoryl)-2-ethylquinolin-6-
yl)amino)pyrimidin-2-yl)amino)-5-
cyclopropoxy-2-methylphenyl)piperidin-4-
yl)piperazin-1-yl)ethyl)-3-methyl-2-oxo-2,3-
dihydro-1H-benzo[d]imidazol-1-yl)piperidine-
2,6-dione

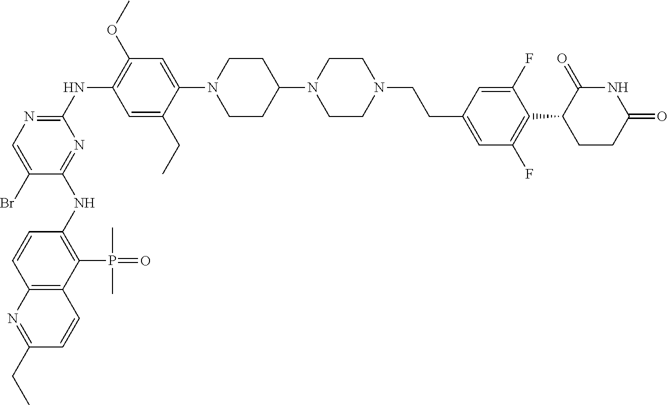 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Br)ccc2n1]

Example 589
(R)-3-(4-(2-(4-(1-(4-((5-bromo-4-((5-
(dimethylphosphoryl)-2-ethylquinazolin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

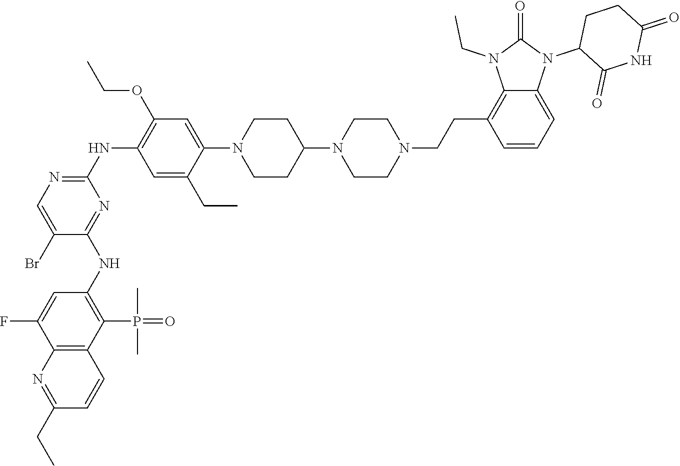 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2cc(F)c3nc(CC)ccc3c2P(C)(C)=O)n1]

Example 643
3-(4-(2-(4-(1-(4-((5-bromo-4-((5-
(dimethylphosphoryl)-2-ethyl-8-fluoroquinolin-
6-yl)amino)pyrimidin-2-yl)amino)-5-ethoxy-2-
ethylphenyl)piperidin-4-yl)piperazin-1-yl)ethyl)-
3-ethyl-2-oxo-2,3-dihydro-1H-
benzo[d]imidazol-1-yl)piperidine-2,6-dione

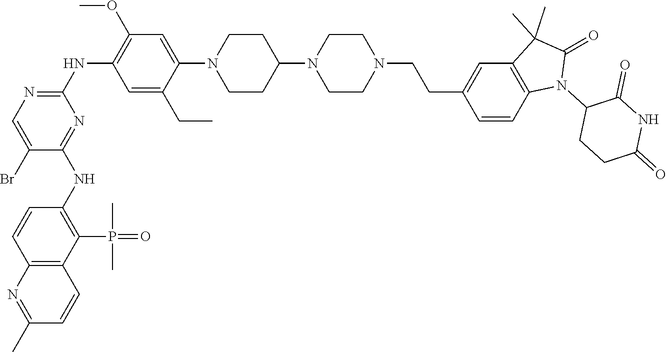 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)C(C)(C)C(=O)N4C3CCC(=O)NC3=O)CC2)CC1]

Example 711
3-(5-(2-(4-(1-(4-((5-bromo-4-((5-
(dimethylphosphoryl)-2-methylquinolin-6-
yl)amino)pyrimidin-2-yl)amino)-2-ethyl-5-
methoxyphenyl)piperidin-4-yl)piperazin-1-
yl)ethyl)-3,3-dimethyl-2-oxoindolin-1-
yl)piperidine-2,6-dione

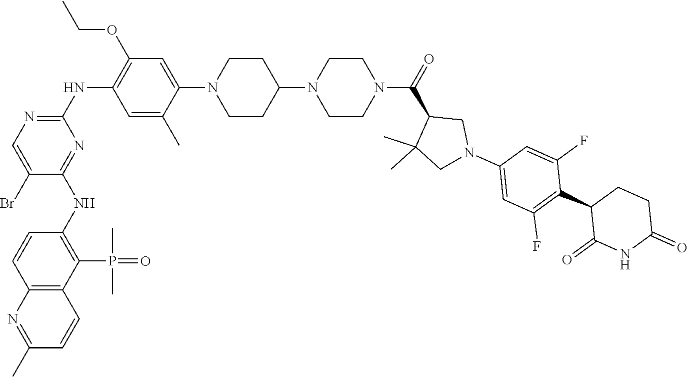 [SMILES: CCOc1cc(N2CCC(N3CCN(C(=O)[C@H]4CN(c5cc(F)c([C@H]6CCC(=O)NC6=O)c(F)c5)CC4(C)C)CC3)CC2)c(C)cc1Nc1ncc(Br)c(Nc2ccc3nc(C)ccc3c2P(C)(C)=O)n1]

Example 727
(R)-3-(4-((R)-4-(4-(1-(4-((5-bromo-4-((5-
(dimethylphosphoryl)-2-methylquinolin-6-
yl)amino)pyrimidin-2-yl)amino)-5-ethoxy-2-
methylphenyl)piperidin-4-yl)piperazine-1-
carbonyl)-3,3-dimethylpyrrolidin-1-yl)-2,6-
difluorophenyl)piperidine-2,6-dione

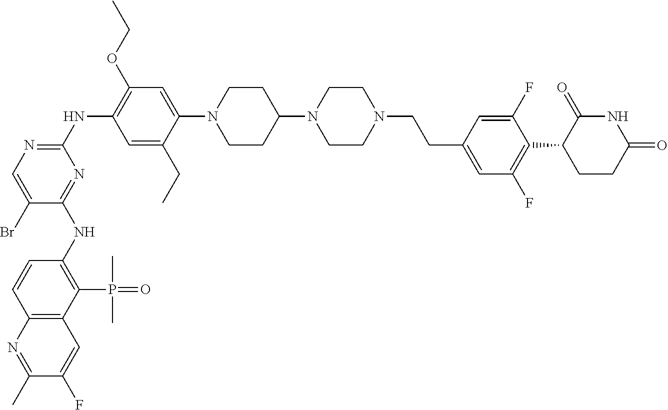 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(C)c(F)cc3c2P(C)(C)=O)n1]

Example 728
methylquinolin-6-yl)amino)pyrimidin-2-
yl)amino)-5-ethoxy-2-ethylphenyl)piperidin-4-
yl)piperazin-1-yl)ethyl)-2,6-
difluorophenyl)piperidine-2,6-dione
(R)-3-(4-(2-(4-(1-(4-((5-bromo-4-((5-
(dimethylphosphoryl)-3-fluoro-2-

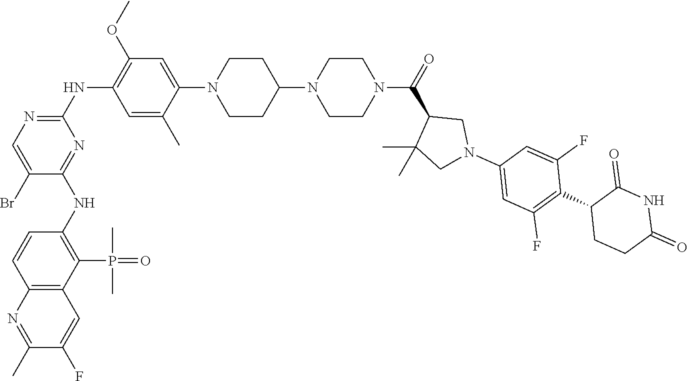 [SMILES: COc1cc(N2CCC(N3CCN(C(=O)[C@H]4CN(c5cc(F)c([C@H]6CCC(=O)NC6=O)c(F)c5)CC4(C)C)CC3)CC2)c(C)cc1Nc1ncc(Br)c(Nc2ccc3nc(C)c(F)cc3c2P(C)(C)=O)n1]

Example 729
(R)-3-(4-((R)-4-(4-(1-(4-((5-bromo-4-((5-
(dimethylphosphoryl)-3-fluoro-2-
methylquinolin-6-yl)amino)pyrimidin-2-
yl)amino)-5-methoxy-2-methylphenyl)piperidin-
4-yl)piperazine-1-carbonyl)-3,3-
dimethylpyrrolidin-1-yl)-2,6-
difluorophenyl)piperidine-2,6-dione

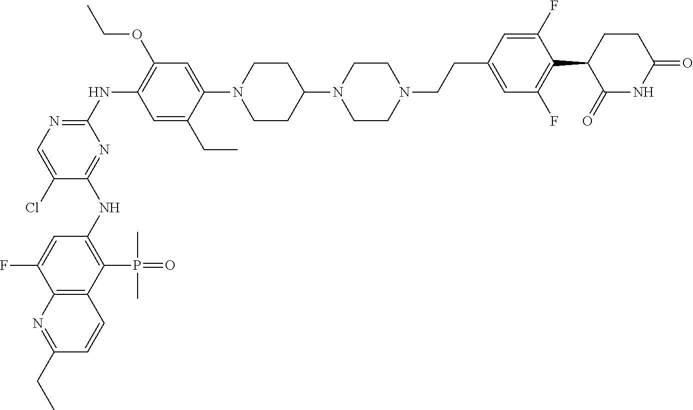 [SMILES: CCOc1cc(N2CCC(N3CCN(CCc4cc(F)c([C@H]5CCC(=O)NC5=O)c(F)c4)CC3)CC2)c(CC)cc1Nc1ncc(Cl)c(Nc2cc(F)c3nc(CC)ccc3c2P(C)(C)=O)n1]

Example 730
(dimethylphosphoryl) -2-ethyl-8-fluoroquinolin-
6-yl)amino)pyrimidin-2-yl)amino)-5-ethoxy-2-
(R)-3-(4-(2-(4-(1-(4-((5-chloro-4-((5-
ethylphenyl)piperidin-4-yl)piperazin-1-yl)ethyl)-
2,6-difluorophenyl)piperidine-2,6-dione

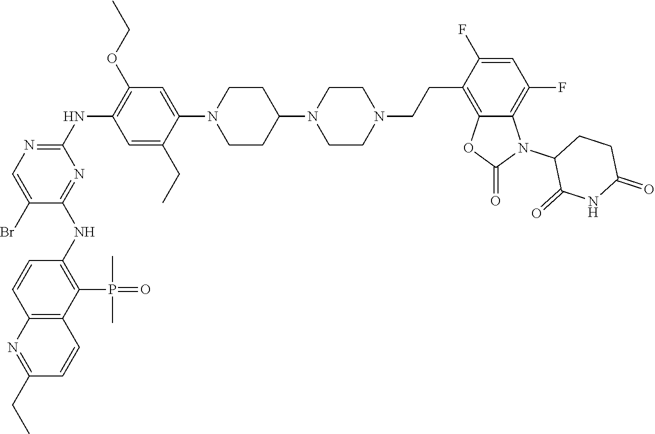 [SMILES: CCOc1cc(N2CCC(N3CCN(CCc4c(F)cc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

Example 731
3-(7-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
4,6-difluoro-2-oxobenzo[d]oxazol-3(2H)-
yl)piperidine-2,6-dione

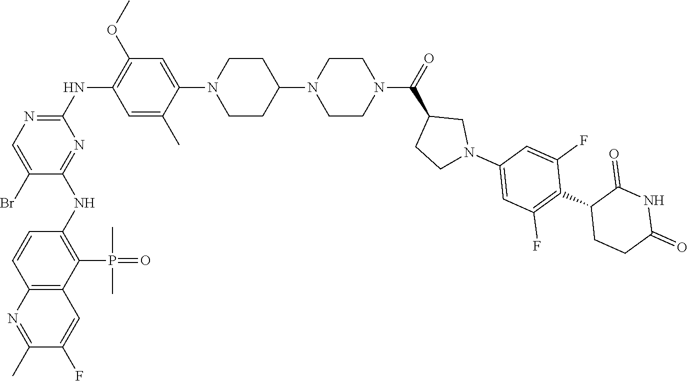 [SMILES: COc1cc(N2CCC(N3CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3nc(C)c(F)cc3c2P(C)(C)=O)n1]

Example 732
(R)-3-(4-((R)-3-(4-(1-(4-((5-bromo-4-((5-
(dimethylphosphoryl)-3-fluoro-2-
methylquinolin-6-yl)amino)pyrimidin-2-
yl)amino)-5-methoxy-2-methylphenyl)piperidin-
4-yl)piperazine-1-carbonyl)pyrrolidin-1-yl)-2,6-
difluorophenyl)piperidine-2,6-dione

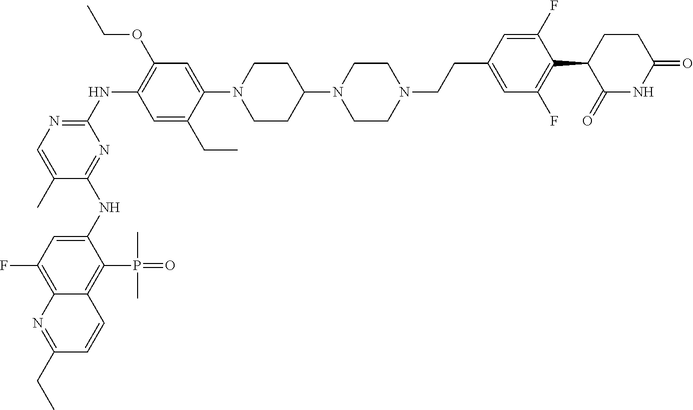 [SMILES: CCOc1cc(N2CCC(N3CCN(CCc4cc(F)c([C@H]5CCC(=O)NC5=O)c(F)c4)CC3)CC2)c(CC)cc1Nc1ncc(C)c(Nc2cc(F)c3nc(CC)ccc3c2P(C)(C)=O)n1]

Example 733
(R)-3-(4-(2-(4-(1-(4-((4-((5-
(dimethylphosphoryl)-2-ethyl-8-fluoroquinolin-
6-yl)amino)-5-methylpyrimidin-2-yl)amino)-5-
ethoxy-2-ethylphenyl)piperidin-4-yl)piperazin-1-
yl)ethyl)-2,6-difluorophenyl)piperidine-2,6-
dione

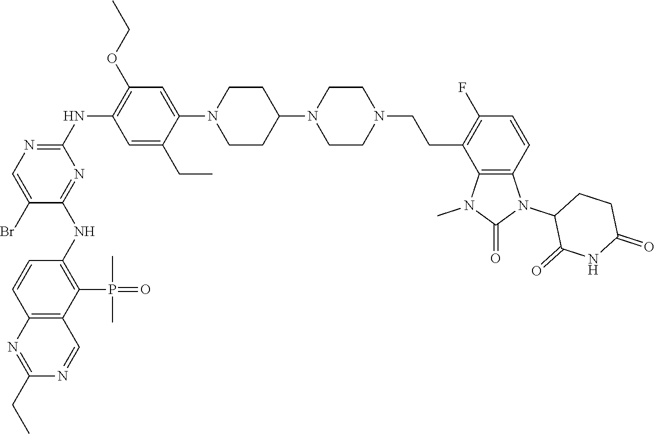 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ncc3c2P(C)(C)=O)n1]

Example 736
3-(4-(2-(4-(1-(4-((5-bromo-4-((5-
(dimethylphosphoryl)-2-ethylquinazolin-6-
yl)amino)pyrimidin-2-yl)amino)-5-ethoxy-2-
ethylphenyl)piperidin-4-yl)piperazin-1-yl)ethyl)-
5-fluoro-3-methyl-2-oxo-2,3-dihydro-1H-
benzo[d]imidazol-1-yl)piperidine-2,6-dione

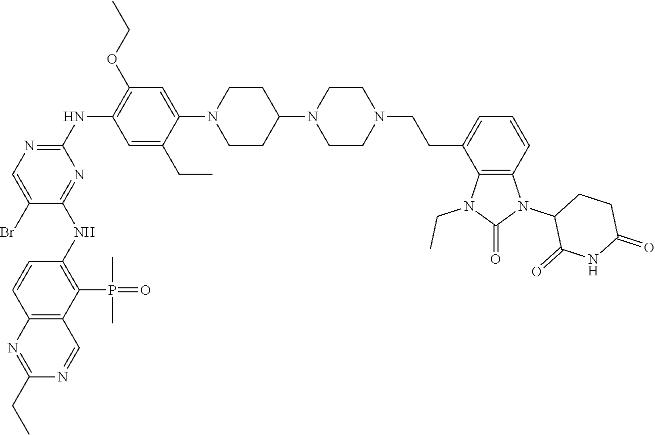 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nc(CC)ncc3c2P(C)(C)=O)n1]

Example 737
3-(4-(2-(4-(1-(4-((5-bromo-4-((5-
(dimethylphosphoryl)-2-ethylquinazolin-6-
yl)amino)pyrimidin-2-yl)amino)-5-ethoxy-2-
ethylphenyl)piperidin-4-yl)piperazin-1-yl)ethyl)-
3-ethyl-2-oxo-2,3-dihydro-1H-
benzo[d]imidazol-1-yl)piperidine-2,6-dione

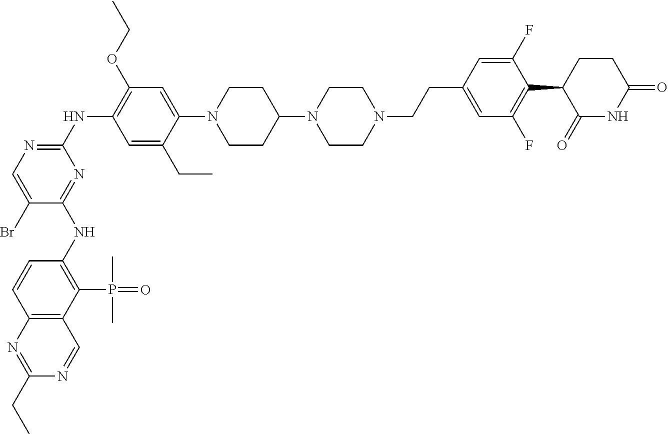 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ncc3c2P(C)(C)=O)n1]

Example 738
(R)-3-(4-(2-(4-(1-(4-((5-bromo-4-((5-
(dimethylphosphoryl)-2-methylquinazolin-6-
yl)amino)pyrimidin-2-yl)amino)-5-ethoxy-2-
ethylphenyl)piperidin-4-yl)piperazin-1-yl)ethyl)-
2,6-difluorophenyl)piperidine-2,6-dione

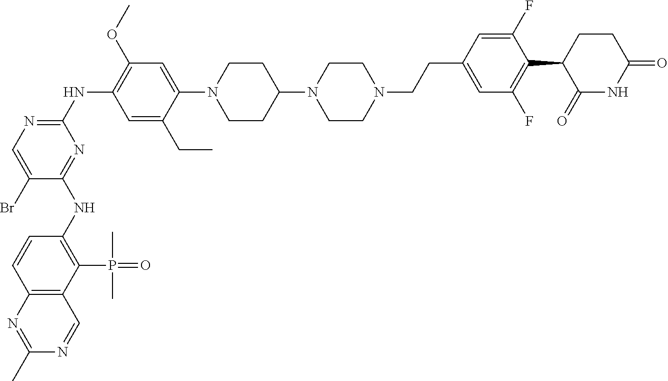 [SMILES: CCc1cc(Nc2ncc(Br)c(Nc3ccc4nc(C)ncc4c3P(C)(C)=O)n2)c(OC)cc1N1CCC(N2CCN(CCc3cc(F)c([C@H]4CCC(=O)NC4=O)c(F)c3)CC2)CC1]

Example 739
(R)-3-(4-(2-(4-(1-(4-((5-bromo-4-((5-
(dimethylphosphoryl)-2-methylquinazolin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

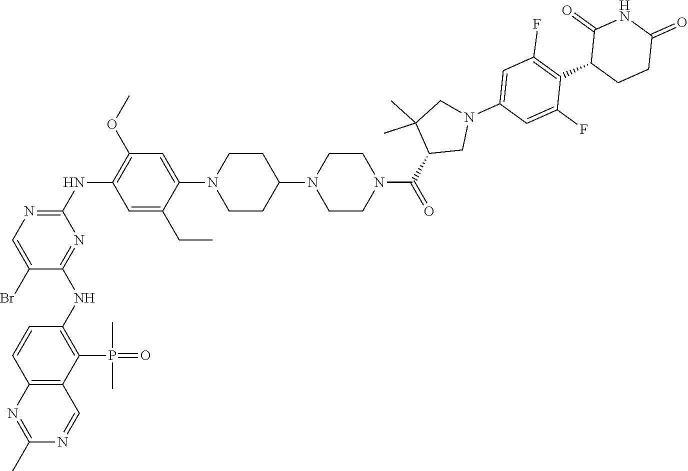 [SMILES: CCc1cc(Nc2ncc(Br)c(Nc3ccc4nc(C)ncc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

Example 740
(R)-3-(4-((R)-4-(4-(1-(4-((5-bromo-4-((5-
(dimethylphosphoryl)-2-methylquinazolin-6-
yl)amino)pyrimidin-2-yl)amino)-2-ethyl-5-
methoxyphenyl)piperidin-4-yl)piperazine-1-
carbonyl)-3,3-dimethylpyrrolidin-1-yl)-2,6-
difluorophenyl)piperidine-2,6-dione

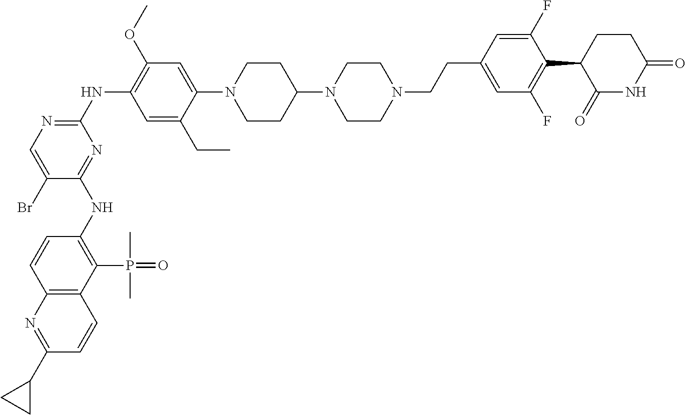 [SMILES: CCc1cc(Nc2ncc(Br)c(Nc3ccc4nc(C5CC5)ccc4c3P(C)(C)=O)n2)c(OC)cc1N1CCC(N2CCN(CCc3cc(F)c([C@H]4CCC(=O)NC4=O)c(F)c3)CC2)CC1]

Example 741
(R)-3-(4-(2-(4-(1-(4-((5-bromo-4-((2-
cyclopropyl-5-(dimethylphosphoryl)quinolin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

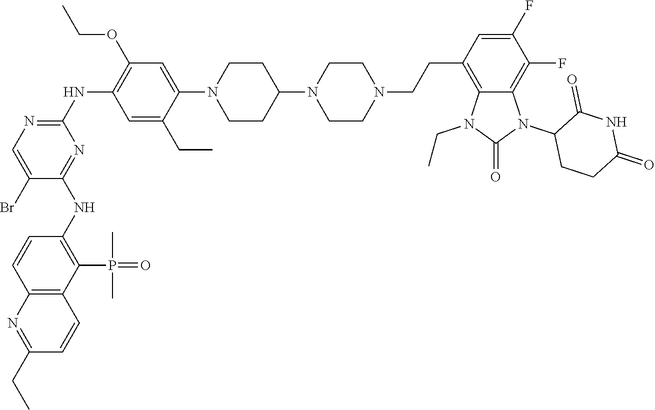 [SMILES: CCOc1cc(N2CCC(N3CCN(CCc4cc(F)c(F)c5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

Example 742
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
3-ethyl-6,7-difluoro-2-oxo-2,3-dihydro-1H-
benzo[d]imidazol-1-yl)piperidine-2,6-dione

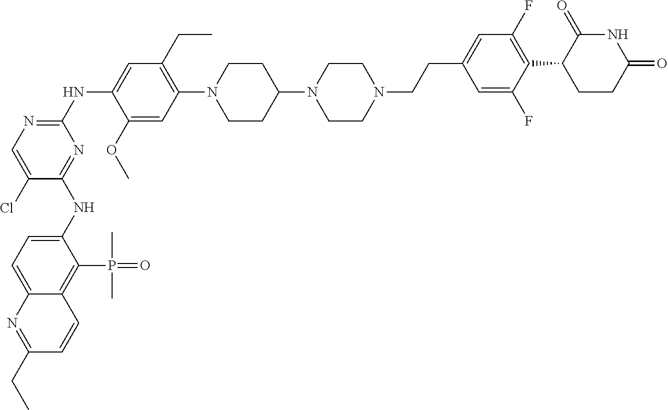 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Cl)ccc2n1]

Example 744
(R)-3-(4-(2-(4-(1-(4-((5-chloro-4-((5-
(dimethylphosphoryl)-2-ethylquinolin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

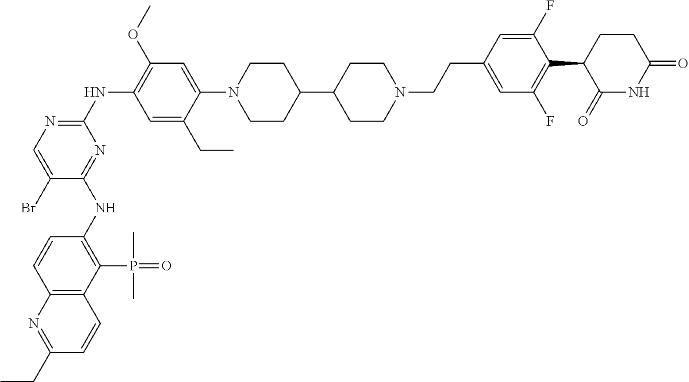 [SMILES: CCc1ccc2c(P(C)(C)=O)c(Nc3nc(Nc4cc(CC)c(N5CCC(C6CCN(CCc7cc(F)c([C@H]8CCC(=O)NC8=O)c(F)c7)CC6)CC5)cc4OC)ncc3Br)ccc2n1]

Example 745
(R)-3-(4-(2-(1′-(4-((5-bromo-4-((5-
(dimethylphosphoryl)-2-ethylquinolin-6-
yl)amino)pyrimidin-2-yl)amino)-2-ethyl-5-
methoxyphenyl)-[4,4′-bipiperidin]-1-yl)ethyl)-
2,6-difluorophenyl)piperidine-2,6-dione

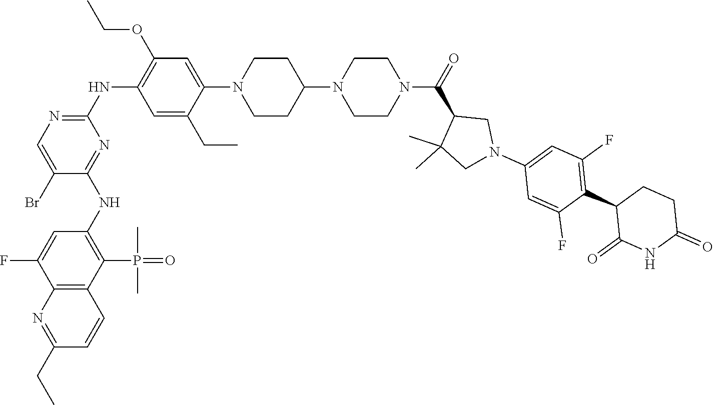 [SMILES: CCOc1cc(N2CCC(N3CCN(C(=O)[C@H]4CN(c5cc(F)c([C@H]6CCC(=O)NC6=O)c(F)c5)CC4(C)C)CC3)CC2)c(CC)cc1Nc1ncc(Br)c(Nc2cc(F)c3nc(CC)ccc3c2P(C)(C)=O)n1]

Example 746
(R)-3-(4-((R)-4-(4-(1-(4-((5-bromo-4-((5-
(dimethylphosphoryl)-2-ethyl-8-fluoroquinolin-
6-yl)amino)pyrimidin-2-yl)amino)-5-ethoxy-2-
ethylphenyl)piperidin-4-yl)piperazine-1-
carbonyl)-3,3-dimethylpyrrolidin-1-yl)-2,6-
difluorophenyl)piperidine-2,6-dione

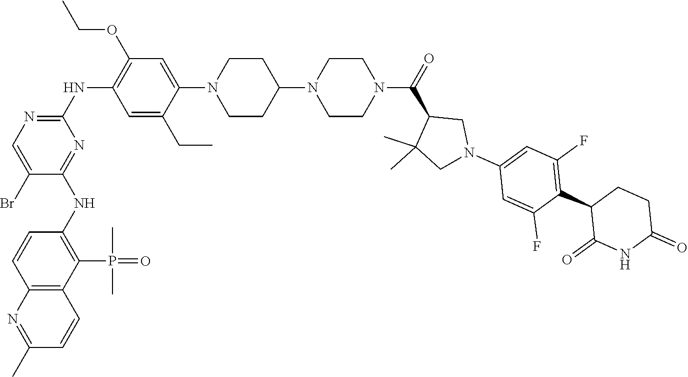 [SMILES: CCOc1cc(N2CCC(N3CCN(C(=O)[C@H]4CN(c5cc(F)c([C@H]6CCC(=O)NC6=O)c(F)c5)CC4(C)C)CC3)CC2)c(CC)cc1Nc1ncc(Br)c(Nc2ccc3nc(C)ccc3c2P(C)(C)=O)n1]

Example 747
(R)-3-(4-((R)-4-(4-(1-(4-((5-bromo-4-((5-
(dimethylphosphoryl)-2-methylquinolin-6-
yl)amino)pyrimidin-2-yl)amino)-5-ethoxy-2-
ethylphenyl)piperidin-4-yl)piperazine-1-
carbonyl)-3,3-dimethylpyrrolidin-1-yl)-2,6-
difluorophenyl)piperidine-2,6-dione

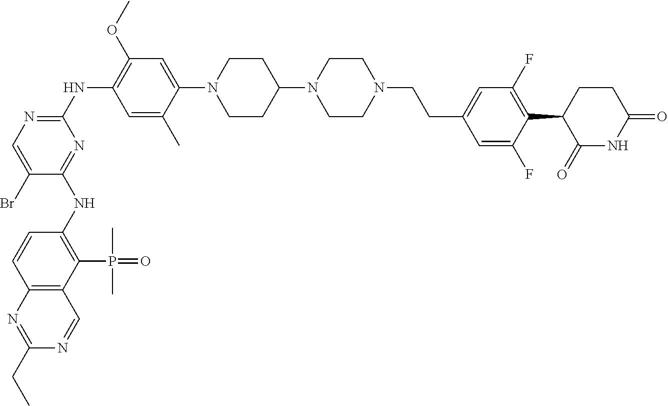 [SMILES: CCc1ncc2c(P(C)(C)=O)c(Nc3nc(Nc4cc(C)c(N5CCC(N6CCN(CCc7cc(F)c([C@H]8CCC(=O)NC8=O)c(F)c7)CC6)CC5)cc4OC)ncc3Br)ccc2n1]

Example 748
(R)-3-(4-(2-(4-(1-(4-((5-bromo-4-((5-
(dimethylphosphoryl)-2-ethylquinazolin-6-
yl)amino)pyrimidin-2-yl)amino)-5-methoxy-2-
methylphenyl)piperidin-4-yl)piperazin-1-
yl)ethyl)-2,6-difluorophenyl)piperidine-2,6-
dione

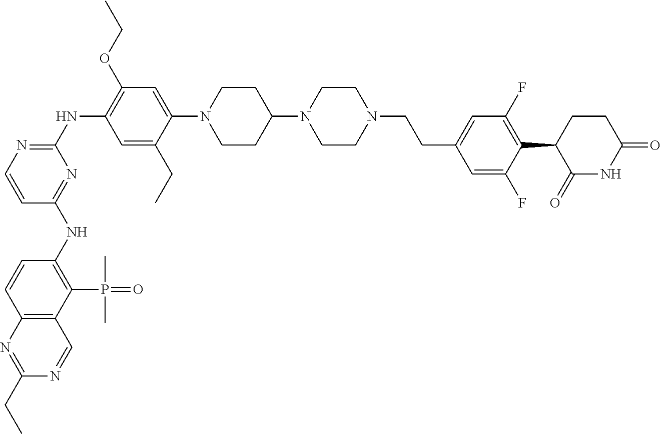 [SMILES: CCOc1cc(N2CCC(N3CCN(CCc4cc(F)c([C@H]5CCC(=O)NC5=O)c(F)c4)CC3)CC2)c(CC)cc1Nc1nccc(Nc2ccc3nc(CC)ncc3c2P(C)(C)=O)n1]

Example 749
(R)-3-(4-(2-(4-(1-(4-((4-((5-
(dimethylphosphoryl)-2-ethylquinazolin-6-
yl)amino)pyrimidin-2-yl)amino)-5-ethoxy-2-
ethylphenyl)piperidin-4-yl)piperazin-1-yl)ethyl)-
2,6-difluorophenyl)piperidine-2,6-dione

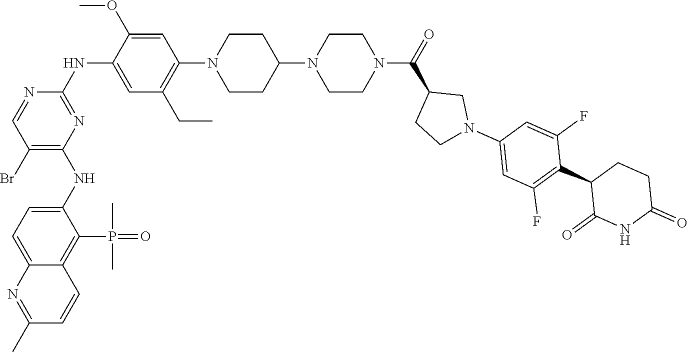 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

Example 750
(R)-3-(4-((R)-3-(4-(1-(4-((5-bromo-4-((5-
(dimethylphosphoryl)-2-methylquinolin-6-
yl)amino)pyrimidin-2-yl)amino)-2-ethyl-5-
methoxyphenyl)piperidin-4-yl)piperazine-1-
carbonyl)pyrrolidin-1-yl)-2,6-
difluorophenyl)piperidine-2,6-dione

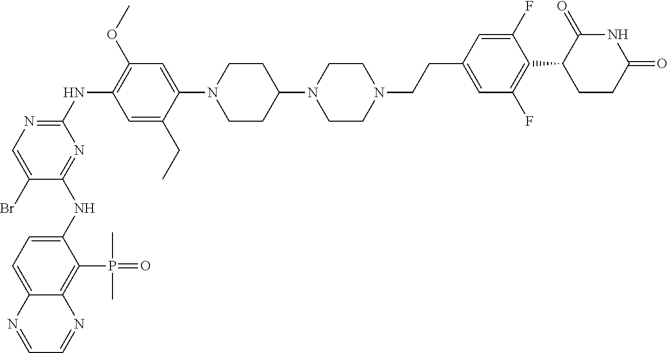 [SMILES: CCc1cc(Nc2ncc(Br)c(Nc3ccc4nccnc4c3P(C)(C)=O)n2)c(OC)cc1N1CCC(N2CCN(CCc3cc(F)c([C@H]4CCC(=O)NC4=O)c(F)c3)CC2)CC1]

Example 751
(R)-3-(4-(2-(4-(1-(4-((5-bromo-4-((5-
(dimethylphosphoryl)quinoxalin-6-
yl)amino)pyrimidin-2-yl)amino)-2-ethyl-5-
methoxyphenyl)piperidin-4-yl)piperazin-1-
yl)ethyl)-2,6-difluorophenyl)piperidine-2,6-
dione

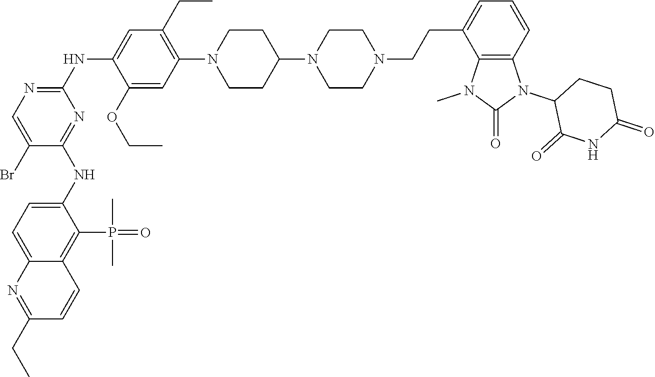 [SMILES: CCOc1cc(N2CCC(N3CCN(CCc4cccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

Example 752
3-(4-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
3-methyl-2-oxo-2,3-dihydro-1H-
benzo[d]imidazol-1-yl)piperidine-2,6-dione

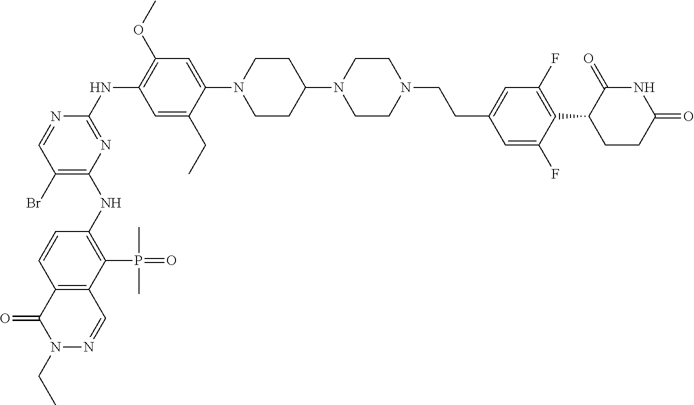 [SMILES: CCc1cc(Nc2ncc(Br)c(Nc3ccc4c(=O)n(CC)ncc4c3P(C)(C)=O)n2)c(OC)cc1N1CCC(N2CCN(CCc3cc(F)c([C@H]4CCC(=O)NC4=O)c(F)c3)CC2)CC1]

Example 753
(R)-3-(4-(2-(4-(1-(4-((5-bromo-4-((5-
(dimethylphosphoryl)-2-ethyl-1-oxo-1,2-
dihydrophthalazin-6-yl)amino)pyrimidin-2-
yl)amino)-2-ethyl-5-methoxyphenyl)piperidin-4-
yl)piperazin-1-yl)ethyl)-2,6-
difluorophenyl)piperidine-2,6-dione

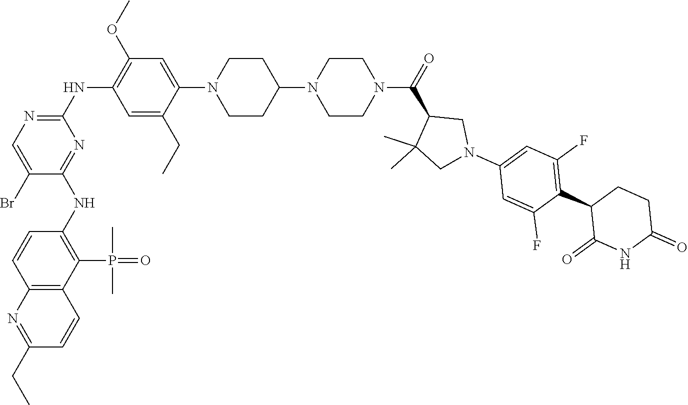 [SMILES: CCc1ccc2c(P(C)(C)=O)c(Nc3nc(Nc4cc(CC)c(N5CCC(N6CCN(C(=O)[C@H]7CN(c8cc(F)c([C@H]9CCC(=O)NC9=O)c(F)c8)CC7(C)C)CC6)CC5)cc4OC)ncc3Br)ccc2n1]

Example 754
(R)-3-(4-((R)-4-(4-(1-(4-((5-bromo-4-((5-
(dimethylphosphoryl)-2-ethylquinolin-6-
yl)amino)pyrimidin-2-yl)amino)-2-ethyl-5-
methoxyphenyl)piperidin-4-yl)piperazine-1-
carbonyl)-3,3-dimethylpyrrolidin-1-yl)-2,6-
difluorophenyl)piperidine-2,6-dione

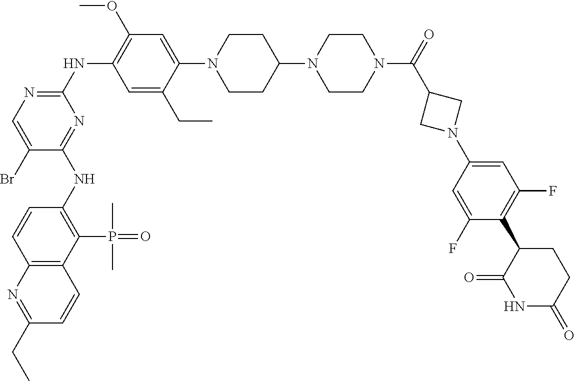 [SMILES: CCc1ccc2c(P(C)(C)=O)c(Nc3nc(Nc4cc(CC)c(N5CCC(N6CCN(C(=O)C7CN(c8cc(F)c([C@H]9CCC(=O)NC9=O)c(F)c8)C7)CC6)CC5)cc4OC)ncc3Br)ccc2n1]

Example 755
(R)-3-(4-(3-(4-(1-(4-((5-bromo-4-((5-
(dimethylphosphoryl)-2-ethylquinolin-6-
yl)amino)pyrimidin-2-yl)amino)-2-ethyl-5-
methoxyphenyl)piperidin-4-yl)piperazine-1-
carbonyl)azetidin-1-yl)-2,6-
difluorophenyl)piperidine-2,6-dione

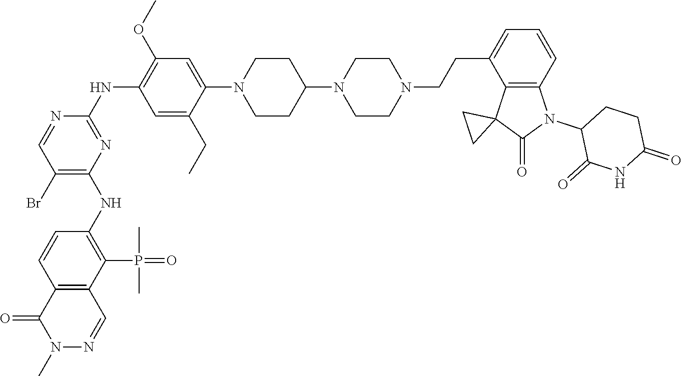 [SMILES: CCc1cc(Nc2ncc(Br)c(Nc3ccc4c(=O)n(C)ncc4c3P(C)(C)=O)n2)c(OC)cc1N1CCC(N2CCN(CCc3cccc4c3C3(CC3)C(=O)N4C3CCC(=O)NC3=O)CC2)CC1]

Example 756
3-(4′-(2-(4-(1-(4-((5-bromo-4-((5-
(dimethylphosphoryl)-2-methyl-1-oxo-1,2-
dihydrophthalazin-6-yl)amino)pyrimidin-2-
yl)amino)-2-ethyl-5-methoxyphenyl)piperidin-4-
yl)piperazin-1-yl)ethyl)-2′-
oxospiro[cyclopropane-1,3′-indolin]-1′-
yl)piperidine-2,6-dione

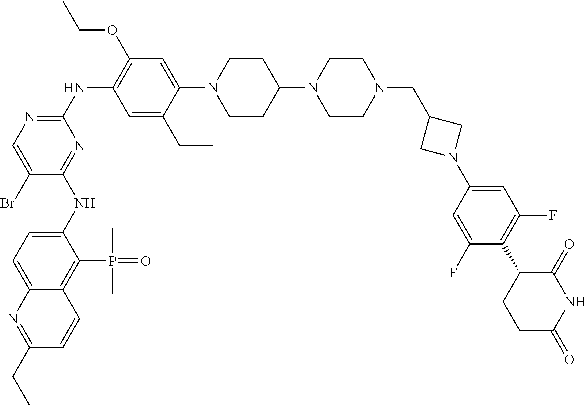 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ccc3c2P(C)(C)=O)n1]

Example 541
(R)-3-(4-(3-((4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-
yl)methyl)azetidin-1-yl)-2,6-
difluorophenyl)piperidine-2,6-dione

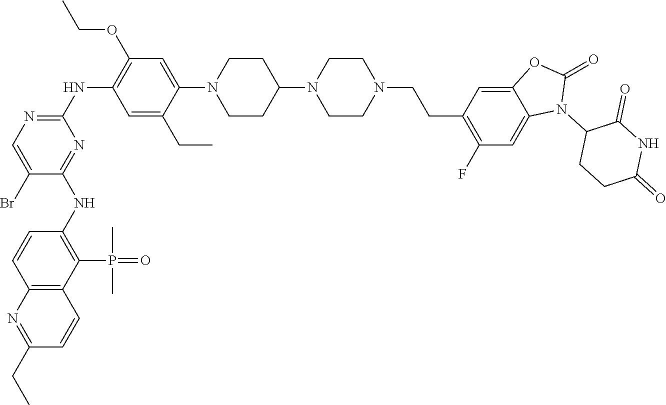 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ccc3c2P(C)(C)=O)n1]

Example 492
3-(6-(2-(4-(1-(4-((5-bromo-4-((5-
(dimethylphosphoryl)-2-ethylquinolin-6-
yl)amino)pyrimidin-2-yl)amino)-5-ethoxy-2-
ethylphenyl)piperidin-4-yl)piperazin-1-yl)ethyl)-
5-fluoro-2-oxobenzo[d]oxazol-3(2H)-
yl)piperidine-2,6-dione

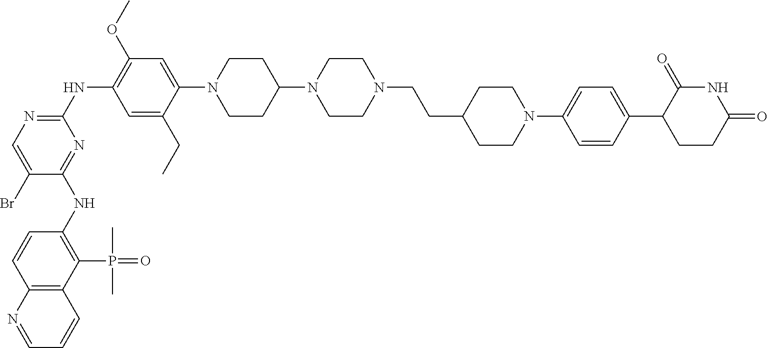 [SMILES: CCc1cc(Nc2ncc(Br)c(Nc3ccc4ncccc4c3P(C)(C)=O)n2)c(OC)cc1N1CCC(N2CCN(CCC3CCN(c4ccc(C5CCC(=O)NC5=O)cc4)CC3)CC2)CC1]

Example 23
3-(4-(4-(2-(4-(1-(4-((5-bromo-4-((5-
(dimethylphosphoryl)quinoxalin-6-
yl)amino)pyrimidin-2-yl)amino)-2-ethyl-5-
methoxyphenyl)piperidin-4-yl)piperazin-1-
yl)ethyl)piperidin-1-yl)phenyl)piperidine-2,6-
dione

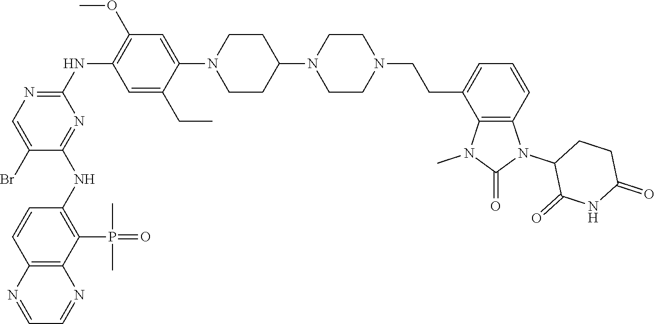 [SMILES: CCc1cc(Nc2ncc(Br)c(Nc3ccc4nccnc4c3P(C)(C)=O)n2)c(OC)cc1N1CCC(N2CCN(CCc3cccc4c3n(C)c(=O)n4C3CCC(=O)NC3=O)CC2)CC1]

Example 318
3-(4-(2-(4-(1-(4-((5-bromo-4-((5-
(dimethylphosphoryl)quinoxalin-6-
yl)amino)pyrimidin-2-yl)amino)-2-ethyl-5-
methoxyphenyl)piperidin-4-yl)piperazin-1-
yl)ethyl)-3-methyl-2-oxo-2,3-dihydro-1H-
benzo[d]imidazol-1-yl)piperidine-2,6-dione

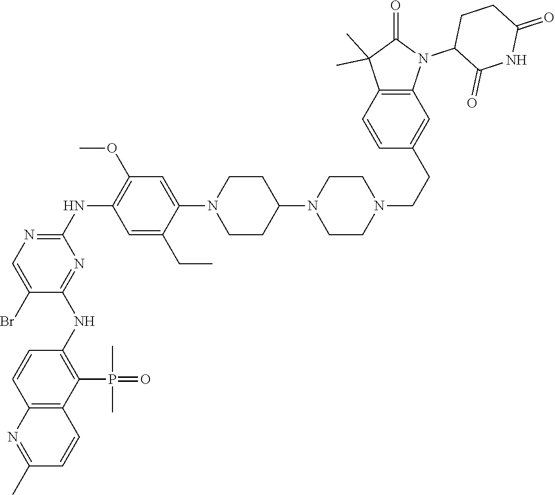 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)N(C3CCC(=O)NC3=O)C(=O)C4(C)C)CC2)CC1]

Example 413
3-(6-(2-(4-(1-(4-((5-bromo-4-((5-
(dimethylphosphoryl)-2-methylquinolin-6-
yl)amino)pyrimidin-2-yl)amino)-2-ethyl-5-
methoxyphenyl)piperidin-4-yl)piperazin-1-
yl)ethyl)-3,3-dimethyl-2-oxoindolin-1-
yl)piperidine-2,6-dione

Aspect 48. A pharmaceutical composition comprising a compound of any one of Aspects 1-47 or a pharmaceutically acceptable salt, stereoisomer, tautomer or prodrug thereof, together with a pharmaceutically acceptable excipient.

Aspect 49. A method of treating a disease that can be affected by EGFR modulation, comprises administrating a subject in need thereof an effective amount of a compound of any one of Aspects 1-47 or a pharmaceutically acceptable salt, stereoisomer, tautomer or prodrug thereof.

Aspect 50. The method of Aspect 49, wherein the disease is selected from cancer, preferred pancreatic cancer, breast cancer, glioblastoma multiforme, head and neck cancer, or non-small cell lung cancer.

Aspect 51. Use of a compound of any one of Aspects 1-47 or a pharmaceutically acceptable salt, stereoisomer, tautomer or prodrug thereof in the preparation of a medicament for treating a disease that can be affected by EGFR modulation.

Aspect 52. The use of Aspect 51, wherein the disease is cancer, preferred pancreatic cancer, breast cancer, glioblastoma multiforme, head and neck cancer, or non-small cell lung cancer.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane.

Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆ and the like.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; ds-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm. Gradient method: Flow: 1.8 mL/min. Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity U) Detector: MWD (190-400 nm), Mass detector: G6125C SQ Mobile phase: A: water with 0.1% Formic acid, B: acetonitrile with 0.1% Formic acid Column: Poroshell 120 BC-C18, 4.6×50 mm, 2.7 pm Gradient method: Flow: 1.2 mL/min Time (min) Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| Aq | Aqueous |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| BTEAC | Benzyltriethylammonium chloride |
| C:40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N,N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCI | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH,Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| TCFH | N,N,N′,N′-Tetramethylchloroformamidinium hexafluorophosphate |
| CDI | 1,1′-Carbonyldiimidazole |

### Example 483: (R)-3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (75 mg, 0.10 mmol, the compound was obtained through the way similar to compound “(6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide”) and (R)-2-(4-(2,6-dioxopiperidin-3-yl)-3,5-difluorophenyl)acetaldehyde (32 mg, 0.12 mmol) in DCM (8 mL) was stirred in a flask at room temperature for 10 min. To the mixture was added sodium triacetoxyborohydride (65 mg, 0.31 mmol) and the reaction was stirred at room temperature for another 0.5 h. Then the mixture was concentrated under vacuum to afford the crude product, which was purified with prep-HPLC chromatography (0.10% FA in water:acetonitrile=90:10˜50:50 gradient elution) to give the product (29 mg, 29.2%). ¹H NMR (500 MHz, DMSO) δ 11.74 (s, 1H), 10.97 (s, 1H), 8.60 (d, J=8.9 Hz, 1H), 8.23 (d, J=6.2 Hz, 2H), 7.93 (s, 1H), 7.87 (d, J=9.3 Hz, 1H), 7.45 (d, J=8.9 Hz, 1H), 7.38 (s, 1H), 7.03 (d, J=10.1 Hz, 2H), 6.70 (s, 1H), 4.20 (dd, J=12.7, 5.0 Hz, 1H), 4.00 (q, J=6.9 Hz, 2H), 3.33-3.25 (m, 2H), 2.95-2.91 (m, 4H), 2.86-2.73 (m, 3H), 2.66-2.63 (m, 7H), 2.48-2.44 (m, 4H), 2.24 (d, J=7.2 Hz, 3H), 2.15-2.12 (m, 1H), 2.03-1.96 (m, 7H), 1.82 (d, J=11.1 Hz, 2H), 1.60-1.44 (m, 2H), 1.32 (t, J=7.6 Hz, 3H), 1.25 (t, J=6.9 Hz, 3H), 0.71 (s, 3H). [M+H]⁺=986.7.

### Example 484: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: ethyl 4-(4-bromo-2,6-difluorophenyl)-4-cyanobutanoate

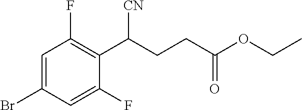 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THF (150 mL) was added LDA (2 M in THF, 24 mL, 48 mmol) dropwise in 20 min at −65° C., the reaction solution was stirred for 1 hour at this temperature, then to this was added ethyl 3-bromopropanoate (9.4 g, 51.7 mmol) in THF (30 mL) dropwise in 10 min. The resulting solution was stirred for 30 min at −65° C., and then allowed to warm to room temperature naturally. The reaction was quenched by the addition of sat. aq. NH₄Cl (50 mL), and extracted with EtOAc (100 mL×3). The combined organic layers were washed with brine, dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

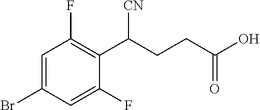 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and extracted with EtOAc (50 mL×2). The pH value of water phase was adjusted to 4-5 with 1 N HCl (10 mL), and extracted with EtOAc (50 mL×3). The combined organic layers were washed with brine (50 mL×3), and dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

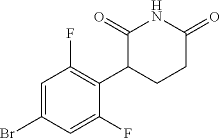 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene (100 mL) was added conc. H₂SO₄ (2 mL, 36.9 mmol). The resulting solution was stirred at 100° C. for 3 h. The reaction mixture was concentrated under vacuum, then the mixture was poured into water. The pH value was adjusted to 7-8 with sat. aq. NaHCO₃ (40 mL), and resulting solution was extracted with EtOAc (50 mL×3). The combined organic layers were washed with water and brine, dried over anhydrous Na₂SO₄, filtered and concentrated to afford the product (8.2 g, 80.4%). [M+H]⁺=304.3.

### Step 4: (R,E)-3-(4-(2-ethoxyvinyl)-2,6-difluorophenyl)piperidine-2,6-dione

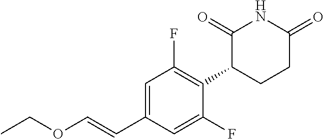 [SMILES: CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf)Cl₂ (883 mg, 1.35 mmol) and CsF (8.2 g, 54.0 mmol). The resulting mixture was stirred for 2 h at 80° C. under nitrogen atmosphere. The reaction solution was diluted with water, extracted with EtOAc (100 mL×2). The combined organic layers were washed with water and brine, dried over anhydrous Na₂SO₄, filtered and concentrated under reduced pressure. The residue was purified by SFC (IH (3*25 cm, 5 um), 13% EtOH/87% CO₂, 100 bar, 100 ml/min) and the title compound corresponded to peak A @ 1.679 min/254 nm. (3.1 g, 39.0%). [M+H]⁺=296.1.

### Step 5: (R)-2-(4-(2,6-dioxopiperidin-3-yl)-3,5-difluorophenyl)acetaldehyde

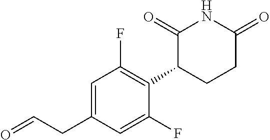 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA (50 mL). The resulting solution was stirred for 2 h at room temperature. The reaction solution was evaporated to dryness to afford the product (2.6 g, 91.8%). [M+H]⁺=268.1.

### Step 6: 6-nitroquinolin-2-ol

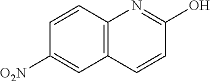 [SMILES: O=[N+]([O-])c1ccc2nc(O)ccc2c1]

To a stirred solution of quinolin-2-ol (6 g, 41.3 mmol) in conc. H₂SO₄ (98%, 50 mL) was added dropwise a solution of conc. HNO₃ (65%, 3.12 g, 49.6 mmol) at 0° C. Then the mixture was stirred at rt for 1 h. The mixture was diluted with water (200 mL) at 0° C. The resulting mixture was filtered and the filter cake was washed with H₂O (500 ml), and dried in vacuum to afford 6-nitroquinolin-2-ol (5.5 g 69.9%) [M+H]⁺=191.1.

### Step 7: 2-chloro-6-nitroquinoline

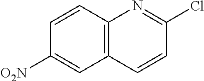 [SMILES: O=[N+]([O-])c1ccc2nc(Cl)ccc2c1]

A solution of 6-nitroquinolin-2-ol (5.5 g, 28.78 mmol) in POCl₃ (50 mL) was stirred at 100° C. for 2 hrs. Then the mixture was cooled to rt, and concentrated in vacuo. The residue was purified by Combi-Flash (silica column, 40 g, DCM:MeOH=15:1) to give 2-chloro-6-nitroquinoline (5 g, 82.9%) [M+H]⁺=209.1.

### Step 8: 6-nitro-2-vinylquinoline

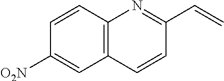

To a suspension of 2-chloro-6-nitroquinoline (5 g, 23.9 mmol) and 4,4,5,5-tetramethyl-2-vinyl-1,3,2-dioxaborolane (7.37 g, 47.8 mmol) in dioxane (40 mL) and water (10 mL) was added K₂CO₃ (9.91 g, 71.8 mmol) and Pd(dppf)Cl₂ (1.74 g, 2.39 mmol) under nitrogen atmosphere. The mixture was warmed to 100° C. and stirred for 16 hrs. Then the mixture was cooled to rt and filtered. The filtrate was concentrated in vacuo. The residue was purified by Combi-Flash (silica column, 40 g, DCM:MeOH=15:1) to give 6-nitro-2-vinylquinoline (4.5 g, 93.9%). [M+H]⁺=201.1.

### Step 9: 2-ethylquinolin-6-amine

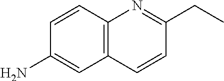

To a suspension of 6-nitro-2-vinylquinoline (4.5 g, 22.38 mmol) in MeOH (20 mL) was added Pd/C (10 wt. %, wet, 1.5 g). The mixture was stirred at rt for 16 hrs under hydrogen atmosphere. Then the mixture was filtered and the solid was washed with MeOH. The filtrate was concentrated in vacuo to afford 2-ethylquinolin-6-amine (3.84 g, 99.2%). [M+H]⁺=173.1.

### Step 10: 2-ethyl-5-iodoquinolin-6-amine

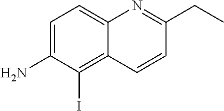

The title compound (4.5 g, 75.3%) was prepared in a manner similar to that in Example 486 step 1 from 2-ethylquinolin-6-amine and ICl. [M+H]⁺=299.1.

### Step 11: (6-amino-2-ethylquinolin-5-yl)dimethylphosphine oxide

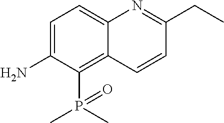 [SMILES: CCc1ccc2c(P(C)(C)=O)c(N)ccc2n1]

The title compound (3.5 g, 93.5%). was prepared in a manner similar to that in Example 486 step 2 from 2-ethyl-5-iodoquinolin-6-amine and dimethylphosphineoxide. [M+H]⁺=249.1.

### Step 12 (6-((5-bromo-2-chloropyrimidin-4-yl)amino)-2-ethylquinolin-5-yl)dimethylphosphine oxide

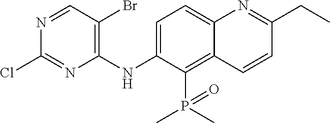 [SMILES: CCc1ccc2c(P(C)(C)=O)c(Nc3nc(Cl)ncc3Br)ccc2n1]

The title compound (2.5 g, 40.4%). was prepared in a manner similar to that in Example 486 step 3 from (6-amino-2-ethylquinolin-5-yl)dimethylphosphine oxide and 5-bromo-2,4-dichloropyrimidine. [M+H]⁺=439.6.

### Step 13: (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide

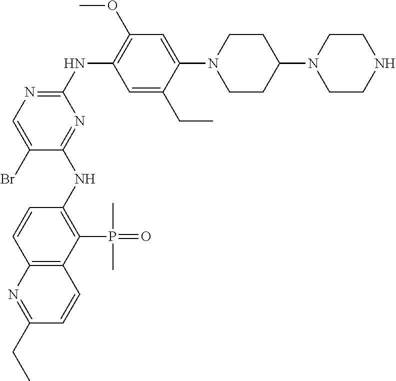 [SMILES: CCc1ccc2c(P(C)(C)=O)c(Nc3nc(Nc4cc(CC)c(N5CCC(N6CCNCC6)CC5)cc4OC)ncc3Br)ccc2n1]

The title compound (2.0 g, 48.8%). was prepared in a manner similar to that in Example 486 step 4 from (6-((5-bromo-2-chloropyrimidin-4-yl)amino)-2-ethylquinolin-5-yl)dimethylphosphine oxide and tert-butyl 4-(1-(4-amino-5-ethoxy-2-ethylphenyl)piperidin-4-yl)piperazine-1-carboxylate. [M+H]⁺=721.5.

### Step 14: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (500 mg, 0.694 mmol) and (R)-2-(4-(2,6-dioxopiperidin-3-yl)-3,5-difluorophenyl)acetaldehyde (222.49 mg, 0.832 mmol) in DCM (8 mL) was stirred in a flask at room temperature for 2 hour. To the mixture was added sodium triacetoxyborohydride (146.34 mg, 0.694 mmol) and the reaction was stirred at room temperature for another 2 h. The resulting mixture was diluted with H₂O (60 mL) and the layers were separated. The aqueous layer was extracted with DCM (3×30 mL). The combined organic layers were washed with brine (50 mL×3), dried over anhydrous Na₂SO₄, filtered and concentrated under reduced pressure. The residue was purified by silica gel column chromatography to afford the crude product (600 mg), which was purified with prep-HPLC chromatography (0.1% FA in water:acetonitrile=90:10˜50:50 gradient elution) to give the product (480 mg, 71.2%). ¹H NMR (500 MHz, DMSO) δ 11.73 (s, 1H), 10.88 (s, 1H), 8.49 (d, J=8.8 Hz, 1H), 8.20 (s, 1H), 8.15 (d, J=12.4 Hz, 1H), 7.94 (s, 1H), 7.80 (d, J=9.4 Hz, 1H), 7.37 (d, J=8.9 Hz, 1H), 7.26 (s, 1H), 6.96 (d, J=10.0 Hz, 2H), 6.68 (s, 1H), 4.13 (dd, J=12.6, 5.0 Hz, 1H), 3.69 (s, 3H), 2.86 (dd, J=15.2, 7.6 Hz, 4H), 2.79-2.65 (m, 4H), 2.59 (t, J=11.3 Hz, 3H), 2.47 (s, 4H), 2.41-2.31 (m, 4H), 2.22 (d, J=4.7 Hz, 3H), 2.05 (s, 2H), 1.92 (d, J=13.3 Hz, 7H), 1.77 (d, J=10.2 Hz, 2H), 1.47 (d, J=8.8 Hz, 2H), 1.25 (t, J=7.6 Hz, 3H), 0.70 (s, 3H). [M+H]⁺=972.7.

### Example 459: (R)-3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione

### Step 1: 1-bromo-2-chloro-4-cyclopropoxy-5-nitrobenzene

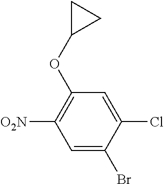 [SMILES: O=[N+]([O-])c1cc(Br)c(Cl)cc1OC1CC1]

To a solution of 1-bromo-2-chloro-4-fluoro-5-nitrobenzene (4 g, 15.7 mmol) in DMSO (50 mL) was added cyclopropanol (912 mg, 15.7 mmol) and K₂CO₃ (4.34 g, 31.4 mmol) at 20° C. Then the mixture was warmed to 70° C. and stirred for 16 hrs. Then the mixture was diluted with EA (200 mL), washed with water (100 mL×2) and brine (100 mL×2). Then the organic layer was dried over anhydrous sodium sulfate, filtered and concentrated in vacuo. The residue was purified by Combi-Flash (silica column, 80 g, PE:EA=10:1) to give the product (3.5 g, 76.2%).

### Step 2: tert-butyl 4-(1-(2-bromo-5-cyclopropoxy-4-nitrophenyl)piperidin-4-yl)piperazine-1-carboxylate

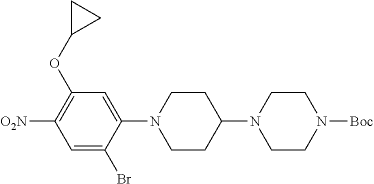 [SMILES: CC(C)(C)OC(=O)N1CCN(C2CCN(c3cc(OC4CC4)c([N+](=O)[O-])cc3Br)CC2)CC1]

To a solution of 1-bromo-2-chloro-4-cyclopropoxy-5-nitrobenzene (3.5 g, 12.0 mmol) in MeCN (50 mL) was added tert-butyl 4-(piperidin-4-yl)piperazine-1-carboxylate (3.56 g, 13.2 mmol) and K₂CO₃ (3.31 g, 24.0 mmol) at 25° C. Then the mixture was stirred at 80° C. for 16 hrs. Then the mixture was cooled to rt and filtered. The solid was washed with EA. Then the filtrate was concentrated in vacuo. The residue was purified by Combi-Flash (silica column, 80 g, DCM:MeOH=30:1) to give tert-butyl 4-(1-(2-bromo-5-cyclopropoxy-4-nitrophenyl)piperidin-4-yl)piperazine-1-carboxylate (4 g, 63.3%). [M+H]⁺=525.3.

### Step 3: tert-butyl 4-(1-(5-cyclopropoxy-4-nitro-2-vinylphenyl)piperidin-4-yl)piperazine-1-carboxylate

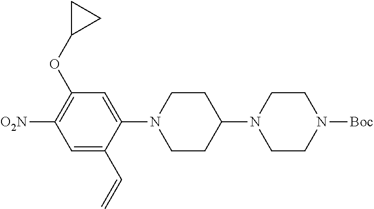 [SMILES: C=Cc1cc([N+](=O)[O-])c(OC2CC2)cc1N1CCC(N2CCN(C(=O)OC(C)(C)C)CC2)CC1]

To a suspension of tert-butyl 4-(1-(2-bromo-5-cyclopropoxy-4-nitrophenyl)piperidin-4-yl)piperazine-1-carboxylate (2 g, 3.8 mmol) and 4,4,5,5-tetramethyl-2-vinyl-1,3,2-dioxaborolane (879 mg, 5.7 mmol) in dioxane (16 mL) and water (4 mL) was added K₂CO₃ (1.57 g, 11.4 mmol) and Pd(dppf)Cl₂ (139 mg, 0.19 mmol). The mixture was warmed to 100° C. and stirred for 16 hrs under nitrogen atmosphere. Then the mixture was cooled to rt and filtered. The filtrate was concentrated in vacuo. The residue was purified by Combi-Flash (silica column, 40 g, DCM:MeOH=15:1) to give tert-butyl 4-(1-(5-cyclopropoxy-4-nitro-2-vinylphenyl)piperidin-4-yl)piperazine-1-carboxylate (1.4 g, 77.9%). [M+H]⁺=473.3.

### Step 4: tert-butyl 4-(1-(4-amino-5-cyclopropoxy-2-ethylphenyl)piperidin-4-yl)piperazine-1-carboxylate

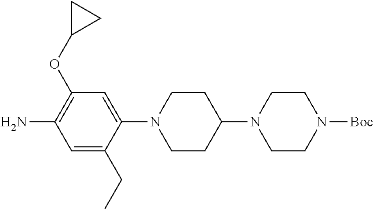 [SMILES: CCc1cc(N)c(OC2CC2)cc1N1CCC(N2CCN(C(=O)OC(C)(C)C)CC2)CC1]

To a suspension of tert-butyl 4-(1-(5-cyclopropoxy-4-nitro-2-vinylphenyl)piperidin-4-yl)piperazine-1-carboxylate (1.4 g, 3.0 mmol) in MeOH (20 mL) was added Pd/C (1 g, 10 wt. %, wet). The mixture was stirred at rt for 16 hrs under hydrogen atmosphere. Then the mixture was filtered and the solid was washed with MeOH. The filtrate was concentrated in vacuo to afford tert-butyl 4-(1-(4-amino-5-cyclopropoxy-2-ethylphenyl)piperidin-4-yl)piperazine-1-carboxylate (1.2 g, 90.0%). [M+H]⁺=445.3.

### Step 5: (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide

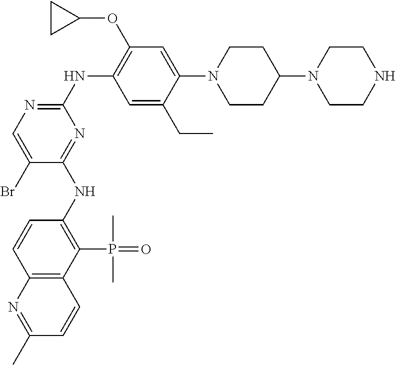 [SMILES: CCc1cc(Nc2ncc(Br)c(Nc3ccc4nc(C)ccc4c3P(C)(C)=O)n2)c(OC2CC2)cc1N1CCC(N2CCNCC2)CC1]

To a solution of (6-((5-bromo-2-chloropyrimidin-4-yl)amino)-2-methylquinolin-5-yl)dimethylphosphine oxide (553 mg, 1.3 mmol) in n-BuOH (10 mL) was added tert-butyl 4-(1-(4-amino-5-cyclopropoxy-2-ethylphenyl)piperidin-4-yl)piperazine-1-carboxylate (600 mg, 1.3 mmol) at 20° C. 4-Methylbenzenesulfonic acid (783 mg, 4.6 mmol) was added to the reaction mixture at 20° C. Then the mixture was stirred at 100° C. for 13 hrs. The mixture was diluted with water (100 mL), adjusted to pH=8 with 5N NaOH solution and then extracted with DCM (150 mL×3). The combined organic layers were washed with brine (150 mL×3), dried over Na₂SO₄, filtered and concentrated in vacuum. The residue was purified by column chromatography (DCM/MeOH (0.5% NH₄OH)=10/1 to 5/1). (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide (500 mg, 52%) was obtained. [M+H]⁺=733.2.

### Step 6: (R)-3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione

The title compound (32 mg, 48%) was prepared in a manner similar to that in Example 484 step 15 from (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide and (R)-2-(4-(2,6-dioxopiperidin-3-yl)-3,5-difluorophenyl)acetaldehyde. ¹H NMR (500 MHz, DMSO) δ 11.74 (s, 1H), 10.95 (s, 1H), 8.58 (d, J=8.5 Hz, 1H), 8.27 (d, J=7.5 Hz, 1H), 8.21 (s, 1H), 7.85-7.84 (m, 2H), 7.43 (d, J=8.5 Hz, 1H), 7.39 (s, 1H), 7.04 (d, J=10.0 Hz, 2H), 6.98 (s, 1H), 4.20 (dd, J=12.5, 5.0 Hz, 1H), 3.81 (dq, J=9.0, 3.0 Hz, 1H), 2.98 (d, J=10.5 Hz, 2H), 2.85-2.76 (m, 4H), 2.75-2.51 (m, 14H), 2.19-2.15 (m, 5H), 1.99-1.95 (m, 9H), 1.69-1.53 (m, 2H), 0.75 (t, J=7.5, 3H), 0.74-0.69 (m, 2H), 0.61-0.56 (m, 2H). [M+H]⁺=984.3.

### Example 485: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (500 mg, 0.694 mmol) and 2-(4-(2,6-dioxopiperidin-3-yl)-3,5-difluorophenyl)acetaldehyde (222.49 mg, 0.832 mmol, the compound was obtained through the similar way with the compound “(R)-2-(4-(2,6-dioxopiperidin-3-yl)-3,5-difluorophenyl)acetaldehyde”) in DCM (8 mL) was stirred in a flask at room temperature for 2 hour. To the mixture was added sodium triacetoxyborohydride (146.34 mg, 0.694 mmol) and the reaction was stirred at room temperature for another 2 h. The resulting mixture was diluted with H₂O (60 mL) and the layers were separated. The aqueous layer was extracted with DCM (3×30 mL). The combined organic layers were washed with brine (50 mL×3), dried over anhydrous Na₂SO₄, filtered and concentrated under reduced pressure. The residue was purified by silica gel column chromatography to afford the crude product (600 mg), which was purified with prep-HPLC chromatography (0.1% FA in water:acetonitrile=90:10˜50:50 gradient elution) to give the product (450 mg, 66.7%). ¹H NMR (500 MHz, DMSO) δ 11.73 (s, 1H), 10.88 (s, 1H), 8.49 (d, J=8.8 Hz, 1H), 8.20 (s, 1H), 8.15 (d, J=12.4 Hz, 1H), 7.94 (s, 1H), 7.80 (d, J=9.4 Hz, 1H), 7.37 (d, J=8.9 Hz, 1H), 7.26 (s, 1H), 6.96 (d, J=10.0 Hz, 2H), 6.68 (s, 1H), 4.13 (dd, J=12.6, 5.0 Hz, 1H), 3.69 (s, 3H), 2.86 (dd, J=15.2, 7.6 Hz, 4H), 2.79-2.65 (m, 4H), 2.59 (t, J=11.3 Hz, 3H), 2.47 (s, 4H), 2.41-2.31 (m, 4H), 2.22 (d, J=4.7 Hz, 3H), 2.05 (s, 2H), 1.92 (d, J=13.3 Hz, 7H), 1.77 (d, J=10.2 Hz, 2H), 1.47 (d, J=8.8 Hz, 2H), 1.25 (t, J=7.6 Hz, 3H), 0.70 (s, 3H). [M+H]⁺=972.7.

### Example 486: (R)-3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

### Step 1: 6-bromo-2-ethyl-8-fluoroquinoline

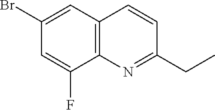

To a stirred mixture of 4-bromo-2-fluoroaniline (10 g, 52.6 mmol) and BTEAC (1 g) in aqueous HCl (6M) was added (E)-pent-2-enal (8.84 g, 105.3 mmol) in toluene (50 mL) dropwise at 100° C. The resulting mixture was stirred for 15 h at 100° C. under nitrogen atmosphere. The reaction was extracted with EA. The aqueous layer was concentrated under reduced pressure. The residue was dissolved in water, and the pH was adjusted to 8-9 with aqueous NaOH (3M). The resulting mixture was extracted with DCM (100 mL×3). The combined organic layers were washed with brine (100 mL×3), dried over anhydrous Na₂SO₄, filtered and concentrated under reduced pressure. The crude product was purified by column chromatography (0-15% EA in PE) to afford product (3.5 g, 26.2%). [M+H]⁺=254.1.

### Step 2: tert-butyl (2-ethyl-8-fluoroquinolin-6-yl)carbamate

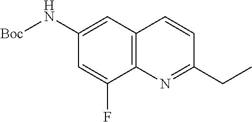 [SMILES: CCc1ccc2cc(NC(=O)OC(C)(C)C)cc(F)c2n1]

A mixture of 6-bromo-2-ethyl-8-fluoroquinoline (3.5 g, 13.8 mmol), BocNH₂ (1.93 g, 16.5 mmol), Pd₂(dba)₃ (631 mg, 0.69 mmol), XantPhos (799 mg, 1.38 mmol), and Cs₂CO₃ (11.2 g, 34.5 mmol) in dioxane (80 mL) was stirred at 100° C. under nitrogen atmosphere for 15 hrs. After cooling to r.t, the reaction mixture was filtered and concentrated in vacuo. The residue was purified by silica gel column (PE:EA=4:1) to afford product (2.8 g, 70%). [M+H]⁺=291.1.

### Step 3: 2-ethyl-8-fluoroquinolin-6-amine

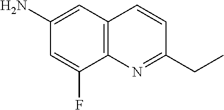

To a stirred solution of tert-butyl (2-ethyl-8-fluoroquinolin-6-yl)carbamate (2.8 g, 9.7 mmol) in DCM (20 mL) was added TFA (10 mL). The reaction mixture was stirred at rt for 2 h and concentrated in vacuum to afford the product (1.8 g, 98.5%) [M+H]⁺=191.1.

### Step 4: 2-ethyl-8-fluoro-5-iodoquinolin-6-amine

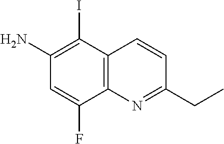

To a solution of 2-ethyl-8-fluoroquinolin-6-amine (1.8 g, 9.5 mmol) in HOAc (20 mL) was added ICl (1.84 g, 11.4 mmol). The mixture was stirred at 20° C. for 3 hrs and then sat. aq. Na₂CO₃ was added to adjust the PH to 8-9. The resulting mixture was extracted with EtOAc (50 mL×3). The combined organic layers were washed with brine (60 mL×3), dried over Na₂SO₄, filtered and concentrated under reduced pressure. 2-Ethyl-8-fluoro-5-iodoquinolin-6-amine (1.7 g, 56.7%) was obtained. [M+H]⁺=317.0.

### Step 5: (6-amino-2-ethyl-8-fluoroquinolin-5-yl)dimethylphosphine oxide

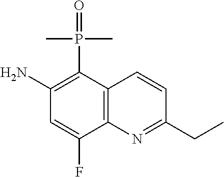 [SMILES: CCc1ccc2c(P(C)(C)=O)c(N)cc(F)c2n1]

A mixture of 2-ethyl-8-fluoro-5-iodoquinolin-6-amine (1.7 g, 5.4 mmol), dimethylphosphineoxide (627 mg, 8 mmol), K₃PO₄ (2.8 g, 13.5 mmol), Pd(OAc)₂ (120 mg, 0.54 mmol) and XantPhos (310 mg, 0.54 mmol) in dioxane (30 mL) was stirred for 3 h at 100° C. under nitrogen atmosphere. After cooling to room temperature, the mixture was concentrated under vacuum, and the residue was purified by column chromatography (0-10% MeOH in DCM) to afford the product (1.2 g, 83.3%). [M+H]⁺=267.1.

### Step 6: (6-((5-bromo-2-chloropyrimidin-4-yl)amino)-2-ethyl-8-fluoroquinolin-5-yl)dimethylphosphine oxide

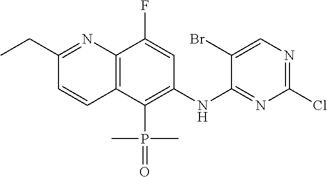 [SMILES: CCc1ccc2c(P(C)(C)=O)c(Nc3nc(Cl)ncc3Br)cc(F)c2n1]

To a solution of (6-amino-2-ethyl-8-fluoroquinolin-5-yl)dimethylphosphine oxide (1.2 g, 4.5 mmol) in THF (30 mL) was added 5-bromo-2,4-dichloropyrimidine (2.6 g, 11.2 mmol). LiHMDS (1 M in THF, 9 mL, 9 mmol) was added to the reaction mixture at 0° C. The mixture was stirred at 20° C. for 3 hrs. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The residue was purified by column chromatography (DCM/MeOH=20/1 to 10/1) to afford product (1.3 g, 63.4%). [M+H]⁺=457.0.

### Step 7: (6-((5-bromo-2-((2-ethoxy-5-ethyl-4-(4-(piperazin-1-yl)piperidin-1-yl)phenyl)amino)pyrimidin-4-yl)amino)-2-ethyl-8-fluoroquinolin-5-yl)dimethylphosphine oxide

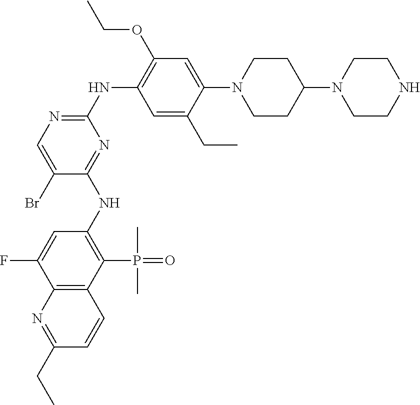 [SMILES: CCOc1cc(N2CCC(N3CCNCC3)CC2)c(CC)cc1Nc1ncc(Br)c(Nc2cc(F)c3nc(CC)ccc3c2P(C)(C)=O)n1]

To a stirred solution of (6-((5-bromo-2-chloropyrimidin-4-yl)amino)-2-ethyl-8-fluoroquinolin-5-yl)dimethylphosphine oxide (800 mg, 1.7 mmol) and tert-butyl 4-(1-(4-amino-5-ethoxy-2-ethylphenyl)piperidin-4-yl)piperazine-1-carboxylate (755 mg, 1.7 mmol) in n-BuOH (40 mL) was added TsOH (877 mg, 5.1 mmol). The resulting mixture was stirred at 95° C. for 16 hours. The reaction mixture was concentrated under vacuum before aqueous NaOH (1M, 10 mL) was added into the mixture. Then the mixture was extracted with DCM (3×20 mL). The combined organic layers were washed with brine (3×20 mL), dried over Na₂SO₄, filtered and concentrated under reduced pressure to afford the crude residue, which was purified with silica gel column chromatography (DCM:MeOH=6:1) to give the title product (790 mg, 60.8%). [M+H]⁺=753.3.

### Step 8: (R)-3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

To a solution of (6-((5-bromo-2-((2-ethoxy-5-ethyl-4-(4-(piperazin-1-yl)piperidin-1-yl)phenyl)amino)pyrimidin-4-yl)amino)-2-ethyl-8-fluoroquinolin-5-yl)dimethylphosphine oxide (50 mg, 0.07 mmol) and (R)-2-(4-(2,6-dioxopiperidin-3-yl)-3,5-difluorophenyl)acetaldehyde (40 mg, 0.15 mmol) in DCM (3 mL) was added STAB (32 mg, 0.15 mmol) at 20° C. The mixture was stirred at 20° C. for 1 hr. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The residue was purified by prep-HPLC (C-18 column chromatography (0.1% FA in water:acetonitrile=90:10˜60:40 gradient elution) to afford the product (24.5 mg, 19.6%). ¹H NMR (500 MHz, DMSO) δ 12.04 (s, 1H), 10.88 (s, 1H), 8.50 (d, J=8.5 Hz, 1H), 8.31-8.11 (m, 2H), 7.97 (s, 1H), 7.47 (d, J=9.0 Hz, 1H), 7.33 (s, 1H), 6.95 (d, J=10.1 Hz, 2H), 6.64 (s, 1H), 4.13 (dd, J=12.6, 4.9 Hz, 1H), 3.94 (q, J=6.9 Hz, 2H), 2.86-2.91 (m, 5H), 2.67-2.78 (m, 3H), 2.45-2.60 (m, 9H), 2.15-2.42 (m, 7H), 2.00-2.10 (m, 1H), 1.93 (d, J=13.3 Hz, 6H), 1.74-1.77 (m, 2H), 1.36-1.46 (m, 2H), 1.25 (t, J=7.6 Hz, 3H), 1.19 (t, J=6.9 Hz, 3H), 0.68 (s, 3H). [M+H]⁺=1004.7.

### Example 488: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

### Step 1: 3-fluoroquinolin-2(1H)-one

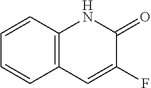 [SMILES: O=c1[nH]c2ccccc2cc1F]

To a solution of quinolin-2(1H)-one (25.0 g, 0.17 mol) in MeCN (30 mL) was added Selectfluor (64.0 g, 0.18 mol) at room temperature. The resulting mixture was stirred at 80° C. overnight. The reaction was concentrated to give the crude residue, which was purified by silica gel column chromatography (DCM:MeOH=100:0˜20:1 gradient elution) to give a mixture (10.4 g, 37%) containing the desired product. [M+H]⁺=164.2.

### Step 2: 3-fluoro-6-nitroquinolin-2(1H)-one

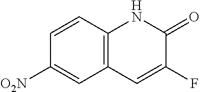 [SMILES: O=c1[nH]c2ccc([N+](=O)[O-])cc2cc1F]

To the mixture from step 1 (10.4 g, 63.4 mmol) in conc. H₂SO₄ (98%, 80 mL) was added conc. HNO₃ (65%, 7.4 g, 76.1 mmol) at 0° C. The resulting mixture was stirred at 0° C. for 1 hour. The reaction was poured into ice water (200 mL) and stirred for 10 mins. The mixture was filtered and the solid was washed with water (50 mL×2), dried under reduced pressure to give the crude product (7.5 g, 56.8%).

¹H NMR (500 MHz, DMSO) δ12.84 (s, 1H), 8.68 (d, J=2.5 Hz, 1H), 8.31 (dd, J=9.1, 2.5 Hz, 1H), 8.09 (d, J=10.7 Hz, 1H), 7.47 (d, J=9.1 Hz, 1H). [M+H]⁺=209.2.

### Step 3: 2-chloro-3-fluoro-6-nitroquinoline

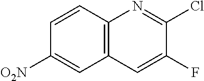 [SMILES: O=[N+]([O-])c1ccc2nc(Cl)c(F)cc2c1]

A solution of 3-fluoro-6-nitroquinolin-2(1H)-one (7.5 g, 35.9 mmol) in POCl₃ (60 mL) was heated at 100° C. overnight. The reaction was concentrated, basified with sat. aq. NaHCO₃, and then extracted with DCM (3×100 mL). The combined organic layers were dried over anhydrous Na₂SO₄, filtered and evaporated under reduced pressure to afford the crude residue, which was purified by silica gel column chromatography (PE:EA=100:0˜10:1 gradient elution) to give the desired product (3.7 g, 32%). [M+H]⁺=227.0.

### Step 4: 3-fluoro-2-methyl-6-nitroquinoline

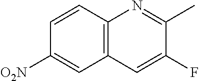

A mixture of 2-chloro-3-fluoro-6-nitroquinoline (3.7 g, 16.3 mmol), 2,4,6-trimethyl-1,3,5,2,4,6-trioxatriborinane (14.0 mL, 48.9 mmol), Pd(dppf)Cl₂ (1.2 g, 1.63 mmol) and K₃PO₄ (6.9 g, 32.6 mmol) in DME (100 mL) and H₂O (20 mL) was stirred in a round bottom flask at 80° C. overnight under nitrogen atmosphere. The mixture was evaporated under reduced pressure to afford the crude product, which was purified with silica gel column chromatography (PE:EA=100:0˜20:1 gradient elution) to give the title product (2.6 g, 77%). [M+H]⁺=207.1.

### Step 5: 3-fluoro-2-methylquinolin-6-amine

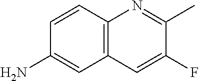

To a solution of 3-fluoro-2-methyl-6-nitroquinoline (2.6 g, 12.5 mmol) in MeOH (50 mL)/DCM (25 mL) was added Pd/C (10 wt. %, wet, 800 mg) at 25° C. under nitrogen atmosphere. The flask was evacuated and backfilled with hydrogen three times. After stirring under hydrogen atmosphere at 25° C. for 5 hours, the mixture was filtered through a pad of Celite and the solid was washed with MeOH (50 mL). The filtrate was concentrated under reduced pressure to afford the desired product (2.2 g, 99%). [M+H]⁺=177.1.

### Step 6: 3-fluoro-5-iodo-2-methylquinolin-6-amine

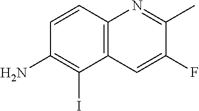

To a solution of 3-fluoro-2-methylquinolin-6-amine (2.2 g, 12.4 mmol) in AcOH (60 mL) was added ICl (16.1 mL, 16.1 mmol) at 20° C. Then the mixture was stirred at 20° C. for 1 hour. Then the mixture was adjusted to pH=8 with sat. aq. NaHCO₃. The resulting mixture was extracted with DCM (3×100 mL). The combined organic layers were washed with brine (3×80 mL), dried over Na₂SO₄, filtered and concentrated under reduced pressure to afford the desired product (3.3 g, 88%). [M+H]⁺=303.0.

### Step 7: (6-amino-3-fluoro-2-methylquinolin-5-yl)dimethylphosphine oxide

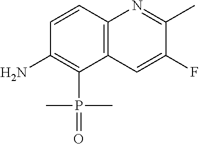 [SMILES: Cc1nc2ccc(N)c(P(C)(C)=O)c2cc1F]

To a solution of 3-fluoro-5-iodo-2-methylquinolin-6-amine (3.3 g, 10.9 mmol) and dimethylphosphine oxide (1.3 g, 16.3 mmol) in dioxane (120 mL) was added K₃PO₄ (6.9 g, 32.7 mmol) at 20° C. Pd(OAc)₂ (244 mg, 1.09 mmol) and Xantphos (1.2 g, 2.18 mmol) were then added to the mixture at the same temperature. The flask was evacuated and backfilled with nitrogen three times, before the mixture was stirred at 100° C. overnight. The mixture was filtered and concentrated under reduced pressure. The residue was purified by silica gel column chromatography (DCM:MeOH=100:0˜10:1 gradient elution) to give the desired product (2.5 g, 91%). [M+H]⁺=253.1.

### Step 8: (6-((5-bromo-2-chloropyrimidin-4-yl)amino)-3-fluoro-2-methylquinolin-5-yl)dimethylphosphine oxide

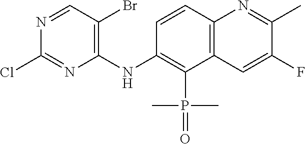 [SMILES: Cc1nc2ccc(Nc3nc(Cl)ncc3Br)c(P(C)(C)=O)c2cc1F]

To a solution of (6-amino-3-fluoro-2-methylquinolin-5-yl)dimethylphosphine oxide (2.5 g, 9.9 mmol) and 5-bromo-2,4-dichloropyrimidine (6.7 g, 29.7 mmol) in n-BuOH (100 mL) was added DIEA (3.8 g, 29.7 mmol) at room temperature. The resulting mixture was stirred at 120° C. overnight. The reaction was concentrated to afford the crude residue, which was purified by silica gel column chromatography (DCM:MeOH=100:0˜15:1 gradient elution) to give the desired product (2.9 g, 66%). [M+H]⁺=443.1.

### Step 9: (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide

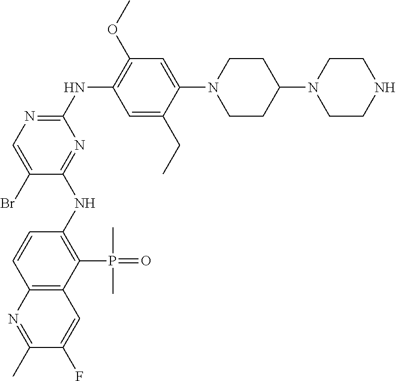 [SMILES: CCc1cc(Nc2ncc(Br)c(Nc3ccc4nc(C)c(F)cc4c3P(C)(C)=O)n2)c(OC)cc1N1CCC(N2CCNCC2)CC1]

To a solution of (6-((5-bromo-2-chloropyrimidin-4-yl)amino)-3-fluoro-2-methylquinolin-5-yl)dimethylphosphine oxide (1.7 g, 3.8 mmol) and tert-butyl 4-(1-(4-amino-2-ethyl-5-methoxyphenyl)piperidin-4-yl)piperazine-1-carboxylate (1.6 g, 3.8 mmol) in n-BuOH (100 mL) was added Ts-OH (2.0 g, 11.4 mmol) at room temperature. The resulting mixture was stirred at 100° C. overnight. The reaction was concentrated and basified with 0.5N NaOH (50 mL). The resulting mixture was extracted with DCM (3×80 mL). The combined organic layers were dried over anhydrous Na₂SO₄, filtered and evaporated in vacuum to afford the crude residue, which was purified by silica gel column chromatography (DCM:MeOH=100:0˜5:1 gradient elution) to give the desired product (960 mg, 35%). [M+H]⁺=725.2.

### Step 10: methyl (R)-1-(4-bromo-3,5-difluorophenyl)pyrrolidine-3-carboxylate

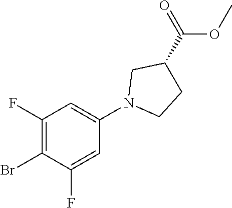 [SMILES: COC(=O)[C@@H]1CCN(c2cc(F)c(Br)c(F)c2)C1]

To the solution of 2-bromo-1,3-difluoro-5-iodobenzene (15 g, 47 mmol), methyl (R)-pyrrolidine-3-carboxylate hydrochloride (8.56 g, 51.7 mmol) and K₃PO₄ (20 g, 94 mmol) in 250 mL DMSO, were added CuI (893 mg, 4.7 mmol) and L-proline (1 g, 9.4 mmol). The mixture was stirred at 80° C. for 16 hours. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (PE:EA=50:1-30:1) to afford the product (4.9 g, 32.5%). [M+H]⁺=320.1.

### Step 11: methyl (R)-1-(4-(2,6-bis(benzyloxy)pyridin-3-yl)-3,5-difluorophenyl)pyrrolidine-3-carboxylate

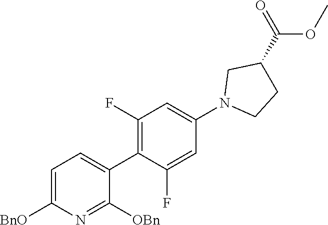 [SMILES: COC(=O)[C@@H]1CCN(c2cc(F)c(-c3ccc(OCc4ccccc4)nc3OCc3ccccc3)c(F)c2)C1]

To the solution of methyl (R)-1-(4-bromo-3,5-difluorophenyl)pyrrolidine-3-carboxylate (4.9 g, 15.3 mmol), 2,6-bis(benzyloxy)-3-(4,4,5,5-tetramethyl-1,3,2-dioxaborolan-2-yl)pyridine (6.7 g, 16 mmol) and CsF (4.6 g, 30.6 mmol) in 150 mL DMF and 15 mL water, was added Pd(dtbpf)Cl₂ (498 mg, 0.8 mmol). The mixture was stirred at 80° C. for 4 hours. After LCMS showed the reaction was completed, the mixture was diluted with water and extracted with EtOAc. The organic layer was washed with brine, dried over anhydrous Na₂SO₄, filtered and concentrated under reduced pressure. The resulting mixture was purified by combi-flash (EA:PE=0-12%) to afford the product (7.9 g, 97.4%). [M+H]⁺=531.30.

### Step 12: (R)-1-(4-(2,6-bis(benzyloxy)pyridin-3-yl)-3,5-difluorophenyl)pyrrolidine-3-carboxylic acid

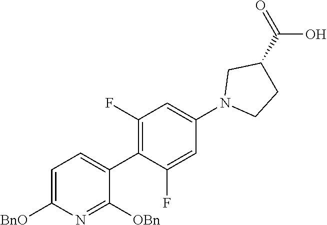 [SMILES: O=C(O)[C@@H]1CCN(c2cc(F)c(-c3ccc(OCc4ccccc4)nc3OCc3ccccc3)c(F)c2)C1]

To the solution of methyl (R)-1-(4-(2,6-bis(benzyloxy)pyridin-3-yl)-3,5-difluorophenyl)pyrrolidine-3-carboxylate (7.9 g, 14.9 mmol) in 100 mL THF and 20 mL water, LiOH (394 mg, 16.4 mmol) in 10 mL water was added dropwise at room temperature. The mixture was stirred at room temperature for 15 minutes. After TLC showed the reaction was completed, the mixture was concentrated in vacuum at room temperature. The residue was diluted with water and adjust pH<5 with 1 N HCl. The liquid was extracted with EtOAc. The organic phase was dried over anhydrous Na₂SO₄, filtered and concentrated in vacuum to afford the product (7.4 g, 96.0%). [M+H]⁺=517.1.

### Step 13: (R)-1-(4-((R)-2,6-dioxopiperidin-3-yl)-3,5-difluorophenyl)pyrrolidine-3-carboxylic acid

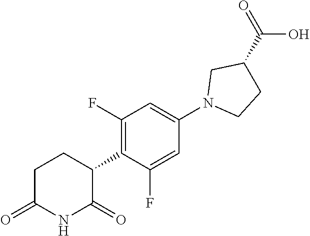 [SMILES: O=C1CC[C@H](c2c(F)cc(N3CC[C@@H](C(=O)O)C3)cc2F)C(=O)N1]

To the solution of (R)-1-(4-(2,6-bis(benzyloxy)pyridin-3-yl)-3,5-difluorophenyl)pyrrolidine-3-carboxylic acid (7.4 g, 14.3 mmol) in 50 mL DCM and 250 mL iPrOH, Pd/C (7.4 g, 10 wt. %, wet) was added. The mixture was stirred at 40° C. for 16 hours under hydrogen atmosphere (balloon). After LCMS showed the reaction was completed, the mixture was cooled to room temperature and filtered by celite directly. The filtrate was concentrated in vacuum to afford the crude product which was purified by SFC (IH (3*25 cm, 5 um), 13% EtOH/87% CO₂, 100 bar, 100 ml/min) and the title compound corresponded to peak A @ 0.655 min/254 nm (1.6 g, 34%). [M+H]⁺=339.2.

### Step 14: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

To a solution of (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide (450 mg, 0.62 mmol), (R)-1-(4-((R)-2,6-dioxopiperidin-3-yl)-3,5-difluorophenyl)pyrrolidine-3-carboxylic acid (230 mg, 0.68 mmol) and DIEA (162 mg, 1.24 mmol) in DCM (30 mL) was added T₃P (788 mg, 1.24 mmol) dropwise at room temperature. The resulting mixture was stirred at room temperature for 1 hour. The reaction was quenched with water (50 mL) and extracted with DCM (2×60 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC (C-18 column chromatography (0.1% FA in water:acetonitrile=90:10˜60:40 gradient elution) to give the desired product (302 mg, 47%). ¹H NMR (500 MHz, DMSO) δ11.30 (s, 1H), 10.84 (s, 1H), 8.63 (d, J=12.6 Hz, 1H), 8.23 (s, 2H), 7.95 (d, J=9.2 Hz, 2H), 7.33 (s, 1H), 6.72 (s, 1H), 6.23 (d, J=12.1 Hz, 2H), 4.02 (dd, J=12.7, 5.0 Hz, 1H), 3.75 (s, 3H), 3.60-3.42 (m, 6H), 3.35 (s, 2H), 3.26 (s, 3H), 2.92 (d, J=10.7 Hz, 211), 2.83-2.73 (m, 111), 2.64 (d, J=2.4 Hz, 5H), 2.56 (s, 3H), 2.37 (d, J=3.6 Hz, 1H), 2.25-2.05 (m, 5H), 1.95 (d, J=13.4 Hz, 7H), 1.82 (d, J=11.0 Hz, 2H), 1.62-1.51 (m, 2H), 0.71 (s, 3H); [M+H]⁺=1045.5.

### Example 487: (R)-3-(4-((R)-4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

### Step 1: methyl (R)-4,4-dimethylpyrrolidine-3-carboxylate

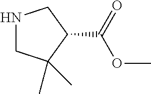

To a stirred solution of (R)-4,4-dimethylpyrrolidine-3-carboxylic acid (2 g, 13.96 mmol) in MeOH (30 mL) was added SOCl₂ (1.66 g, 13.96 mmol) dropwise at 0° C. under nitrogen atmosphere. The resulting mixture was stirred for 2 hrs at 60° C. temperature. The resulting mixture was concentrated under reduced pressure to afford the product (2.1 g, 95.8%) which was used for next step without further purification. [M+H]⁺=158.1

### Step 2: methyl (R)-1-(4-(2,6-bis(benzyloxy)pyridin-3-yl)-3,5-difluorophenyl)-4,4-dimethylpyrrolidine-3-carboxylate

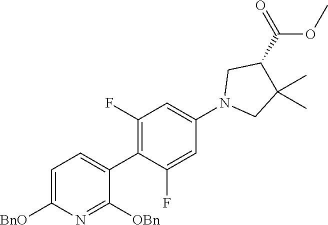 [SMILES: COC(=O)[C@H]1CN(c2cc(F)c(-c3ccc(OCc4ccccc4)nc3OCc3ccccc3)c(F)c2)CC1(C)C]

To a stirred solution of 2,6-bis(benzyloxy)-3-(4-bromo-2,6-difluorophenyl)pyridine (9.25 g, 20.25 mmol) and methyl (R)-4,4-dimethylpyrrolidine-3-carboxylate (2.1 g, 13.35 mmol) in dioxane (50 mL) were added Cs₂CO₃ (10.95 g, 33.37 mmol), Xantphos (1.54 g, 2.67 mmol) and Pd₂(dba)₃ (1.22 g, 1.35 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (500 mL), washed with water (3×200 mL) and brine (200 mL). The organic layer was dried over anhydrous Na₂SO₄, filtered and concentrated under reduced pressure. The residue was purified by silica gel column chromatography, eluted with PE/EtOAc (5:1) to afford the product (4.5 g, 60.8%); [M+H]⁺=559.6

### Step 3: (R)-1-(4-(2,6-bis(benzyloxylpyridin-3-yl)-3,5-difluorophenyl)-4,4-dimethylpyrrolidine-3-carboxylic acid

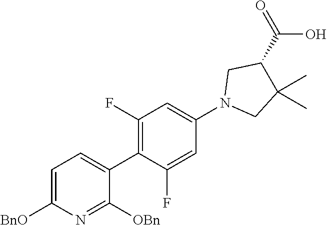 [SMILES: CC1(C)CN(c2cc(F)c(-c3ccc(OCc4ccccc4)nc3OCc3ccccc3)c(F)c2)C[C@@H]1C(=O)O]

To a solution of methyl (R)-1-(4-(2,6-bis(benzyloxy)pyridin-3-yl)-3,5-difluorophenyl)-4,4-dimethylpyrrolidine-3-carboxylate (4.5 g, 8.05 mmol) in THF (40 mL) and H₂O (10 mL) was added lithium hydroxide hydrate (337.9 mg, 8.05 mmol) at 25° C. The resulting mixture was stirred at 25° C. for 5 h. The reaction was quenched with HCl (1 N) at 0° C. until pH=6 and the resulting mixture was extracted with EA (2×40 mL). The combined organic layers were washed with brine, dried over anhydrous Na₂SO₄, filtered and concentrated under vacuum to afford the crude product (4.05 g, 92.46%), which was used for next step without further purification. [M+H]⁺=545.6

### Step 4: (R)-1-(4-((R)-2,6-dioxopiperidin-3-yl)-3,5-difluorophenyl)-4,4-dimethylpyrrolidine-3-carboxylic acid

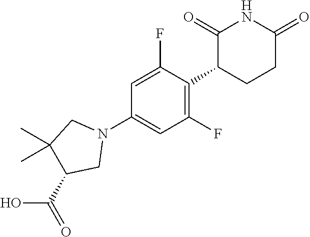 [SMILES: CC1(C)CN(c2cc(F)c([C@H]3CCC(=O)NC3=O)c(F)c2)C[C@@H]1C(=O)O]

(R)-1-(4-(2,6-bis(benzyloxy)pyridin-3-yl)-3,5-difluorophenyl)-4,4-dimethylpyrrolidine-3-carboxylic acid (4.5 g, 8.25 mmol) was dissolved in DCM (30 mL) and iPr-OH (30 mL). Pd/C (1 g, 10 wt. %, wet) was added to the solution in one portion. The resulting mixture was stirred under hydrogen atmosphere (1 atm) at room temperature overnight. The solid was filtered off and the filtrate was concentrated to give the crude product. The crude was triturated with MTBE to give the desired product which was purified by HPLC (IF (2*25 cm, 5 um), 60% MtBE/40% MeOH:DCM=1:1, 80 bar, 20 ml/min) and corresponded to peak A @ 1.216 min/254 nm (1.13 g, 25%). [M+H]⁺=367.4.

### Step 5: (R)-3-(4-((R)-4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.0708 mmol), (R)-1-(4-((R)-2,6-dioxopiperidin-3-yl)-3,5-difluorophenyl)-4,4-dimethylpyrrolidine-3-carboxylic acid (28.5 mg, 0.0779 mmol), T3P (67.45 mg, 0.212 mmol) and DIEA (27.39 mg, 0.212 mmol) in DCM (4 mL) was stirred in a flask at room temperature for 1 hours. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The crude (30 mg) product was purified with prep-HPLC chromatography (0.1% FA in water:acetonitrile=90:10˜50:50 gradient elution) to give the product (30 mg, 40.21%). ¹H NMR (500 MHz, DMSO) δ 11.76 (s, 1H), 10.84 (s, 1H), 8.56 (d, J=8.9 Hz, 1H), 8.30 (s, 1H), 8.21 (s, 1H), 7.98 (s, 1H), 7.87 (d, J=9.3 Hz, 1H), 7.42 (d, J=8.9 Hz, 1H), 7.38 (s, 1H), 6.74 (s, 1H), 6.15 (d, J=12.2 Hz, 2H), 4.01 (dd, J=12.4, 5.0 Hz, 1H), 3.76 (s, 3H), 3.46-3.41 (m, 8H), 3.09 (dd, J=18.7, 9.1 Hz, 2H), 2.95 (d, J=10.7 Hz, 2H), 2.84-2.72 (m, 1H), 2.65 (s, 5H), 2.54 (s, 2H), 2.41-2.24 (m, 3H), 2.08 (dd, J=23.4, 13.7 Hz, 1H), 1.98 (d, J=13.3 Hz, 8H), 1.83 (d, J=11.2 Hz, 2H), 1.57 (d, J=11.0 Hz, 2H), 1.23 (s, 1H), 1.18 (s, 3H), 0.99 (s, 3H), 0.77 (s, 3H). [M+H]⁺=1055.4.

### Example 473: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (75 mg, 0.10 mmol) and 2-(3-(2,6-dioxopiperidin-3-yl)-2-oxo-2,3-dihydrobenzo[d]oxazol-7-yl)acetaldehyde (35 mg, 0.12 mmol) in DCM (8 mL) was stirred in a flask at room temperature for 10 min. To the mixture was added sodium triacetoxyborohydride (65 mg, 0.31 mmol) and the reaction was stirred at room temperature for another 0.5 h. Then the mixture was concentrated under vacuum to afford the crude product, which was purified with prep-HPLC chromatography (0.10% FA in water:acetonitrile=90:10˜50:50 gradient elution) to give the product (31 mg, 30.0%). ¹H NMR (500 MHz, DMSO) δ 11.72 (s, 1H), 11.21 (s, 1H), 8.59 (d, J=9.0 Hz, 1H), 8.24 (s, 1H), 8.22 (s, 1H), 7.91 (s, 1H), 7.88 (d, J=9.3 Hz, 1H), 7.45 (d, J=8.9 Hz, 1H), 7.38 (s, 1H), 7.13-7.09 (m, 3H), 6.71 (s, 1H), 5.36 (dd, J=12.9, 5.3 Hz, 1H), 4.00 (q, J=7.0 Hz, 2H), 3.29-3.21 (m, 4H), 2.93-2.87 (m, 7H), 2.74-2.52 (m, 10H), 2.28-2.25 (m, 3H), 2.19-2.12 (m, 1H), 1.98 (d, J=13.3 Hz, 6H), 1.83 (d, J=11.7 Hz, 2H), 1.53 (dd, J=20.3, 11.3 Hz, 2H), 1.32 (t, J=7.6 Hz, 3H), 1.25 (t, J=6.9 Hz, 3H), 0.71 (s, 3H). [M+H]⁺=1007.7

### Example 460: (R)-3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

### Step 1: 2-ethyl-6-nitroquinazoline

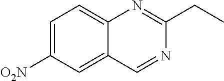

To a solution of 2-fluoro-5-nitrobenzaldehyde (10.0 g, 59.17 mmol) in MeCN (150 mL) was added propionimidamide hydrochloride (9.59 g, 88.75 mmol) and K₂CO₃ (20.4 g, 147.93 mmol) at room temperature. The resulting mixture was stirred at 80° C. overnight. The reaction was concentrated to give the crude residue, which was purified by silica gel column chromatography (DCM:MeOH=100:0˜98:2 gradient elution) to give a mixture (3.6 g, 30%) containing the desired product. [M+H]⁺=204.2.

### Step 2: 2-ethylquinazolin-6-amine

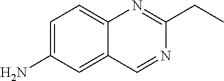

To a solution of 2-ethyl-6-nitroquinazoline (3.6 g, 17.73 mmol) in THF (100 mL)/H₂O (20 mL) was added Fe (4.96 g, 88.67 mmol) and NH₄Cl (4.7 g, 88.67 mmol) at 25° C. Then the mixture was stirred at 25° C. overnight. The mixture was filtered through a pad of Celite and washed with EA (150 mL) and H₂O (60 mL). The filtrate was separated and the organic layer was concentrated to give the crude residue, which was purified by silica gel column chromatography (DCM:MeOH=100:0˜20:1 gradient elution) to give a mixture (1.8 g, 58%) containing the desired product. [M+H]⁺=174.2.

### Step 3: 2-ethyl-5-iodoquinazolin-6-amine

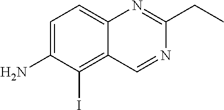

To a solution of 2-ethylquinazolin-6-amine (1.8 g, 10.4 mmol) in AcOH (30 mL) was added ICl (15.6 mL, 15.6 mmol) at 20° C. Then the mixture was stirred at 20° C. for 3 hours. Then the mixture was adjusted to pH=8 with sat. aq. NaHCO₃ and extracted with DCM (2×100 mL). The organic phase was washed with brine (80 mL), dried over Na₂SO₄, filtered and concentrated in vacuum to afford the desired product (2.6 g, 84%). [M+H]⁺=300.1.

### Step 4: (6-amino-2-ethylquinazolin-5-yl)dimethylphosphine oxide

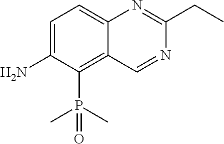 [SMILES: CCc1ncc2c(P(C)(C)=O)c(N)ccc2n1]

To a solution of 2-ethyl-5-iodoquinazolin-6-amine (2.6 g, 8.69 mmol) and dimethylphosphine oxide (1.36 g, 17.39 mmol) in dioxane (100 mL) was added K₃PO₄ (4.6 g, 21.73 mmol) at 20° C. Pd(OAc)₂ (390 mg, 1.74 mmol) and Xantphos (1.0 g, 1.74 mmol) were added to the mixture at 20° C. The suspension was degassed under vacuum and purged with N₂ three times. Then the mixture was stirred at 100° C. overnight. The mixture was filtered and concentrated in vacuum. The residue was purified by silica gel column chromatography (DCM:MeOH=100:0˜10:1 gradient elution) to give the desired product (2.1 g, 97%). [M+H]⁺=250.1.

### Step 5: (6-((5-bromo-2-chloropyrimidin-4-yl)amino)-2-ethylquinazolin-5-yl)dimethylphosphine oxide

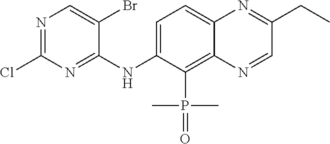 [SMILES: CCc1cnc2c(P(C)(C)=O)c(Nc3nc(Cl)ncc3Br)ccc2n1]

To a solution of (6-amino-2-ethylquinazolin-5-yl)dimethylphosphine oxide (2.1 g, 8.4 mmol) and 5-bromo-2,4-dichloropyrimidine (5.7 g, 25.2 mmol) in n-BuOH (90 mL) was added DIEA (3.3 g, 25.2 mmol) at room temperature. The resulting mixture was stirred at 120° C. overnight. The reaction was concentrated to afford the crude residue, which was purified by silica gel column chromatography (DCM:MeOH=100:0˜15:1 gradient elution) to give the desired product (2.3 g, 62%).

¹H NMR (500 MHz, DMSO) δ 12.44 (s, 1H), 9.83 (s, 1H), 8.59 (s, 1H), 8.56 (dd, J=9.3, 3.8 Hz, 1H), 8.12 (d, J=9.3 Hz, 1H), 3.07 (q, J=7.6 Hz, 2H), 2.12-2.08 (m, 6H), 1.38 (t, J=7.6 Hz, 3H). [M+H]⁺=440.1.

### Step 6: 4-ethoxy-1-ethyl-2-fluorobenzene

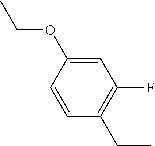

To a solution of 4-ethyl-3-fluorophenol (35 g, 0.25 mol) in DMF (200 mL) was added K₂CO₃ (69 g, 0.5 mol), and EtI (50.7 g, 0.32 mol). The mixture was stirred at 20-30° C. for 18 hours. The reaction was quenched by H₂O (200 mL) and extracted with EA (150 mL×2). The combined organic phases were washed with brine (300 mL×3), dried over anhydrous Na₂SO₄, filtered and concentrated in vacuum. The residue was purified by column chromatography with pure PE to give product. (35 g, 83.3%). [M+H]⁺=168.2.

### Step 7: 1-ethoxy-4-ethyl-5-fluoro-2-nitrobenzene

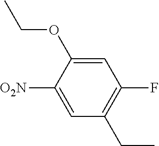

To a solution of 4-ethoxy-1-ethyl-2-fluorobenzene (35 g, 0.2 mol) in Ac₂O (100 mL) was added HNO₃ (23.4 g, 0.26 mol) dropwise at 0° C. The mixture was stirred at r.t. for 2 hs. The reaction was then quenched with Na₂CO₃ solution (500 mL). The layers were separated and the organic layer was concentrated to afford the product (25 g, 58.7%) which was used in the next step without further purification. ¹H NMR (500 MHz, DMSO) δ 7.90 (d, J=8.0 Hz, 1H), 7.26 (d, J=12.0 Hz, 1H), 4.2 (q, J=7.0 Hz, 2H), 2.60 (q, J=7.5 Hz, 2H), 1.33 (t, J=7.0 Hz, 3H), 1.15 (t, J=7.5 Hz, 3H).

### Step 8: tert-butyl 4-(1-(5-ethoxy-2-ethyl-4-nitrophenyl)piperidin-4-yl)piperazine-1-carboxylate

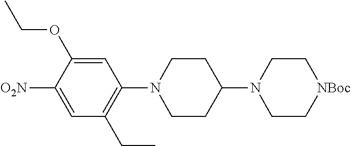 [SMILES: CCOc1cc(N2CCC(N3CCN(C(=O)OC(C)(C)C)CC3)CC2)c(CC)cc1[N+](=O)[O-]]

To a solution of 1-ethoxy-4-ethyl-5-fluoro-2-nitrobenzene (20 g, 94 mmol) in DMF (300 mL) was added tert-butyl 4-(piperidin-4-yl)piperazine-1-carboxylate (30 g, 112 mmol), and K₂CO₃ (32 g, 235 mmol). The mixture was stirred at 120° C. for 28 hours. The mixture was poured into ice water. The product (20 g, 46.1%) was isolated by filtration, which was used in the next step without further purification. ¹H NMR (500 MHz, DMSO) δ 7.74 (s, 1H), 6.73 (s, 1H), 4.19 (q, J=7.0 Hz, 2H), 3.30 (m, 4H), 3.23 (d, J=11.0 Hz, 2H), 2.71 (t, J=11.5 Hz, 2H), 2.57 (q, J=7.5 Hz, 2H), 2.47 (br s, 4H), 2.39 (t, J=11.0 Hz, 1H), 1.84 (d, J=11.5 Hz, 2H), 1.58 (q, J=10.5 Hz, 2H), 1.39 (s, 9H), 1.34 (t, J=7.5 Hz, 3H), 1.19 (t, J=7.5 Hz, 3H).

### Step 9: tert-butyl 4-(1-(4-amino-5-ethoxy-2-ethylphenyl)piperidin-4-yl)piperazine-1-carboxylate

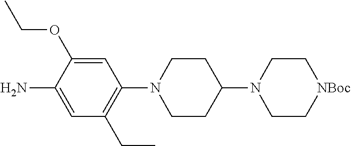 [SMILES: CCOc1cc(N2CCC(N3CCN(C(=O)OC(C)(C)C)CC3)CC2)c(CC)cc1N]

To a solution of tert-butyl 4-(1-(5-ethoxy-2-ethyl-4-nitrophenyl)piperidin-4-yl)piperazine-1-carboxylate (20 g, 94 mmol) in THF (150 mL) was added Pd/C (2 g, 10 wt. %, wet). The mixture was stirred at r.t. under hydrogen atmosphere (1 atm) for 48 h. The solid was filtered off. The filtrate was concentrated for next step directly without further purification. [M+H]⁺=433.4.

### Step 10: (6-((5-bromo-2-((2-ethoxy-5-ethyl-4-(4-(piperazin-1-yl)piperidin-1-yl)phenyl)amino)pyrimidin-4-yl)amino)-2-ethylquinazolin-5-yl)dimethylphosphine oxide

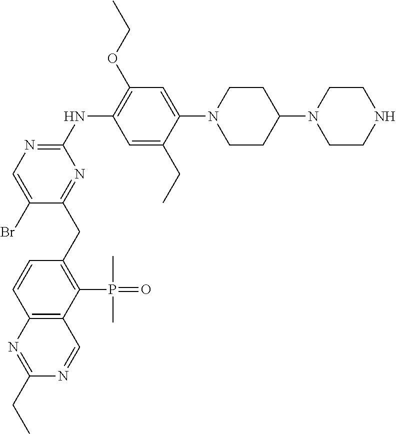 [SMILES: CCOc1cc(N2CCC(N3CCNCC3)CC2)c(CC)cc1Nc1ncc(Br)c(Cc2ccc3nc(CC)ncc3c2P(C)(C)=O)n1]

To a solution of (6-((5-bromo-2-chloropyrimidin-4-yl)amino)-2-ethylquinazolin-5-yl)dimethylphosphine oxide (1.0 g, 2.27 mmol) and tert-butyl 4-(1-(4-amino-5-ethoxy-2-ethylphenyl)piperidin-4-yl)piperazine-1-carboxylate (1.18 g, 2.73 mmol) in n-BuOH (30 mL) was added Ts-OH (1.17 g, 6.81 mmol) at room temperature. The resulting mixture was stirred at 100° C. overnight.

The reaction was concentrated and basified with 0.5N NaOH (40 mL), then extracted with DCM (3×80 mL). The combined organic layers were dried over anhydrous Na₂SO₄, filtered and evaporated in vacuum to afford the crude residue, which was purified by silica gel column chromatography (DCM:MeOH=100:0˜5:1 gradient elution) to give the desired product (720 mg, 43%). [M+H]⁺=736.2.

### Step 11: (R)-3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

To a solution of (6-((5-bromo-2-((2-ethoxy-5-ethyl-4-(4-(piperazin-1-yl)piperidin-1-yl)phenyl)amino)pyrimidin-4-yl)amino)-2-ethylquinazolin-5-yl)dimethylphosphine oxide (60 mg, 0.082 mmol) and (R)-2-(4-(2,6-dioxopiperidin-3-yl)-3,5-difluorophenyl)acetaldehyde (33 mg, 0.123 mmol) in DCM (5 mL) was added sodium triacetoxyborohydride (60 mg, 0.28 mmol) at room temperature. The resulting mixture was stirred at room temperature for 1 hour. The reaction was quenched with water (10 mL) and extracted with DCM (2×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC to give the desired product (13.54 mg, 16%).

¹H NMR (500 MHz, DMSO) δ 11.52 (s, 1H), 10.95 (s, 1H), 9.91 (s, 1H), 8.42 (s, 1H), 8.25 (d, J=19.9 Hz, 1H), 7.94 (s, 1H), 7.85 (d, J=9.3 Hz, 1H), 7.33 (s, 1H), 7.03 (d, J=10.2 Hz, 2H), 6.70 (s, 1H), 4.20 (dd, J=12.9, 5.2 Hz, 1H), 3.99 (q, J=6.9 Hz, 2H), 3.05 (q, J=7.5 Hz, 2H), 2.91 (d, J=10.3 Hz, 2H), 2.81-2.74 (m, 3H), 2.65-2.57 (m, 3H), 2.54-2.49 (m, 7H), 2.46 (s, 3H), 2.27-2.21 (m, 3H), 2.13 (d, J=9.6 Hz, 1H), 2.02 (d, J=13.4 Hz, 7H), 1.82 (d, J=10.9 Hz, 2H), 1.52 (d, J=9.1 Hz, 2H), 1.38 (t, J=7.6 Hz, 3H), 1.25 (t, J=6.9 Hz, 3H), 0.68 (s, 3H); [M+H]⁺=987.7.

### Example 489: (R)-3-(4-((R)-3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (1.15 g, 1.60 mmol), (R)-1-(4-((R)-2,6-dioxopiperidin-3-yl)-3,5-difluorophenyl)pyrrolidine-3-carboxylic acid (595 mg, 1.76 mmol), DIEA (411 mg, 3.19 mmol) and T₃P (763 mg, 2.4 mmol) in DCM (8 mL) was stirred in a flask at room temperature for 0.5 h. Then the mixture was evaporated in vacuum to afford the crude product, which was purified with prep-HPLC chromatography (0.1% FA in water:acetonitrile=90:10˜50:50 gradient elution) to give the product (800 mg, 48.3%).

¹H NMR (500 MHz, DMSO) δ 11.80 (s, 1H), 10.84 (s, 1H), 8.56 (d, J=8.9 Hz, 1H), 8.27 (s, 1H), 8.21 (s, 1H), 8.00 (s, 1H), 7.88 (d, J=9.1 Hz, 1H), 7.44 (d, J=8.9 Hz, 1H), 7.34 (s, 1H), 6.75 (s, 1H), 6.23 (d, J=12.2 Hz, 2H), 4.02 (dd, J=12.3, 4.7 Hz, 1H), 3.76 (s, 3H), 3.65-3.41 (m, 7H), 3.31-3.23 (m, 4H), 3.01-2.88 (m, 4H), 2.84-2.74 (m, 1H), 2.68 (t, J=11.1 Hz, 2H), 2.59-2.55 (m, 3H), 2.39-2.35 (m, 1H), 2.31-2.28 (m, 2H), 2.19-2.06 (m, 3H), 1.98 (d, J=13.3 Hz, 6H), 1.96-1.91 (m, 1H), 1.84 (d, J=10.1 Hz, 2H), 1.57 (d, J=9.9 Hz, 2H), 1.32 (t, J=7.5 Hz, 3H), 0.77 (s, 3H). [M+H]⁺=1041.7

### Example 750: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

The title compound was prepared in a procedure similar to that in Example 489. ¹H NMR (500 MHz, DMSO) δ 11.76 (s, 1H), 10.84 (s, 1H), 8.56 (d, J=8.9 Hz, 1H), 8.31 (d, J=5.4 Hz, 1H), 8.21 (s, 1H), 7.98 (s, 1H), 7.87 (d, J=9.2 Hz, 1H), 7.42 (d, J=8.9 Hz, 1H), 7.38 (s, 1H), 6.74 (s, 1H), 6.23 (d, J=12.2 Hz, 2H), 4.02 (dd, J=12.5, 4.9 Hz, 1H), 3.76 (s, 3H), 3.59-3.42 (m, 6H), 3.36-3.32 (m, 1H), 3.31-3.22 (m, 2H), 2.95 (d, J=10.6 Hz, 2H), 2.83-2.73 (m, 1H), 2.71-2.62 (m, 5H), 2.61-2.51 (m, 3H), 2.48-2.44 (m, 2H), 2.42-2.35 (m, 1H), 2.35-2.25 (m, 2H), 2.20-2.04 (m, 3H), 2.02-1.91 (m, 7H), 1.84 (d, J=10.2 Hz, 2H), 1.65-1.51 (m, 2H), 0.77 (s, 3H). [M+H]⁺=1027.7.

### Example 491 3-(6-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (72 mg, 0.10 mmol) and 2-(3-(2,6-dioxopiperidin-3-yl)-2-oxo-2,3-dihydrobenzo[d]oxazol-6-yl)acetaldehyde (35 mg, 0.12 mmol, the compound was obtained through the similar way with example 492) in DCM (8 mL) was stirred in a flask at room temperature for 10 min. To the mixture was added NaBH₃CN (65 mg, 0.31 mmol) and the reaction was stirred at room temperature for another 0.5 h. Then the mixture was evaporated in vacuum to afford the crude product, which was purified with prep-HPLC chromatography (0.1% FA in water:acetonitrile=90:10˜50:50 gradient elution) to give the product (23 mg, 23.3%). ¹H NMR (500 MHz, DMSO) δ 11.77 (s, 1H), 11.21 (s, 1H), 8.58 (d, J=8.9 Hz, 1H), 8.27 (s, 1H), 8.21 (s, 1H), 8.00 (s, 1H), 7.88 (d, J=9.2 Hz, 1H), 7.45 (d, J=8.9 Hz, 1H), 7.36 (s, 1H), 7.33 (s, 1H), 7.18 (d, J=8.1 Hz, 1H), 7.10 (d, J=7.9 Hz, 1H), 6.75 (s, 1H), 5.35 (dd, J=12.9, 5.3 Hz, 1H), 3.76 (s, 3H), 3.31-3.20 (m, 6H), 2.93-2.88 (m, 9H), 2.76-2.56 (m, 8H), 2.31-2.28 (m, 2H), 2.19-2.11 (m, 1H), 1.98-1.95 (m, 7H), 1.61 (s, 2H), 1.32 (t, J=7.6 Hz, 3H), 0.76 (s, 3H). [M+H]⁺=993.7.

### Example 492: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

### Step 1: 6-bromo-5-fluorobenzo[d]oxazol-2(3H)-one

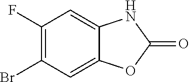 [SMILES: O=c1[nH]c2cc(F)c(Br)cc2o1]

A mixture of 2-amino-5-bromo-4-fluorophenol (3 g, 14.63 mmol), CDI (2.84 g, 17.6 mmol) in THF (50 mL) was stirred at 80° C. for 3 hrs. After cooling to rt, the reaction mixture was concentrated in vacuo. The residue was dissolved in EA (60 mL), which was washed with water (30 mL) and brine (30 mL), dried over Na₂SO₄, filtered and concentrated in vacuum to afford 6-bromo-5-fluorobenzo[d]oxazol-2(3H)-one (3.2 g, 94.1%). [M+H]⁺=231.9.

### Step 2: 3-(2,6-bis(benzyloxy)pyridin-3-yl)-6-bromo-5-fluorobenzo[d]oxazol-2(3H)-one

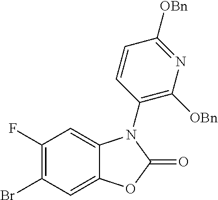 [SMILES: O=c1oc2cc(Br)c(F)cc2n1-c1ccc(OCc2ccccc2)nc1OCc1ccccc1]

To a mixture of 6-bromo-5-fluorobenzo[d]oxazol-2(3H)-one (3 g, 12.9 mmol), 2,6-bis(benzyloxy)-3-(4,4,5,5-tetramethyl-1,3,2-dioxaborolan-2-yl)pyridine (6.47 g, 15.5 mmol) in dioxane (30 mL) was added pyridine (10.2 g, 129 mmol), Cu(OAc)₂ (2.58 g, 12.9 mmol) and 4A molecular sieve (2.5 g). The mixture was stirred at 80° C. under oxygen atmosphere for 3 ds. After cooling to r.t, the mixture was filtered and concentrated in vacuo. The residue was purified by silica gel column (PE:EA=8:1) to afford the product (3.3 g, 49.1%). [M+H]⁺=521.1.

### Step 3: 6-(2-(benzyloxy)ethyl)-3-(2,6-bis(benzyloxy)pyridin-3-yl)-5-fluorobenzo[d]oxazol-2(3H)-one

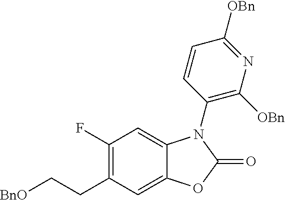 [SMILES: O=c1oc2cc(CCOCc3ccccc3)c(F)cc2n1-c1ccc(OCc2ccccc2)nc1OCc1ccccc1]

To a mixture of 3-(2,6-bis(benzyloxy)pyridin-3-yl)-6-bromo-5-fluorobenzo[d]oxazol-2(3H)-one (1 g, 1.92 mmol), NiI₂ (120 mg, 0.38 mmol), picolinimidamide hydrochloride (60 mg, 0.38 mmol), NaI (144 mg, 0.96 mmol), and Mn (317 mg, 5.76 mmol) in DMA (20 mL), was added TFA (66 mg, 0.58 mmol) under nitrogen atmosphere. The resulting mixture was stirred at 100° C. for 3 hrs. After cooling to r.t, the reaction was diluted with EA (60 mL) and then washed with brine (20 mL×3), dried over Na₂SO₄, filtered and concentrated in vacuum. The residue was purified by silica gel column (PE:EA=10:1) to afford 6-(2-(benzyloxy)ethyl)-3-(2,6-bis(benzyloxy)pyridin-3-yl)-5-fluorobenzo[d]oxazol-2(3H)-one (330 mg, 29.8%). [M+H]⁺=577.2.

### Step 4: 3-(5-fluoro-6-(2-hydroxyethyl)-2-oxobenzo[d]oxazol-3(2H)-yl)piperidine-2,6-dione

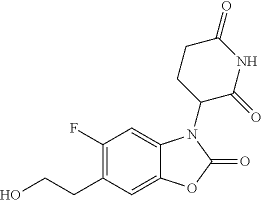 [SMILES: O=C1CCC(n2c(=O)oc3cc(CCO)c(F)cc32)C(=O)N1]

To a mixture of 6-(2-(benzyloxy)ethyl)-3-(2,6-bis(benzyloxy)pyridin-3-yl)-5-fluorobenzo[d]oxazol-2(3H)-one (330 mg, 0.57 mmol) in DMF (8 mL) and EtOH (2 mL) was added Pd/C (150 mg, 10 wt. %, wet). The resulting mixture was stirred at r.t under hydrogen atmosphere for 16 hrs. The reaction mixture was filtered and concentrated in vacuo to afford 3-(5-fluoro-6-(2-hydroxyethyl)-2-oxobenzo[d]oxazol-3(2H)-yl)piperidine-2,6-dione (160 mg, 91%). [M+H]⁺=309.1.

### Step 5: 2-(3-(2,6-dioxopiperidin-3-yl)-5-fluoro-2-oxo-2,3-dihydrobenzo[d]oxazol-6-yl)acetaldehyde

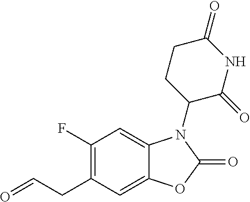 [SMILES: O=CCc1cc2oc(=O)n(C3CCC(=O)NC3=O)c2cc1F]

To a solution of 3-(5-fluoro-6-(2-hydroxyethyl)-2-oxobenzo[d]oxazol-3(2H)-yl)piperidine-2,6-dione (160 mg, 0.52 mmol) in THF (3 mL) and DCM (3 mL) was added DMP (329 mg, 0.78 mmol). After stirring at r. t. for 2 hs, the reaction was diluted with water (6 mL), and the layers were separated. The aqueous layer was extracted with DCM (10 mL×3). The combined organic layers were washed with brine (30 mL×3), dried over Na₂SO₄, filtered and concentrated in vacuum. The residue was purified by silica gel column (DCM:CH3OH=15:1) to afford 2-(3-(2,6-dioxopiperidin-3-yl)-5-fluoro-2-oxo-2,3-dihydrobenzo[d]oxazol-6-yl)acetaldehyde (100 mg, 62.5%). [M+H]⁺=307.1.

### Step 6: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

To a solution of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (50 mg, 0.068 mmol, the compound was obtained similar to example 484), 2-(3-(2,6-dioxopiperidin-3-yl)-5-fluoro-2-oxo-2,3-dihydrobenzo[d]oxazol-6-yl)acetaldehyde (25 mg, 0.082 mmol) in DCM (4 mL) was added STAB (28.8 mg, 0.136 mmol). After stirring at r. t. for 2 h, the reaction was diluted with water (6 mL), and the layers were separated. The aqueous layer was extracted with DCM (10 mL×3). The combined organic layers were washed with brine (30 mL×3), dried over Na₂SO₄, filtered and concentrated in vacuum. The residue was purified by prep-HPLC (C-18 column chromatography (0.1% FA in water:acetonitrile=90:10˜60:40 gradient elution) to afford the product (16.7 mg, 24%). ¹H NMR (500 MHz, DMSO) δ 11.73 (s, 1H), 11.21 (s, 1H), 8.59 (d, J=8.9 Hz, 1H), 8.26-8.24 (m, 1H), 8.23 (s, 1H), 8.00-7.77 (m, 2H), 7.53-7.36 (m, 3H), 7.29-7.27 (m, 1H), 6.65 (s, 1H), 5.36-5.31 (m, 1H), 4.06-3.92 (m, 2H), 2.96-2.91 (m, 4H), 2.89-2.81 (m, 1H), 2.81-2.74 (m, 2H), 2.73-2.70 (m, 1H), 2.65-2.61 (m, 4H), 2.53-2.51 (m, 3H), 2.49-2.42 (m, 4H), 2.27-2.23 (m, 4H), 2.19-2.10 (m, 1H), 2.00 (s, 3H), 1.97 (s, 3H), 1.82 (d, J=12.3 Hz, 2H), 1.54-1.51 (m, 2H), 1.33-1.30 (m, 3H), 1.27-1.24 (m, 4H), 0.75-0.69 (m, 3H). [M+H]⁺=1025.9.

### Example 23: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)phenyl)piperidine-2,6-dione

### Step 1: tert-butyl 4-(1-(2-bromo-5-methoxy-4-nitrophenyl)piperidin-4-yl)piperazine-1-carboxylate

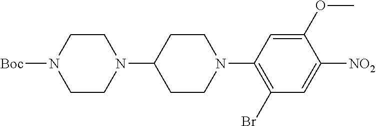 [SMILES: COc1cc(N2CCC(N3CCN(C(=O)OC(C)(C)C)CC3)CC2)c(Br)cc1[N+](=O)[O-]]

A mixture of 1-bromo-2-fluoro-4-methoxy-5-nitrobenzene (4 g, 16 mmol), tert-butyl 4-(piperidin-4-yl)piperazine-1-carboxylate (6.4 g, 24 mmol), K₂CO₃ (4.4 g, 32 mmol) in DMF (50 mL) was stirred in a flask at 80° C. overnight. The reaction mixture was allowed to cool down to room temperature. The resulting mixture was diluted with water and extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (7 g, 90%). [M+H]⁺=499.0.

### Step 2: tert-butyl 4-(1-(5-methoxy-4-nitro-2-vinylphenyl)piperidin-4-yl)piperazine-1-carboxylate

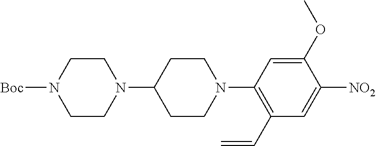 [SMILES: C=Cc1cc([N+](=O)[O-])c(OC)cc1N1CCC(N2CCN(C(=O)OC(C)(C)C)CC2)CC1]

A mixture of tert-butyl 4-(1-(2-bromo-5-methoxy-4-nitrophenyl)piperidin-4-yl)piperazine-1-carboxylate (7 g, 14 mmol), 4,4,5,5-tetramethyl-2-vinyl-1,3,2-dioxaborolane (4.3 g, 28 mmol), Pd(dppf)Cl₂ (1.1 g, 1.4 mmol) and K₃PO₄ (8.9 g, 42 mmol) in DMF (160 mL) and water (20 mL) was stirred in a flask at 90° C. under nitrogen atmosphere for 16 hrs. The reaction mixture was allowed to cool down to room temperature. The resulting mixture was extracted with EtOAc (3×1000 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (5 g, 80%). [M+H]⁺=447.0.

### Step 3: tert-butyl 4-(1-(4-amino-2-ethyl-5-methoxyphenyl)piperidin-4-yl)piperazine-1-carboxylate

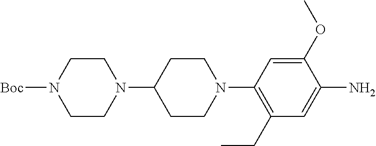 [SMILES: CCc1cc(N)c(OC)cc1N1CCC(N2CCN(C(=O)OC(C)(C)C)CC2)CC1]

To a stirred solution of tert-butyl 4-(1-(5-methoxy-4-nitro-2-vinylphenyl)piperidin-4-yl)piperazine-1-carboxylate (5 g, 11.2 mmol) in MeOH (100 mL) and DCM (20.00 mL) was added Pd/C (wet, 10%) (1 g) under nitrogen atmosphere. The resulting mixture was stirred for 16 hrs at room temperature under hydrogen atmosphere. The resulting mixture was filtered, the filter cake was washed with DCM/CH₃OH (10:1, 200 mL). The filtrate was concentrated under reduced pressure to afford the product (4.0 g, 85.3%). [M+H]⁺=419.1.

### Step 4: (6-aminoquinoxalin-5-yl)dimethylphosphine oxide

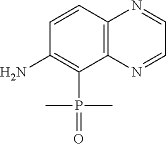

A mixture of 5-bromoquinoxalin-6-amine (10 g, 44.8 mmol), dimethylphosphine oxide (10.5 g, 134.5 mmol), Pd(OAc)₂ (1.0 g, 4.5 mmol) Xantphos (5.2 g, 9 mmol) and K₃PO₄ (28 g, 134 mmol) in DMF (250 mL) and water (50 mL) was stirred in a flask at 130° C. under nitrogen atmosphere for 16 hrs. The reaction mixture was allowed to cool down to room temperature. The resulting mixture was extracted with DCM (3×1000 mL). The combined organic layers were washed with brine (1000 mL), dried over anhydrous Na₂SO₄. After filtration, the filtrate was concentrated under reduced pressure. The residue was purified by silica gel column chromatography, eluted with DCM/MeOH (10:1) to afford the product (6 g, 60%), [M+H]⁺=222.0.

### Step 5: (6-((5-bromo-2-chloropyrimidin-4-yl)amino)quinoxalin-5-yl)dimethyl phosphine oxide

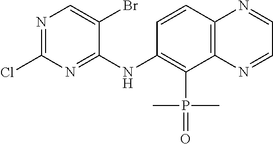 [SMILES: CP(C)(=O)c1c(Nc2nc(Cl)ncc2Br)ccc2nccnc12]

A mixture of (6-aminoquinoxalin-5-yl)dimethylphosphine oxide (6 g, 27.3 mmol), 5-bromo-2,4-dichloropyrimidine (12.3 g, 54.6 mmol) in THF (200 mL) was stirred in a flask at 0° C. under nitrogen atmosphere, 54 mL KHMDS (1M in THF) was added. The reaction mixture was allowed to warm up to room temperature for 2 hours. The reaction was quenched with water and the mixture was extracted with DCM, washed three times with saturated brine, dried over anhydrous Na₂SO₄. After filtration, the filtrate was concentrated under reduced pressure. The residue was purified by silica gel column chromatography, eluted with DCM/MeOH (10:1) to afford the product (4 g, 35%). [M+H]⁺=412.0.

### Step 6: (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide

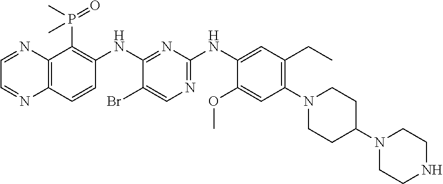 [SMILES: CCc1cc(Nc2ncc(Br)c(Nc3ccc4nccnc4c3P(C)(C)=O)n2)c(OC)cc1N1CCC(N2CCNCC2)CC1]

A mixture of (6-((5-bromo-2-chloropyrimidin-4-yl)amino)quinoxalin-5-yl)dimethyl phosphine oxide (2 g, 4.8 mmol), tert-butyl 4-(1-(4-amino-2-ethyl-5-methoxyphenyl)piperidin-4-yl)piperazine-1-carboxylate (2.6 g, 6.3 mmol) and MsOH (184 mg, 1.92 mmol) in t-BuOH (20 mL) was stirred in a flask at 90° C. under nitrogen atmosphere for overnight. The reaction mixture was concentrated under reduced pressure. The residue was purified by silica gel column chromatography to afford the product (2 g, 60%). [M+H]⁺=694.0.

### Step 7: 2,6-bis(benzyloxy)-3-(4-bromophenyl)pyridine

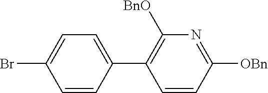 [SMILES: Brc1ccc(-c2ccc(OCc3ccccc3)nc2OCc2ccccc2)cc1]

To a stirred mixture of 2,6-bis(benzyloxy)-3-(4,4,5,5-tetramethyl-1,3,2-dioxaborolan-2-yl)pyridine (The intermediate can be prepared according to the way described in WO2017197046) (25 g, 59.9 mmol) and 4-bromoiodobenzene (20.3 g, 71.9 mmol) in dioxane (250 mL) and H₂O (50 mL) were added K₂CO₃ (16.6 g, 120 mmol) and Pd(dppf)Cl₂ (4.4 g, 6.0 mmol) at room temperature under nitrogen atmosphere. The resulting mixture was stirred for 16 h at 80° C. under nitrogen atmosphere. The reaction mixture was allowed to cool down to room temperature. The resulting mixture was extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (10:1) to afford the product (23 g, 86%); [M+H]⁺=446.2.

### Step 8: ethyl 2-(1-[4-[2,6-bis(benzyloxy)pyridin-3-yl]phenyl]piperidin-4-yl)acetate

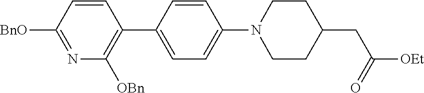 [SMILES: CCOC(=O)CC1CCN(c2ccc(-c3ccc(OCc4ccccc4)nc3OCc3ccccc3)cc2)CC1]

To a stirred solution of 2,6-bis(benzyloxy)-3-(4-bromophenyl)pyridine (15 g, 33.6 mmol) and ethyl 2-(piperidin-4-yl)acetate (8.6 g, 50.4 mmol) in 2-methyl-THF (150 mL) and H₂O (15 mL) were added Cs₂CO₃ (32.9 g, 100.8 mmol), DavePhos (2.7 g, 6.7 mmol) and Pd₂(dba)₃ (3.1 g, 3.4 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (500 mL), washed with water (3×200 mL) and brine (200 mL). The organic layer was dried over anhydrous Na₂SO₄. After filtration, the filtrate was concentrated under reduced pressure. The residue was purified by silica gel column chromatography, eluted with PE/EtOAc (1:1) to afford the product (14 g, 78%); [M+H]⁺=537.3.

### Step 9: 2-(1-(4-(2,6-bis(benzyloxy)pyridin-3-yl)phenyl)piperidin-4-yl)ethan-1-ol

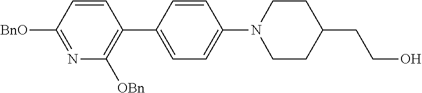 [SMILES: OCCC1CCN(c2ccc(-c3ccc(OCc4ccccc4)nc3OCc3ccccc3)cc2)CC1]

To a stirred solution of ethyl 2-(1-[4-[2,6-bis(benzyloxy)pyridin-3-yl]phenyl]piperidin-4-yl)acetate (13 g, 24.2 mmol) in THF (130 mL) was added LiAlH₄ (1 g, 26.6 mmol) in portions at 0° C. The resulting mixture was stirred for 2 h at room temperature. The reaction was quenched by the addition of water/ice (50 mL) at 0° C. The resulting mixture was extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with PE/EtOAc (1:2) to afford the product (11 g, 92%); [M+H]⁺=495.3.

### Step 10: 3-[4-[4-(2-hydroxyethyl)piperidin-1-yl]phenyl]piperidine-2,6-dione

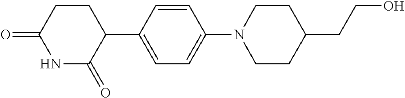 [SMILES: O=C1CCC(c2ccc(N3CCC(CCO)CC3)cc2)C(=O)N1]

To a stirred solution of 2-(1-(4-(2,6-bis(benzyloxy)pyridin-3-yl)phenyl)piperidin-4-yl)ethan-1-ol (10.5 g, 21.2 mmol) in EtOH (100 mL), EtOAc (100 mL) and DCM (20 mL) was added Pd/C (wet, 10%) (5 g) under nitrogen atmosphere. The resulting mixture was stirred for 16 h at room temperature under hydrogen atmosphere. The resulting mixture was filtered, the filter cake was washed with DCM/CH₃OH (10:1, 200 mL). The filtrate was concentrated under reduced pressure. The residue was purified by silica gel column chromatography, eluted with MeOH/DCM (1:10) to afford the product (5.1 g, 76%). [M+H]⁺=317.1.

### Step 11: 2-(1-(4-(2,6-dioxopiperidin-3-yl)phenyl)piperidin-4-yl)acetaldehyde

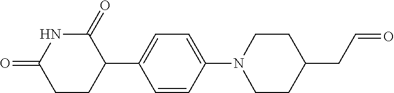 [SMILES: O=CCC1CCN(c2ccc(C3CCC(=O)NC3=O)cc2)CC1]

A mixture of 3-[4-[4-(2-hydroxyethyl)piperidin-1-yl]phenyl]piperidine-2,6-dione (100 mg, 0.32 mmol) and IBX (132 mg, 0.47 mmol) in DMSO (10 mL) was stirred in a flask at room temperature overnight. The reaction was quenched with water and the mixture was extracted with EtOAc, washed three times with saturated aqueous NaCl and twice with saturated aqueous NaHCO₃. The organic layer was dried over anhydrous Na₂SO₄ and evaporated in vacuum to afford the product (70 mg, 70%). [M+H]⁺=315.2.

### Step 12: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)phenyl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.057 mmol), 2-(1-(4-(2,6-dioxopiperidin-3-yl)phenyl)piperidin-4-yl)acetaldehyde (21 mg, 0.069 mmol) and NaOAc (14 mg, 0.17 mmol) in dichloromethane (4 mL) and EtOH (0.5 mL) was stirred in a flask at room temperature for 2 hour. To the mixture was added NaBH₃CN (10 mg, 0.17 mmol) and the mixture was stirred in a flask at room temperature for 2 h. Then the mixture was concentrated in vacuum to afford the crude product, which was purified with prep-HPLC chromatography (0.1% FA in water:acetonitrile=90:10˜50:50 gradient elution) to give the product (15 mg, 27%). ¹H NMR (500 MHz, DMSO) δ 12.65 (s, 1H), 10.79 (s, 111), 8.87 (d, J=9.1 Hz, 3H), 8.29 (d, J=12.4 Hz, 2H), 7.91 (s, 1H), 7.38 (s, 1H), 7.03 (d, J=7.8 Hz, 2H), 6.89 (d, J=8.4 Hz, 2H), 6.81 (s, 1H), 3.77 (s, 3H), 3.76-3.62 (m, 3H), 3.02 (s, 6H), 2.80-2.53 (m, 12H), 2.44-2.40 (m, 1H), 2.11 (s, 2H), 2.02 (d, J=14.3 Hz, 7H), 1.88 (s, 2H), 1.75 (d, J=11.0 Hz, 2H), 1.56-1.48 (m, 5H), 1.30-1.28 (m, 3H), 0.95-0.93 (m, 3H); [M+H]⁺=992.5.

### Example 751: (R)-3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

The title compound was prepared in a procedure similar to that in Example 486.

¹H NMR (500 MHz, DMSO) δ 12.65 (s, 1H), 10.96 (s, 1H), 8.86 (dt, J=22.8, 11.4 Hz, 3H), 8.28 (d, J=9.1 Hz, 2H), 7.90 (d, J=8.9 Hz, 1H), 7.37 (s, 1H), 7.03 (d, J=10.0 Hz, 2H), 6.81 (s, 1H), 4.20 (dd, J=12.8, 5.0 Hz, 1H), 3.77 (s, 3H), 3.01 (d, J=11.5 Hz, 2H), 2.76 (m, 6H), 2.54 (d, J=1.8 Hz, 6H), 2.48 (s, 5H), 2.36 (s, 2H), 2.13 (d, J=9.6 Hz, 1H), 2.02 (d, J=14.4 Hz, 7H), 1.87 (d, J=11.4 Hz, 2H), 1.58 (d, J=8.8 Hz, 2H), 0.93 (t, J=7.2 Hz, 3H); [M+H]⁺=945.4.

### Example 493: 3-(7-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

The title compound (31 mg, 56%) was prepared in a manner similar to that in Example 492. ¹H NMR (500 MHz, DMSO) δ 12.10 (s, 1H), 11.21 (s, 1H), 8.56 (d, J=8.8 Hz, 1H), 8.39-8.10 (m, 2H), 8.04 (s, 1H), 7.54 (d, J=9.0 Hz, 1H), 7.39 (s, 1H), 7.17-7.01 (m, 3H), 6.71 (s, 1H), 5.36 (dd, J=12.8, 5.2 Hz, 1H), 4.01 (q, J=6.9 Hz, 2H), 3.01-2.81 (m, 7H), 2.77-2.52 (m, 13H), 2.38-2.08 (m, 5H), 2.00 (d, J=13.3 Hz, 6H), 1.85-1.83 (m, 2H), 1.57-1.50 (m, 2H), 1.32 (t, J=7.6 Hz, 3H), 1.26 (t, J=6.9 Hz, 3H), 0.75 (s, 3H). [M+H]⁺=1025.8.

### Example 318: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

### Step 1: 7-bromo-1-methyl-1,3-dihydro-2H-benzo[d]imidazol-2-one

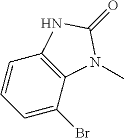

To a solution of 6-bromo-N1-methylbenzene-1,2-diamine (4 g, 19.9 mmol) in CH₃CN (50 mL) was added CDI (6.4 g, 39.8 mmol). The resulting solution was stirred for 6 h at 90° C. under nitrogen atmosphere. The solid was collected by filtration. This resulted in 7-bromo-1-methyl-1,3-dihydro-2H-benzo[d]imidazol-2-one (4.1 g, 90.7%). [M+H]⁺=227.0.

### Step 2: 3-(4-bromo-3-methyl-2-oxo-2,3-dihydro-1H-benzo[d]imidazol-1-yl)-1-(4-methoxybenzyl)piperidine-2,6-dione

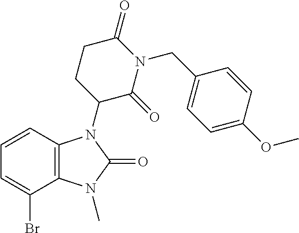 [SMILES: COc1ccc(CN2C(=O)CCC(n3c(=O)n(C)c4c(Br)cccc43)C2=O)cc1]

To a solution of 7-bromo-1-methyl-1,3-dihydro-2H-benzo[d]imidazol-2-one (600 mg, 2.6 mmol) in THF (10 mL) was added t-BuOK (1M in THF, 3.2 mL, 3.2 mmol) dropwise in 10 min at 0° C., the reaction solution was stirred for 30 min at this temperature, then to this was added 1-(4-methoxybenzyl)-2,6-dioxopiperidin-3-yl trifluoromethanesulfonate (1.1 g, 2.9 mmol) in THF (5 mL) dropwise in 10 min. The resulting solution was stirred for 2 h at 0-10° C. The reaction was quenched by the addition of sat. aq. NH₄Cl solution and the layers were separated. The aqueous layer was extracted with EtOAc (10 mL×3). The combined organic layers were washed with brine, dried over anhydrous Na₂SO₄. after filtration, the filtrate was concentrated under reduced pressure. The residue was purified by a silica gel column, eluted with PE/EtOAc to afford product (910 mg, 75.2%). [M+H]⁺=458.1.

### Step 3: 3-(4-bromo-3-methyl-2-oxo-2,3-dihydro-1H-benzo[d]imidazol-1-yl)piperidine-2,6-dione

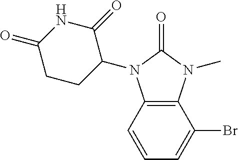 [SMILES: Cn1c(=O)n(C2CCC(=O)NC2=O)c2cccc(Br)c21]

3-(4-bromo-3-methyl-2-oxo-2,3-dihydro-1H-benzo[d]imidazol-1-yl)-1-(4-methoxybenzyl)piperidine-2,6-dione (800 mg, 1.75 mmol) was dissolved in MeSO2H/toluene (2 mL/6 mL). The resulting mixture was stirred for 3 h at 100° C. Solvent was removed and the residue was poured into ice/water. The solid was collected by filtration. 3-(4-bromo-3-methyl-2-oxo-2,3-dihydro-1H-benzo[d]imidazol-1-yl)piperidine-2,6-dione was obtained (510 mg, 86.4%). [M+H]⁺=338.1.

### Step 4: (E)-3-(4-(2-ethoxyvinyl)-3-methyl-2-oxo-2,3-dihydro-TH-benzo[d]imidazol-1-yl)piperidine-26-dione

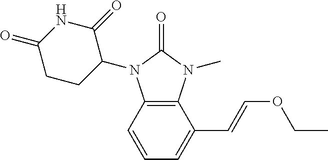 [SMILES: CCO/C=C/c1cccc2c1n(C)c(=O)n2C1CCC(=O)NC1=O]

To a stirred solution of 3-(4-bromo-3-methyl-2-oxo-2,3-dihydro-1H-benzo[d]imidazol-1-yl)piperidine-2,6-dione (250 mg, 0.74 mmol) and (E)-2-(2-ethoxyvinyl)-4,4,5,5-tetramethyl-1,3,2-dioxaborolane (176 mg, 0.89 mmol) in DMF/H₂O (8 mL/2 mL) were added Pd(dtbpf)Cl₂ (48 mg, 0.074 mmol) and CsF (225 mg, 1.48 mmol). The resulting mixture was stirred for 2 h at 80° C. under nitrogen atmosphere. The reaction solution was diluted with water, and extracted with EtOAc (10 mL×3). The combined organic layers were washed with water and brine, dried over anhydrous Na₂SO₄ which was evaporated to dryness. The residue was purified by a silica gel column, eluted with PE/EtOAc=1:1 to afford the product. (180 mg, 73.8%). m/z [M+H]⁺=330.2.

### Step 5: 2-(1-(2,6-dioxopiperidin-3-yl)-3-methyl-2-oxo-2,3-dihydro-1H-benzo[d]imidazol-4-yl)acetaldehyde

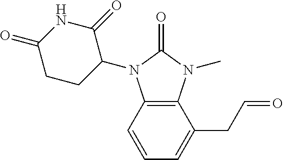 [SMILES: Cn1c(=O)n(C2CCC(=O)NC2=O)c2cccc(CC=O)c21]

(E)-3-(4-(2-ethoxyvinyl)-3-methyl-2-oxo-2,3-dihydro-1H-benzo[d]imidazol-1-yl)piperidine-2,6-dione (180 mg, 0.55 mmol) was dissolved in HCOOH (2 mL). The resulting solution was stirred for 2 h at room temperature. The reaction solution was evaporated to dryness to afford product (125 mg, 75.3%) which was used directly in the next step. m/z [M+H]⁺=302.1.

### Step 6: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (50 mg, 0.056 mmol), 2-(1-(2,6-dioxopiperidin-3-yl)-3-methyl-2-oxo-2,3-dihydro-1H-benzo[d]imidazol-4-yl)acetaldehyde (17 mg, 0.070 mmol) and NaBH(OAc)₃ (24 mg, 0.11 mmol) in DCE (3 mL) was stirred in a round bottom flask at room temperature for 12 hours. The reaction was diluted with DCM, washed with brine (2×5 mL), dried over Na₂SO₄ and concentrated under vacuum to afford the crude product, which was purified with pre-HPLC (0.1% FA in water:acetonitrile=90:1˜50:50 gradient elution) to give the title product (12 mg, 28%). ¹H NMR (500 MHz, DMSO) δ12.58 (s, 1H), 11.03 (s, 1H), 8.78 (d, J=23.1 Hz, 3H), 8.20 (s, 2H), 7.85 (s, 1H), 7.32 (s, 1H), 7.07-6.68 (m, 4H), 5.32 (s, 1H), 3.70 (s, 4H), 3.52 (s, 5H), 3.11-2.78 (m, 8H), 2.62-2.58 (m, 8H), 2.25 (s, 2H), 2.09-1.76 (m, 10H), 1.53 (s, 2H), 0.86 (s, 3H). [M+H]⁺=979.4.

### Example 752: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-methyl-2-oxo-2,3-dihydro-1H-benzo[d]imidazol-1-yl)piperidine-2,6-dione

The title compound was prepared in a procedure similar to that in Example 318. ¹H NMR (500 MHz, DMSO) δ 11.73 (s, 1H), 11.10 (s, 1H), 8.60 (d, J=8.9 Hz, 1H), 8.29-8.17 (m, 2H), 7.92 (s, 1H), 7.88 (d, J=9.3 Hz, 1H), 7.45 (d, J=8.9 Hz, 1H), 7.39 (s, 1H), 7.05-6.95 (m, 2H), 6.90 (dd, J=6.3, 2.5 Hz, 1H), 6.71 (s, 1H), 5.37 (dd, J=12.7, 5.3 Hz, 1H), 4.00 (q, J=7.0 Hz, 2H), 3.58 (s, 3H), 3.22-3.20 (m, 2H), 3.09-3.03 (m, 2H), 2.96-2.85 (m, 5H), 2.76-2.51 (m, 12H), 2.27-2.23 (m, 3H), 2.02-1.93 (m, 7H), 1.83 (d, J=10.7 Hz, 2H), 1.53-1.49 (m, 2H), 1.32 (t, J=7.6 Hz, 3H), 1.26 (t, J=6.9 Hz, 3H), 0.71 (s, 3H). [M+H]⁺=1020.5.

### Example 499: 3-(6-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

### Step 1: (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide

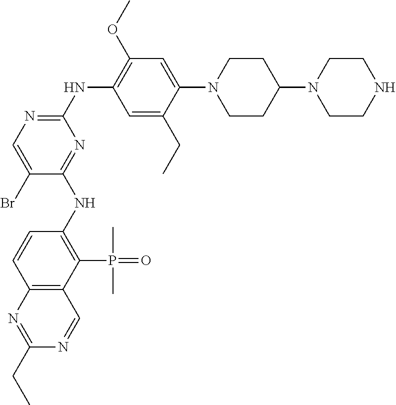 [SMILES: CCc1ncc2c(P(C)(C)=O)c(Nc3nc(Nc4cc(CC)c(N5CCC(N6CCNCC6)CC5)cc4OC)ncc3Br)ccc2n1]

To a solution of (6-((5-bromo-2-chloropyrimidin-4-yl)amino)-2-ethylquinazolin-5-yl)dimethylphosphine oxide (300 mg, 0.68 mmol) and tert-butyl 4-(1-(4-amino-2-ethyl-5-methoxyphenyl)piperidin-4-yl)piperazine-1-carboxylate (342 mg, 0.82 mmol) in n-BuOH (10 mL) was added Ts-OH (350 mg, 2.04 mmol) at room temperature. The resulting mixture was stirred at 100° C. overnight. The reaction was concentrated and basified with 0.5N NaOH (10 mL), then extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜5:1 gradient elution) to give the desired product (230 mg, 46%). [M+H]⁺=722.2.

### Step 2: 3-(6-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

To a solution of (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide (60 mg, 0.083 mmol) and 2-(3-(2,6-dioxopiperidin-3-yl)-2-oxo-2,3-dihydrobenzo[d]oxazol-6-yl)acetaldehyde (36 mg, 0.125 mmol) in DCM (5 mL) was added sodium triacetoxyborohydride (60 mg, 0.28 mmol) at room temperature. The resulting mixture was stirred at room temperature for 1 hour. The reaction was quenched with water (10 mL) and extracted with DCM (2×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC to give the desired product (14.62 mg, 17.8%). ¹H NMR (500 MHz, DMSO) δ 11.60 (s, 1H), 11.20 (s, 1H), 9.86 (s, 1H), 8.45 (s, 1H), 8.23 (s, 1H), 8.05 (s, 1H), 7.85 (d, J=9.2 Hz, 1H), 7.29 (d, J=13.2 Hz, 2H), 7.16 (d, J=8.0 Hz, 1H), 7.08 (d, J=8.1 Hz, 1H), 6.74 (s, 1H), 5.35 (dd, J=12.6, 5.1 Hz, 1H), 3.75 (s, 3H), 3.05 (q, J=7.5 Hz, 2H), 2.96-2.86 (m, 3H), 2.77 (t, J=7.3 Hz, 2H), 2.71-2.63 (m, 5H), 2.57-2.51 (s, 6H), 2.47-2.43 (m, 2H), 2.26 (s, 3H), 2.17-2.13 (m, 1H), 2.03 (d, J=13.4 Hz, 7H), 1.84 (d, J=11.5 Hz, 2H), 1.54 (d, J=10.2 Hz, 2H), 1.38 (t, J=7.5 Hz, 3H), 0.75 (s, 3H); [M+H]⁺=994.7.

### Example 753: (R)-3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

The title compound was prepared in a procedure similar to that in Example 486. ¹H NMR (500 MHz, DMSO) δ 12.17 (s, 1H), 10.95 (s, 1H), 8.62 (s, 1H), 8.54 (s, 1H), 8.24 (s, 1H), 8.18 (d, J=6.0 Hz, 2H), 7.31 (s, 1H), 7.04 (s, 1H), 7.02 (s, 1H), 6.76 (s, 1H), 4.22-4.15 (m, 3H), 3.75 (s, 3H), 2.98 (d, J=10.6 Hz, 2H), 2.85-2.74 (m, 4H), 2.67 (t, J=11.1 Hz, 3H), 2.55-2.53 (m, 7H), 2.48-2.38 (m, 5H), 2.32 (t, J=11.1 Hz, 1H), 2.17-2.10 (m, 1H), 2.03 (d, J=13.4 Hz, 6H), 1.85 (d, J=10.9 Hz, 2H), 1.56 (d, J=11.4 Hz, 2H), 1.31 (t, J=7.2 Hz, 3H), 0.91 (s, 3H). [M+H]⁺=989.7.

### Example 501: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3,3-dimethyl-2-oxoindolin-1-yl)piperidine-2,6-dione

### Step 1: 6-bromo-2-ethylphthalazin-1(2H)-one

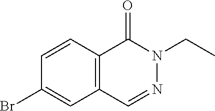

To a stirred solution of 6-bromophthalazin-1(2H)-one (10 g, 44.6 mmol) and Cs₂CO₃ (29.1 g, 89.2 mmol) in DMF (200 mL) was added iodoethane (10.4 g, 66.9 mmol). The resulting mixture was stirred at rt for 16 hours. The mixture was filtrated and the filtrate was concentrated under vacuum to afford the crude product, which was purified with silica gel column chromatography (DCM:MeOH=100:0˜100:10 gradient elution) to give the title product (10 g, 88.5%). [M+H]⁺=253.2.

### Step 2: 6-((diphenylmethylene)amino)-2-ethylphthalazin-1(2H)-one

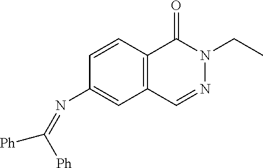 [SMILES: CCn1ncc2cc(N=C(c3ccccc3)c3ccccc3)ccc2c1=O]

To a stirred solution of 6-bromo-2-ethylphthalazin-1(2H)-one (10 g, 39.7 mmol), Cs₂CO₃ (25.9 g, 79.4 mmol), Pd₂(dba)₃ (3.6 g, 4.0 mmol), and BINAP (4.9 g, 7.9 mmol) in dioxane (200 mL) was added diphenylmethanimine (14.4 g, 79.4 mmol). The resulting mixture was stirred at 100° C. for 16 hours under nitrogen atmosphere. The mixture was filtrated and the filtrate was concentrated under vacuum to afford the crude product, which was purified with silica gel column chromatography (PE:EA=100:0˜1:1 gradient elution) to give the title product (12 g, 85.6%). [M+H]⁺=354.4.

### Step 3: 6-amino-2-ethylphthalazin-1(2H)-one

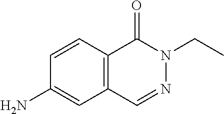

A solution of 6-((diphenylmethylene)amino)-2-ethylphthalazin-1(2H)-one (12 g, 34 mmol) in 4N HCl in 1,4-dioxane (150 mL) was stirred in a round bottom flask at room temperature overnight. The mixture was concentrated under reduced pressure to afford the crude product (6 g, 93%), which was used for next step without further purification. [M+H]⁺=190.0.

### Step 4: 6-amino-2-ethyl-5-iodophthalazin-1(2H)-one

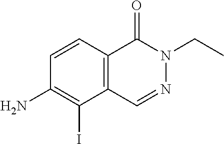

To a stirred solution of 6-amino-2-ethylphthalazin-1(2H)-one (6 g, 31.6 mmol) in AcOH (100 mL) was added ICl (7.7 g, 47.7 mmol) at 20° C. Then the mixture was stirred at 20° C. for 1 hour. Then the mixture was adjusted to pH=8 with sat. aq. NaHCO₃ and extracted with DCM (3×100 mL). The combined organic layers were washed with brine (3×80 mL), dried over Na₂SO₄, filtered and concentrated in vacuum to afford the crude product, which was purified with silica gel column chromatography (DCM:MeOH=100:0˜10:1 gradient elution) to give the title product (8 g, 80%). [M+H]⁺=316.0.

### Step 5: 6-amino-5-(dimethylphosphoryl)-2-ethylphthalazin-1(2H)-one

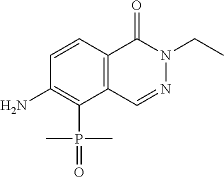 [SMILES: CCn1ncc2c(P(C)(C)=O)c(N)ccc2c1=O]

To a solution of 6-amino-2-ethyl-5-iodophthalazin-1(2H)-one (8 g, 25.4 mmol) and dimethylphosphine oxide (3.9 g, 50.8 mmol) in dioxane (120 mL) was added K₃PO₄ (10.8 g, 50.8 mmol) at 20° C. Pd(OAc)₂ (569 mg, 2.54 mmol) and Xantphos (2.8 g, 5.1 mmol) were added to the mixture at 20° C. The flask was evacuated and backfilled with nitrogen three times. Then the mixture was stirred at 100° C. overnight. The mixture was filtered and concentrated in vacuum. The residue was purified by silica gel column chromatography (DCM:MeOH=100:0˜10:1 gradient elution) to give the desired product (6 g, 89%). [M+H]⁺=266.0.

### Step 6: 6-((5-bromo-2-chloropyrimidin-4-yl)amino)-5-(dimethylphosphoryl)-2-ethylphthalazin-1(2H)-one

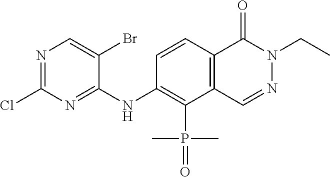 [SMILES: CCn1ncc2c(P(C)(C)=O)c(Nc3nc(Cl)ncc3Br)ccc2c1=O]

To a solution of 6-amino-5-(dimethylphosphoryl)-2-ethylphthalazin-1(2H)-one (6 g, 22.6 mmol) and 5-bromo-2,4-dichloropyrimidine (10.2 g, 45.2 mmol) in THF (100 mL) was added LiHMDS (1 M in THF, 45.2 mL, 45.2 mmol) at room temperature. The resulting mixture was stirred at rt for 1 hour. The reaction was concentrated to afford the crude residue, which was purified by silica gel column chromatography (DCM:MeOH=100:0˜15:1 gradient elution) to give the desired product (8 g, 78%). [M+H]⁺=456.2.

### Step 7: 6-((5-bromo-2-((2-ethoxy-5-ethyl-4-(4-(piperazin-1-yl)piperidin-1-yl)phenyl)amino)pyrimidin-4-yl)amino)-5-(dimethylphosphoryl)-2-ethylphthalazin-1(2H)-one

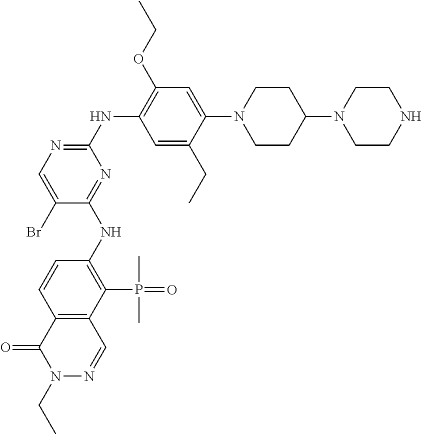 [SMILES: CCOc1cc(N2CCC(N3CCNCC3)CC2)c(CC)cc1Nc1ncc(Br)c(Nc2ccc3c(=O)n(CC)ncc3c2P(C)(C)=O)n1]

To a solution of 6-((5-bromo-2-chloropyrimidin-4-yl)amino)-5-(dimethylphosphoryl)-2-ethylphthalazin-1(2H)-one (3 g, 6.6 mmol) and tert-butyl 4-(1-(4-amino-5-ethoxy-2-ethylphenyl)piperidin-4-yl)piperazine-1-carboxylate (2.9 g, 6.6 mmol) in n-BuOH (100 mL) was added Ts-OH (3.5 g, 19.8 mmol) at room temperature. The resulting mixture was stirred at 100° C. overnight. The reaction was concentrated and basified with 0.5N NaOH (50 mL), then extracted with DCM (3×80 mL). The combined organic layers were dried over anhydrous Na₂SO₄, filtered and evaporated in vacuum to afford the crude residue, which was purified by silica gel column chromatography (DCM:MeOH=100:0˜5:1 gradient elution) to give the desired product (3 g, 61%). [M+H]⁺=752.2.

### Step 8: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3,3-dimethyl-2-oxoindolin-1-yl)piperidine-2,6-dione

To a solution of 6-((5-bromo-2-((2-ethoxy-5-ethyl-4-(4-(piperazin-1-yl)piperidin-1-yl)phenyl)amino)pyrimidin-4-yl)amino)-5-(dimethylphosphoryl)-2-ethylphthalazin-1(2H)-one (50 mg, 0.06 mmol) and 2-(1-(2,6-dioxopiperidin-3-yl)-3,3-dimethyl-2-oxoindolin-4-yl)acetaldehyde (23 mg, 0.07 mmol, the compound was obtained through the similar way with example 413) in DCM (10 mL) was added NaBH(OAc)₃ (30 mg, 0.14 mmol) at room temperature. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give the product (20 mg, 32%). ¹H NMR (500 MHz, DMSO) δ 12.11 (s, 1H), 11.06 (s, 1H), 8.64 (s, 1H), 8.53 (s, 1H), 8.27 (s, 1H), 8.19 (d, J=9.3 Hz, 1H), 8.10 (s, 1H), 7.33 (s, 1H), 7.18 (t, J=7.8 Hz, 1H), 6.92 (d, J=7.7 Hz, 1H), 6.83 (s, 1H), 6.74 (s, 1H), 5.21 (s, 1H), 4.17 (q, J=7.2 Hz, 2H), 4.00 (q, J=6.9 Hz, 2H), 2.98 (d, J=10.5 Hz, 2H), 2.84 (s, 4H), 2.72-2.53 (m, 11H), 2.40 (d, J=7.4 Hz, 4H), 2.04 (d, J=13.4 Hz, 6H), 1.95-1.90 (m, 4H), 1.58 (s, 2H), 1.38 (d, J=4.1 Hz, 6H), 1.32 (t, J=7.2 Hz, 3H), 1.25 (t, J=6.8 Hz, 3H), 0.89 (s, 3H). [M+H]⁺=1050.4.

### Example 413: 3-(6-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

### Step 1: 1-(tert-butyl) 3-methyl 2-(4-bromo-2-nitrophenyl)malonate

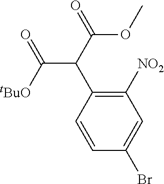 [SMILES: COC(=O)C(C(=O)OC(C)(C)C)c1ccc(Br)cc1[N+](=O)[O-]]

To a stirred solution of 1-tert-butyl 3-methyl propanedioate (30.88 g, 177.275 mmol) and 4-bromo-1-fluoro-2-nitrobenzene (32.50 g, 147.729 mmol) in DMF (200.00 mL) was added Cs₂CO₃ (96.27 g, 295.459 mmol) dropwise at 0° C. under nitrogen atmosphere. The resulting mixture was stirred for 16 h at 70° C. The resulting mixture was filtered, and the filter cake was washed with EtOAc (3×500 mL). The solution was acidified to pH 7-8 with HCl (aq., 1 M). The resulting mixture was diluted with water (1.5 L). The resulting solution was extracted with EA (3×1000 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (40:1) to afford product (47.1 g, 85.21%). [M+H]⁺=373.9

### Step 2: methyl 2-(4-bromo-2-nitrophenyl)acetate

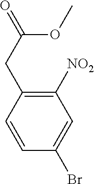

To a stirred solution of 1-(tert-butyl) 3-methyl 2-(4-bromo-2-nitrophenyl)malonate (46.10 g, 123.200 mmol) in toluene (200.00 mL) was added TsOH·H₂O (11.72 g, 61.600 mmol) at 110° C. under nitrogen atmosphere for 16 h. The resulting mixture was concentrated under vacuum. The resulting mixture was extracted with EtOAc (3×200 mL). The combined organic layers were washed with aqueous NaHCO₃ (3×100 mL), dried over anhydrous Na₂SO₄. After filtration, the filtrate was concentrated under reduced pressure. The residue was purified by silica gel column chromatography, eluted with 1˜ 30% EtOAc in PE to afford product (26.5 g, 78.48%). [M+H]⁺=273.9

### Step 3: methyl 2-(4-bromo-2-nitrophenyl)-2-methylpropanoate

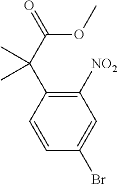 [SMILES: COC(=O)C(C)(C)c1ccc(Br)cc1[N+](=O)[O-]]

To a stirred solution of methyl 2-(4-bromo-2-nitrophenyl)acetate (12.00 g, 43.784 mmol) in ACN (240.00 mL) was added Cs₂CO₃ (28.53 g, 87.569 mmol) at 0° C. under nitrogen atmosphere at ambient temperature. To the mixture was added CH₃I (31.07 g, 218.922 mmol) dropwise over 20 min. The resulting mixture was stirred for 16 h at 80° C. After cooling down to ambient temperature, the resulting mixture was filtered, and the filtrate was washed with EtOAc (3×200 mL). The combined organic layers were concentrated under reduced pressure. The residue was purified by silica gel column chromatography, eluted with 1˜40% EtOAc in PE to afford product (5.6 g, 42.33%). [M+H]⁺=301.9.

### Step 4: 6-bromo-3,3-dimethylindolin-2-one

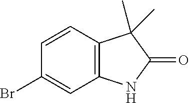

To a stirred solution of methyl 2-(4-bromo-2-nitrophenyl)-2-methylpropanoate (5.50 g, 10.923 mmol) in AcOH (50.00 mL) was added Fe (2.44 g, 43.692 mmol) at room temperature. The resulting mixture was stirred for 2 h at ambient temperature. The resulting mixture was concentrated under reduced pressure. The residue was purified by silica gel column chromatography, eluted with 10˜ 50% EtOAc in PE to afford product (1.95 g, 74.36%). [M+H]⁺=239.9.

### Step 5: 3-(6-bromo-3,3-dimethyl-2-oxoindolin-1-yl)-1-(4-methoxybenzyl)piperidine-2,6-dione

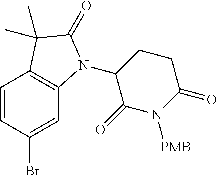

To a stirred mixture of 6-bromo-3,3-dimethylindolin-2-one (1.80 g, 7.497 mmol) in THF (160.00 mL) was added t-BuOK (0.93 g, 8.247 mmol) in portions at 0° C. under nitrogen atmosphere. The resulting mixture was stirred for 1 h at room temperature under nitrogen atmosphere. To the mixture was added 1-(4-methoxybenzyl)-2,6-dioxopiperidin-3-yl trifluoromethanesulfonate (This intermediate was prepared according the same manner described in WO 2020113233A1) (3.14 g, 8.247 mmol) in THF (20 mL) at 0° C. The resulting mixture was stirred for additional 2 h at room temperature. The reaction was quenched with sat. aq. NH₄Cl (30 mL) at 0° C. and diluted with water (1 L). The resulting mixture was extracted with EtOAc (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EtOAc (1:1) to afford product (2.6 g, 73.58%). [M+H]⁺=471.1.

### Step 6: 3-(6-bromo-3,3-dimethyl-2-oxoindolin-1-yl)piperidine-2,6-dione

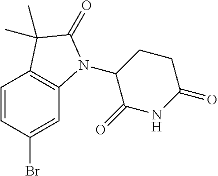 [SMILES: CC1(C)C(=O)N(C2CCC(=O)NC2=O)c2cc(Br)ccc21]

A solution of 3-(6-bromo-3,3-dimethyl-2-oxoindolin-1-yl)-1-(4-methoxybenzyl)piperidine-2,6-dione (2.60 g, 5.516 mmol) and CH₃SO₃H (10.60 g, 110.321 mmol) in Toluene (260.00 mL) was placed in a flask. The resulting mixture was stirred for 2 h at 110° C. under nitrogen atmosphere. The resulting mixture was concentrated under reduced pressure. The resulting mixture was diluted with EtOAc (400 mL). The combined organic layers were washed with brine (3×200 mL), dried over anhydrous Na₂SO₄. After filtration, the filtrate was concentrated under reduced pressure. The residue was purified by silica gel column chromatography, eluted with 1˜50% EtOAc in PE to afford 3-(6-bromo-3,3-dimethyl-2-oxoindolin-1-yl)piperidine-2,6-dione (1.4 g, 72.27%). [M+H]⁺=351.05.

### Step 7: 3-(6-allyl-3,3-dimethyl-2-oxoindolin-1-yl)piperidine-2,6-dione

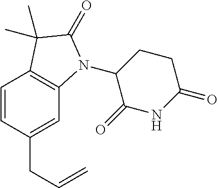 [SMILES: C=CCc1ccc2c(c1)N(C1CCC(=O)NC1=O)C(=O)C2(C)C]

To a stirred solution of 3-(6-bromo-3,3-dimethyl-2-oxoindolin-1-yl)piperidine-2,6-dione (800.00 mg, 2.278 mmol) and tributyl(prop-2-en-1-yl)stannane (1131.39 mg, 3.417 mmol) in DMF (50.00 mL) was added Pd(PPh₃)₂Cl₂ (239.83 mg, 0.342 mmol) at ambient temperature under nitrogen atmosphere. The resulting mixture was stirred for 2 h at 105° C. under nitrogen atmosphere. The resulting mixture was diluted with water (200 mL). The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with 1˜ 40% EtOAc in petroleum ether to afford product (500 mg, 70.27%). [M+H]⁺=313.20

### Step 8: 2-(1-(2,6-dioxopiperidin-3-yl)-3,3-dimethyl-2-oxoindolin-6-yl)acetaldehyde

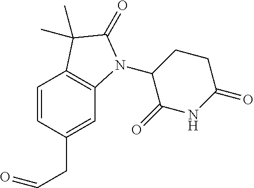 [SMILES: CC1(C)C(=O)N(C2CCC(=O)NC2=O)c2cc(CC=O)ccc21]

To a stirred solution of 3-(6-allyl-3,3-dimethyl-2-oxoindolin-1-yl)piperidine-2,6-dione (500.00 mg, 1.601 mmol) in dioxane (60.00 mL) and water (12.00 mL) were added K₂OsO₄·2H₂O (88.47 mg, 0.240 mmol) and NaIO₄ (1369.48 mg, 6.403 mmol) at 0° C. under nitrogen atmosphere. The resulting mixture was stirred for 12 h at room temperature under nitrogen atmosphere. The resulting mixture was diluted with water (20 mL). The resulting mixture was extracted with EtOAc (3×200 mL). The combined organic layers were washed with brine (3×100 mL), dried over anhydrous Na₂SO₄. After filtration, the filtrate was concentrated under reduced pressure. The residue was purified by reverse flash chromatography with the following conditions: column, C18; mobile phase, ACN in water (0.1% FA), 10% to 50% gradient in 10 min; Detector, UV 220 nm; to afford product (300.1 mg, 59.64%). [M+H]⁺=315.20.

### Step 9: 3-(6-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

A mixture of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50.0 mg, 0.071 mmol), 2-(1-(2,6-dioxopiperidin-3-yl)-3,3-dimethyl-2-oxoindolin-6-yl)acetaldehyde (22.2 mg, 0.071 mmol) and AcOH (0.1 mL) in a mixture of DCM (4 mL) and MeOH (4 mL) was stirred at rt for 2 h. Then NaBH(OAc)₃ (111 mg, 0.425 mmol) was added to the mixture. The resulting mixture was stirred at rt for 2 h. The mixture was diluted with water (50 mL), extracted with DCM (3×50 mL). The combined organic layers were dried over Na₂SO₄, filtered and concentrated under vacuum. The residue was purified by Prep-HPLC with the following conditions: Anal Column: SunFire PrepC18 OBD, 5 μm, 19×150 mm, Column Temp: Room temperature, Mobile Phase A: H₂O (0.1% FA), Mobile Phase B: ACN, Gradient Table: Mobile Phase B (15-35%), Time (min): 0-17 min to afford the product (10.8 mg, 15.2%). ¹H NMR (500 MHz, DMSO) δ 11.77 (s, 1H), 11.06 (s, 1H), 8.56 (d, J=8.9 Hz, 1H), 8.30 (s, 1H), 8.20 (d, J=13.1 Hz, 3H), 7.98 (s, 1H), 7.87 (d, J=9.2 Hz, 1H), 7.42 (d, J=8.9 Hz, 1H), 7.37 (s, 1H), 7.18 (t, J=7.9 Hz, 1H), 6.91 (d, J=7.7 Hz, 1H), 6.87-6.80 (m, 1H), 6.74 (s, 1H), 5.22 (s, 1H), 3.77 (d, J=12.0 Hz, 3H), 3.00-2.75 (m, 6H), 2.72-2.52 (m, 15H), 2.60-2.30 (m, 3H), 1.98 (d, J=13.3 Hz, 8H), 1.84 (d, J=11.2 Hz, 2H), 1.59-1.45 (m, 2H), 1.38 (d, J=4.2 Hz, 7H), 0.78 (s, 3H). [M+H]⁺=1004.4.

### Example 502: (R)-3-(4-(2-(4-(1-(4-((5-bromo-4-((5-(dimethylphosphoryl)-2-methyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

The title compound (497 mg, 60%) was prepared in a manner similar to that in Example 484. ¹H NMR (500 MHz, DMSO) δ 12.11 (s, 1H), 10.85 (s, 1H), 8.64 (s, 1H), 8.52 (s, 1H), 8.26 (s, 1H), 8.18 (d, J=8.9 Hz, 1H), 8.09 (s, 1H), 7.34 (s, 1H), 6.73 (s, 1H), 6.10 (d, J=11.1 Hz, 2H), 4.17 (d, J=7.1 Hz, 2H), 4.00 (d, J=6.9 Hz, 3H), 3.93 (s, 2H), 3.47 (s, 3H), 3.00-2.87 (m, 3H), 2.81-2.73 (m, 1H), 2.64 (t, J=11.1 Hz, 3H), 2.55 (d, J=6.8 Hz, 4H), 2.37-2.32 (m, 6H), 2.03-2.01 (m, 7H), 1.97-1.91 (m, 1H), 1.83 (d, J=11.0 Hz, 2H), 1.55 (dd, J=20.3, 11.3 Hz, 2H), 1.31 (t, J=7.1 Hz, 3H), 1.25 (t, J=6.9 Hz, 3H). [M+H]⁺=989.4.

### Example 503: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-3-methyl-2-oxo-2,3-dihydro-1H-benzo[d]imidazol-1-yl)piperidine-2,6-dione

The title compound (40 mg, 32.2%) was prepared in a manner similar to that in Example 318. ¹H NMR (500 MHz, DMSO) δ 11.69 (s, 1H), 11.11 (s, 1H), 8.63 (d, J=8.7 Hz, 1H), 8.26-8.21 (m, 2H), 7.89 (d, J=12.0 Hz, 2H), 7.46 (d, J=8.9 Hz, 1H), 7.42 (s, 1H), 7.02 (s, 1H), 6.91 (t, J=9.5 Hz, 1H), 6.72 (s, 1H), 5.38 (dd, J=12.7, 5.5 Hz, 1H), 4.01 (d, J=7.0 Hz, 2H), 3.60 (s, 3H), 3.32-3.24 (m, 7H), 3.10 (s, 3H), 2.98-2.83 (m, 6H), 2.67-2.64 (m, 7H), 2.27-2.24 (m, 2H), 2.01-1.97 (m, 8H), 1.60-1.57 (m, 2H), 1.33 (t, J=7.6 Hz, 3H), 1.27 (t, J=6.9 Hz, 3H), 0.70 (s, 3H). [M+H]⁺=1038.7.

### Example 504: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-ethyl-2-oxo-2,3-dihydro-1H-benzo[d]imidazol-1-yl)piperidine-2,6-dione

The title compound (24 mg, 28.1%) was prepared in a manner similar to that in Example 318. ¹H NMR (500 MHz, DMSO) δ 11.73 (s, 1H), 11.11 (s, 1H), 8.60 (d, J=9.0 Hz, 1H), 8.24 (s, 1H), 8.22 (s, 1H), 7.93 (s, 1H), 7.88 (d, J=9.3 Hz, 1H), 7.45 (d, J=8.9 Hz, 1H), 7.38 (s, 1H), 6.99 (d, J=5.3 Hz, 2H), 6.95-6.88 (m, 1H), 6.71 (s, 1H), 5.37 (dd, J=12.9, 5.2 Hz, 1H), 4.03-4.00 (m, 4H), 3.02-2.87 (m, 8H), 2.59-2.55 (m, 13H), 2.29-2.26 (m, 3H), 2.03-2.00 (m, 1H), 1.98 (d, J=13.3 Hz, 6H), 1.83 (d, J=10.8 Hz, 2H), 1.55-1.51 (m, 2H), 1.32 (t, J=7.6 Hz, 3H), 1.27-1.23 (m, 6H), 0.71 (s, 3H). [M+H]⁺=1034.7.

### Example 549: (R)-3-(4-((R)-3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

The title compound (26 mg, 56%) was prepared in a manner similar to that in Example 488. ¹H NMR (500 MHz, DMSO) δ 11.60 (s, 1H), 10.84 (s, 1H), 9.86 (s, 1H), 8.46 (s, 1H), 8.24 (s, 1H), 8.05 (s, 1H), 7.86 (d, J=9.1 Hz, 1H), 7.28 (s, 1H), 6.75 (s, 1H), 6.23 (d, J=12.2 Hz, 2H), 4.02 (dd, J=12.4, 4.9 Hz, 1H), 3.75 (s, 3H), 3.62-3.55 (m, 4H), 3.53-3.48 (m, 3H), 3.33-3.29 (m, 2H), 3.27-3.22 (m, 1H), 3.08-3.03 (m, 2H), 2.95 (d, J=10.0 Hz, 2H), 2.77 (d, J=12.3 Hz, 1H), 2.68 (d, J=11.3 Hz, 2H), 2.57 (s, 3H), 2.38 (s, 1H), 2.27 (s, 2H), 2.17 (d, J=9.0 Hz, 1H), 2.08 (d, J=8.5 Hz, 2H), 2.03 (d, J=13.4 Hz, 7H), 1.97-1.92 (m, 1H), 1.84 (d, J=10.1 Hz, 2H), 1.57 (d, J=9.8 Hz, 2H), 1.38 (t, J=7.5 Hz, 3H), 0.75 (s, 3H); [M+H]⁺=1042.7.

### Example 505: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4-fluoro-2-oxobenzo[d]oxazol-3(2H)-yl)piperidine-2,6-dione

### Step 1: 2-amino-6-bromo-3-fluorophenol

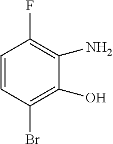

To a solution of 6-bromo-3-fluoro-2-nitrophenol (5 g, 21.3 mmol) in MeOH (100 mL) was added Raney Ni (5 g) at room temperature under hydrogen atmosphere. The resulting mixture was stirred at rt for 2 hours. The reaction was filtered and concentrated under reduced pressure to afford the desired product (4 g, 92%). [M+H]⁺=206.2.

### Step 2: 7-bromo-4-fluorobenzo[d]oxazol-2(3H)-one

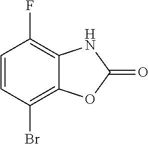 [SMILES: O=c1[nH]c2c(F)ccc(Br)c2o1]

To a solution of 2-amino-6-bromo-3-fluorophenol (4 g, 19.5 mmol) in THF (100 mL) was added CDI (4 g, 23.4 mmol) at room temperature. The resulting mixture was stirred at rt for 2 hours. The reaction was diluted with water (100 mL) and extracted with EA (3×30 mL). The combined organic layers were dried over anhydrous Na₂SO₄, filtered and evaporated in vacuum to afford the crude residue, which was purified by silica gel column chromatography (PE/EA=100:0˜1:1 gradient elution) to give the desired product (3.5 g, 77%). [M+H]⁺=232.3.

### Step 3: 3-(7-bromo-4-fluoro-2-oxobenzo[d]oxazol-3(2H)-yl)piperidine-2,6-dione

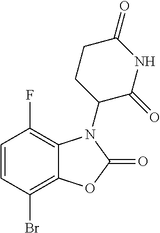 [SMILES: O=C1CCC(n2c(=O)oc3c(Br)ccc(F)c32)C(=O)N1]

To a solution of 7-bromo-4-fluorobenzo[d]oxazol-2(3H)-one (3.5 g, 15.2 mmol) and 3-bromopiperidine-2,6-dione (5.8 g, 30.4 mmol) in DMF (50 mL) was added Cs₂CO₃ (9.9 g, 30.4 mmol) at room temperature. The resulting mixture was stirred at 60° C. overnight. The mixture was filtered, concentrated under reduced pressure and diluted with water (100 mL). The aqueous layer was extracted with DCM (3×30 mL). The combined organic layers were dried over anhydrous Na₂SO₄, filtered and evaporated in vacuum to afford the crude residue, which was mashed with MeOH to give the desired product (1.5 g, 29%). [M+H]⁺=343.2.

### Step 4: (E)-3-(7-(2-ethoxyvinyl)-4-fluoro-2-oxobenzo[d]oxazol-3(2H)-yl)piperidine-2,6-dione

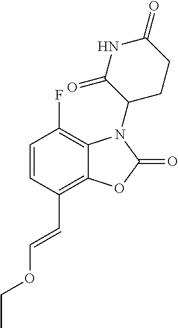 [SMILES: CCO/C=C/c1ccc(F)c2c1oc(=O)n2C1CCC(=O)NC1=O]

To a solution of 3-(7-bromo-4-fluoro-2-oxobenzo[d]oxazol-3(2H)-yl)piperidine-2,6-dione (1.5 g, 4.4 mmol) and (E)-2-(2-ethoxyvinyl)-4,4,5,5-tetramethyl-1,3,2-dioxaborolane (1.7 g, 8.8 mmol) in DMF (30 mL) was added Pd(dtbpf)Cl₂ (286 mg, 0.44 mmol) and CsF (1.3 g, 8.8 mmol) at 20° C. The flask was evacuated and backfilled with nitrogen three times. Then the mixture was stirred at 100° C. overnight. The mixture was filtered and concentrated in vacuum. The residue was purified by silica gel column chromatography (PE:EA=100:0˜0:100 gradient elution) to give the desired product (1.0 g, 68%). [M+H]⁺=335.4.

### Step 5: 2-(3-(2,6-dioxopiperidin-3-yl)-4-fluoro-2-oxo-2,3-dihydrobenzo[d]oxazol-7-yl)acetaldehyde

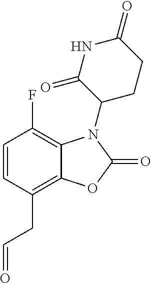 [SMILES: O=CCc1ccc(F)c2c1oc(=O)n2C1CCC(=O)NC1=O]

A solution of (E)-3-(7-(2-ethoxyvinyl)-4-fluoro-2-oxobenzo[d]oxazol-3(2H)-yl)piperidine-2,6-dione (1 g, 3.0 mmol) in FA (20 mL) was stirred at rt for 4 hours. The mixture was concentrated under reduced pressure to afford the desired product (600 mg, 65%). [M+H]⁺=307.1.

### Step 6: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4-fluoro-2-oxobenzo[d]oxazol-3(2H)-yl)piperidine-2,6-dione

To a solution of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (50 mg, 0.07 mmol) and 2-(3-(2,6-dioxopiperidin-3-yl)-4-fluoro-2-oxo-2,3-dihydrobenzo[d]oxazol-7-yl)acetaldehyde (26 mg, 0.08 mmol) in DCM (10 mL) was added NaBH(OAc)₃ (30 mg, 0.14 mmol) at room temperature. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC to give the desired product (12 mg, 17%). ¹H NMR (500 MHz, DMSO) δ 11.73 (s, 1H), 11.24 (s, 1H), 8.60 (d, J=8.8 Hz, 1H), 8.23 (d, J=9.9 Hz, 2H), 7.95-7.83 (m, 2H), 7.45 (d, J=8.9 Hz, 1H), 7.38 (s, 1H), 7.15-7.07 (m, 2H), 6.71 (s, 1H), 5.44 (s, 1H), 3.99 (t, J=7.0 Hz, 2H), 2.93 (dd, J=15.0, 7.5 Hz, 6H), 2.84 (t, J=7.5 Hz, 2H), 2.68-2.53 (m, 11H), 2.32-2.14 (m, 6H), 1.98 (d, J=13.3 Hz, 6H), 1.82 (d, J=11.1 Hz, 2H), 1.52 (d, J=8.7 Hz, 2H), 1.32 (t, J=7.6 Hz, 3H), 1.25 (t, J=6.9 Hz, 3H), 0.71 (s, 3H). [M+H]⁺=1025.4.

### Example 559: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3-ethyl-5-fluoro-2-oxo-2,3-dihydro-1H-benzo[d]imidazol-1-yl)piperidine-2,6-dione

The title compound (6.73 mg, 6%) was prepared in a manner similar to that in Example 318. ¹H NMR (500 MHz, DMSO) δ 12.11 (s, 1H), 11.11 (s, 1H), 8.66 (s, 1H), 8.53 (d, J=5.9 Hz, 1H), 8.28 (s, 1H), 8.19 (d, J=8.9 Hz, 1H), 8.12 (s, 1H), 7.37 (s, 1H), 7.04 (s, 1H), 6.93 (t, J=9.6 Hz, 1H), 6.75 (s, 1H), 5.39 (dd, J=12.5, 4.9 Hz, 1H), 4.18 (q, J=7.0 Hz, 2H), 4.12-3.91 (m, 4H), 3.73-3.47 (m, 2H), 3.32 (s, 3H), 3.19-2.95 (m, 7H), 2.94-2.83 (m, 1H), 2.71-2.67 (m, 6H), 2.55 (d, J=10.0 Hz, 2H), 2.46-2.32 (m, 2H), 2.19 (dd, J=6.9, 0.8 Hz, 1H), 2.03 (t, J=10.8 Hz, 7H), 1.92-1.77 (m, 2H), 1.32 (t, J=7.2 Hz, 3H), 1.30-1.20 (m, 6H), 0.89 (t, J=6.5 Hz, 3H). [M+H]⁺=1070.9.

### Example 754: (R)-3-(4-((R)-4-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

The title compound was prepared in a procedure similar to that in Example 486. ¹H NMR (500 MHz, DMSO) δ 11.79 (s, 1H), 10.84 (s, 1H), 8.56 (d, J=8.7 Hz, 1H), 8.27 (s, 1H), 8.21 (s, 1H), 8.01 (s, 1H), 7.87 (d, J=9.4 Hz, 1H), 7.44 (d, J=8.9 Hz, 1H), 7.33 (s, 1H), 6.75 (s, 1H), 6.15 (d, J=12.2 Hz, 2H), 4.01 (dd, J=12.4, 4.9 Hz, 1H), 3.76 (s, 3H), 3.60 (s, 2H), 3.52 (d, J=13.0 Hz, 2H), 3.45 (d, J=5.3 Hz, 2H), 3.42-3.35 (m, 4H), 3.15-3.05 (m, 2H), 3.00-2.89 (m, 4H), 2.83-2.73 (m, 1H), 2.68 (t, J=11.3 Hz, 2H), 2.55-2.50 (m, 2H), 2.37 (s, 1H), 2.29 (d, J=6.6 Hz, 2H), 2.12-2.08 (m, 1H), 1.98 (d, J=13.3 Hz, 7H), 1.84 (d, J=10.5 Hz, 2H), 1.57 (d, J=11.2 Hz, 2H), 1.32 (s, 3H), 1.19 (s, 3H), 0.99 (s, 3H), 0.77 (s, 3H). [M+H]⁺=1069.4.

### Example 563: 3-(7-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

The title compound (26 mg, 56%) was prepared in a manner similar to that in Example 492. ¹H NMR (500 MHz, DMSO) δ 11.54 (s, 1H), 11.21 (s, 1H), 9.90 (s, 1H), 8.43 (s, 1H), 8.25 (s, 1H), 7.96 (s, 1H), 7.85 (d, J=9.3 Hz, 1H), 7.32 (s, 1H), 7.15-7.10 (m, 2H), 7.07 (d, J=7.3 Hz, 1H), 6.71 (s, 1H), 5.36 (dd, J=12.7, 5.2 Hz, 1H), 4.00 (t, J=6.9 Hz, 2H), 3.29 (s, 1H), 3.08-3.03 (m, 2H), 2.95-2.82 (m, 5H), 2.67 (s, 1H), 2.63 (s, 3H), 2.61-2.54 (m, 5H), 2.36 (s, 1H), 2.24-2.19 (m, 2H), 2.17-2.13 (m, 1H), 2.03 (d, J=13.4 Hz, 7H), 1.82 (s, 2H), 1.54 (s, 2H), 1.38 (t, J=7.6 Hz, 3H), 1.25 (t, J=6.9 Hz, 6H), 0.70 (s, 3H); [M+H]⁺=1008.7.

### Example 755: (R)-3-(4-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)azetidin-1-yl)-2,6-difluorophenyl)piperidine-2,6-dione

The title compound was prepared in a procedure similar to that in Example 488. ¹H NMR (500 MHz, DMSO) δ 11.80 (s, 1H), 10.86 (s, 1H), 8.56 (d, J=8.9 Hz, 1H), 8.28 (d, J=7.3 Hz, 1H), 8.21 (s, 1H), 8.00 (s, 1H), 7.87 (d, J=9.2 Hz, 1H), 7.44 (d, J=8.9 Hz, 1H), 7.34 (s, 1H), 6.75 (s, 1H), 6.17 (d, J=11.1 Hz, 2H), 4.07-4.00 (m, 3H), 3.92 (t, J=6.0 Hz, 2H), 3.87-3.79 (m, 1H), 3.76 (s, 3H), 3.49 (s, 2H), 3.32 (s, 3H), 3.00-2.88 (m, 4H), 2.84-2.73 (m, 1H), 2.72-2.63 (m, 2H), 2.51-2.44 (m, 4H), 2.42-2.34 (m, 1H), 2.34-2.25 (m, 2H), 2.13-2.03 (m, 1H), 2.02-1.92 (m, 7H), 1.82 (d, J=10.5 Hz, 2H), 1.63-1.51 (m, 2H), 1.32 (t, J=7.6 Hz, 3H), 0.77 (s, 3H). [M+H]⁺=1027.7.

### Example 571: 3-(4′-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2′-oxospiro[cyclopropane-1,3′-indolin]-1′-yl)piperidine-2,6-dione

The title compound (14 mg, 27%) was prepared in a manner similar to that in Example 492. ¹H NMR (500 MHz, DMSO) δ 11.67 (s, 1H), 11.09 (s, 1H), 8.64 (s, 1H), 8.24 (s, 2H), 7.90 (s, 2H), 7.46 (d, J=8.9 Hz, 2H), 7.18 (s, 1H), 6.91 (d, J=7.8 Hz, 2H), 6.72 (s, 1H), 5.36-5.31 (m, 1H), 4.02 (q, J=6.9 Hz, 2H), 3.58-3.53 (m, 2H), 3.33-3.29 (m, 4H), 3.20-2.83 (m, 9H), 2.76-2.51 (m, 8H), 2.36 (s, 1H), 2.23 (s, 2H), 2.13 (s, 1H), 1.99-1.95 (m, 9H), 1.75 (s, 1H), 1.56-1.51 (m, 2H), 1.33 (t, J=7.6 Hz, 3H), 1.27 (t, J=6.9 Hz, 3H), 0.69 (s, 3H). [M+H]⁺=1031.7.

### Example 572: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3,3-dimethyl-2-oxoindolin-1-yl)piperidine-2,6-dione

The title compound (15 mg, 25%) was prepared in a manner similar to that in Example 413. ¹H NMR (500 MHz, DMSO) δ 11.72 (s, 1H), 11.07 (s, 1H), 8.60 (d, J=9.0 Hz, 1H), 8.25 (s, 1H), 8.22 (s, 1H), 7.92 (s, 1H), 7.88 (d, J=9.2 Hz, 1H), 7.45 (d, J=8.9 Hz, 1H), 7.38 (s, 1H), 7.18 (t, J=7.8 Hz, 1H), 6.91 (d, J=7.8 Hz, 1H), 6.83 (s, 1H), 6.71 (s, 1H), 5.21 (s, 1H), 4.00 (q, J=6.9 Hz, 2H), 3.26-3.16 (m, 2H), 2.90-2.87 (m, 8H), 2.64-2.60 (m, 11H), 2.32-2.18 (m, 3H), 2.01-1.96 (m, 7H), 1.83 (d, J=10.7 Hz, 2H), 1.57-1.51 (m, 2H), 1.38 (s, 6H), 1.32 (t, J=7.6 Hz, 3H), 1.26 (t, J=6.9 Hz, 3H), 0.71 (s, 3H). [M+H]⁺=1033.7.

### Example 573: (R)-3-(4-(3-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2-oxoethyl)azetidin-1-yl)-2,6-difluorophenyl)piperidine-2,6-dione

The title compound (10.39 mg, 10%) was prepared in a manner similar to that in Example 488 step 14 from. The product was purified by HPLC (IF (2*25 cm, 5 um), 60% MtBE/40% MeOH:DCM=1:1, 80 bar, 20 ml/min) and the title compound corresponded to peak A @ 1.634 min/254 nm. ¹H NMR (500 MHz, DMSO) δ 11.77 (s, 1H), 10.87 (s, 1H), 8.56 (d, J=8.9 Hz, 1H), 8.30 (dd, J=10.0, 3.9 Hz, 1H), 8.21 (s, 1H), 7.99 (s, 1H), 7.87 (d, J=9.4 Hz, 1H), 7.42 (d, J=8.9 Hz, 1H), 7.37 (s, 1H), 6.74 (s, 1H), 6.10 (d, J=11.2 Hz, 2H), 4.06-3.92 (m, 3H), 3.75 (s, 3H), 3.52-3.39 (m, 6H), 3.02-2.90 (m, 4H), 2.83-2.71 (m, 3H), 2.71-2.61 (m, 5H), 2.49-2.45 (m, 4H), 2.33-2.29 (m, 3H), 2.14-1.90 (m, 8H), 1.82 (d, J=10.8 Hz, 2H), 1.58-1.53 (m, 2H), 0.82-0.71 (m, 3H). [M+H]⁺=1028.4.

### Example 576: (R)-3-(4-((R)-4-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

The title compound (21 mg, 39%) was prepared in a manner similar to that in Example 488 step 14 from (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide and (R)-1-(4-((R)-2,6-dioxopiperidin-3-yl)-3,5-difluorophenyl)-4,4-dimethylpyrrolidine-3-carboxylic acid. ¹H NMR (500 MHz, DMSO) δ11.28 (s, 1H), 10.84 (s, 1H), 8.64 (d, J=12.3 Hz, 1H), 8.22 (s, 1H), 8.18 (s, 1H), 7.95 (d, J=9.2 Hz, 2H), 7.33 (s, 1H), 6.71 (s, 1H), 6.15 (d, J=12.2 Hz, 2H), 4.01 (dd, J=12.4, 5.0 Hz, 1H), 3.75 (s, 3H), 3.66-3.39 (m, 10H), 3.12-3.05 (m, 2H), 2.91 (d, J=11.0 Hz, 2H), 2.83-2.73 (m, 1H), 2.68-2.54 (m, 7H), 2.35 (d, J=11.9 Hz, 1H), 2.19 (s, 2H), 2.07 (t, J=11.0 Hz, 1H), 1.98-1.92 (m, 7H), 1.82 (d, J=11.3 Hz, 2H), 1.55 (d, J=11.1 Hz, 2H), 1.18 (s, 3H), 0.99 (s, 3H), 0.71 (s, 3H). [M+H]⁺=1073.6.

### Example 589: (R)-3-(4-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

The title compound was prepared in a procedure similar to that in Example 486. 1H NMR (400 MHz, DMSO) δ 11.53 (s, 1H), 10.88 (s, 1H), 9.79 (s, 1H), 8.38 (s, 1H), 8.16 (s, 1H), 7.98 (s, 1H), 7.78 (d, J=8.8 Hz, 1H), 7.21 (s, 1H), 6.96 (d, J=10.6 Hz, 2H), 6.67 (s, 1H), 4.15-4.11 (m, 1H), 3.68 (s, 3H), 3.00-2.96 (m, 2H), 2.86 (d, J=9.2 Hz, 2H), 2.77-2.74 (m, 1H), 2.70-2.67 (m, 2H), 2.61-2.56 (m, 3H), 2.48-2.45 (m, 7H), 2.40 (s, 2H), 2.22-2.16 (m, 3H), 2.10-2.03 (m, 2H), 1.96 (d, J=13.3 Hz, 7H), 1.77 (d, J=11.4 Hz, 2H), 1.47 (d, J=11.0 Hz, 2H), 1.31 (t, J=7.4 Hz, 3H), 0.68 (s, 3H); [M+H]⁺=973.1.

### Example 711: 3-(5-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

The title compound was prepared in a procedure similar to that in Example 413. ¹H NMR (500 MHz, DMSO) δ 11.70 (s, 1H), 10.99 (s, 1H), 8.49 (d, J=8.7 Hz, 1H), 8.21 (s, 1H), 8.14 (s, 1H), 7.91 (s, 1H), 7.80 (d, J=9.3 Hz, 1H), 7.36 (d, J=8.9 Hz, 1H), 7.31 (s, 1H), 7.18 (s, 1H), 7.00 (d, J=7.4 Hz, 1H), 6.80 (d, J=8.0 Hz, 1H), 6.67 (s, 1H), 5.12 (s, 1H), 3.69 (s, 3H), 2.87 (d, J=10.9 Hz, 4H), 2.68-2.61 (m, 3H), 2.60-2.54 (m, 7H), 2.49 (d, J=18.1 Hz, 4H), 2.40 (s, 4H), 2.23 (d, J=7.3 Hz, 3H), 1.91 (d, J=13.3 Hz, 7H), 1.77 (d, J=11.0 Hz, 2H), 1.48 (d, J=8.7 Hz, 2H), 1.21 (d, J=2.9 Hz, 6H), 0.70 (s, 3H). [M+H]⁺=1006.4

### Example 584: 3-(4-(2-(4-(1-(4-((5-bromo-4-((5-(dimethylphosphoryl)-2-ethylquinolin-6-yl)amino)pyrimidin-2-yl)amino)-5-cyclopropoxy-2-methylphenyl)piperidin-4-yl)piperazin-1-yl)ethyl)-3-methyl-2-oxo-2,3-dihydro-1H-benzo[d]imidazol-1-yl)piperidine-2,6-dione

The title compound (32 mg, 46%) was prepared in a manner similar to that in Example 318. ¹H NMR (500 MHz, DMSO) δ 11.88 (s, 1H), 11.09 (s, 1H), 8.55 (d, J=8.8 Hz, 1H), 8.34 (s, 1H), 8.19 (s, 1H), 7.91-7.84 (m, 211), 7.44 (d, J=8.9 Hz, 1H), 7.30 (s, 1H), 7.01-6.94 (m, 3H), 6.91 (dd, J=6.2, 2.6 Hz, 1H), 5.37 (dd, J=12.8, 5.4 Hz, 1H), 3.81 (tt, J=5.9, 2.9 Hz, 1H), 3.59 (s, 311), 3.12-3.01 (m, 4H), 2.97-2.84 (m, 3H), 2.78-2.52 (m, 13H), 2.31 (t, J=11.2 Hz, 111), 2.07-1.91 (m, 8H), 1.94-1.81 (m, 5H), 1.57 (dd, J=20.2, 11.2 Hz, 211), 1.32 (t, J=7.6 Hz, 3H), 0.70 (q, J=6.0 Hz, 2H), 0.61-0.55 (m, 2H). [M+H]⁺=1018.3

### Example 551: (R)-3-(4-(3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)azetidin-1-yl)-2,6-difluorophenyl)piperidine-2,6-dione

The title compound (30 mg, 41%) was prepared in a manner similar to that in Example 484. ¹H NMR (500 MHz, DMSO) δ 12.11 (s, 1H), 10.85 (s, 1H), 8.64 (s, 1H), 8.52 (s, 1H), 8.26 (s, 1H), 8.18 (d, J=8.9 Hz, 1H), 8.09 (s, 1H), 7.34 (s, 1H), 6.73 (s, 1H), 6.12 (s, 1H), 6.09 (s, 1H), 4.17 (d, J=7.1 Hz, 2H), 4.00 (d, J=6.9 Hz, 3H), 3.93 (s, 2H), 3.47 (s, 3H), 3.00-2.86 (m, 3H), 2.82-2.73 (m, 1H), 2.64 (t, J=11.1 Hz, 2H), 2.55 (d, J=6.8 Hz, 6H), 2.39-2.34 (m, 8H), 2.03 (d, J=13.4 Hz, 6H), 1.96-1.90 (m, 2H), 1.83 (d, J=11.0 Hz, 2H), 1.55 (d, J=11.3 Hz, 2H), 1.31 (t, J=7.1 Hz, 3H), 1.25 (t, J=6.9 Hz, 3H), 0.87 (s, 3H). [M+H]⁺=1044.4.

### Example 577: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-5-ethoxy-2-ethylphenyl)piperidin-4-yl)piperazine-1-carbonyl)pyrrolidin-1-yl)-2,6-difluorophenyl)piperidine-2,6-dione

The title compound (27 mg, 55%) was prepared in a manner similar to that in Example 488. ¹H NMR (500 MHz, DMSO) δ 11.20 (s, 1H), 10.84 (s, 1H), 8.70 (s, 1H), 8.23 (s, 1H), 8.15 (s, 1H), 7.95 (d, J=9.1 Hz, 1H), 7.85 (s, 1H), 7.38 (s, 1H), 6.68 (s, 111), 6.23 (d, J=12.2 Hz, 211), 4.00 (d, J=6.9 Hz, 311), 3.58-3.43 (m, 8H), 3.29-3.23 (m, 3H), 2.89 (d, J=10.4 Hz, 2H), 2.82-2.74 (m, 1H), 2.65-2.63 (m, 8H), 2.34 (d, J=11.2 Hz, 1H), 2.21-2.03 (m, 5H), 1.98-1.91 (m, 7H), 1.81 (d, J=11.2 Hz, 2H), 1.60-1.49 (m, 2H), 1.27 (t, J=6.9 Hz, 3H), 0.64 (s, 3H). [M+H]⁺=1059.6.

### Example 566: 3-(7-(3-((4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)methyl)azetidin-1-yl)-2-oxobenzol[d]oxazol-3(2H)-yl)piperidine-2,6-dione

The title compound (26 mg, 56%) was prepared in a manner similar to that in Example 492. ¹H NMR (500 MHz, DMSO) δ 11.53 (s, 1H), 11.18 (s, 1H), 9.90 (s, 1H), 8.43 (s, 1H), 8.25 (s, 1H), 7.96 (s, 1H), 7.86 (d, J=9.2 Hz, 1H), 7.32 (s, 1H), 7.01 (t, J=8.1 Hz, 1H), 6.71 (s, 1H), 6.57 (d, J=7.8 Hz, 1H), 6.25 (d, J=8.1 Hz, 1H), 5.29 (dd, J=12.9, 5.2 Hz, 1H), 4.11 (t, J=7.4 Hz, 2H), 4.01-3.97 (m, 2H), 3.66 (s, 2H), 3.29 (s, 2H), 3.08-3.04 (m, 2H), 2.91 (d, J=10.8 Hz, 3H), 2.89-2.83 (m, 1H), 2.68-2.57 (m, 7H), 2.54 (s, 1H), 2.40 (s, 3H), 2.36 (s, 1H), 2.20 (s, 2H), 2.14-2.10 (m, 1H), 2.03 (d, J=13.4 Hz, 7H), 1.81 (s, 2H), 1.54 (s, 2H), 1.38 (t, J=7.6 Hz, 3H), 1.25 (t, J=6.9 Hz, 3H), 0.69 (s, 3H); [M+H]⁺=1049.7.

### Example 558: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-ethyl-5-fluoro-2-oxo-2,3-dihydro-1H-benzo[d]imidazol-1-yl)piperidine-2,6-dione

The title compound (10.53 mg, 10%) was prepared in a manner similar to that in Example 318. ¹H NMR (500 MHz, DMSO) δ 12.11 (s, 1H), 11.11 (s, 1H), 8.73 (d, J=5.9 Hz, 1H), 8.29 (s, 1H), 7.97 (s, 1H), 7.52 (d, J=8.9 Hz, 1H), 7.43 (s, 1H), 7.10 (d, J=9.2 Hz, 2H), 6.96 (d, J=8.6 Hz, 2H), 6.73 (s, 1H), 5.39 (dd, J=12.5, 4.9 Hz, 1H), 4.12-3.91 (m, 4H), 3.73-3.47 (m, 2H), 3.32 (m, 4H), 3.19-2.95 (m, 7H), 2.94-2.83 (m, 1H), 2.72-2.68 (m, 6H), 2.55 (d, J=8.9 Hz, 3H), 2.33-2.21 (m, 2H), 2.22-2.18 (m, 1H), 2.03 (t, J=10.8 Hz, 7H), 1.92-1.77 (m, 2H), 1.32 (t, J=7.2 Hz, 3H), 1.30-1.20 (m, 6H), 0.70 (t, J=6.7 Hz, 3H). [M+H]⁺=1052.3.

### Example 462 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-6-fluoro-3-methyl-2-oxo-2,3-dihydro-1H-benzo[d]imidazol-1-yl)piperidine-2,6-dione

The title compound (46 mg, 43%) was prepared in a manner similar to that in Example 318. ¹H NMR (500 MHz, DMSO) δ 11.73 (s, 1H), 11.10 (s, 1H), 8.60 (d, J=9.1 Hz, 1H), 8.27 (s, 1H), 8.22 (s, 1H), 8.01-7.82 (m, 2H), 7.45 (d, J=8.9 Hz, 1H), 7.38 (s, 1H), 7.01 (d, J=7.2 Hz, 1H), 6.80 (dd, J=11.0, 2.3 Hz, 1H), 6.71 (s, 1H), 5.35 (dd, J=12.4, 5.3 Hz, 1H), 4.00 (d, J=7.0 Hz, 2H), 3.57 (s, 3H), 3.11-3.03 (m, 2H), 2.93 (d, J=7.5 Hz, 4H), 2.84 (d, J=12.4 Hz, 1H), 2.73 (dt, J=17.1, 10.7 Hz, 2H), 2.62-2.55 (m, 12H), 2.26 (s, 3H), 2.02-1.92 (m, 7H), 1.83 (d, J=10.2 Hz, 2H), 1.61-1.41 (m, 2H), 1.32 (t, J=7.6 Hz, 3H), 1.25 (t, J=6.9 Hz, 3H), 0.71 (s, 3H). [M+H]⁺=1038.4

### Example 643: 3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-3-ethyl-2-oxo-2,3-dihydro-1H-benzo[d]imidazol-1-yl)piperidine-2,6-dione

The title compound was prepared in a procedure similar to that in Example 318. 1H NMR (500 MHz, DMSO) δ 12.03 (s, 1H), 11.03 (s, 1H), 8.50 (d, J=8.4 Hz, 1H), 8.25 (d, J=8.7 Hz, 1H), 8.16-8.14 (m, 1H), 7.97 (s, 1H), 7.47 (d, J=9.0 Hz, 1H), 7.33 (s, 1H), 6.96-6.82 (m, 3H), 6.65 (s, 1H), 5.32-5.28 (m, 1H), 3.95-3.93 (m, 4H), 2.96-2.77 (m, 8H), 2.70-2.46 (m, 13H), 2.30-2.20 (m, 3H), 1.93 (d, J=13.3 Hz, 7H), 1.78-1.75 (m, 2H), 1.50-1.43 (m, 2H), 1.28-1.13 (m, 9H), 0.68 (s, 3H). [M+H]⁺=1052.7.

### Example 745: (R)-3-(4-(2-(1′-(4-((5-bromo-4-((5-(dimethylphosphoryl)-2-ethylquinolin-6-yl)amino)pyrimidin-2-yl)amino)-2-ethyl-5-methoxyphenyl)-[4,4′-bipiperidin]-1-yl)ethyl)-2,6-difluorophenyl)piperidine-2,6-dione

The title compound was prepared in a procedure similar to that in Example 486. ¹H NMR (500 MHz, DMSO) δ 11.79 (s, 1H), 10.96 (s, 1H), 8.56 (d, J=8.9 Hz, 1H), 8.27 (s, 1H), 8.21 (s, 1H), 8.01 (s, 1H), 7.87 (d, J=9.3 Hz, 1H), 7.44 (d, J=8.9 Hz, 1H), 7.33 (s, 1H), 7.07 (d, J=10.0 Hz, 2H), 6.74 (s, 1H), 4.22 (dd, J=12.6, 4.9 Hz, 1H), 3.76 (s, 3H), 2.93 (s, 7H), 2.80 (dd, J=13.2, 5.0 Hz, 1H), 2.63 (t, J=10.9 Hz, 3H), 2.54-2.50 (m, 6H), 2.29 (d, J=6.7 Hz, 2H), 2.13-2.09 (m, 1H), 1.98 (d, J=13.3 Hz, 7H), 1.87-1.73 (m, 4H), 1.32 (t, J=7.6 Hz, 7H), 1.23 (s, 1H), 0.77 (s, 3H). [M+H]⁺=971.3.

### Example 744: (R)-3-(4-(2-(4-(1-(4-((5-chloro-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

The title compound was prepared in a procedure similar to that in Example 486. ¹H NMR (500 MHz, DMSO) δ 12.04 (s, 1H), 10.96 (s, 1H), 8.56 (s, 1H), 8.45 (s, 1H), 8.14 (d, J=6.9 Hz, 1H), 8.03 (s, 1H), 7.89 (d, J=9.0 Hz, 1H), 7.45 (d, J=9.0 Hz, 2H), 7.06 (d, J=10.1 Hz, 2H), 6.76 (s, 1H), 4.21 (d, J=8.5 Hz, 1H), 3.77 (s, 3H), 3.54 (s, 1H), 3.01 (s, 4H), 2.93 (q, J=7.5 Hz, 2H), 2.86-2.76 (m, 2H), 2.74-2.67 (m, 4H), 2.55 (s, 2H), 2.52 (s, 6H), 2.45-2.30 (m, 3H), 2.21-2.10 (m, 2H), 2.00 (d, J=13.3 Hz, 7H), 1.81 (s, 1H), 1.58 (s, 1H), 1.32 (t, J=7.6 Hz, 3H), 0.82 (s, 3H). [M+H]⁺=928.4.

### Example 756: 3-(4′-(2-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2′-oxospiro[cyclopropane-1,3′-indolin]-1′-yl)piperidine-2,6-dione

The title compound was prepared in a procedure similar to that in Example 413. ¹H NMR (500 MHz, DMSO) δ 12.21 (s, 1H), 11.08 (s, 1H), 8.63-8.51 (m, 2H), 8.23-8.16 (m, 3H), 7.34 (s, 1H), 7.15 (t, J=7.8 Hz, 1H), 6.88 (d, J=7.7 Hz, 2H), 6.76 (s, 1H), 5.31 (s, 1H), 3.75 (s, 3H), 3.72 (s, 3H), 2.99 (d, J=10.3 Hz, 2H), 2.88 (t, J=12.5 Hz, 1H), 2.71-2.60 (m, 4H), 2.57-2.54 (m, 6H), 2.49-2.37 (m, 9H), 2.32 (t, J=11.0 Hz, 1H), 2.03 (d, J=13.4 Hz, 6H), 1.95 (d, J=3.4 Hz, 2H), 1.85 (d, J=10.8 Hz, 2H), 1.57 (d, J=11.4 Hz, 2H), 1.48 (d, J=3.7 Hz, 2H), 0.93 (s, 3H). [M+H]⁺=1020.7.

### Example 748: (R)-3-(4-(2-(4-(1-(4-((5-bromo-4-((5-(dimethylphosphoryl)-2-ethylquinazolin-6-yl)amino)pyrimidin-2-yl)amino)-5-methoxy-2-methylphenyl)piperidin-4-yl)piperazin-1-yl)ethyl)-2,6-difluorophenyl)piperidine-2,6-dione

The title compound was prepared in a procedure similar to that in Example 460. ¹H NMR (400 MHz, DMSO) δ 11.74 (s, 1H), 10.95 (s, 1H), 9.82 (s, 1H), 8.48 (s, 1H), 8.22 (s, 1H), 8.05 (s, 1H), 7.84 (s, 1H), 7.23 (s, 1H), 7.02 (d, J=10.0 Hz, 2H), 6.67 (s, 1H), 4.20 (dd, J=12.6, 5.0 Hz, 1H), 3.75 (s, 3H), 3.01 (s, 4H), 2.85-2.82 (m, 1H), 2.77-2.74 (m, 3H), 2.62-2.57 (m, 3H), 2.55-2.53 (m, 6H), 2.48-2.45 (m, 2H), 2.27 (s, 1H), 2.19-2.10 (m, 2H), 2.03-1.98 (m, 6H), 1.89-1.82 (m, 6H), 1.54 (d, J=9.7 Hz, 2H), 1.36 (d, J=6.8 Hz, 3H). [M+H]⁺=959.0.

### Example 749: (R)-3-(4-(2-(4-(1-(4-((4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

The title compound was prepared in a procedure similar to that in Example 460. ¹H NMR (400 MHz, DMSO) δ 11.01 (s, 1H), 10.95 (s, 1H), 10.18 (s, 1H), 8.57 (s, 1H), 8.07 (d, J=5.6 Hz, 1H), 7.95 (d, J=9.1 Hz, 1H), 7.69 (s, 1H), 7.57 (s, 1H), 7.04 (d, J=10.1 Hz, 2H), 6.71 (s, 1H), 6.18 (d, J=5.6 Hz, 1H), 4.20 (dd, J=12.6, 4.9 Hz, 1H), 4.05-4.01 (m, 2H), 3.07-3.03 (m, 2H), 2.92 (d, J=10.2 Hz, 2H), 2.83-2.79 (m, 1H), 2.78-2.75 (m, 3H), 2.66-2.59 (m, 5H), 2.55-2.52 (m, 7H), 2.24-2.21 (m, 2H), 2.13 (dd, J=13.4, 3.7 Hz, 1H), 1.99 (d, J=13.4 Hz, 8H), 1.89 (s, 2H), 1.57 (s, 2H), 1.37 (t, J=7.6 Hz, 3H), 1.30 (t, J=6.9 Hz, 3H), 0.72 (s, 3H). [M+H]⁺=909.2.

### Example 746: (R)-3-(4-((R)-4-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazine-1-carbonyl)-3,3-dimethylpyrrolidin-1-yl)-2,6-difluorophenyl)piperidine-2,6-dione

The title compound was prepared in a procedure similar to that in Example 488. ¹H NMR (500 MHz, DMSO) δ 12.10 (s, 1H), 10.84 (s, 1H), 8.57 (d, J=7.0 Hz, 1H), 8.32-8.27 (m, 2H), 8.05 (s, 1H), 7.54 (d, J=9.0 Hz, 1H), 7.39 (s, 1H), 6.71 (s, 1H), 6.15 (d, J=12.2 Hz, 2H), 4.01 (d, J=7.0 Hz, 3H), 3.70-3.57 (m, 8H), 3.15-3.05 (m, 3H), 2.95 (d, J=7.6 Hz, 4H), 2.84-2.73 (m, 2H), 2.68-2.59 (m, 4H), 2.40-2.23 (m, 3H), 2.00 (d, J=13.3 Hz, 8H), 1.83 (d, J=10.6 Hz, 2H), 1.55 (d, J=10.8 Hz, 2H), 1.32 (t, J=7.6 Hz, 3H), 1.27 (d, J=6.9 Hz, 3H), 1.18 (s, 3H), 0.99 (s, 3H), 0.74 (s, 3H). [M+H]⁺=1101.4.

### Example 747: (R)-3-(4-((R)-4-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazine-1-carbonyl)-3,3-dimethylpyrrolidin-1-yl)-2,6-difluorophenyl)piperidine-2,6-dione

The title compound was prepared in a procedure similar to that in Example 488. ¹H NMR (500 MHz, DMSO) δ 11.62 (s, 1H), 10.77 (s, 1H), 8.53 (d, J=8.9 Hz, 1H), 8.18 (d, J=21.2 Hz, 2H), 7.95-7.77 (m, 2H), 7.36 (d, J=8.7 Hz, 2H), 6.63 (s, 1H), 6.09 (d, J=12.2 Hz, 2H), 3.93 (q, J=6.9 Hz, 3H), 3.60-3.45 (m, 8H), 3.02 (dd, J=18.7, 9.2 Hz, 2H), 2.86 (d, J=10.7 Hz, 2H), 2.76-2.67 (m, 2H), 2.58 (s, 6H), 2.29 (s, 1H), 2.19 (d, J=5.8 Hz, 2H), 2.06-1.85 (m, 9H), 1.75 (d, J=10.6 Hz, 2H), 1.49 (d, J=10.7 Hz, 2H), 1.19 (t, J=6.9 Hz, 4H), 1.10 (d, J=10.9 Hz, 3H), 0.92 (s, 3H), 0.64 (s, 3H). [M+H]⁺=1069.4.

### Example 742: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-ethyl-6,7-difluoro-2-oxo-2,3-dihydro-1H-benzo[d]imidazol-1-yl)piperidine-2,6-dione

The title compound was prepared in a procedure similar to that in Example 318. ¹H NMR (500 MHz, DMSO) δ 11.72 (s, 1H), 11.16 (s, 1H), 8.60 (d, J=8.8 Hz, 1H), 8.22 (s, 2H), 7.91 (s, 1H), 7.88 (d, J=9.2 Hz, 1H), 7.45 (d, J=8.9 Hz, 1H), 7.38 (s, 1H), 7.09 (dd, J=12.5, 7.8 Hz, 1H), 6.71 (s, 1H), 5.57 (dd, J=12.9, 5.2 Hz, 1H), 4.08-3.94 (m, 4H), 2.94 (dt, J=14.4, 7.1 Hz, 8H), 2.71-2.60 (m, 4H), 2.59-2.52 (m, 8H), 2.25 (s, 4H), 2.13 (s, 1H), 1.98 (d, J=13.3 Hz, 6H), 1.83 (d, J=11.2 Hz, 2H), 1.53-1.47 (m, 2H), 1.32 (t, J=7.6 Hz, 3H), 1.26 (t, J=6.9 Hz, 6H), 0.71 (s, 3H). [M+H]⁺=1070.4

### Example 740: (R)-3-(4-((R)-4-(4-(1-(4-((5-bromo-4-((5-(dimethylphosphoryl)-2-methylquinazolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

The title compound was prepared in a procedure similar to that in Example 488. ¹H NMR (500 MHz, DMSO) δ 11.53 (s, 1H), 10.84 (s, 1H), 9.86 (s, 1H), 8.47 (s, 1H), 8.24 (s, 1H), 8.02 (s, 1H), 7.85 (d, J=8.2 Hz, 1H), 7.32 (s, 1H), 6.73 (s, 1H), 6.15 (d, J=12.2 Hz, 2H), 4.01 (dd, J=12.5, 4.9 Hz, 1H), 3.75 (s, 3H), 3.47-3.21 (m, 9H), 3.09-3.02 (m, 2H), 2.93 (d, J=10.3 Hz, 2H), 2.84-2.73 (m, 4H), 2.65 (t, J=10.8 Hz, 2H), 2.54-2.49 (m, 3H), 2.36-2.31 (m, 1H), 2.26-2.21 (m, 2H), 2.14-1.92 (m, 8H), 1.83 (d, J=10.4 Hz, 2H), 1.56 (d, J=10.6 Hz, 2H), 1.18 (s, 3H), 0.99 (s, 3H), 0.75 (s, 3H). [M+H]⁺=1056.4.

### Example 741: (R)-3-(4-(2-(4-(1-(4-((5-bromo-4-((2-cyclopropyl-5-(dimethylphosphoryl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

The title compound was prepared in a procedure similar to that in Example 486. ¹H NMR (500 MHz, DMSO) δ 11.75 (s, 1H), 10.95 (s, 1H), 8.49 (d, J=8.9 Hz, 1H), 8.21-8.17 (m, 2H), 8.03 (s, 1H), 7.74 (d, J=9.4 Hz, 1H), 7.44 (d, J=9.0 Hz, 1H), 7.27 (s, 1H), 7.03 (d, J=10.0 Hz, 2H), 6.76 (s, 1H), 4.20 (dd, J=12.6, 5.0 Hz, 1H), 3.76 (s, 3H), 3.29-3.24 (m, 2H), 2.95 (d, J=10.9 Hz, 2H), 2.86-2.72 (m, 3H), 2.67 (t, J=11.2 Hz, 2H), 2.54-2.38 (m, 7H), 2.48-2.39 (m, 2H), 2.34-2.23 (m, 4H), 2.13 (dt, J=12.9, 9.0 Hz, 1H), 1.99-1.89 (m, 7H), 1.86 (d, J=11.8 Hz, 2H), 1.55-1.51 (m, 2H), 1.06 (d, J=6.4 Hz, 4H), 0.78 (s, 3H). [M+H]⁺=984.3.

### Example 738: (R)-3-(4-(2-(4-(1-(4-((5-bromo-4-((5-(dimethylphosphoryl)-2-methylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

The title compound was prepared in a procedure similar to that in Example 460. ¹H NMR (500 MHz, DMSO) δ 11.44 (s, 1H), 10.95 (s, 1H), 9.91 (s, 1H), 8.43 (s, 1H), 8.25 (s, 1H), 7.93 (s, 1H), 7.86 (d, J=9.2 Hz, 1H), 7.36 (s, 1H), 7.04 (d, J=10.1 Hz, 2H), 6.69 (s, 1H), 4.20 (dd, J=12.7, 5.0 Hz, 1H), 4.00 (q, J=6.9 Hz, 2H), 3.31-3.28 (m, 2H), 2.91 (d, J=9.5 Hz, 2H), 2.86-2.70 (m, 7H), 2.68-2.51 (m, 9H), 2.36 (s, 2H), 2.15-2.11 (m, 3H), 2.01-1.92 (m, 7H), 1.86 (s, 2H), 1.56-1.51 (m, 2H), 1.25 (t, J=6.9 Hz, 3H), 0.68 (s, 3H). [M+H]⁺=973.3.

### Example 739: (R)-3-(4-(2-(4-(1-(4-((5-bromo-4-((5-(dimethylphosphoryl)-2-methylquinazolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

The title compound was prepared in a procedure similar to that in Example 460. ¹H NMR (500 MHz, DMSO) δ 11.46 (s, 1H), 10.88 (s, 1H), 9.79 (s, 1H), 8.40 (s, 1H), 8.17 (s, 1H), 7.95 (s, 1H), 7.78 (d, J=9.2 Hz, 1H), 7.24 (s, 1H), 6.96 (d, J=10.1 Hz, 2H), 6.66 (s, 1H), 4.13 (dd, J=12.7, 4.9 Hz, 1H), 3.68 (s, 3H), 3.25-3.09 (m, 2H), 2.85 (d, J=10.6 Hz, 2H), 2.79-2.65 (m, 6H), 2.57 (t, J=11.1 Hz, 2H), 2.47-2.32 (m, 6H), 2.39-2.36 (m, 3H), 2.19 (s, 3H), 2.06 (dt, J=13.3, 9.7 Hz, 1H), 1.94-1.86 (m, 7H), 1.76 (d, J=10.9 Hz, 2H), 1.47-1.42 (m, 2H), 0.68 (s, 3H). [M+H]⁺=959.3.

### Example 736: 3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-5-fluoro-3-methyl-2-oxo-2,3-dihydro-1H-benzo[d]imidazol-1-yl)piperidine-2,6-dione

The title compound was prepared in a procedure similar to that in Example 318. ¹H NMR (500 MHz, DMSO) δ 11.46 (s, 1H), 11.11 (s, 1H), 9.94 (s, 1H), 8.42 (s, 1H), 8.26 (s, 1H), 7.94 (s, 1H), 7.87 (d, J=8.8 Hz, 1H), 7.35 (s, 1H), 7.02 (s, 1H), 6.91 (t, J=9.9 Hz, 1H), 6.71 (s, 1H), 5.38 (dd, J=12.7, 5.0 Hz, 1H), 4.01 (q, J=6.9 Hz, 2H), 3.60 (s, 3H), 3.31-3.28 (m, 2H), 3.08-3.04 (m, 5H), 2.89-2.86 (m, 4H), 2.68-2.62 (m, 8H), 2.49-2.45 (m, 5H), 2.20 (s, 2H), 2.04-2.01 (m, 8H), 1.62-1.61 (m, 1H), 1.39 (t, J=7.6 Hz, 3H), 1.25 (dd, J=13.4, 6.5 Hz, 3H), 0.68 (s, 3H). [M+H]⁺=1039.4.

### Example 737: 3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-3-ethyl-2-oxo-2,3-dihydro-1H-benzo[d]imidazol-1-yl)piperidine-2,6-dione

The title compound was prepared in a procedure similar to that in Example 318. ¹H NMR (500 MHz, DMSO) δ 11.46 (s, 1H), 11.10 (s, 1H), 9.94 (s, 1H), 8.42 (s, 1H), 8.26 (s, 1H), 7.95 (s, 1H), 7.87 (d, J=9.4 Hz, 1H), 7.34 (s, 1H), 7.01 (s, 2H), 6.94 (dd, J=5.9, 3.0 Hz, 1H), 6.71 (s, 1H), 5.37 (dd, J=12.8, 5.3 Hz, 1H), 4.01-3.96 (m, 4H), 3.30-3.26 (m, 2H), 3.13-2.84 (m, 10H), 2.79-2.57 (m, 8H), 2.50-2.47 (m, 2H), 2.19-2.16 (m, 2H), 1.99-1.85 (m, 9H), 1.62-1.58 (m, 2H), 1.39 (t, J=7.6 Hz, 3H), 1.25-1.13 (m, 6H), 0.68 (s, 3H). [M+H]⁺=1035.4.

### Example 732: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-5-methoxy-2-methylphenyl)piperidin-4-yl)piperazine-1-carbonyl)pyrrolidin-1-yl)-2,6-difluorophenyl)piperidine-2,6-dione

The title compound was prepared in a procedure similar to that in Example 488. ¹H NMR (500 MHz, DMSO) δ 11.50 (s, 1H), 10.84 (s, 1H), 8.53 (d, J=12.4 Hz, 1H), 8.27 (s, 1H), 8.22 (s, 1H), 7.95 (d, J=11.9 Hz, 2H), 7.30 (s, 1H), 6.67 (s, 1H), 6.23 (d, J=12.1 Hz, 2H), 4.02 (dd, J=12.4, 5.0 Hz, 1H), 3.75 (s, 3H), 3.60-3.41 (m, 8H), 3.29-3.22 (m, 3H), 3.01 (d, J=10.7 Hz, 2H), 2.82-2.73 (m, 1H), 2.66-2.54 (m, 8H), 2.36 (s, 1H), 2.19-2.04 (m, 3H), 1.99-1.93 (m, 7H), 1.87-1.80 (m, 5H), 1.58 (d, J=9.8 Hz, 2H). [M+H]⁺=1031.5.

### Example 733: (R)-3-(4-(2-(4-(1-(4-((4-((5-(dimethylphosphoryl)-2-ethyl-8-fluoroquinolin-6-yl)amino)-5-methylpyrimidin-2-yl)amino)-5-ethoxy-2-ethylphenyl)piperidin-4-yl)piperazin-1-yl)ethyl)-2,6-difluorophenyl)piperidine-2,6-dione

The title compound was prepared in a procedure similar to that in Example 486. ¹H NMR (500 MHz, DMSO) δ 12.17 (s, 1H), 10.95 (s, 1H), 8.79 (d, J=14.1 Hz, 1H), 8.46 (d, J=9.5 Hz, 1H), 7.97 (s, 1H), 7.67 (s, 1H), 7.62 (s, 1H), 7.53 (d, J=9.0 Hz, 1H), 7.03 (d, J=10.0 Hz, 2H), 6.74 (s, 1H), 4.20 (dd, J=12.6, 5.0 Hz, 1H), 4.05 (q, J=7.0 Hz, 2H), 2.99-2.91 (m, 4H), 2.86-2.71 (m, 311), 2.65-2.62 (m, 3H), 2.58-2.55 (m, 7H), 2.48-2.35 (m, 5H), 2.29-2.26 (m, 1H), 2.19-2.08 (m, 4H), 2.03-2.01 (m, 7H), 1.86-1.83 (m, 2H), 1.56-1.54 (m, 2H), 1.34-1.27 (m, 6H), 0.85 (t, J=7.4 Hz, 3H). [M+H]⁺=940.3.

### Example 730: (R)-3-(4-(2-(4-(1-(4-((5-chloro-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

The title compound was prepared in a procedure similar to that in Example 484. ¹H NMR (500 MHz, DMSO) δ 12.43 (s, 1H), 10.95 (s, 1H), 8.53 (d, J=8.8 Hz, 2H), 8.17 (s, 1H), 8.10 (s, 1H), 7.54 (d, J=9.0 Hz, 1H), 7.39 (s, 11H), 7.03 (d, J=10.0 Hz, 2H), 6.73 (s, 1H), 4.20 (dd, J=12.8, 5.3 Hz, 1H), 4.02 (q, J=6.9 Hz, 2H), 3.01-2.91 (m, 4H), 2.85-2.71 (m, 4H), 2.66-2.64 (m, 5H), 2.56-2.53 (m, 6H), 2.40-2.33 (m, 2H), 2.30-2.28 (m, 1H), 2.18-2.07 (m, 2H), 2.01-1.99 (m, 7H), 1.88-1.81 (m, 2H), 1.60-1.49 (m, 2H), 1.32 (t, J=7.6 Hz, 3H), 1.25 (t, J=6.9 Hz, 3H), 0.85-0.82 (m, 3H). [M+H]⁺=960.3.

### Example 731: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4,6-difluoro-2-oxobenzo[d]oxazol-3(2H)-yl)piperidine-2,6-dione

The title compound was prepared in a procedure similar to that in Example 492. ¹H NMR (500 MHz, DMSO) δ 11.72 (s, 1H), 11.26 (s, 1H), 8.60 (d, J=8.8 Hz, 1H), 8.22 (s, 2H), 7.91 (s, 1H), 7.87 (d, J=9.3 Hz, 1H), 7.45 (d, J=8.9 Hz, 1H), 7.38 (s, 1H), 7.27 (t, J=10.9 Hz, 1H), 6.70 (s, 1H), 5.43 (s, 1H), 4.00 (d, J=7.0 Hz, 2H), 2.98-2.83 (m, 8H), 2.70-2.52 (m, 8H), 2.38-2.34 (m, 4H), 2.32-2.16 (m, 5H), 1.98 (d, J=13.3 Hz, 6H), 1.82 (d, J=10.7 Hz, 2H), 1.56-1.48 (m, 2H), 1.32 (t, J=7.6 Hz, 3H), 1.25 (t, J=6.9 Hz, 3H), 0.71 (s, 3H). [M+H]⁺=1043.5.

### Example 728: (R)-3-(4-(2-(4-(1-(4-((5-bromo-4-((5-(dimethylphosphoryl)-3-fluoro-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

The title compound was prepared in a procedure similar to that in Example 484. ¹H NMR (500 MHz, DMSO) δ11.19 (s, 1H), 10.95 (s, 1H), 8.69 (d, J=12.0 Hz, 1H), 8.23 (s, 1H), 8.15 (s, 1H), 7.95 (d, J=9.1 Hz, 1H), 7.85 (s, 1H), 7.37 (s, 1H), 7.02 (d, J=10.1 Hz, 2H), 6.68 (s, 1H), 4.20 (dd, J=12.7, 4.9 Hz, 1H), 3.99 (d, J=6.9 Hz, 2H), 2.90-2.73 (m, 6H), 2.66-2.53 (m, 11H), 2.48-2.40 (m, 3H), 2.26 (s, 1H), 2.17-2.08 (m, 3H), 2.02-1.92 (m, 8H), 1.81 (d, J=10.9 Hz, 2H), 1.56-1.47 (m, 2H), 1.26 (t, J=6.9 Hz, 3H), 0.64 (s, 3H). [M+H]⁺=990.5.

### Example 729: (R)-3-(4-((R)-4-(4-(1-(4-((5-bromo-4-((5-(dimethylphosphoryl)-3-fluoro-2-methylquinolin-6-yl)amino)pyrimidin-2-yl)amino)-5-methoxy-2-methylphenyl)piperidin-4-yl)piperazine-1-carbonyl)-3,3-dimethylpyrrolidin-1-yl)-2,6-difluorophenyl)piperidine-2,6-dione

The title compound was prepared in a procedure similar to that in Example 488. ¹H NMR (500 MHz, DMSO) δ 11.51 (s, 1H), 10.84 (s, 1H), 8.53 (d, J=12.0 Hz, 1H), 8.26 (s, 1H), 8.22 (s, 1H), 7.95 (d, J=9.0 Hz, 2H), 7.30 (s, 1H), 6.66 (s, 1H), 6.15 (d, J=12.2 Hz, 2H), 4.01 (dd, J=12.7, 4.8 Hz, 1H), 3.75 (s, 3H), 3.60 (s, 2H), 3.53-3.38 (m, 6H), 3.09 (dd, J=18.4, 9.1 Hz, 2H), 3.00 (d, J=10.7 Hz, 2H), 2.83-2.74 (m, 1H), 2.66-2.54 (m, 8H), 2.36 (s, 1H), 2.07 (t, J=12.7 Hz, 1H), 1.99-1.93 (m, 8H), 1.86-1.80 (m, 5H), 1.57 (d, J=11.4 Hz, 2H), 1.18 (s, 3H), 0.99 (s, 3H). [M+H]⁺=1059.6.

### Example 727: (R)-3-(4-((R)-4-(4-(1-(4-((5-bromo-4-((5-(dimethylphosphoryl)-2-methylquinolin-6-yl)amino)pyrimidin-2-yl)amino)-5-ethoxy-2-methylphenyl)piperidin-4-yl)piperazine-1-carbonyl)-3,3-dimethylpyrrolidin-1-yl)-2,6-difluorophenyl)piperidine-2,6-dione

The title compound was prepared in a procedure similar to that in Example 488. ¹H NMR (500 MHz, DMSO) δ 11.87 (s, 1H), 10.84 (s, 1H), 8.55 (d, J=8.9 Hz, 1H), 8.37 (d, J=5.5 Hz, 1H), 8.22 (s, 1H), 7.93-7.84 (m, 2H), 7.49-7.37 (m, 2H), 6.65 (s, 1H), 6.15 (d, J=12.2 Hz, 2H), 4.01 (q, J=7.0 Hz, 3H), 3.64-3.38 (m, 7H), 3.12-2.98 (m, 4H), 2.74-2.82 (m, 1H), 2.65 (s, 3H), 2.54-2.61 (m, 7H), 2.36 (s, 1H), 2.14-2.03 (m, 1H), 2.03-1.92 (m, 7H), 1.92-1.78 (m, 5H), 1.58 (q, J=12.4 Hz, 2H), 1.26 (t, J=6.9 Hz, 3H), 1.18 (s, 3H), 0.99 (s, 3H). [M+H]⁺=1055.0.

### Example 725: (3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)-2,6-dioxopiperidin-1-yl)methyl pivalate

To a stirred solution of 3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione (50 mg, 0.05 mmol), TBAI (5 mg, 0.014 mmol) and Cs₂CO₃ (33 mg, 0.1 mmol) in DMF (2 mL) was added chloromethyl pivalate (8 mg, 0.05 mmol). The resulting mixture was stirred at 25° C. for 16 hours. The reaction mixture was poured into ice-water, and then extracted with DCM (2×10.0 mL). The combined organic layers were washed with brine (2×10.0 mL), dried over Na₂SO₄ and concentrated under vacuum to afford the crude residue, which was purified with silica gel column chromatography (DCM:MeOH=20:1) to give the title product (19.5 mg, 38.5%). ¹H NMR (500 MHz, DMSO) δ 12.10 (s, 1H), 8.57 (d, J=9.0 Hz, 1H), 8.34-8.32 (m, 1H), 8.24 (s, 1H), 8.05 (s, 1H), 7.54 (d, J=9.0 Hz, 1H), 7.40 (s, 1H), 7.06 (d, J=10.2 Hz, 2H), 6.71 (s, 1H), 5.69 (s, 2H), 4.43-4.39 (m, 1H), 4.03-3.99 (m, 2H), 3.22-3.11 (m, 2H), 3.06-2.90 (m, 5H), 2.78-2.74 (m, 3H), 2.64-2.60 (m, 6H), 2.46-2.10 (m, 5H), 2.08-1.94 (m, 7H), 1.89-1.77 (m, 2H), 1.60-1.54 (m, 3H), 1.35-1.21 (m, 6H), 1.11 (s, 9H), 0.94 (t, J=7.3 Hz, 3H), 0.75 (s, 3H). [M+H]⁺=1118.0.

### Example 542: 3-(4-((5-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)pentyl)oxy)-1-oxoisoindolin-2-yl)piperidine-2,6-dione

The title compound (55 mg, 54%) was prepared according to the procedure shown in WO2021036922A. ¹H NMR (500 MHz, DMSO) δ 12.68 (s, 1H), 10.98 (s, 1H), 8.87 (d, J=1.8 Hz, 1H), 8.84 (d, J=1.8 Hz, 1H), 8.26 (s, 2H), 8.24 (s, 1H), 7.92 (d, J=9.1 Hz, 1H), 7.48 (t, J=7.8 Hz, 1H), 7.35-7.29 (m, 2H), 7.25 (d, J=8.1 Hz, 1H), 6.81 (s, 1H), 5.12 (dd, J=13.3, 5.1 Hz, 1H), 4.38 (d, J=17.3 Hz, 1H), 4.23 (d, J=17.3 Hz, 1H), 4.13 (t, J=6.3 Hz, 2H), 3.78 (s, 3H), 2.97-2.87 (m, 1H), 2.81 (s, 4H), 2.59 (d, J=17.8 Hz, 3H), 2.44 (dd, J=13.1, 4.5 Hz, 4H), 2.08 (s, 3H), 2.06-2.01 (m, 8H), 1.83-1.71 (m, 2H), 1.59-1.56 (m, 10H), 1.49-1.41 (m, 2H). [M+H]⁺=993.4.

### Example 570: 3-(4-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione-3,5,5-d3

### Step 1: (E)-3-(4-(2-ethoxyvinyl)-2,6-difluorophenyl)piperidine-2,6-dione-3,5,5-d3

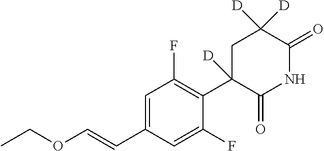 [SMILES: [2H]C1([2H])CC([2H])(c2c(F)cc(/C=C/OCC)cc2F)C(=O)NC1=O]

To a mixture of (E)-3-(4-(2-ethoxyvinyl)-2,6-difluorophenyl)piperidine-2,6-dione (0.4 g, 1.356 mmol, 1 eq, the compound was obtained through the similar way with example 484) and Et₃N (2.2 g, 21.67 mmol, 16 eq) in 10 mL of ACN was added TMS-C₁ (1.18 g, 10.85 mmol, 8 eq) dropwise. The mixture was stirred at 80° C. in a sealed tube for 12 hours. After cooled to 0° C., 5 mL of D₂O was added dropwise. The resulting mixture was stirred at room temperature for 2 hours. The mixture was extracted with DCM (3×50 mL) and concentrated to dryness. The residue was purified with silica gel column, eluting with MeOH in DCM (0%-5%) to afford the target compound (0.305 g, 75.5%). ¹H NMR (500 MHz, DMSO) δ 7.98 (s, 1H), 7.00 (d, J=12.9 Hz, 1H), 6.76 (d, J=10.1 Hz, 2H), 5.72 (d, J=12.9 Hz, 1H), 3.91 (q, J=7.0 Hz, 2H), 2.32 (d, J=13.4 Hz, 1H), 2.20-2.08 (m, 1H), 1.35 (t, J=7.0 Hz, 3H); [M+H]⁺=299.1.

### Step 2: 2-(4-(2,6-dioxopiperidin-3-yl-3,5,5-d3)-3,5-difluorophenyl)acetaldehyde

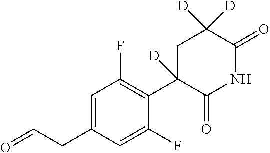 [SMILES: [2H]C1([2H])CC([2H])(c2c(F)cc(CC=O)cc2F)C(=O)NC1=O]

A solution of (E)-3-(4-(2-ethoxyvinyl)-2,6-difluorophenyl)piperidine-2,6-dione-3,5,5-d3 (60 mg, 0.2 mmol) in FA (3 mL) was stirred for 2 hrs at room temperature. The mixture was concentrated in vacuum, and 2-(4-(2,6-dioxopiperidin-3-yl-3,5,5-d3)-3,5-difluorophenyl)acetaldehyde (70 mg, crude) was obtained, which was used in the next step without further purification. [M+H]⁺=271.2.

### Step 3: 3-(4-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione-3,5,5-d3

To a solution of 2-(4-(2,6-dioxopiperidin-3-yl-3,5,5-d3)-3,5-difluorophenyl)acetaldehyde (70 mg crude, 0.2 mmol) in DCM (8 mL) was added (6-((5-bromo-2-((2-ethoxy-5-ethyl-4-(4-(piperazin-1-yl)piperidin-1-yl)phenyl)amino)pyrimidin-4-yl)amino)-2-methylquinolin-5-yl)dimethylphosphine oxide (72 mg, 0.1 mmol) at room temperature. After 1 h, NaBH(OAc)₃ (42.4 mg, 0.2 mmol) was added to the mixture. The resulting mixture was stirred overnight at room temperature. The mixture was diluted with water (10 mL) and extraction with DCM (3×50 mL). The combined organic phase was washed with brine (20 mL), dried over Na₂SO₄, filtered and concentrated in vacuum. The residue was purified by prep-HPLC (ACN in water with 0.1% of FA, 0% to 90%) to afford the product (7.2 mg, 7.4%). ¹H NMR (500 MHz, DMSO) δ11.71 (s, 1H), 10.97 (s, 1H), 8.60 (d, J=10.3 Hz, 1H), 8.30-8.24 (m, 1H), 8.22 (s, 1H), 7.91-7.85 (m, 2H), 7.43 (d, J=10.2 Hz, 2H), 7.03 (d, J=10.2 Hz, 2H), 6.07 (s, 1H), 4.00 (dd, J=25, 10.4 Hz, 2H), 2.91 (d, J=10.4 Hz, 2H), 2.75-2.66 (m, 2H), 2.64-2.51 (m, 9H), 2.49-2.08 (m, 9H), 2.00 (s, 3H), 1.96 (s, 3H), 1.83-1.79 (m, 2H), 1.58-1.49 (m, 2H), 1.26 (t, J=10.2 Hz, 3H), 0.71 (t, J=10.1 Hz, 3H); [M+H]⁺=975.2.

### (R)-2-(4-(2,6-dioxopiperidin-3-yl)-3,5-difluorophenyl)acetaldehyde

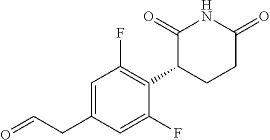 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA (50 mL). The resulting solution was stirred for 2 h at room temperature. The reaction solution was evaporated to dryness to afford the product (2.6 g, 91.8%). [M+H]⁺=268.1.

### (6-((5-bromo-2-chloropyrimidin-4-yl)amino)-2-ethylquinolin-5-yl)dimethylphosphine oxide

### Step 1: 6-nitroquinolin-2-ol

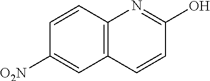 [SMILES: O=[N+]([O-])c1ccc2nc(O)ccc2c1]

To a stirred solution of quinolin-2-ol (6 g, 41.3 mmol) in conc. H₂SO₄ (98%, 50 mL) was added dropwise a solution of conc. HNO₃ (65%, 3.12 g, 49.6 mmol) at 0° C. Then the mixture was stirred at rt for 1 h. The mixture was diluted with water (200 mL) at 0° C. The resulting mixture was filtered and the filter cake was washed with H₂O (500 ml), and dried in vacuum to afford 6-nitroquinolin-2-ol (5.5 g 69.9%) [M+H]⁺=191.1.

### Step 2: 2-chloro-6-nitroquinoline

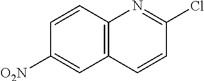 [SMILES: O=[N+]([O-])c1ccc2nc(Cl)ccc2c1]

A solution of 6-nitroquinolin-2-ol (5.5 g, 28.78 mmol) in POCl₃ (50 mL) was stirred at 100° C. for 2 hrs. Then the mixture was cooled to rt, and concentrated in vacuo. The residue was purified by Combi-Flash (silica column, 40 g, DCM:MeOH=15:1) to give 2-chloro-6-nitroquinoline (5 g, 82.9%) [M+H]⁺=209.1.

### Step 3: 6-nitro-2-vinylquinoline

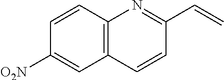

To a suspension of 2-chloro-6-nitroquinoline (5 g, 23.9 mmol) and 4,4,5,5-tetramethyl-2-vinyl-1,3,2-dioxaborolane (7.37 g, 47.8 mmol) in dioxane (40 mL) and water (10 mL) was added K₂CO₃ (9.91 g, 71.8 mmol) and Pd(dppf)Cl₂ (1.74 g, 2.39 mmol) under nitrogen atmosphere. The mixture was warmed to 100° C. and stirred for 16 hrs. Then the mixture was cooled to rt and filtered. The filtrate was concentrated in vacuo. The residue was purified by Combi-Flash (silica column, 40 g, DCM:MeOH=15:1) to give 6-nitro-2-vinylquinoline (4.5 g, 93.9%). [M+H]⁺=201.1.

### Step 4: 2-ethylquinolin-6-amine

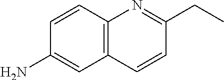

To a suspension of 6-nitro-2-vinylquinoline (4.5 g, 22.38 mmol) in MeOH (20 mL) was added Pd/C (10 wt. %, wet, 1.5 g). The mixture was stirred at rt for 16 hrs under hydrogen atmosphere. Then the mixture was filtered and the solid was washed with MeOH. The filtrate was concentrated in vacuo to afford 2-ethylquinolin-6-amine (3.84 g, 99.2%). [M+H]⁺=173.1.

### Step 5: 2-ethyl-5-iodoquinolin-6-amine

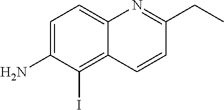

The title compound (4.5 g, 75.3%) was prepared in a manner similar to that in Example 486 step 4 from 2-ethylquinolin-6-amine and ICl. [M+H]⁺=299.1.

### Step 6: (6-amino-2-ethylquinolin-5-yl)dimethylphosphine oxide

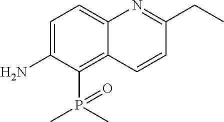 [SMILES: CCc1ccc2c(P(C)(C)=O)c(N)ccc2n1]

The title compound (3.5 g, 93.5%). was prepared in a manner similar to that in Example 486 step 5 from 2-ethyl-5-iodoquinolin-6-amine and dimethylphosphineoxide. [M+H]⁺=249.1.

### Step 7: (6-((5-bromo-2-chloropyrimidin-4-yl)amino)-2-ethylquinolin-5-yl)dimethylphosphine oxide

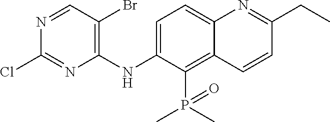 [SMILES: CCc1ccc2c(P(C)(C)=O)c(Nc3nc(Cl)ncc3Br)ccc2n1]

The title compound (2.5 g, 40.4%). was prepared in a manner similar to that in Example 486 step 6 from (6-amino-2-ethylquinolin-5-yl)dimethylphosphine oxide and 5-bromo-2,4-dichloropyrimidine. [M+H]⁺=439.6.

### (6-((5-bromo-2-chloropyrimidin-4-yl)amino)-2-methylquinolin-5-yl)dimethylphosphine oxide

### Step 1: 5-iodo-2-methylquinolin-6-amine

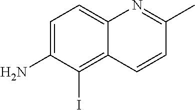

To a solution of 2-methylquinolin-6-amine (5 g, 31.62 mmol) in AcOH (50 mL) was added dropwise a solution of ICl (8.85 g, 37.95 mmol) in AcOH (10 mL) was stirred at 5° C.˜10° C. Then the mixture was stirred at rt for 1 h. The reaction solution was concentrated to dryness and the mixture was diluted with water (200 mL), neutralized with solid K₂CO₃. The mixture was extracted with DCM (3×150 mL). The combined organic phase was washed with brine (2×100 mL), dried over Na₂SO₄, filtered, and concentrated in vacuum.

The residue was purified by column chromatography (DCM:MeOH=20:1) to afford product (7.1 g, 79.06%). [M+H]⁺=285.2.

### Step 2: (6-amino-2-methylquinolin-5-yl)dimethylphosphine oxide

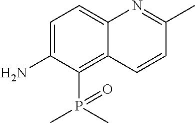 [SMILES: Cc1ccc2c(P(C)(C)=O)c(N)ccc2n1]

To a solution of 5-iodo-2-methylquinolin-6-amine (7.1 g, 24.99 mmol) and dimethylphosphine oxide (2.93 g, 37.49 mmol) in dioxane (100 mL) was added Pd(OAc)₂ (0.55 g, 2.45 mmol), Xantphos (2.89 g, 4.99 mmol), K₃PO₄ (10.61 g, 49.98 mmol) under N₂ atmosphere. The mixture was degassed under vacuum and purged with N₂ several times. The mixture was stirred under N₂ balloon at 100° C. for 6 h. The reaction mixture was extracted with DCM (3×50 mL). The combined organic phase was washed with brine (100 mL), dried over Na₂SO₄ and concentrated in vacuum. The residue was purified by column chromatography (DCM:MeOH=15:1) to afford the product (4.5 g, 76.9%). [M+H]⁺=235.2.

### Step 3: (6-((5-bromo-2-chloropyrimidin-4-yl)amino)-2-methylquinolin-5-yl)dimethylphosphine oxide

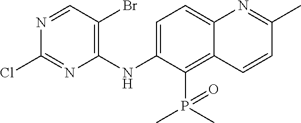 [SMILES: Cc1ccc2c(P(C)(C)=O)c(Nc3nc(Cl)ncc3Br)ccc2n1]

A solution of (6-amino-2-methylquinolin-5-yl)dimethylphosphine oxide (4.5 g, 19.21 mmol), 5-bromo-2,4-dichloropyrimidine (13.13 g, 57.64 mmol) and DIEA (7.45 g, 57.64 mmol) in n-BuOH (100 mL) was stirred at 120° C. for 12 h. The reaction solution was concentrated to dryness, then the crude product was purified by re-crystallization from EA:PE=5:1 (50 mL). The mixture was filtered and the filter cake was washed with DCM (3×50 mL). The combined organic phase was washed with brine (2×100 mL), dried over Na₂SO₄ and concentrated in vacuum to afford product (5.5 g, 67.3%). [M+H]⁺=425.2.

### Step 4: (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide

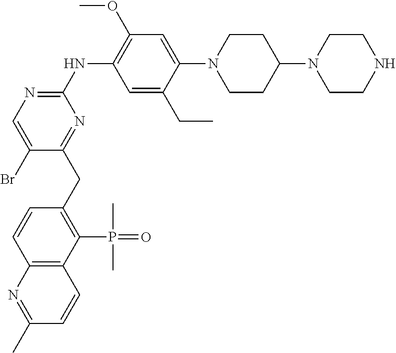 [SMILES: CCc1cc(Nc2ncc(Br)c(Cc3ccc4nc(C)ccc4c3P(C)(C)=O)n2)c(OC)cc1N1CCC(N2CCNCC2)CC1]

A solution of (6-((5-bromo-2-chloropyrimidin-4-yl)amino)-2-methylquinolin-5-yl)dimethylphosphine oxide (5.5 g, 12.91 mmol), TsOH (6.67 g, 38.73 mmol) and tert-butyl 4-(1-(4-amino-2-ethyl-5-methoxyphenyl)piperidin-4-yl)piperazine-1-carboxylate (5.67 g, 13.56 mmol) in n-BuOH (80 mL) was stirred at 100° C. for 12 h. The reaction mixture was adjusted to pH=8 with 1M NaOH, and then extracted with DCM (3×80 mL). The combined organic phase was washed with brine (2×100 mL), dried over Na₂SO₄ and concentrated in vacuum, The residue was purified by column chromatograph (DCM:MeOH=8:1) to afford the product (5.2 g, 57.01%). [M+H]⁺=707.3.

### 2,6-bis(benzyloxy)-3-(4-bromo-2,6-difluorophenyl)pyridine

### Step 1: 2,6-bis(benzyloxy)-3-(4,4,5,5-tetramethyl-1,3,2-dioxaborolan-2-yl)pyridine

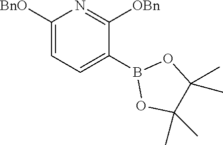 [SMILES: CC1(C)OB(c2ccc(OCc3ccccc3)nc2OCc2ccccc2)OC1(C)C]

A mixture of 2,6-bis(benzyloxy)-3-bromopyridine (15 g, 40.65 mmol) and 4,4,4′,4′,5,5,5′,5′-octamethyl-2,2′-bi(1,3,2-dioxaborolane) (12.6 g, 49.61 mmol), Pd(dppf)Cl₂ (3.32 g, 4.07 mmol), KOAc (12 g, 122.45 mmol) in dioxane (200 mL) was stirred overnight at 100° C. under nitrogen atmosphere. The resulting mixture was filtered, the filter cake was washed with MeOH and DCM. The filtrate was concentrated under reduced pressure. The residue was purified by silica gel column chromatography, eluted with PE/EtOAc (8:1) to afford the product (9.00 g, 53%). m/z[M+H]⁺=418.3.

### Step 2: 2,6-bis(benzyloxy)-3-(4-bromo-2,6-difluorophenyl)pyridine

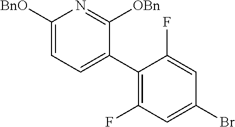 [SMILES: Fc1cc(Br)cc(F)c1-c1ccc(OCc2ccccc2)nc1OCc1ccccc1]

A mixture of 2,6-bis(benzyloxy)-3-(4,4,5,5-tetramethyl-1,3,2-dioxaborolan-2-yl)pyridine (9.00 g, 21.56 mmol) and 5-bromo-1,3-difluoro-2-iodobenzene (6.88 g, 21.57 mmol), K₂CO₃ (10.43 g, 75.48 mmol), Pd(dppf)Cl₂ (789 mg, 1.078 mmol) in dioxane (90 mL) and H₂O (30 mL) was stirred for 16 h at 100° C. under nitrogen atmosphere. The resulting mixture was extracted with EtOAc (50 mL×3). The combined organic layers were washed with brine, dried over anhydrous Na₂SO₄. After filtration, the filtrate was concentrated under reduced pressure. The residue was purified by silica gel column chromatography, eluted with PE/EtOAc (5:1) to afford the product (4 g, 38%). [M+H]⁺=482.4.

### 2-(3-(2,6-dioxopiperidin-3-yl)-2-oxo-2,3-dihydrobenzo[d]oxazol-7-yl)acetaldehyde

### Step 1: 3-(7-bromo-2-oxobenzo[d]oxazol-3(2H)-yl)piperidine-2,6-dione

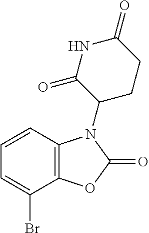 [SMILES: O=C1CCC(n2c(=O)oc3c(Br)cccc32)C(=O)N1]

A mixture of 7-bromobenzo[d]oxazol-2(3H)-one (2.13 g, 10 mmol), 3-bromopiperidine-2,6-dione (3.8 g, 20 mmol) and Cs₂CO₃ (6.5 g, 20 mmol) in DMF (50 mL) was stirred in a round bottom flask at 50° C. for 16 hours. The reaction was quenched with water (300 mL) and the mixture was extracted with DCM (100 mL×3). The combined organic layers were washed with brine (60 mL×3), dried over anhydrous Na₂SO₄, filtered and concentrated under reduced pressure. The residue was purified by re-crystallization from MeOH to afford the product (2 g, 62%), [M+H]⁺=325.1.

### Step 2: (E)-3-(7-(2-ethoxyvinyl)-2-oxobenzo[d]oxazol-3(2H)-yl)piperidine-2,6-dione

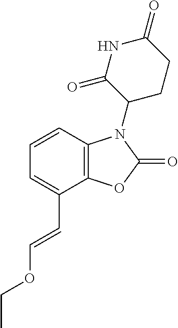 [SMILES: CCO/C=C/c1cccc2c1oc(=O)n2C1CCC(=O)NC1=O]

A mixture of 3-(7-bromo-2-oxobenzo[d]oxazol-3(2H)-yl)piperidine-2,6-dione (3.2 g, 10 mmol), (E)-2-(2-ethoxyvinyl)-4,4,5,5-tetramethyl-1,3,2-dioxaborolane (3 g, 15 mmol), Pd(Dtbpf)Cl₂ (0.6 g, 1 mmol) and CsF (4.5 g, 30 mmol) in DMF (40 mL) was stirred in a round bottom flask at 100° C. overnight under nitrogen atmosphere. The reaction was quenched with water (300 mL) and the mixture was extracted with DCM (100 mL×3). The combined organic layers were washed with brine (60 mL×3), dried over anhydrous Na₂SO₄, filtered and concentrated under reduced pressure. The residue was purified by silica gel column chromatography (DCM:MeOH=100:0˜10:1 gradient elution) to afford the product (2.1 g, 68%), [M+H]⁺=317.1.

### Step 3: 2-(3-(2,6-dioxopiperidin-3-yl)-2-oxo-2,3-dihydrobenzo[d]oxazol-7-yl)acetaldehyde

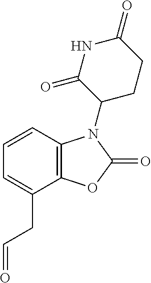 [SMILES: O=CCc1cccc2c1oc(=O)n2C1CCC(=O)NC1=O]

(E)-3-(7-(2-ethoxyvinyl)-2-oxobenzo[d]oxazol-3(2H)-yl)piperidine-2,6-dione (1.6 g, 5 mmol) in HCOOH (20 mL) was stirred in a round bottom flask at 25° C. for 2 hours. After concentration, the crude product was used directly for the next step without purification. [M+H]⁺=289.1. tert-butyl-4-(1-(4-amino-5-ethoxy-2-methylphenyl)piperidin-4-yl)piperazine-1-carboxylate

### Step 1: tert-butyl 4-(1-(5-ethoxy-2-methyl-4-nitrophenyl)piperidin-4-yl)piperazine-1-carboxylate

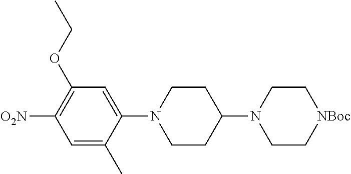 [SMILES: CCOc1cc(N2CCC(N3CCN(C(=O)OC(C)(C)C)CC3)CC2)c(C)cc1[N+](=O)[O-]]

The titled compound (740 mg, 88%) was prepared in a manner similar to that in Example 460 step 8 from 1-ethoxy-5-fluoro-4-methyl-2-nitrobenzene and tert-butyl 4-(piperidin-4-yl)piperazine-1-carboxylate. [M+H]⁺=449.3.

### Step 2: tert-butyl 4-(1-(4-amino-5-ethoxy-2-methylphenyl)piperidin-4-yl)piperazine-1-carboxylate

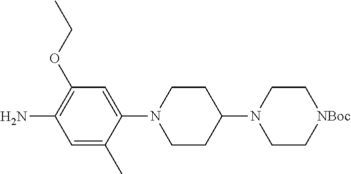 [SMILES: CCOc1cc(N2CCC(N3CCN(C(=O)OC(C)(C)C)CC3)CC2)c(C)cc1N]

The titled compound (520 mg, 78%) was prepared in a manner similar to that in Example 460 step 9 from tert-butyl 4-(1-(5-ethoxy-2-methyl-4-nitrophenyl)piperidin-4-yl)piperazine-1-carboxylate and Pd/C. [M+H]⁺=419.3.

Tert-butyl-4-(1-(4-amino-5-methoxy-2-methylphenyl)piperidin-4-yl)piperazine-1-carboxylate

### Step 1: tert-butyl 4-(1-(5-methoxy-2-methyl-4-nitrophenyl)piperidin-4-yl)piperazine-1-carboxylate

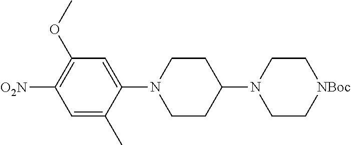 [SMILES: COc1cc(N2CCC(N3CCN(C(=O)OC(C)(C)C)CC3)CC2)c(C)cc1[N+](=O)[O-]]

The titled compound (840 mg, 86%) was prepared in a manner similar to that in Example 460 step 8 from 1-fluoro-5-methoxy-2-methyl-4-nitrobenzene and tert-butyl 4-(piperidin-4-yl)piperazine-1-carboxylate. [M+H]⁺=435.3.

### Step 2: tert-butyl 4-(1-(4-amino-5-methoxy-2-methylphenyl)piperidin-4-yl)piperazine-1-carboxylate

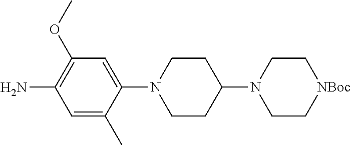 [SMILES: COc1cc(N2CCC(N3CCN(C(=O)OC(C)(C)C)CC3)CC2)c(C)cc1N]

The titled compound (720 mg, 88%) was prepared in a manner similar to that in Example 460 step 9 from tert-butyl 4-(1-(5-methoxy-2-methyl-4-nitrophenyl)piperidin-4-yl)piperazine-1-carboxylate and Pd/C. [M+H]⁺=405.3.

Cell Degradation

Cell Line Generation H1975-clone #28(Del19/T790M/C797S) and H1975-clone #23(Del19/C797S). EGFR-Del19/T790M/C797S and EGFR-Del19/C797S were stably expressed in H1975 cell lines by lentivirus-mediated over-expression, respectively. The EGFR over-expressed cells then underwent gene knockout, in which the EGFR targeting sgRNA was designed to only target the endogenous EGFR copies and preserve the exogenous EGFR copies. Followed by the gene knockout, the edited H1975 cells were seeded in 96 well plates at the concentration of 1 cell/well, cultured for about 2 weeks to allow single clones formation. The formed clones were screened by DNA sequencing and whole exon sequencing analysis for the desired edition. H1975-clone #28 and H1975-clone #23 were finally confirmed as homozygous Del19/T790M/C797S EGFR and Del19/C797S EGFR clones, respectively.

Cell Treatment

1a). BaF3-LTC(L858R/T790M/C797S) and BaF3 LC(L858R/C797S) cells are seeded at 50000 cells/well (WT) or 20000 cells/well (LTC,DTC&LC) in cell culture medium [RPMI1640 (Gibco, phenol red free, Cat #11835-030), 10% heat-inactive FBS, 1% PS (Gibco, Cat #10378)] in Corning 96 well plate (Cat #3799).

1b). On day 1, H1975-clone #28(Del19/T790M/C797S) and H1975-clone #23(Del19/C797S) cells are seeded at 20000 cells/well, 30000 cells/well, 10000 cells/well or 5000 cells/well correspondingly in cell culture medium [RPMI1640 (Gibco, Cat #72400-047), 10% heat-inactive FBS, 1% PS (Gibco, Cat #10378)] in Corning 96 well plate (Cat #3599).

BaF3-LTC(L858R/T790M/C797S) cells and BaF3 LC(L858R/C797S) cells are treated with compounds diluted in 0.2% DMSO cell culture medium and incubate for 16 h, 37° C., 5% CO₂. the final concentration of compounds in all assay is start with 10 uM, 5-fold dilution, total 8 doses were included.

HTRF Assay

After 16 h treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal−low control)/(High control−low control), wherein signal=each test compound group

Low control=only lysis buffer without cells, indicating that EGFR is completely degraded;

High control=Cell group with added DMSO and without compound, indicating microplate readings without EGFR degradation;

Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (H1975 # 28 DTC and BaF3-LTC) result for Example 459
to Example 756

| | H1975 # 28 DTC | | BaF3-LTC | |
|---|---|---|---|---|
| Example | DC₅₀(nM) | Dmax (%) | DC₅₀(nM) | Dmax (%) |
| 459 | 0.851 | 86.08 | 7.54 | 86.94 |
| 460 | 2.43 | 86 | 16.4 | 82.54 |
| 462 | 1.12 | 78.37 | 19.19 | 84.53 |
| 473 | 0.616 | 83.08 | 9.51 | 94.64 |
| 483 | 2.02 | 81.31 | 26.62 | 86.92 |
| 484 | 0.781 | 89.6 | 6.77 | 95.43 |
| 485 | 2.78 | 70.65 | 26.89 | 88.53 |
| 486 | 0.802 | 85.73 | 5.83 | 87.82 |
| 487 | 2.22 | 85.45 | 5.25 | 98.02 |
| 488 | 0.97 | 84.91 | 3.52 | 96.59 |
| 489 | 1.37 | 82.72 | 8.99 | 98.64 |
| 491 | 0.647 | 78.48 | 2.72 | 92.69 |
| 493 | 0.588 | 70.43 | 2.71 | 95.18 |
| 499 | 0.185 | 77.41 | 1.95 | 87.82 |
| 501 | 0.759 | 85.93 | 3.34 | 83.49 |
| 502 | 0.404 | 88.96 | 3.02 | 96.68 |
| 503 | 1.34 | 79.81 | 7 | 90.24 |
| 504 | 0.962 | 75.99 | 6.56 | 89.03 |
| 505 | 0.925 | 77.6 | 9.43 | 75.06 |
| 541 | 2.08 | 88.77 | 17.78 | 95.63 |
| 542 | 0.75 | 45.37 | 11.24 | 95.6 |
| 549 | 0.601 | 88.85 | 3.1 | 90.37 |
| 551 | 1.62 | 90.57 | 6.19 | 89.72 |
| 558 | 2.59 | 85.18 | 13.09 | 95.88 |
| 559 | 0.364 | 80.93 | 12.03 | 92.42 |
| 563 | 0.732 | 81.88 | 7.69 | 92.58 |
| 566 | 0.616 | 84.02 | 5.25 | 93.94 |
| 570 | 3.03 | 76.54 | 20.47 | 91.68 |
| 571 | 0.946 | 77.08 | 3.49 | 93.17 |
| 572 | 1.81 | 83.86 | 3.21 | 90.33 |
| 576 | 2.8 | 81.93 | 12.14 | 92.21 |
| 577 | 1.08 | 87.91 | 8.4 | 88.6 |
| 584 | 0.634 | 79.5 | 2.72 | 95.57 |
| 589 | 0.844 | 86.23 | 4.34 | 85.94 |
| 643 | 2.48 | 65.15 | 3.86 | 91.19 |
| 727 | 4.29 | 77.39 | 8.61 | 97.51 |
| 728 | 0.803 | 87.57 | 9.14 | 95.35 |
| 729 | 1.13 | 88.94 | 5.59 | 97.07 |
| 730 | 8.9 | 81.41 | 81.14 | 83.73 |
| 731 | 4.55 | 93.44 | 42.51 | 80.31 |
| 732 | 0.624 | 86.49 | 4.07 | 95.45 |
| 733 | 7.25 | 84.52 | 89.87 | 89.45 |
| 736 | 0.564 | 81.93 | 4.01 | 97.03 |
| 737 | 0.944 | 78.21 | 3.72 | 97.71 |
| 738 | 0.64 | 84.3 | 6.73 | 95.67 |
| 739 | 0.614 | 85.55 | 2.89 | 95.69 |
| 740 | 0.717 | 87.27 | 4.11 | 97.19 |
| 741 | 0.761 | 80.67 | 11.19 | 96.56 |
| 742 | 3.52 | 68.91 | 36.52 | 78.08 |
| 744 | 0.653 | 84.24 | 5.87 | 92.31 |
| 745 | 2.25 | 75.21 | 40.27 | 88.73 |
| 746 | 11.04 | 77.61 | 30.85 | 95.54 |
| 747 | 2.91 | 76.51 | 19.75 | 96.2 |
| 748 | 0.455 | 91.78 | 5.75 | 93.6 |
| 749 | 14.77 | 66.03 | 293.7 | 35.87 |
| 750 | 1.17 | 84.8 | 5.23 | 95.42 |
| 751 | 0.759 | 92.19 | 9.43 | 94.61 |
| 752 | 1.0 | 77.12 | 6.34 | 94.69 |
| 753 | 6.28 | 87.49 | 83.65 | 93.22 |
| 754 | 3.22 | 86.62 | 13.41 | 96.29 |
| 755 | 0.57 | 84.62 | 4.13 | 98.58 |
| 756 | 0.608 | 80.03 | 2.31 | 97.05 |

TABLE 2
Degradation (H1975 # 23 DC and BaF3-LC) result for Example 459
to Example 756

| | H1975 #23 DC | | BaF3 LC | |
|---|---|---|---|---|
| Example | DC₅₀(nM) | Dmax (%) | DC₅₀(nM) | Dmax (%) |
| 459 | 6.01 | 91.45 | 20.66 | 76.61 |
| 460 | 3.11 | 82.75 | 37.27 | 88.23 |
| 462 | 3.19 | 77.67 | 67.71 | 89.89 |
| 473 | 12.76 | 82.45 | 73.39 | 85.48 |
| 483 | 7.19 | 70.71 | 68.97 | 83.21 |
| 484 | 3.54 | 82.99 | 11.92 | 91.56 |
| 485 | 10.4 | 82.7 | 35.1 | 88.3 |
| 486 | 4.73 | 80.02 | 16.59 | 87.83 |
| 487 | 4.02 | 74.41 | 10.12 | 95.61 |
| 488 | 2.23 | 67.73 | 17.86 | 92.82 |
| 489 | 4.72 | 59.94 | 19.73 | 86.62 |
| 491 | 0.85 | 58.85 | 2.34 | 94.72 |
| 493 | 11.15 | 63.11 | 24.32 | 87.08 |
| 499 | 0.646 | 61.96 | 0.955 | 90.95 |
| 501 | 3.29 | 65.52 | 1.49 | 93.48 |
| 502 | 5.49 | 75.93 | 57.17 | 87.59 |
| 503 | 1.57 | 81.03 | 7.53 | 88.86 |
| 504 | 2.95 | 78.25 | 12.45 | 86.05 |
| 505 | 3.17 | 68.68 | 8.88 | 83.25 |
| 542 | >10000.0 | 31.04 | 16.25 | 89.95 |
| 549 | 2.96 | 63.66 | 1.59 | 87.15 |
| 551 | 5.64 | 84.14 | 46.47 | 86.8 |
| 559 | 5.38 | 73.67 | 26.91 | 90.43 |
| 571 | 3.05 | 75.83 | 13.19 | 87.65 |
| 572 | 4.74 | 69.16 | 3.8 | 91.75 |
| 576 | 3.59 | 77.19 | 15.82 | 95.93 |
| 577 | 7.94 | 64.13 | 53.78 | 89.1 |
| 584 | 2.7 | 70.66 | 4.9 | 92.75 |
| 589 | 2.2 | 84.51 | 20.27 | 92.36 |
| 643 | 1.45 | 73.67 | 3.97 | 94.1 |
| 750 | 6.52 | 74.02 | 26.13 | 94.35 |
| 752 | 1.76 | 78.99 | 2.23 | 93.97 |
| 753 | 2.43 | 78.45 | 20.07 | 88.89 |
| 754 | 5.6 | 71.73 | 36.07 | 87.35 |
| 755 | 7.68 | 69.09 | 16.38 | 91.74 |
| 756 | 1.69 | 73.8 | 10.06 | 94.5 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 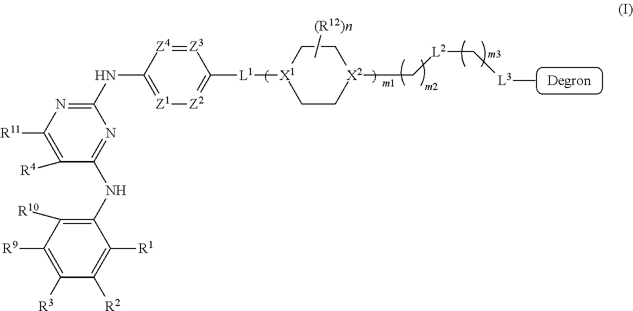
or a N-oxide thereof, or a pharmaceutically acceptable salt thereof, or a stereoisomer thereof, or a deuterated analog thereof, or a prodrug thereof,
wherein:
R¹ is selected from —P(O)R^{1a}R^{1b};
R^{1a} and R^{1b} are each independently selected from hydrogen, —C₁-C₈alkyl or C₃-C₈cycloalkyl, said —C₁-C₈alkyl or C₃-C₈cycloalkyl is optionally substituted with at least one halogen;
R² and R³ are each independently selected from hydrogen, halogen, —C₁-C₈alkyl, —C₃-C₈cycloalkyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, or —NR^{2a}SO₂R^{2b}; each of —C₁- C₈alkyl, C₃-C₈cycloalkyl, 3- to 8-membered heterocyclyl, C₆-C₁₂aryl or 5- to 12-membered heteroaryl is optionally substituted with at least one substituent R^{2d}, or
R² and R³ together with the carbon atoms to which they are attached, form a 5 or 6-membered unsaturated or saturated ring, said ring comprising 0-3 heteroatoms independently selected from nitrogen, oxygen or sulfur; said ring is optionally substituted with at least one substituent R^{2e};
R^{2e}, at each occurrence, is independently hydrogen, halogen, —C₁-C₈alkyl, —C₁-C₈alkoxy, —C₃-C₈cycloalkyl, 3- to 8-membered heterocyclyl, C₆-C₁₂aryl, 5- to 12-membered heteroaryl, oxo (═O), —OR^{2a}, thioxo (═S), —SR^{2a}, —CN, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b} or —NR^{2a}SO₂R^{2b}; each of —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent R^{2d};
R^{2a} and R^{2b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{2d}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is selected from hydrogen, halogen, —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl, 5- to 12-membered heteroaryl, —CN, —SO₂R^{4a}, —SO₂NR^{4a}R^{4b}, —COR^{4a}, —CO₂R^{4a}, —CONR^{4a}R^{4b}, —NR^{4a}R^{4b}, —NR^{4a}COR^{4b}, —NR^{4a}CO₂R^{4b} or —NR^{4a}SO₂R^{4b}; each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{4c}, —SO₂R^{4c}, —SO₂NR^{4c}R^{4d}, —COR^{4c}, —CO₂R^{4c}, —CONR^{4c}R^{4d}, —NR^{4c}R^{4d}, —NR^{4c}COR^{4d}, —NR^{4c}CO₂R^{4d} or —NR^{4c}SO₂R^{4d};
R^{4a}R^{4b}, R^{4c} and R^{4d} are each independently hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁹, R¹⁰, R¹¹ and R¹² are each independently selected from hydrogen, halogen, —C₁-C₈alkyl, —NR^{9a}R^{9b}, —OR^{9a}, —C₃-C₈cycloalkyl, 3- to 8-membered heterocyclyl, —C₆-C₁₂aryl, 5- to 12-membered heteroaryl, oxo (═O) or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{9c}; or
two R¹² together with the carbon atoms to which they are attached, form a 3- to 12-membered ring, said ring comprising 0-3 heteroatoms independently selected from nitrogen, oxygen or sulfur; said ring is optionally substituted with at least one substituent R^{9c};
R^{9a} and R^{9b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{9d}; or
R^{9c} and R^{9d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;
Z¹, Z², Z³ and Z⁴ are each independently selected from —CR^{Z}, or N;
R^{Z}, at each occurrence, is independently selected from hydrogen, halogen, —C₁-C₈alkyl, —NR^{Za}R^{Zb}, —OR^{Za}, —SR^{Za}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc};
R^{Za} and R^{Zb} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Za};
R^{Zc} and R^{Zd} are each independently halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
L¹ is selected from a single bond, —O—, —SO₂—, —C(O)—, —NR^{L1a}—, —C₃-C₈cycloalkylene-, *^{L1}—O—C₁-C₈alkylene-**^{L1}, *^{L1}—C₁-C₈alkylene-O—**^{L1}, *^{L1}—SO₂—C₁-C₈alkylene-**^{L1}, *^{L1}—C₁-C₈alkylene-SO₂—**^{L1}, *^{L1}—CO—C₁-C₈alkylene-**^{L1}, *^{L1}—C₁-C₈alkylene-CO—**^{L1}, *^{L1}—NR^{L1a}—C₁-C₈alkylene-**^{L1}, *^{L1}—C₁-C₈alkylene-NR^{L1a}—**^{L1}, *^{L1}—NR^{La}C(O)—**^{L1}, *^{L1}—C(O)NR^{L1a}—**^{L1}, —C₁-C₈alkylene-, —C₂-C₈alkenylene-, —C₂-C₈alkynylene-, —[O(CR^{L1a}R^{L1b})ₘ₄]ₘ₅—, 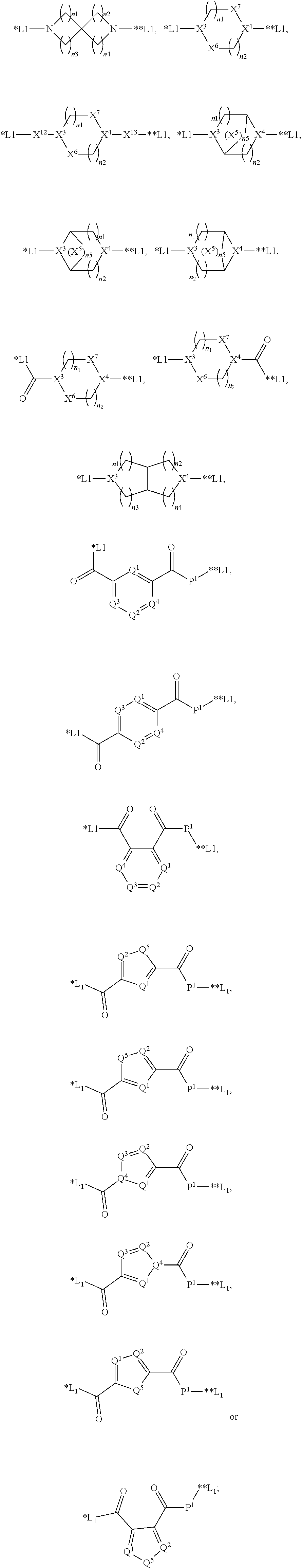
wherein each of said —C₃-C₈cycloalkylene-, *^{L1}—O—C₁-C₈alkylene-**^{L1}, *^{L1}—C₁-C₈-alkylene-O—**^{L1}, *^{L1}—SO₂—C₁-C₈alkylene-**^{L1}, *^{L1}—C₁-C₈alkylene-SO₂—**^{L1}, *^{L1}—CO—C₁-C₈alkylene-**^{L1}, *^{L1}—C₁-C₈alkylene- CO—**^{L1}, *^{L1}—NR^{L1a}—C₁-C₈alkylene-**^{L1}, *^{L1}—C₁-C₈alkylene-NR^{L1a}—**^{L1}—, —C₁-C₈alkylene-, —C₂-C₈alkenylene-, —C₂-C₈alkynylene-, 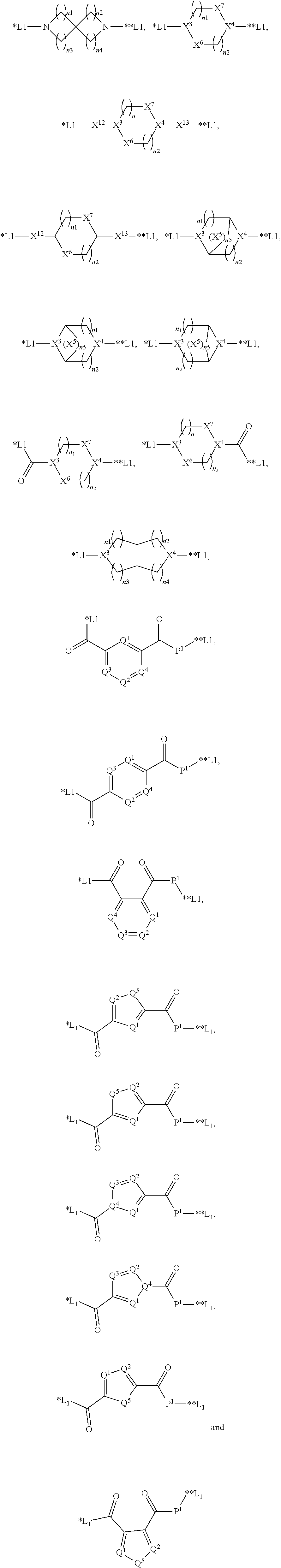
is optionally substituted with at least one R^{L1c};
wherein *^{L1} refers to the position attached to the 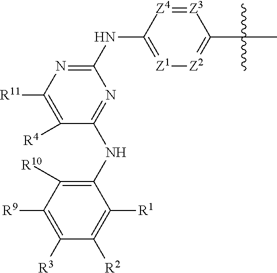
moiety, and **^{L1} refers to the position attached to the 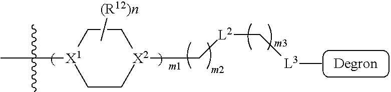
moiety;
R^{L1a} and R^{L1b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L1d};
each of said R^{L1c} and R^{L1d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
L² is selected from a single bond, —O—, —SO₂—, —CO—, —NR^{L2}—, —C₃-C₈cycloalkylene-, *^{L2}—O—C₁-C₈alkylene-**^{L2}, *^{L2}—C₁-C₈alkylene-O—**^{L2}, *^{L2}—SO₂—C₁-C₈alkylene-**^{L2}, *^{L2}—C₁-C₈alkylene-SO₂—**^{L2}, *^{L2}—CO—C₁-C₈alkylene-**^{L2}, *^{L2}—C₁-C₈alkylene-CO—**^{L2}, *^{L2}—NR^{L2a}C₁-C₈alkylene-**^{L2}, *^{L2}—C₁-C₈alkylene-NR^{L2a}—**^{L2}, *^{L2}—NR^{L2a}C(O)—**^{L2}, *^{L2}—C(O)NR^{L2a}—**^{L2}, —C₁-C₈alkylene-, —C₂-C₈alkenylene-, —C₂-C₈alkynylene-, —[O(CR^{L2a}R^{L2b})ₘ₄]ₘ₅—, 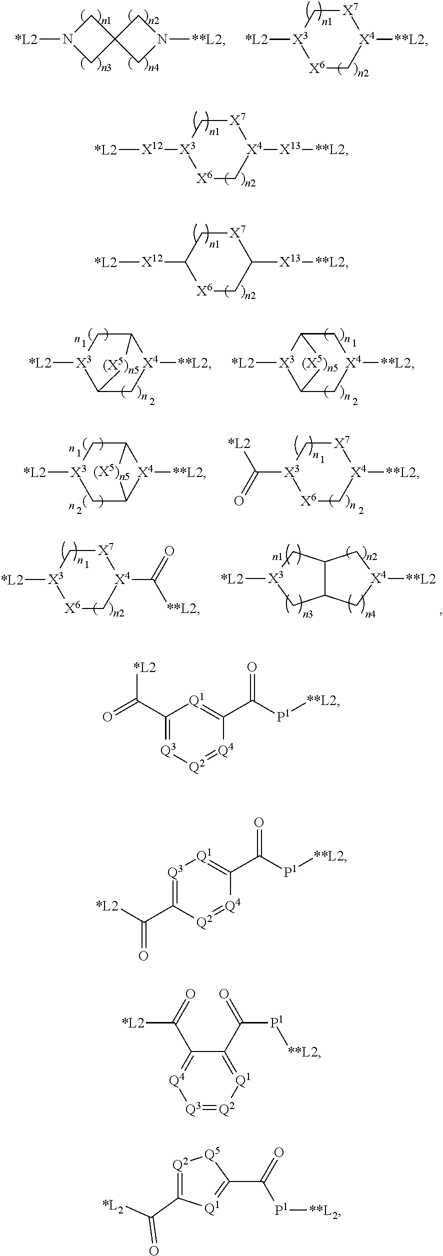 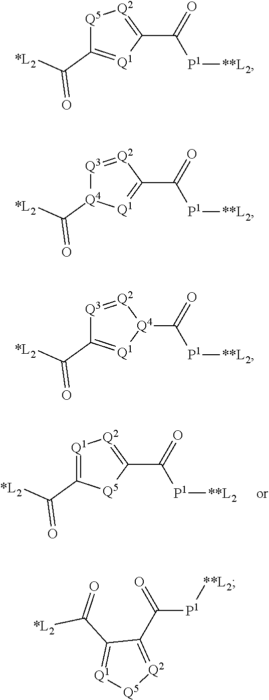
wherein each of said —C₃-C₈cycloalkylene-, *^{L2}—O—C₁-C₈alkylene-**^{L2}, *^{L2}—C₁-C₈alkylene-O—**^{L2}, *^{L2}—SO₂—C₁-C₈alkylene-**^{L2}, *^{L2}—C₁-C₈alkylene-SO₂—**^{L2}, *^{L1}—CO—C₁-C₈alkylene-**^{L2}, *^{L1}—C₁-C₈alkylene- CO—**^{L2}, *^{L2}—NR^{L2a}C₁-C₈alkylene-**^{L2}, *^{L2}—C₁-C₈alkylene-NR^{L2a}—**^{L2}, —C₁-C₈alkylene-, —C₂-C₈alkenylene-, —C₂-C₈alkynylene-, 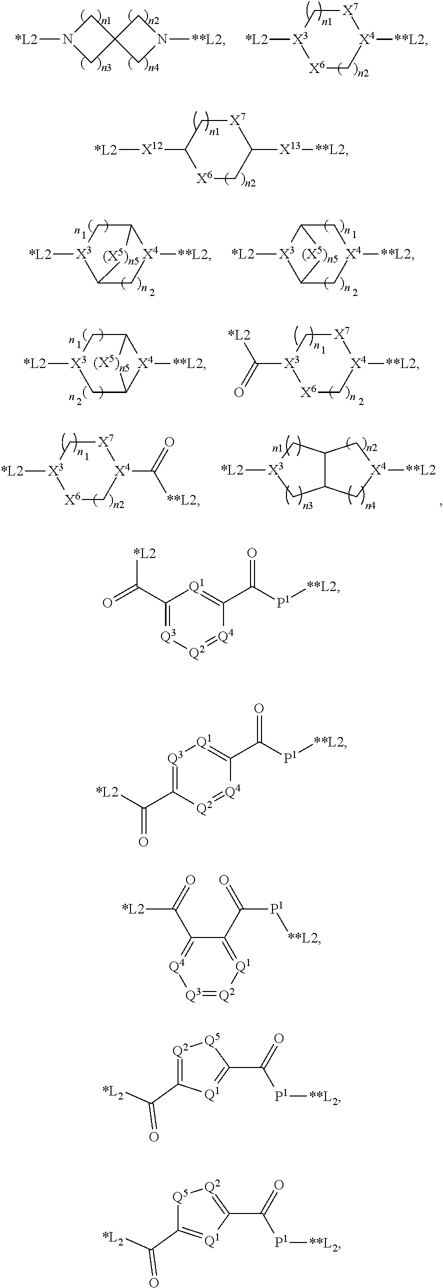 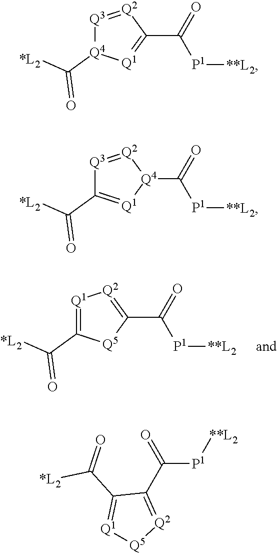
is optionally substituted with at least one substituent R^{L2c};
wherein *^{L2} refers to the position attached to 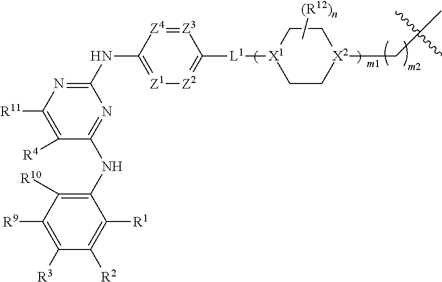
moiety, and **^{L2} refers to the position attached to the 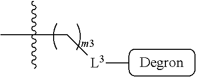
moiety;
R^{L2a} and R^{L2b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L2d};
each of said R^{L2c} and R^{L2d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L2}c together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
L³ is selected from a single bond, —O—, —SO₂—, —CO—, —NR^{L3a}—, —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂—**^{L3}, *^{L3}—CO—C₁-C₈alkylene-**^{L3}, *^{L3}—C₁-C₈alkylene-CO—**^{L3}, *^{L3}—NR^{L3a}C₁-C₈alkylene-**^{L3}, *^{L3}—C₁-C₈alkylene-NR^{L3a}—**^{L3}, *^{L3}—NR^{L3a}C(O)—**^{L3}, *^{L3}—C(O)NR^{L3a}—**^{L3}, —C₁-C₈alkylene-, —C₂-C₈alkenylene-, —C₂-C₈alkylene-, —[O(CR^{L3a}R^{L3b})ₘ₄]ₘ₅—, 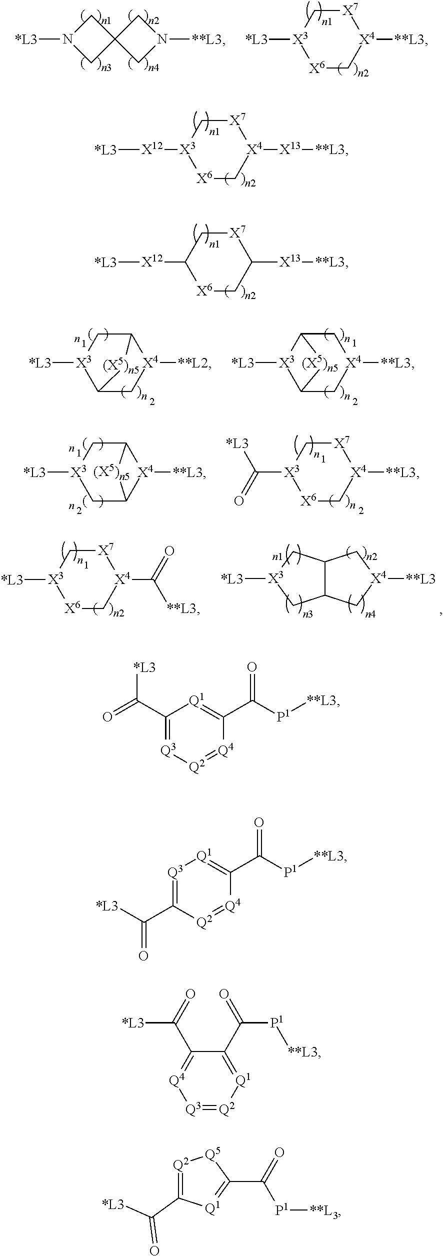 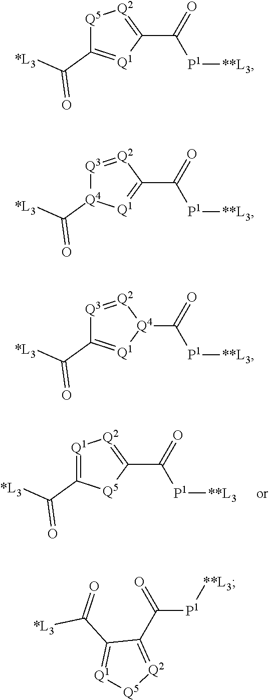
wherein each of said —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂—**^{L3}, *^{L3}—CO—C₁-C₈alkylene-**^{L3}, *^{L3}—C₁-C₈alkylene- CO—**^{L3}, *^{L3}—NR^{L3a}C₁-C₈alkylene-**^{L3}, *^{L3}—C₁-C₈alkylene-NR^{L3a}—**^{L3}, —C₁-C₈alkylene-, —C₂-C₈alkenylene-, —C₂-C₈alkynylene-, 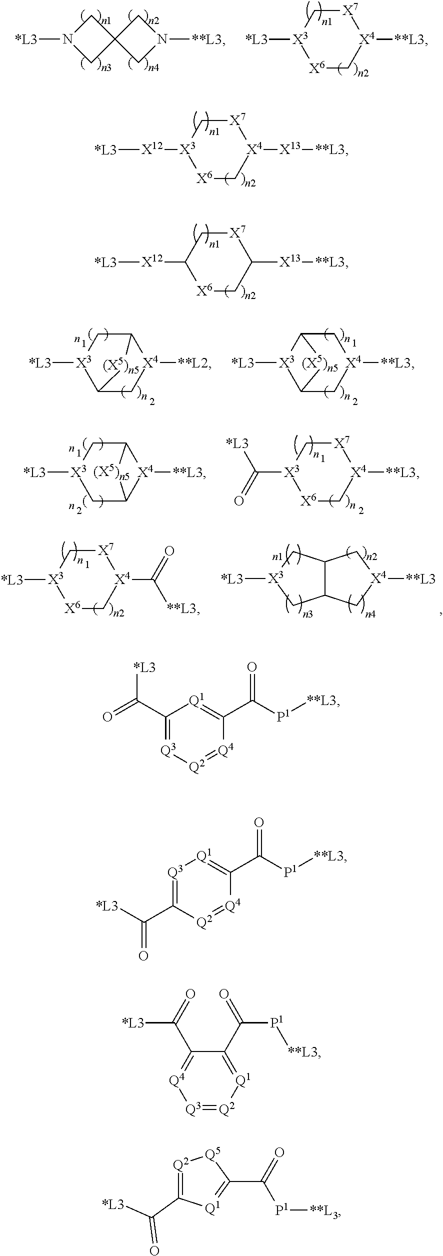 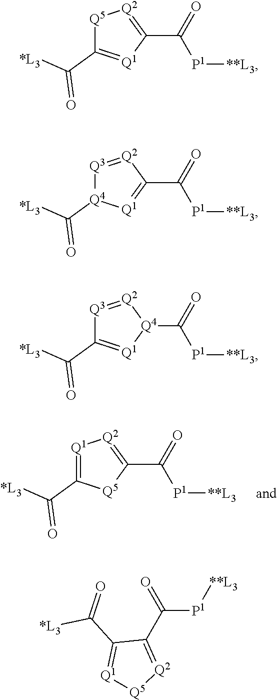
is optionally substituted with at least one substituent R^{L3c};
wherein *^{L3} refers to the position attached to 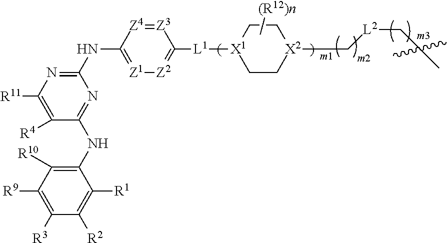
moiety, and **^{L3} refers to the position attached to the 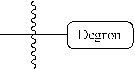
moiety;
R^{L3a} and R^{L3b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L3d};
each of said R^{L3c} and R^{L3d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl; 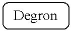
is selected from 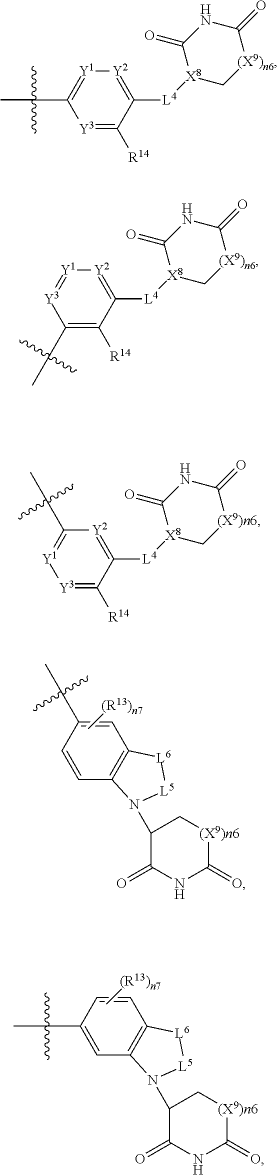 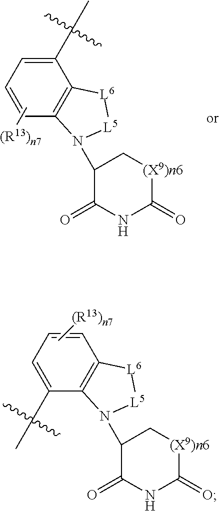
R¹³ and R¹⁴ are each independently selected from hydrogen, halogen, CN, —C₁-C₈alkyl, —C₁-C₈alkoxy, C₃-C₈cycloalkyl, 3- to 8-membered heterocyclyl, C₆-C₁₂aryl or 5- to 12-membered heteroaryl; said each —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent halogen, —C₁-C₈alkyl, C₁-C₈alkoxy-C₁-C₈alkyl-, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
X¹, X², X³, X⁴ and X⁸ are each independently selected from —CR^{a}, or N;
X⁵, X⁶, X⁷ and X⁹ are each independently selected from —NR^{a}—, —O—, —S— and —CR^{a}R^{b}—;
X¹² and X¹³ are each independently selected from —C(O)—, —NR^{a}— and —O—;
L⁴, L⁵ and L⁶ are each independently selected from a single bond, —O—, —NR^{a}—, —(CR^{a}R^{b})n₈˜, —O(CR^{a}R^{b})n₈-, —NR^{a}(CR^{a}R^{b})n₈- or —C(O)—;
Q¹, Q², Q³, Q⁴, Y¹, Y², Y³ are each independently selected from CR^{a} or N;
Q⁵ is each independently selected from —O—, —NR^{a}—, —CR^{a}R^{b}—, —S— or —C(O)—;
P¹ is a single bond, —O—, —NR^{a}—, —CR^{a}R^{b}—, —S—, —SO— or —SO₂—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C8alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₂aryl or 5- to 12-membered heteroaryl;
m₁ is 0, 1 or 2;
m₂ and m₃ are each independently 0, 1, 2, 3, 4, 5, 6, 7 or 8;
m₄ and m₅ are each independently 0, 1, 2 or 3;
n, n₁, n₂, n₃, n₄ and n₅ are each independently 0, 1, 2 or 3; and
n₆, n₇, n₈ and n₉ are each independently 0, 1, 2, 3 or 4.

2. The compound of claim 1, wherein the compound is selected from formula (II) or (III) 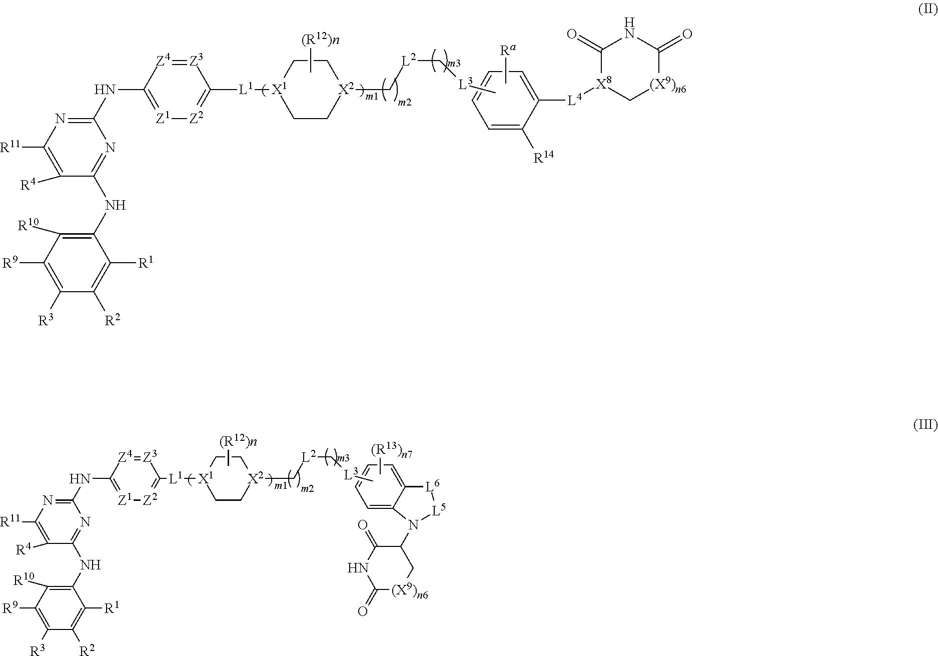
R¹, R², R³, R⁴, R⁹, R¹⁰, R¹¹, R¹², R¹³, R¹⁴, R^{a}, Z¹, Z², Z³, Z⁴, L¹, L², L³, L⁴, L⁵, L⁶, X¹, X², X⁸, X⁹, n, n6, n7, m1, m2 and m3 are each independently defined as claim 1.

3. The compound of claim 1-2, wherein R¹ is selected from —P(O)R^{1a}R^{1b}, wherein R^{1a} and R^{1b} are each independently selected from hydrogen, —C₁-C₈alkyl (preferably —CH₃, —C₂H₅, —C₃H₇, —C₄H₉ or —C₅H₁₁; more preferably —CH₃, —CH₂CH₃, —CH₂CH₂CH₃, -iso-C₃H₇, —CH₂CH₂CH₂CH₃, -iso-C₄H₉, -sec-C₄H₉ or -tert-C₄H₉) or C₃-C₈cycloalkyl (preferably cyclopropyl, cyclobutyl or cyclopentyl).

4. The compound of any one of claims 1-3, wherein R₁ is —P(O)(CH₃)₂.

5. The compound of any one of claims 1-4, wherein R² and R³ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, or —NR^{2a}SO₂R^{2b}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl is optionally substituted with at least one substituent R^{2d}, or
R² and R³ together with the carbon atoms to which they are attached, form a 5 or 6-membered unsaturated or saturated ring, said ring comprising 0, 1, 2 or 3 heteroatoms independently selected from nitrogen, oxygen or sulfur; said ring is optionally substituted with at least one substituent R^{2e};
R^{2e}, at each occurrence, is independently —H, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl, oxo (═O), —CN, CF₃, CHF₂, CH₂F, thioxo (═S), —SCF₃, —SCHF₂, —SCH₂F, —SCH₂CF₃, —SCF₂CH₃, —SCF₂CF₃, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b} or —NR^{2a}SO₂R²¹; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl is optionally substituted with at least one substituent R^{2d};
R^{2a} and R^{2b} are each independently selected from hydrogen, methyl, ethyl, propyl, butyl, pentyl, hexyl, heptyl, octyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{2d}, at each occurrence, is independently halogen, —OH, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

6. The compound of any one of claims 1-5, wherein R² and R³ together with the carbon atoms to which they are attached, form a 5 or 6-membered unsaturated (preferred aromatic) or saturated ring, said ring comprising 1 or 2 nitrogen heteroatoms; said ring is optionally substituted with at least one substituent —H, —F, —Cl, —Br, —I, methyl, ethyl, propyl (n- or iso-), butyl, pentyl, methoxy, ethoxy, propoxy, butoxy, pentoxy, cyclopropyl, cyclobutyl, cyclopentyl, cyclohexyl, cycloheptyl, cyclooctyl, —CH₂OH, —SCH₃, —SC₂H₅, oxo (═O), thioxo (═S), —CF₃, —CHF₂, —CH₂F, —SCF₃, —OMe, —OC₂H₅, —CN, —C(O)CH₃,

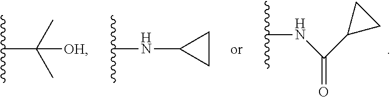

7. The compound of any one of claims 1-6, wherein R² and R³ together with the carbon atoms to which they are attached, form a 6-membered unsaturated (preferred aromatic) ring, said ring comprising 1 or 2 nitrogen heteroatoms; said ring is optionally substituted with one substituent —H, —F, —Cl, —Br, —I, methyl, ethyl or cyclopropyl.

8. The compound of any one of claims 1-7, wherein R⁴ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl, —C₂-C₈alkynyl or —C₁-C₈alkoxy; each of methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl or —C₂-C₈alkynyl is optionally substituted with —F, —Cl, —Br, —I, oxo (═O), or —CN.

9. The compound of any one of claims 1-8, wherein R⁴ is hydrogen, —F, —Cl, —Br, —I, —CH₃, —CF₃, —CH₂F, or —CHF₂.

10. The compound of any one of claims 1-9, wherein R⁴ is hydrogen, —F, —Cl, —Br or —I.

11. The compound of any one of claims 1-10, wherein R⁹, R¹⁰, R¹¹ and R¹² are each independently selected from hydrogen, —F, —Cl, —Br, —I, methyl, ethyl, propyl, butyl, pentyl, hexyl, heptyl, octyl, —NR^{9a}R^{9b}, —OR^{9a}, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), or —CN; each of—methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9c};
R^{9a} and R^{9b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9d};
R^{9c} and R^{9d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

12. The compound of any one of claims 1-11, wherein R⁹, R¹⁰, R¹¹ and R¹² are each independently selected from hydrogen, —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, cyclohexyl, —CH₂OH, —CH₂OMe, oxo (═O), or —CN.

13. The compound of any one of claims 1-12, wherein R⁹, R¹⁰, R¹¹ and R¹² are each independently selected from hydrogen, —CH₃, —F, —Cl, —Br or —I.

14. The compound of any one of claims 1-10, wherein two R¹² together with the carbon atoms to which they are attached, form a 3, 4, 5, 6, 7 or 8-membered ring, said ring comprising 0, 1, 2 or 3 heteroatoms independently selected from nitrogen, oxygen or sulfur; said ring is optionally substituted with at least one substituent R^{9c};
R^{9c} is independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

15. The compound of any one of claims 1-10, wherein two R¹² together with the carbon atoms to which they are attached, form a 3, 4, 5, 6, 7 or 8-membered ring, preferably form a 3, 4, 5 or 6-membered ring, said ring comprising 0, 1, 2 or 3 heteroatoms independently selected from nitrogen, oxygen or sulfur; said ring is optionally substituted with at least one substituent —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, or cyclohexyl.

16. The compound of any one of claims 1-4, wherein the 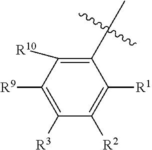
moiety is 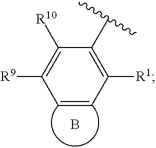
Ring B is a 5 or 6-membered unsaturated or saturated ring, said ring comprising 0, 1, 2 or 3 heteroatoms; said heteroatoms are independently selected from N, NR^{2e}, O or S;
said ring is optionally substituted with at least one substituent R^{2e}.

17. The compound of any one of claims 1-16, wherein the 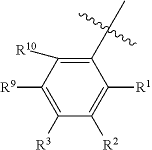
moiety is selected from 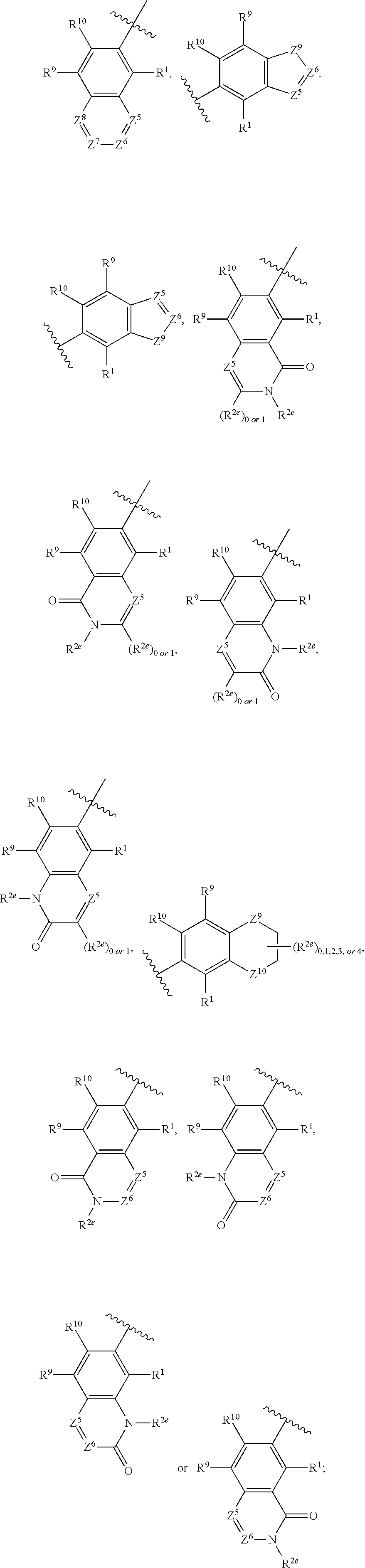
Z⁵, Z⁶, Z⁷ and Z⁸ are each independently selected from N, CH or CR^{2e};
Z⁹ and Z¹⁰ are each independently selected from O, S, NH or NR^{2e}.

18. The compound of any one of claims 1-17, wherein the 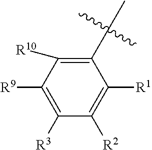
moiety is selected from

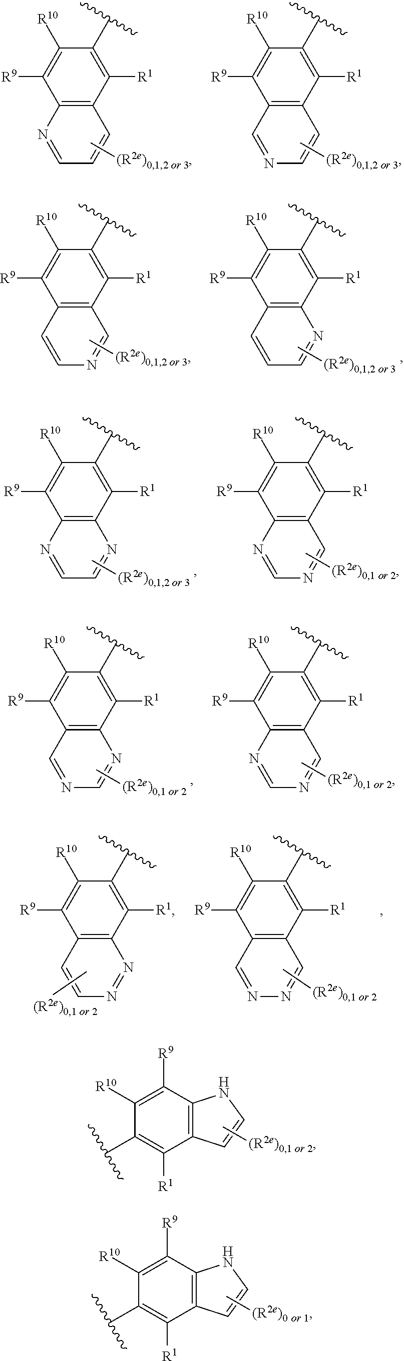

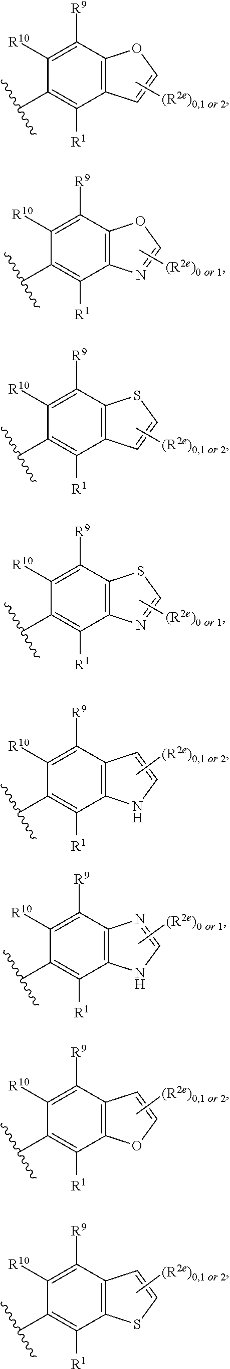

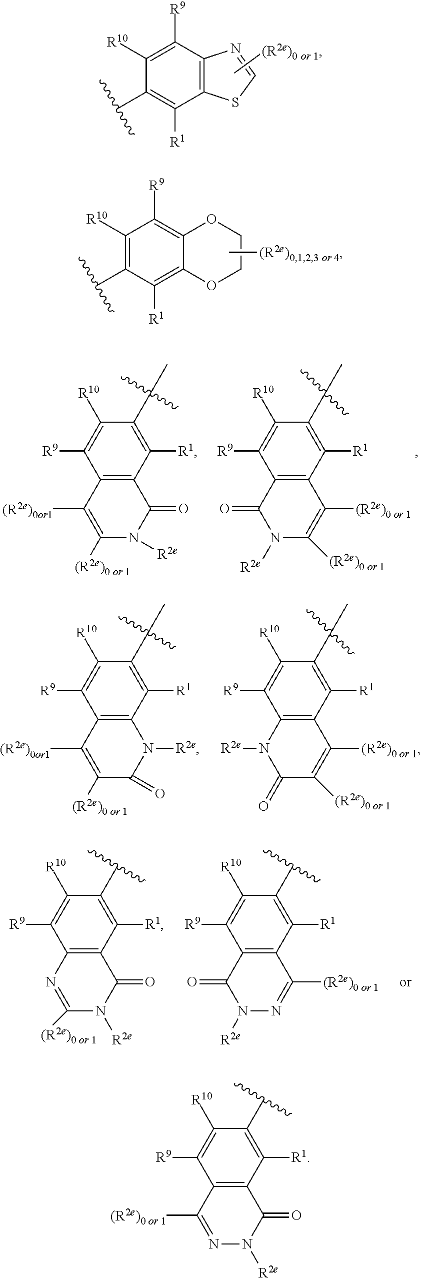

19. The compound of any one of claims 1-18, wherein the 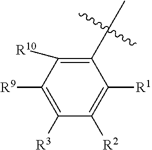
moiety is selected from

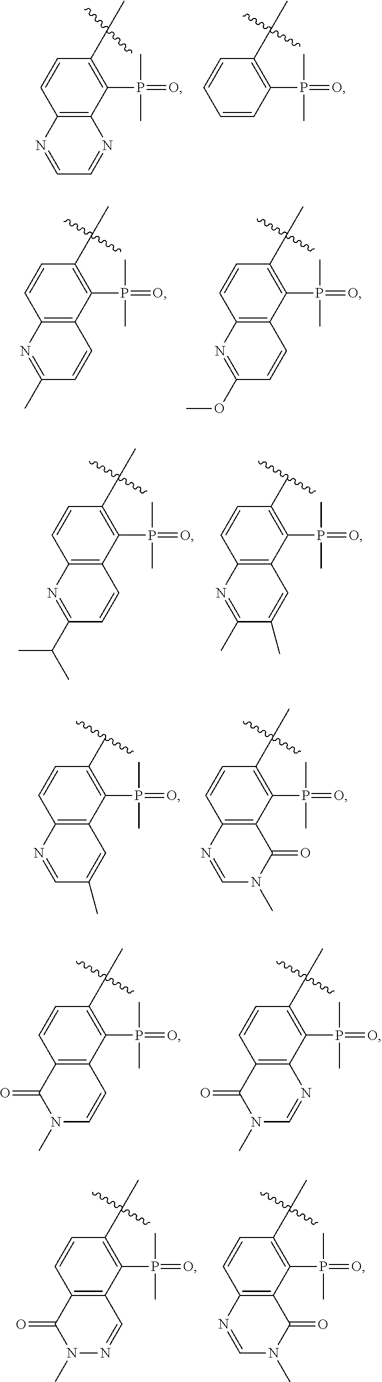

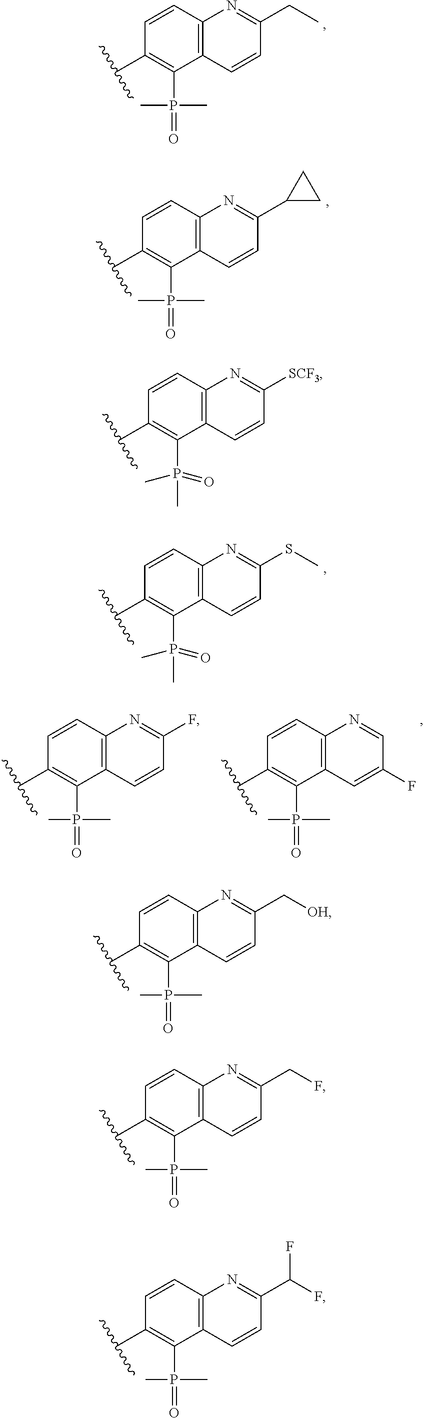

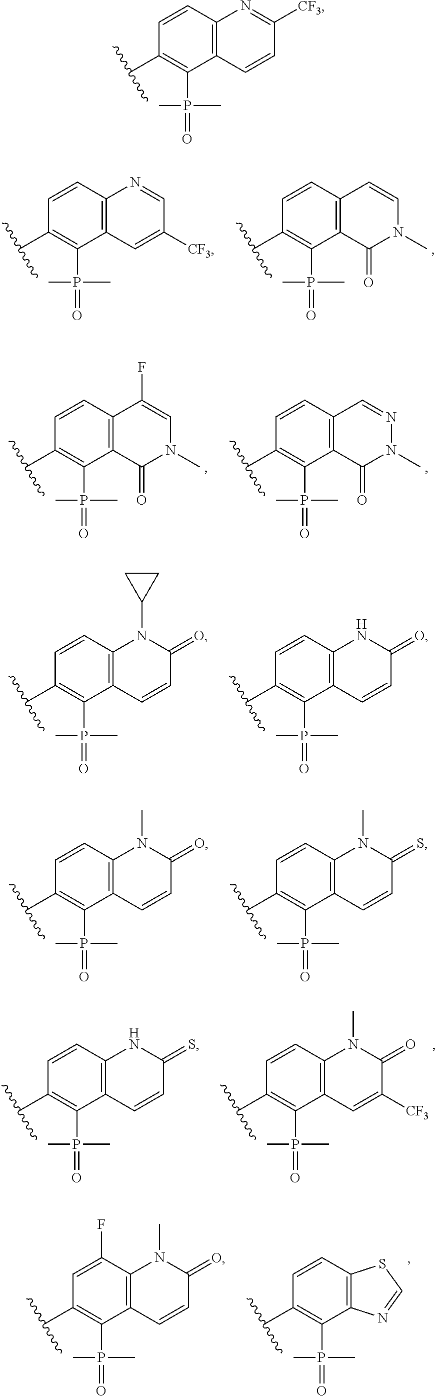

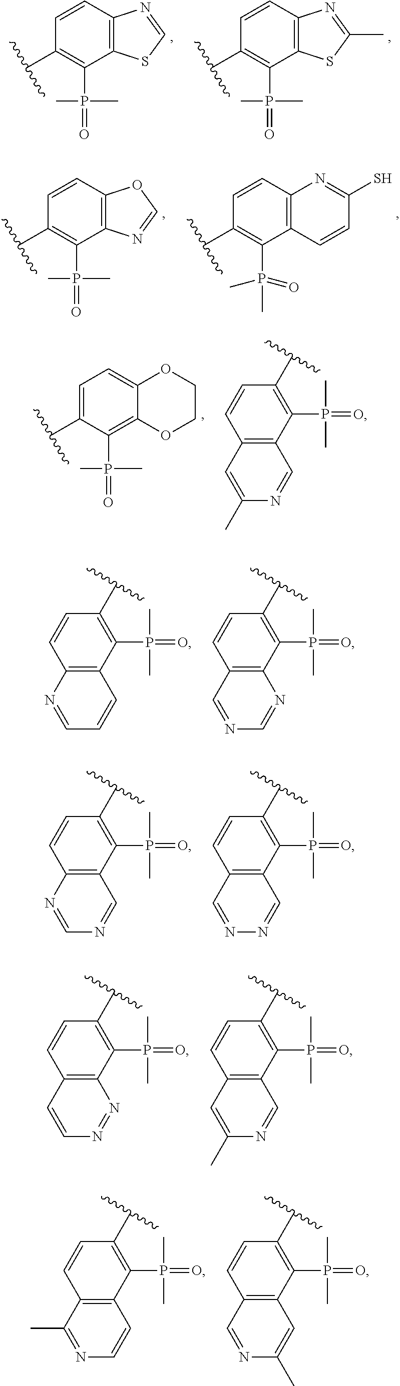

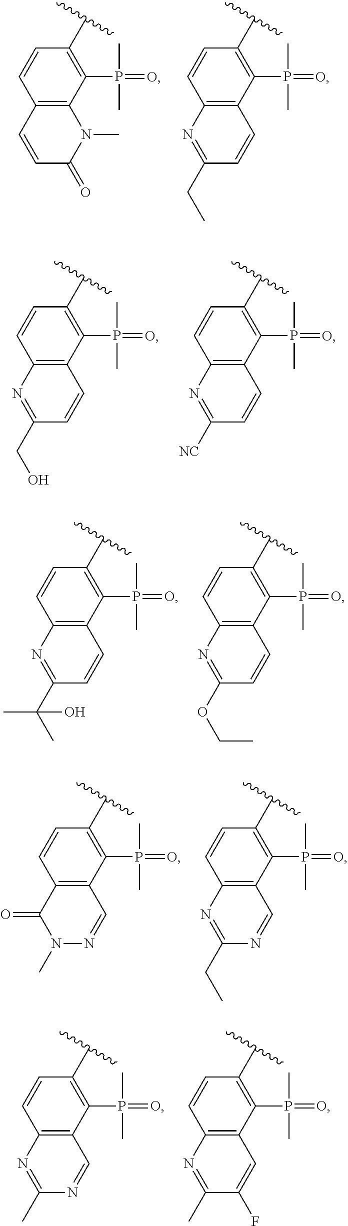

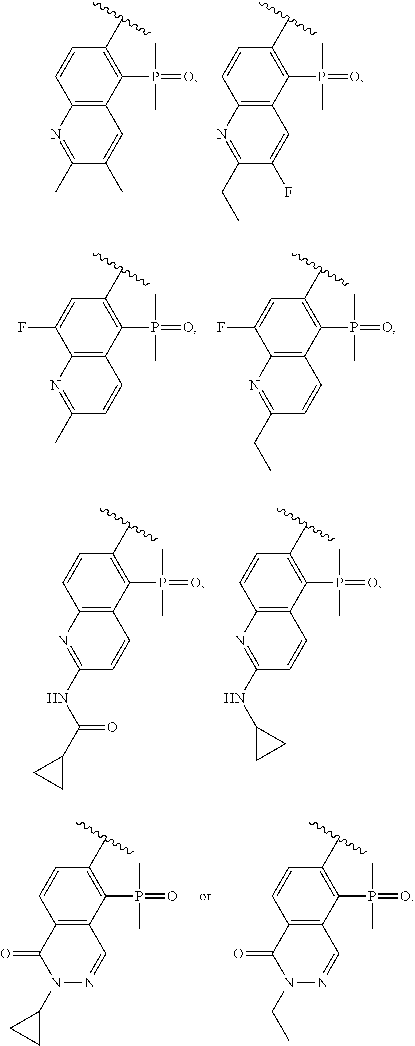

20. The compound of any one of claims 1-19, wherein L¹ is selected from a single bond, —C₁-C₈alkylene- (preferably —CH₂—, —C₂H₄—, —C₃H₆—), —C(O)—C₁-C₈alkylene- (preferably —C(O)—CH₂—, —C(O)—C₂H₄—, —C(O)—C₃H₆—), —C₁-C₈alkylene-C(O)— (preferably —CH₂—C(O)—, —C₂H₄—C(O)—, —C₃H₆—C(O)—), —CO—, —O—, —N(CH₃)—, —NH—, 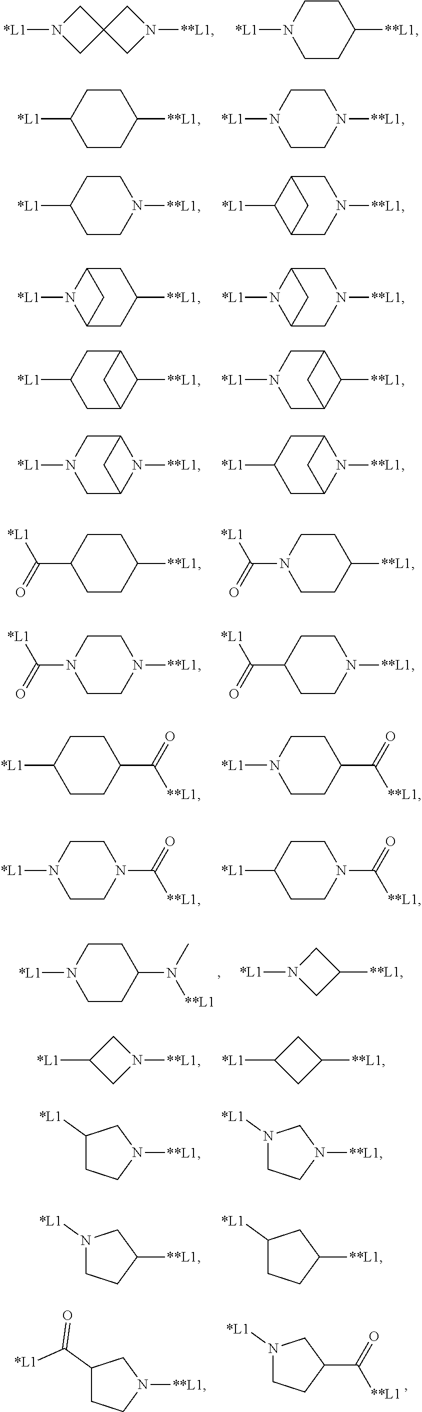 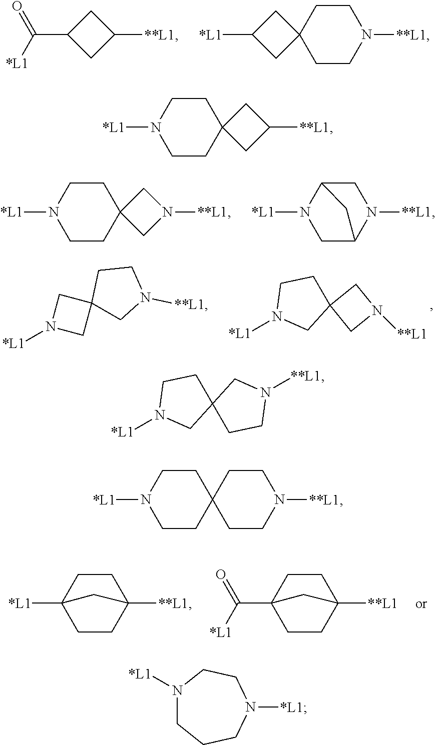
wherein each of said C₁-C₈alkylene- (preferably —CH₂—, —C₂H₄—, —C₃H₆—), *^{L1}—C(O)—C₁-C₈alkylene-**^{L1} (preferably *^{L1}—C(O)—CH₂—**^{L1}, *^{L1}—C(O)—C₂H₄—**^{L1}, *^{L1}—C(O)—C₃H₆—**^{L1}), *^{L1}—C₁-C₈alkylene-C(O)—**^{L1} (preferably *^{L1}—CH₂—C(O)—**^{L1}, *^{L1}—C₂H₄—C(O)—**^{L1}, *^{L1}—C₃H₆—C(O)—**^{L1}), —N(CH₃)—, —NH—, 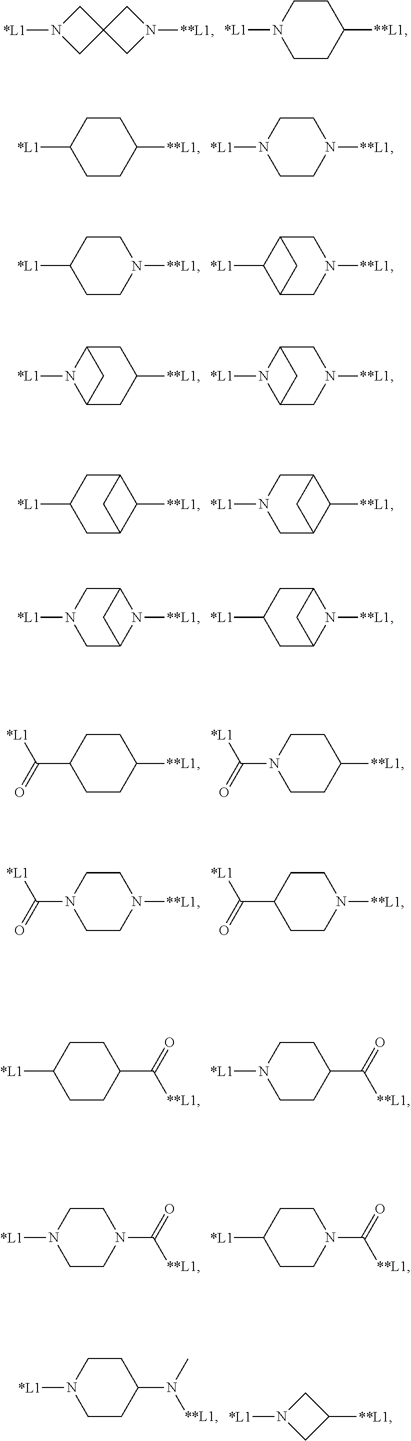 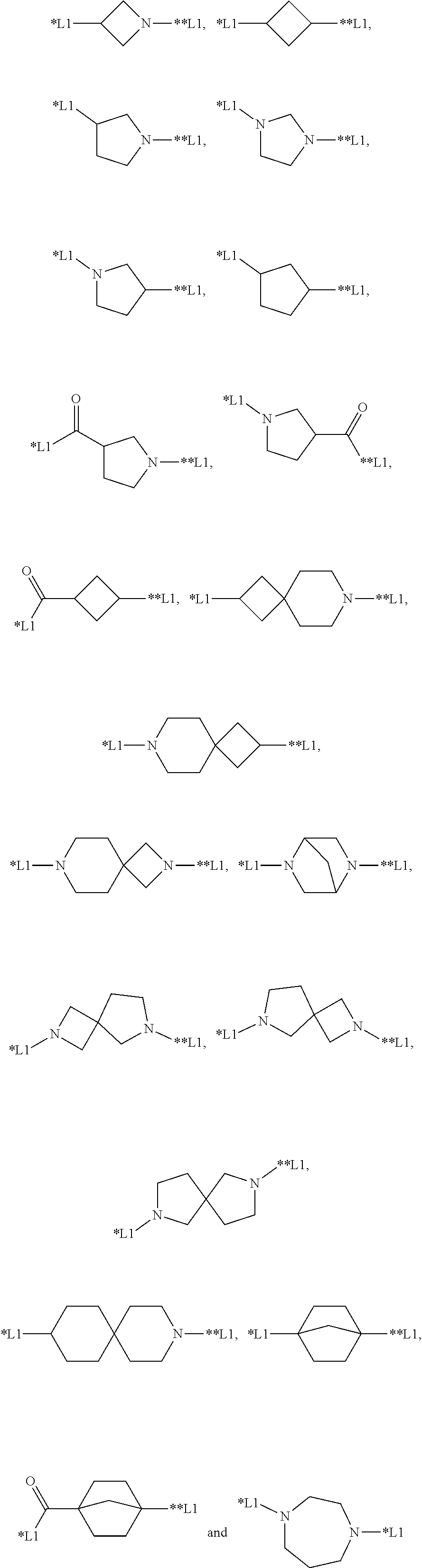
is optionally substituted with at least one R^{L1c};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl.

21. The compound of any one of claims 1-20, wherein L¹ is selected from a single bond, —C₁-C₈alkylene- (preferably —CH₂—, —C₂H₄—, —C₃H₆—), —CO—, —O—, —N(CH₃)—, —NH—,

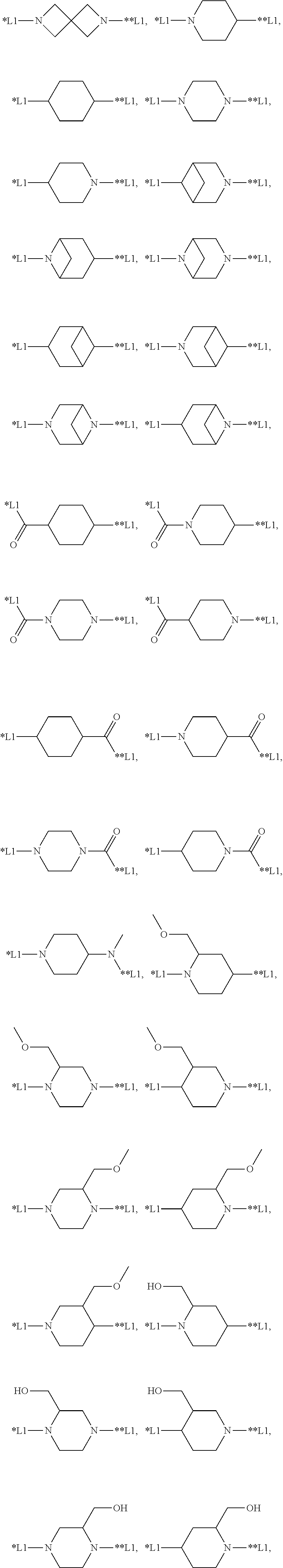

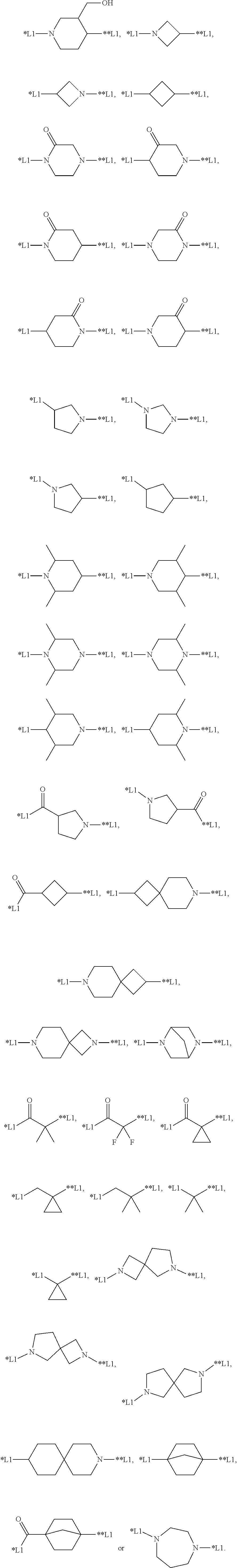

22. The compound of any one of claims 1-21, wherein X¹ and X² are each independently selected from —CR^{a} or N;
R^{a} is selected from hydrogen, —F, —Cl, —Br, —I, CN, methyl, ethyl, methoxy, ethoxy, cyclopropyl, each of said methyl, ethyl, methoxy, ethoxy, cyclopropyl, is optionally substituted with at least one substituent —F, —Cl, —Br, —I, hydroxy, methyl, ethyl, (preferably, X¹ and X² are each independently selected from CH, C(F), C(CH₃) or N);
m1=1 or 0;
R¹² is hydrogen, oxo (═O), methoxymethyl, hydroxymethyl, —CN or —CH₃.

23. The compound of any one of claims 1-22, wherein m1 is 1; preferably, 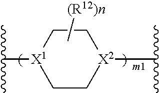
moiety is 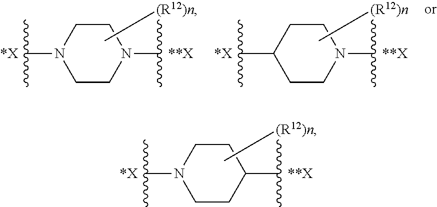
wherein *^{X} refers to the position attached to 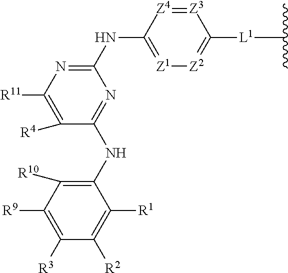
moiety, and **^{X} refers to the position attached to the 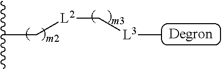
moiety.

24. The compound of an one of claims 1-23, wherein m1 is 1; preferably 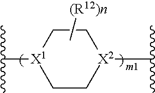
moiety is 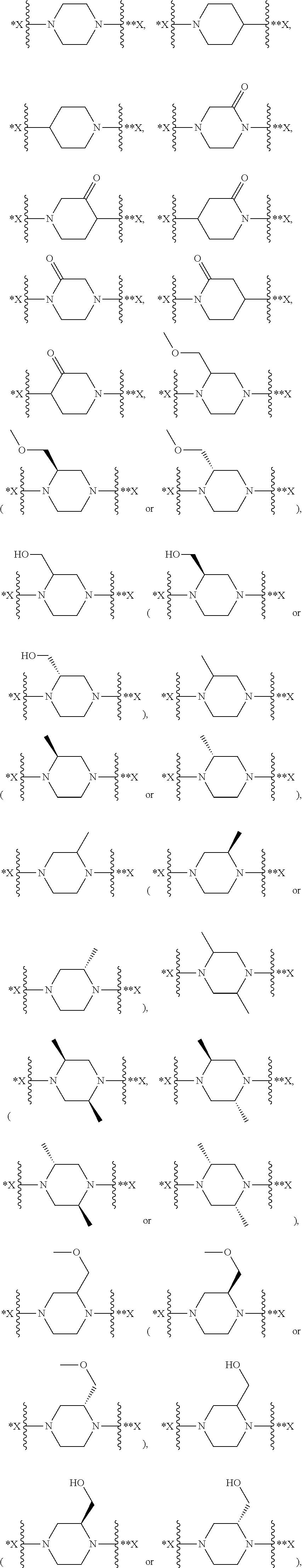
wherein *^{X} refers to the position attached to 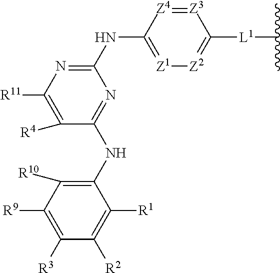
moiety, and **^{X} refers to the position attached to the 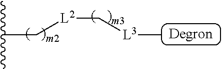
moiety.

25. The compound of any one of claims 1-24, wherein m1 is 1, 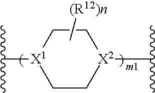
moiety is 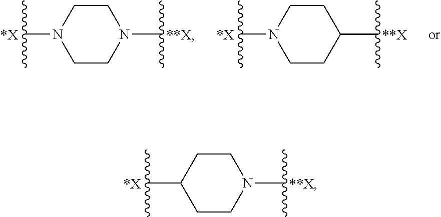 [SMILES: *N1CCN(*)CC1 ; *C1CCN(*)CC1 ; *C1CCN(*)CC1]
wherein *^{X} refers to the position attached td 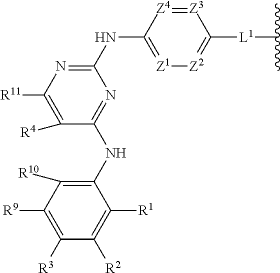
moiety, and **^{X} refers to the position attached to the 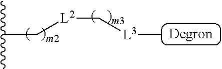
moiety.

26. The compound of any one of claims 1-25, wherein L² is selected from a single bond, —C₁-C₈alkylene-(preferably —CH₂—, —C₂H₄—, —C₃H₆—), *^{L2}—CO—C₁-C₈alkylene-**^{L2} (preferably *²—CO—CH₂—**^{L2}, *^{L2}—CO—C₂H₄—**^{L2}, *^{L2}—CO—C₃H₆—**^{L2}), *^{L2}—C₁-C₈alkylene-CO—**^{L2} (preferably *^{L2}—CH₂—CO—**^{L2}, *^{L2}—C₂H₄—CO—**^{L2}, *^{L1}—C₃H₆—CO—**^{L2}), —CO—, —O—, —N(CH₃)—, —NH—, 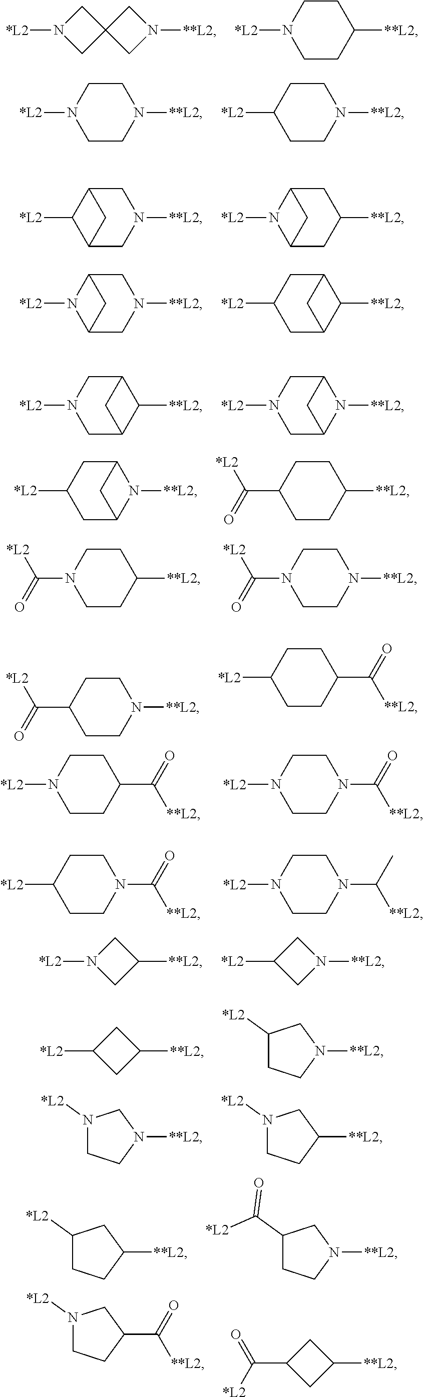 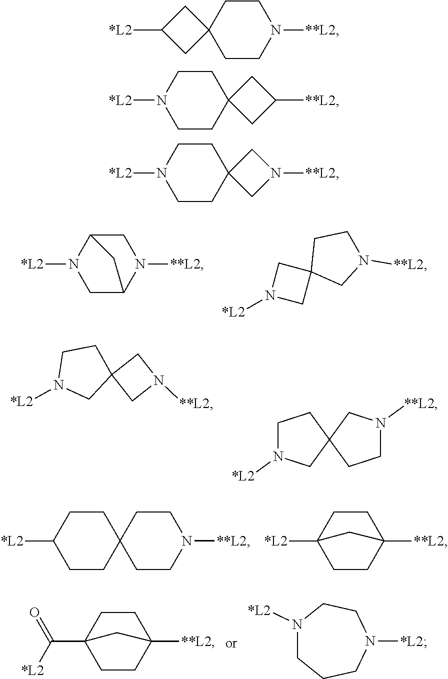
wherein each of said —C₁-C₈alkylene-(preferably —CH₂—, —C₂H₄—, —C₃H₆—), *^{L2}—CO—C₁-C₈alkylene-**^{L2} (preferably *^{L2}—CO—CH₂—**^{L2}, *^{L2}—CO—C₂H₄—**^{L2}, *^{L2}—CO—C₃H₆—**^{L2}), *^{L2}—C₁-C₈alkylene-CO—**^{L2} (preferably *^{L2}—CH₂—CO—**^{L2}, *^{L2}—C₂H₄—CO—**^{L2}, *^{L2}—C₃H₆—CO—**^{L2}), —N(CH₃)—, —NH—, 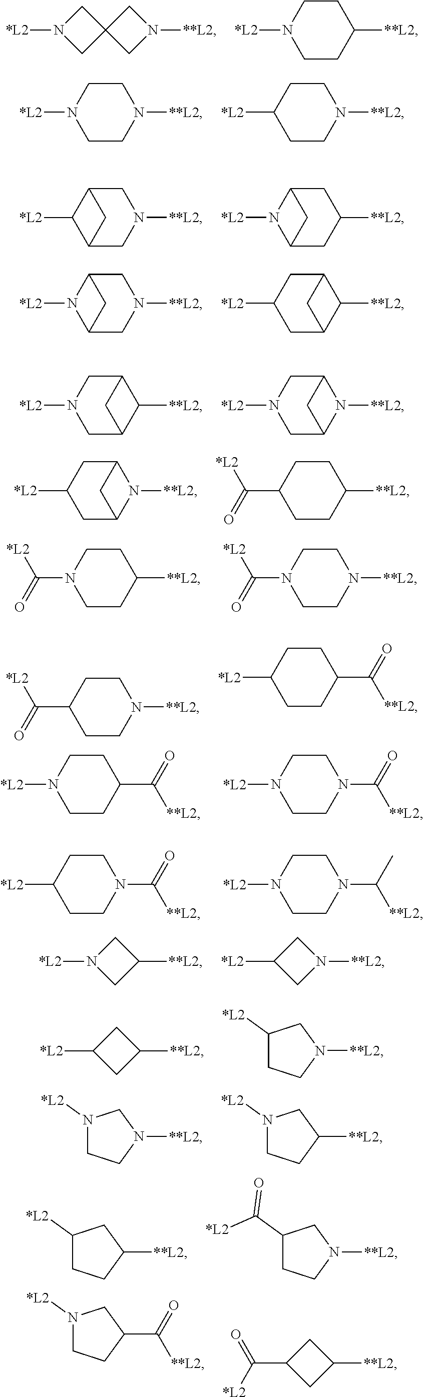 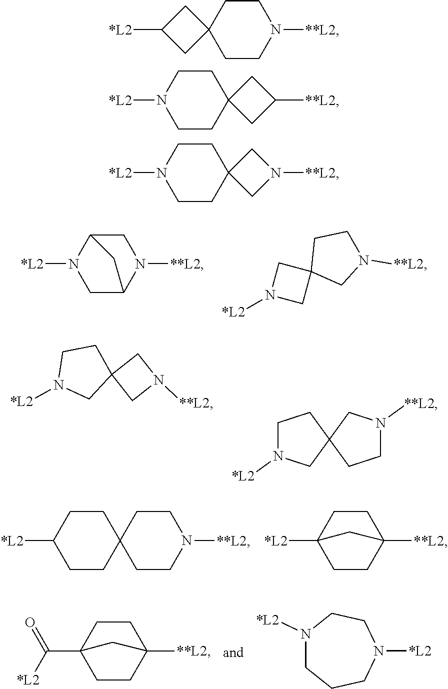
is optionally substituted with at least one R^{L1c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

27. The compound of any one of claims 1-26, wherein L² is selected from a single bond, —C₁-C₈alkylene-(preferably —CH₂—, —C₂H₄—, —C₃H₆—), —CO—, —O—, —N(CH₃)—, —NH—,

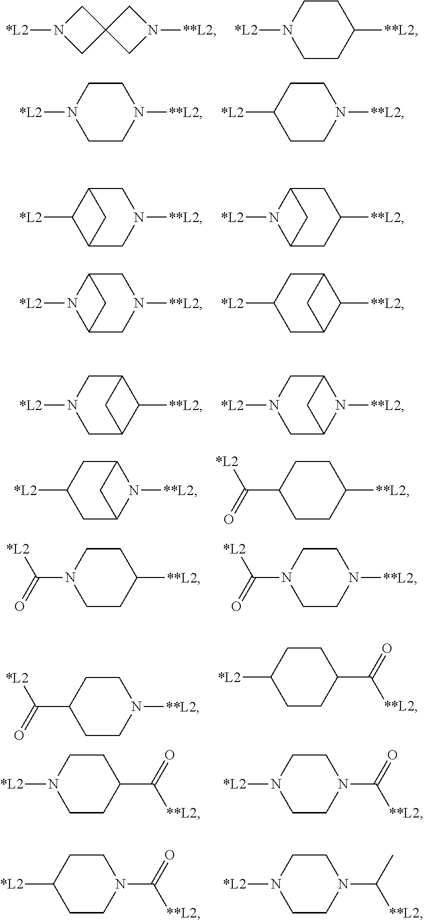

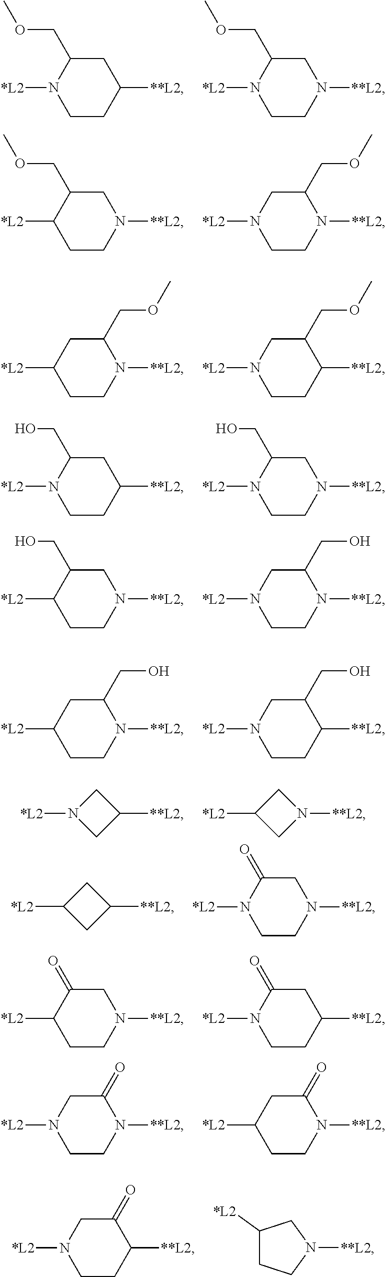

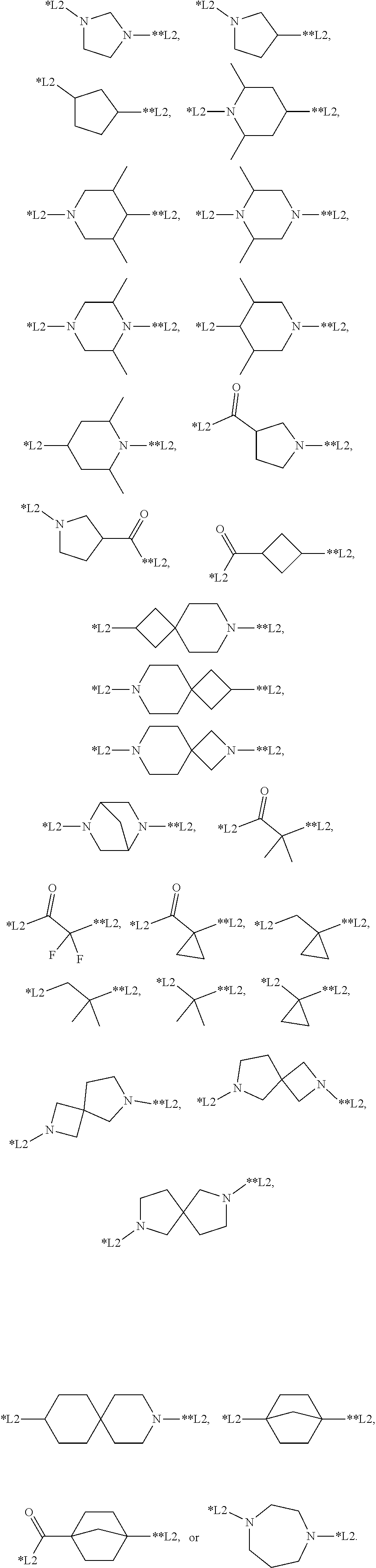

28. The compound of any one of claims 1-27, wherein L³ is selected from single bond, —C₁-C₈alkylene-(preferably —CH₂—, —C₂H₄—, —C₃H₆—), *^{L3}—CO—C₁-C₈alkylene-**^{L3} (preferably *^{L3}—CO—CH₂—**^{L3}, *^{L3}—CO—C₂H₄—**^{L3}, *^{L3}—CO—C₃H₆—**^{L3}), *^{L3}—C₁-C₈alkylene-CO—**^{L3} (preferably *^{L3}—CH₂—CO—**^{L3}, *^{L3}—C₂H₄—CO—**^{L3}, **^{L3}—C₃H₆—CO—*^{L3}), —CO—, —O—, —N(CH₃)—, —NH—, 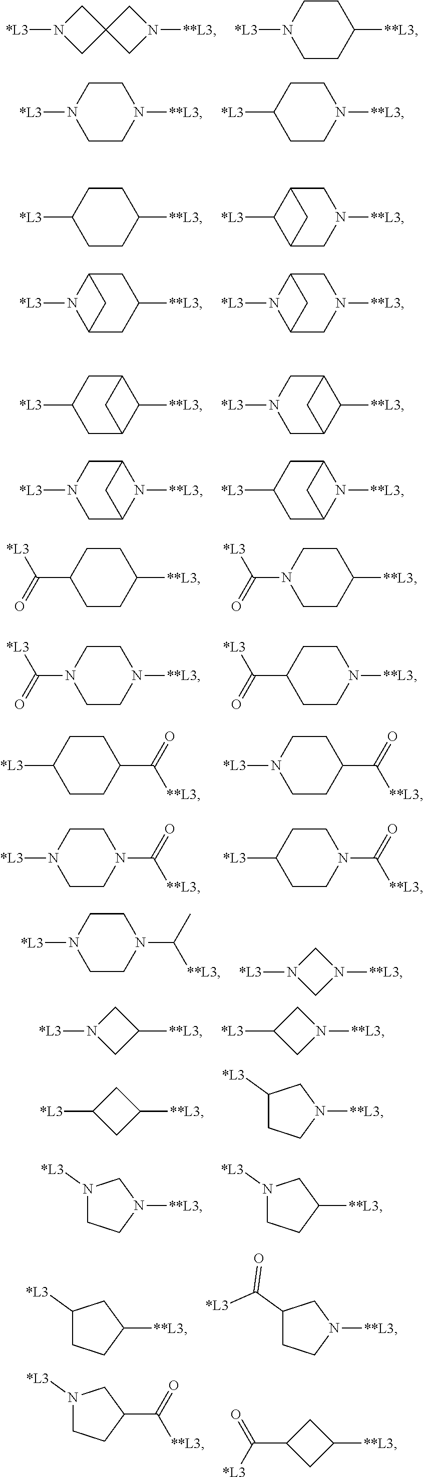 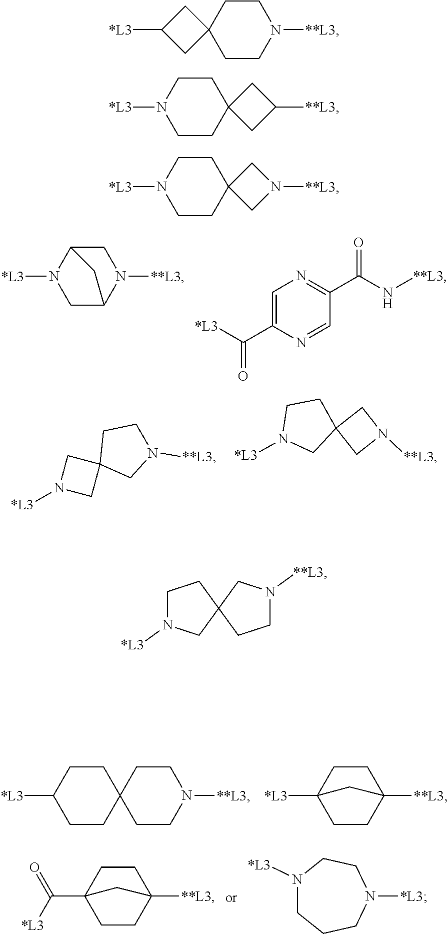
wherein each of said —C₁-C₈alkylene-(preferably —CH₂—, —C₂H₄—, —C₃H₆—), *^{L3}—CO—C₁-C₈alkylene-**^{L3} (preferably *^{L3}—CO—CH₂—**^{L3}, *^{L3}—CO—C₂H₄—**^{L3}, *^{L3}—CO—C₃H₆—**^{L3}), *^{L3}—C₁-C₈alkylene-CO—**^{L3} (preferably *^{L3}—CH₂—CO—**^{L3}, *^{L3}—C₂H₄—CO—**^{L3}, *^{L3}—C₃H₆—CO—**^{L3}), —N(CH₃)—, —NH—, 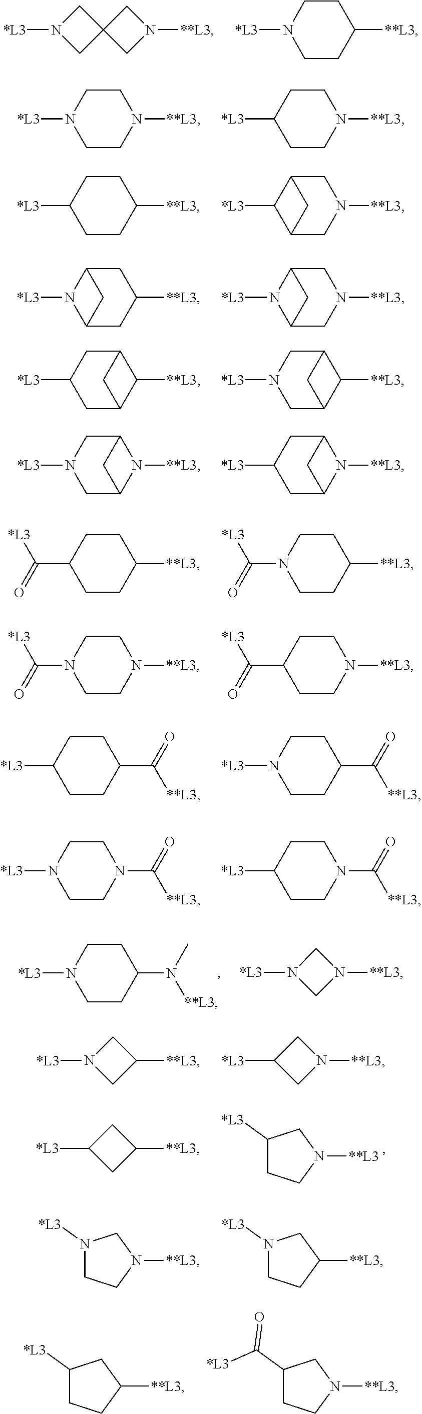 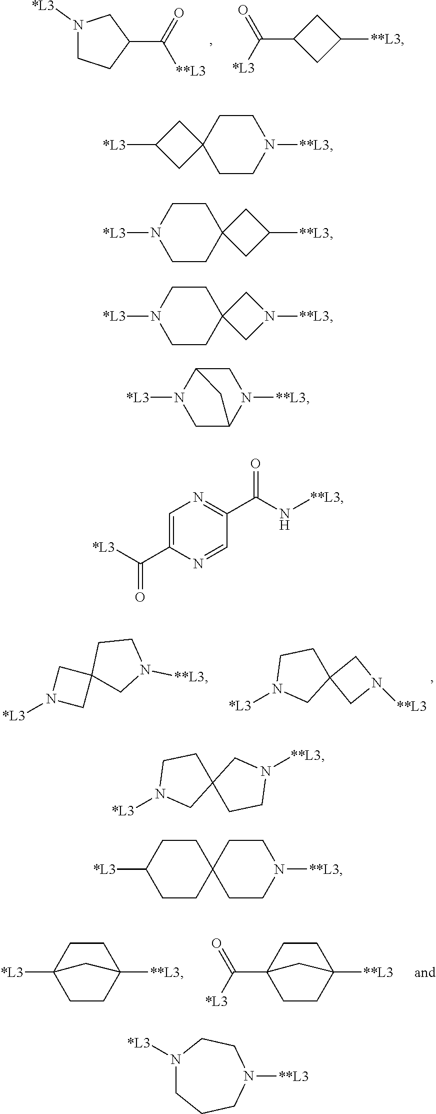
is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

29. The compound of any one of claims 1-28, wherein L³ is selected from a single bond, —C₁-C₈alkylene- (preferably —CH₂—, —C₂H₄—, —C₃H₆—), —CO—, —O—, —N(CH₃)—, —NH—,

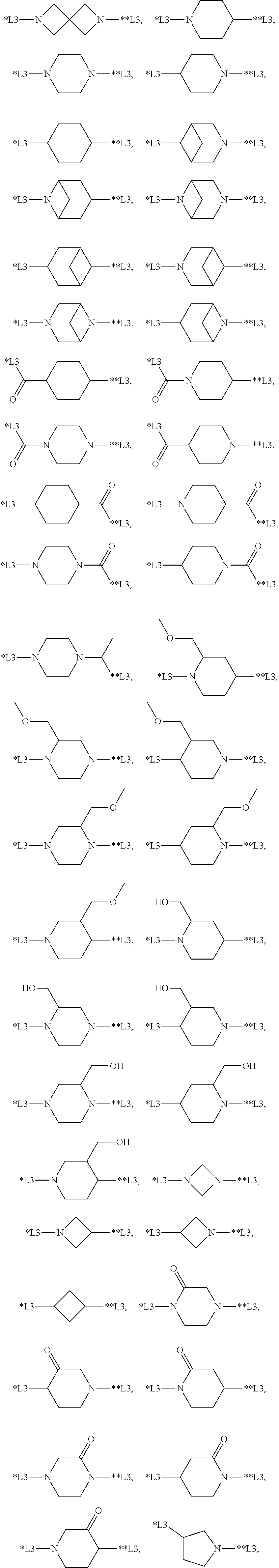

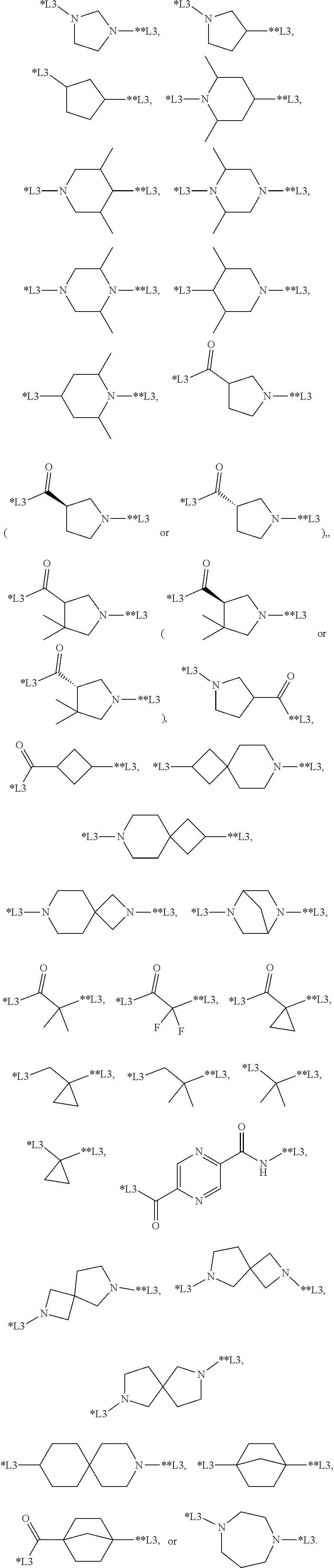

30. The compound of any one of claims 1-29, wherein L² is a single bond, L³ is a single bond, or L² and L3 are both single bond.

31. The compound of any one of claims 1-30, wherein 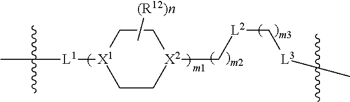
is selected from

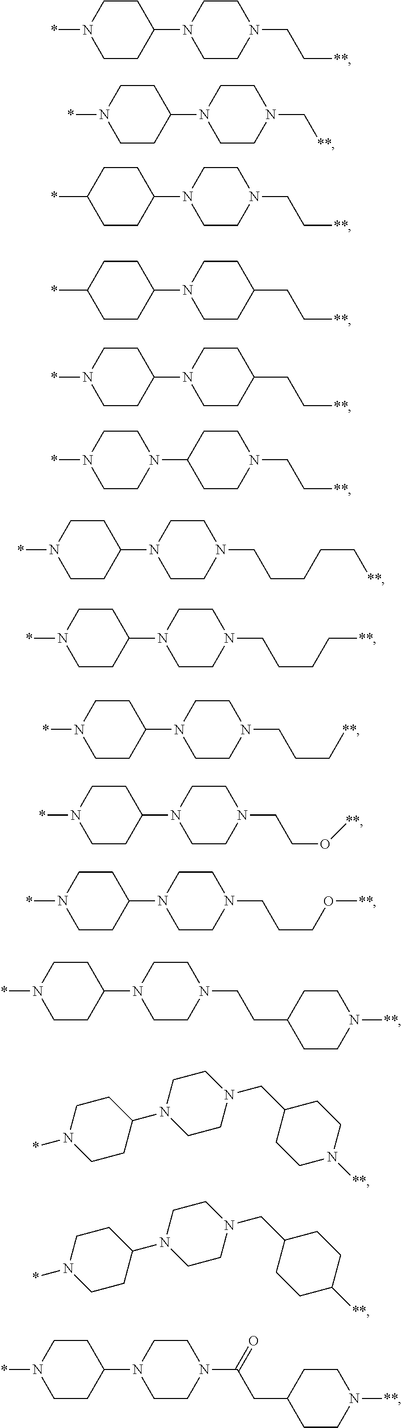

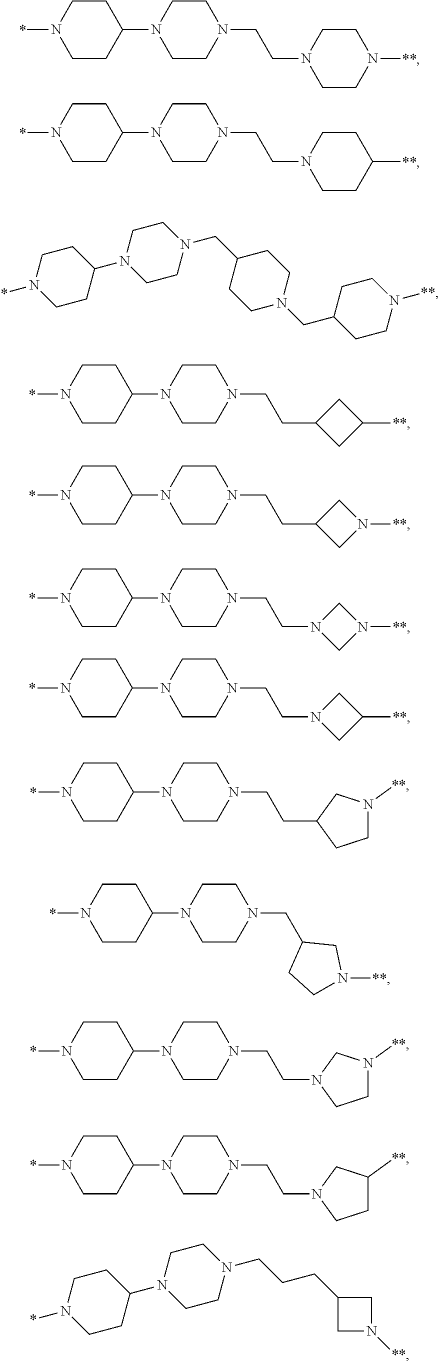

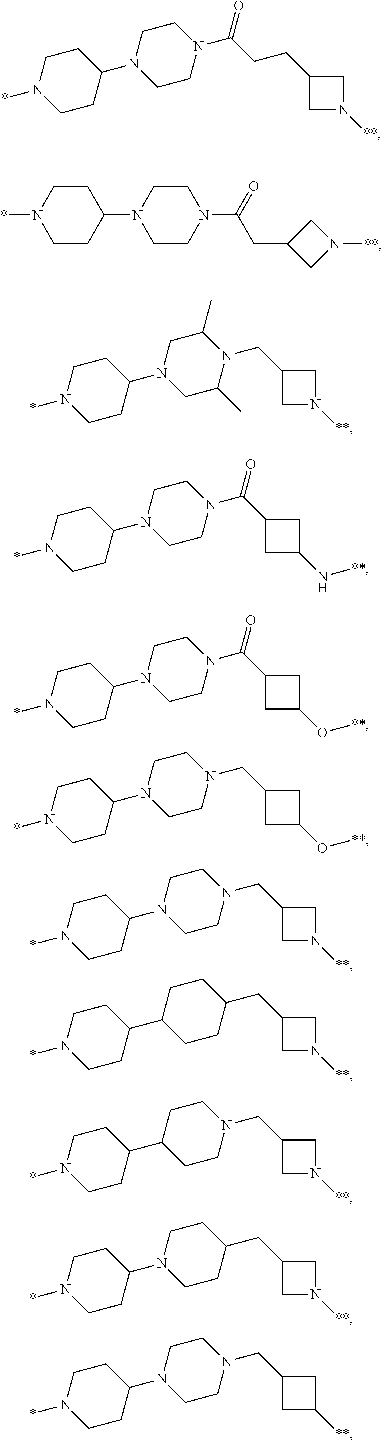

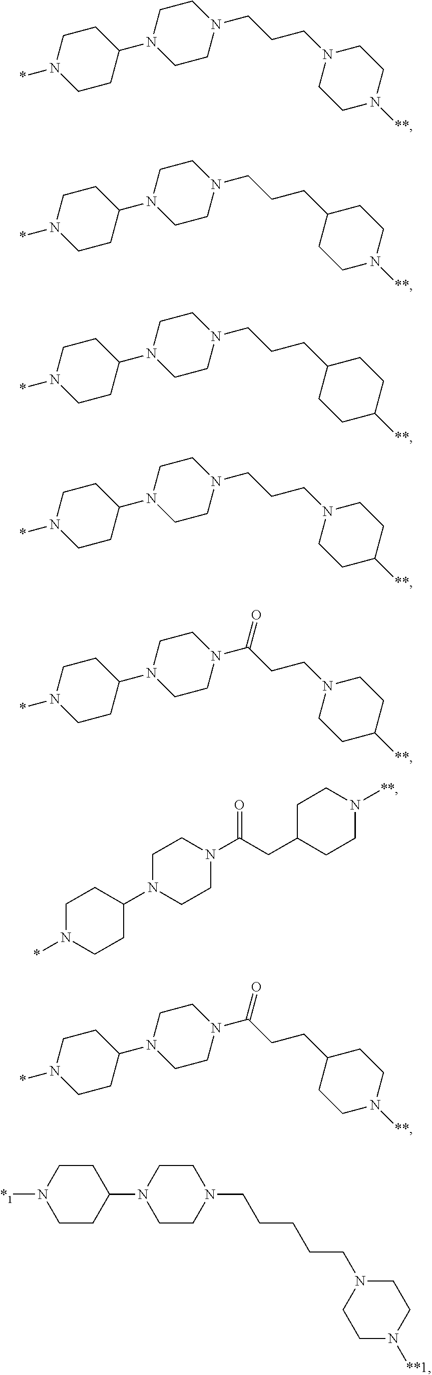

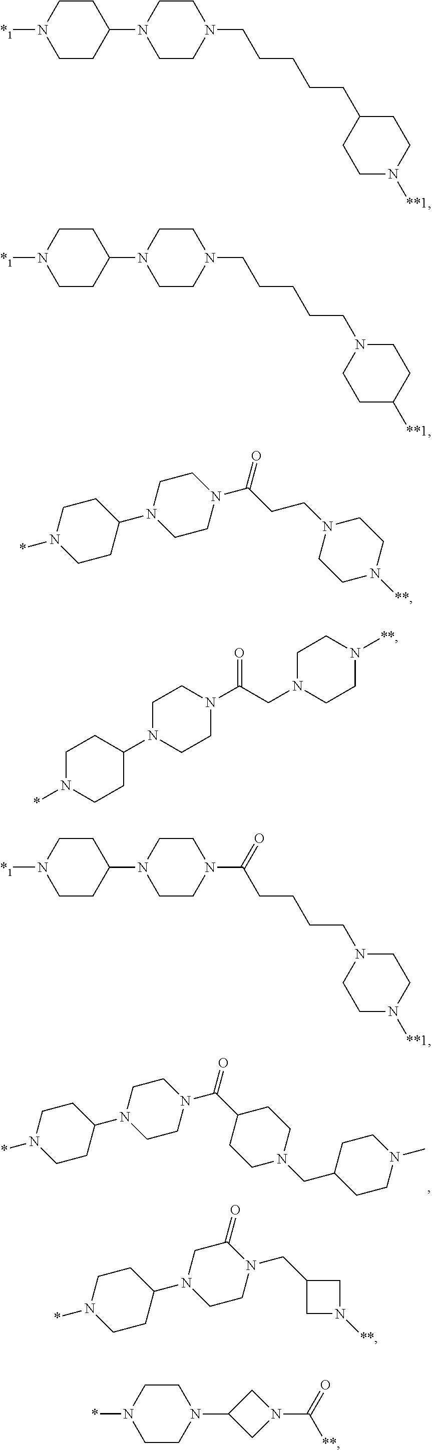

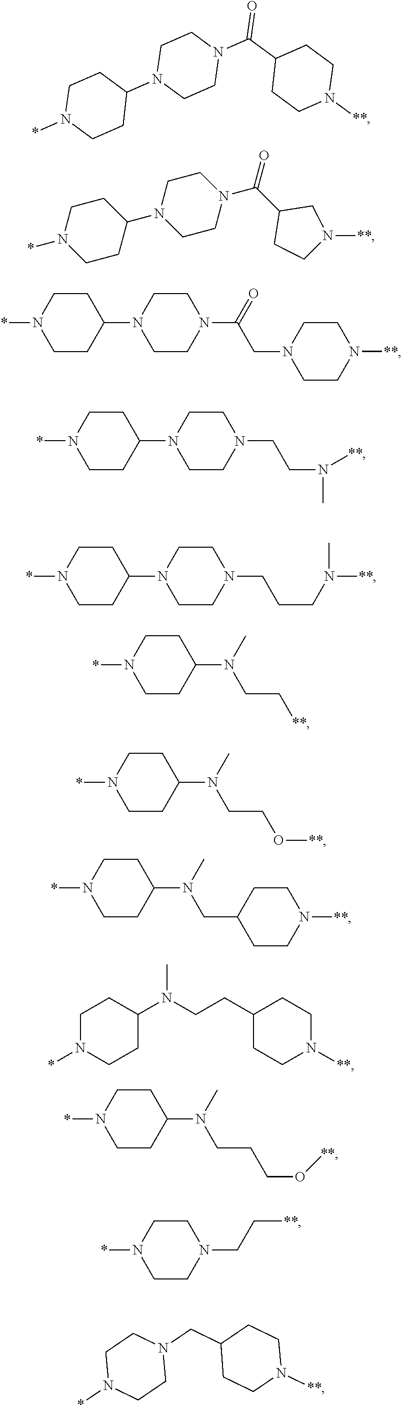

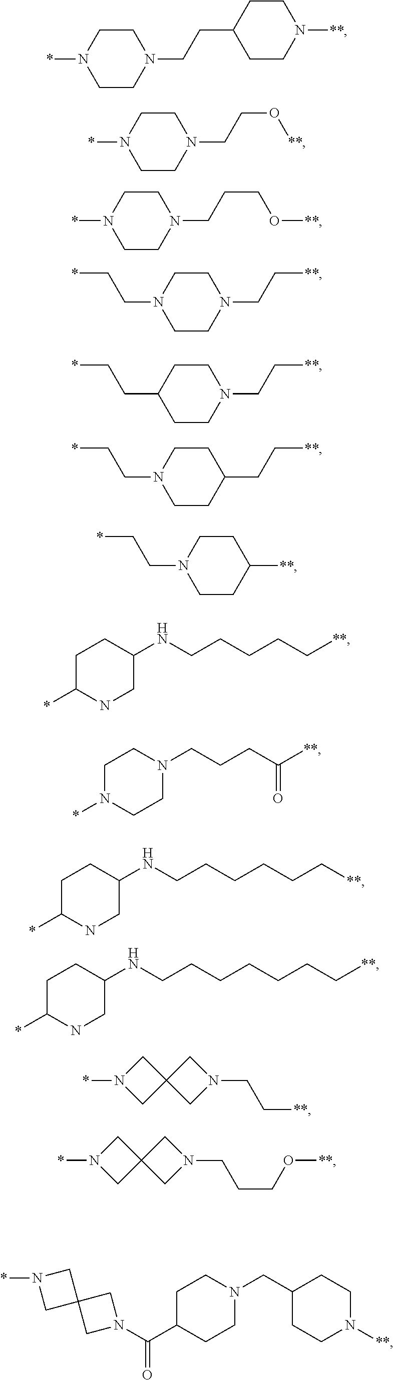

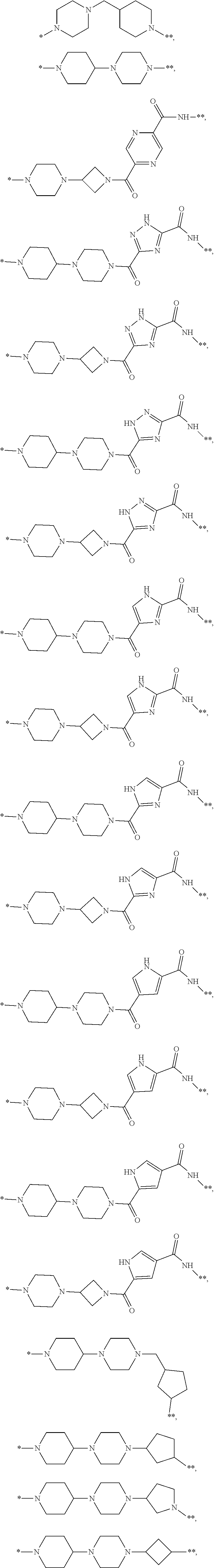

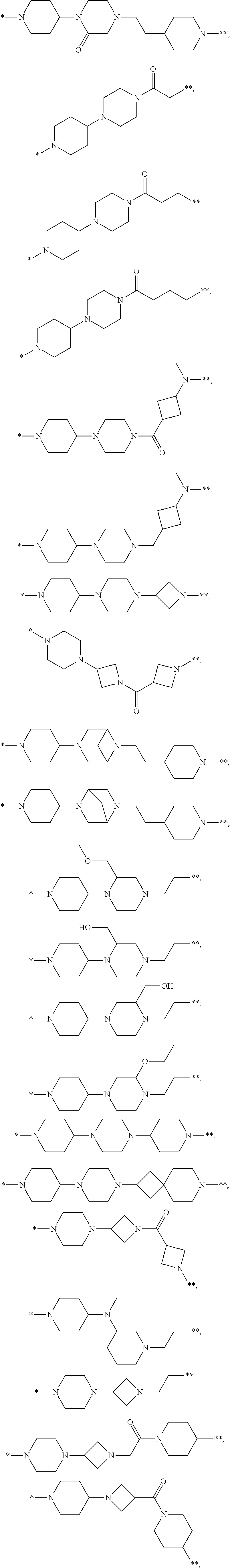

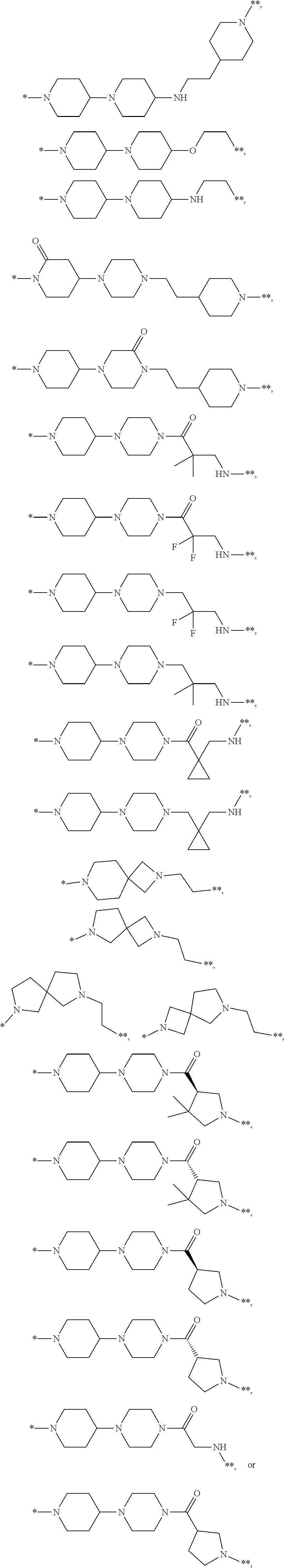

32. The compound of any one of claims 1-30, wherein R¹³, R¹⁴, R¹⁵, R¹⁶ and R¹⁷ are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl; said each methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, methyl, ethyl, propyl, butyl, pentyl, hexyl, heptyl, octyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl.

33. The compound of any one of claims 1-32, wherein at each occurrence, R^{a} and R^{b} are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or
R^{a} and R^{b} together with the carbon atoms to which they are attached, form a 3, 4, 5, 6, 7 or 8-membered ring, said ring comprising 0-3 heteroatoms independently selected from nitrogen, oxygen or sulfur; said ring is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl.

34. The compound of any one of claims 1-33, wherein 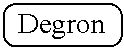
is selected from 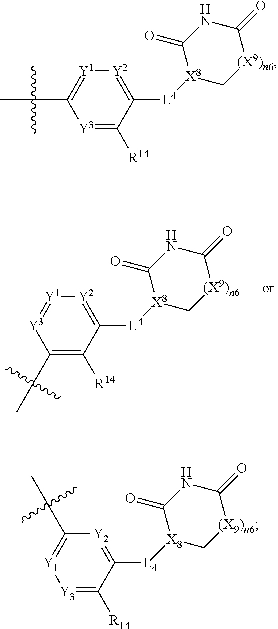
R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen or —C₁-C₈alkyl; preferably R¹⁴ is independently selected from H, F, Cl, Br, I, CH₃, —OCH₃, CH₂F, CN, CHF₂, or CF₃;
X⁸ is independently selected from CH, CD, C(CH₃), C(C₂H₅), C(C₃H₇), C(F) or N;
L⁴ is independently selected from a single bond, —O—, —NH—, —CH₂—, —CHF—, or —CF₂—;
Y¹, Y², and Y³ are each independently selected from CR^{a} or N;
X⁹ is CH₂;
R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen, hydroxy, —C₁-C₈alkyl, or —C₁-C₈alkoxy; and
n6 is independently 0, 1 or 2.

35. The compound of any one of claims 1-34, wherein 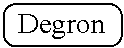
is selected from 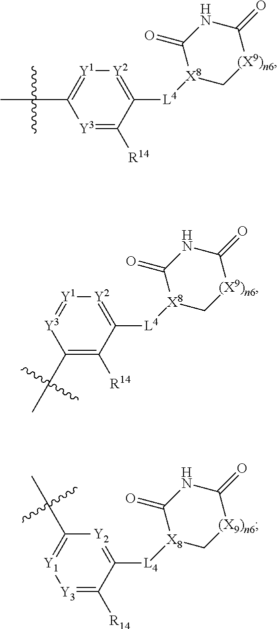
R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, CH₂F, CN, CHF₂, or CF₃;
X⁸ is independently selected from CH, CD, C(CH₃), C(C₂H₅), C(C₃H₇), C(F) or N;
L⁴ is a single bond;
Y¹, Y², and Y³ are each independently selected from CR^{a} or N;
X⁹ is CH₂;
R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen; and
n6 is 1.

36. The compound of any one of claims 1-35, wherein 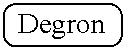
is selected from 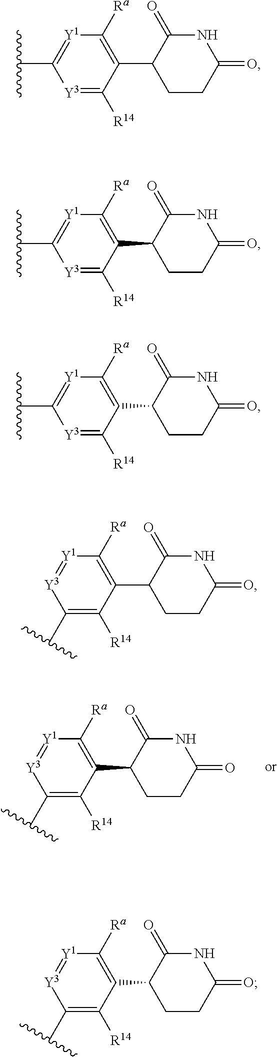
R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, CH₂F, CN, CHF₂, or CF₃;
Y¹ and Y³ are each independently selected from CH or N;
R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen.

37. The compound of any one of claims 1-36, wherein 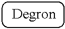
is selected from 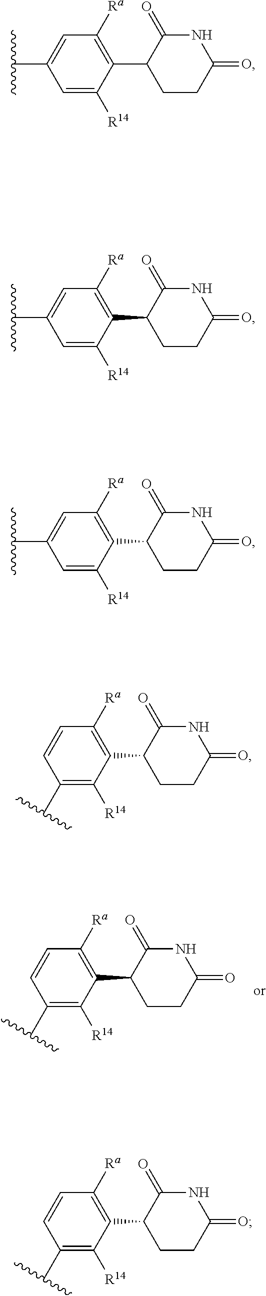
R¹⁴ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, or CN; said each methyl, ethyl, propyl, butyl, pentyl, hexyl, heptyl, octyl, methoxy, ethoxy, propoxy, butoxy, pentoxy, hexoxy, heptyloxy, octyloxy is optionally substituted by one or more F, Cl, Br, I; preferably R¹⁴ is independently selected from H, F, Cl, Br, I, CH₃, —OCH₃, CH₂F, CN, CHF₂, or CF₃;
R^{a} is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, each of said methyl, ethyl, propyl, butyl, pentyl, hexyl, heptyl, octyl, methoxy, ethoxy, propoxy, butoxy, pentoxy, hexoxy, heptyloxy, octyloxy is optionally substituted with at least one or more F, Cl, Br, I.

38. The compound of any one of claims 1-37, wherein 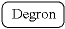
is selected from 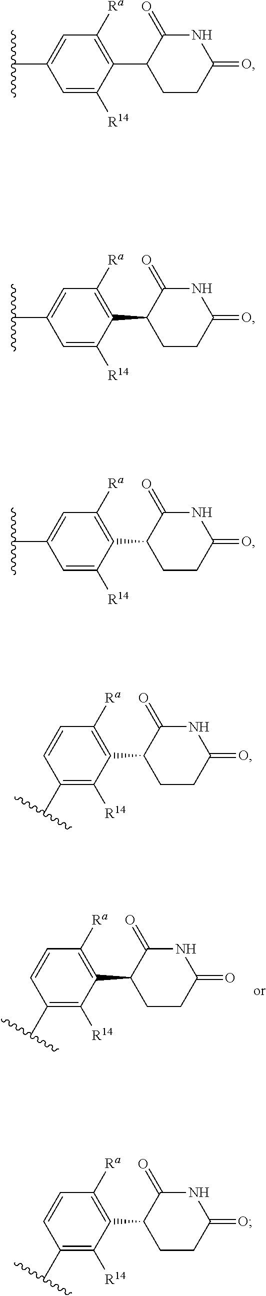
R¹⁴ is independently selected from F, Cl, Br, I, —CH₃, —OCH₃, CH₂F, CN, CHF₂, or CF₃;
R^{a} is each independently selected from F, Cl, Br, I, —CH₃, —OCH₃, CH₂F, CN, CHF₂, or CF₃.

39. The compound of any one of claims 1-38, wherein 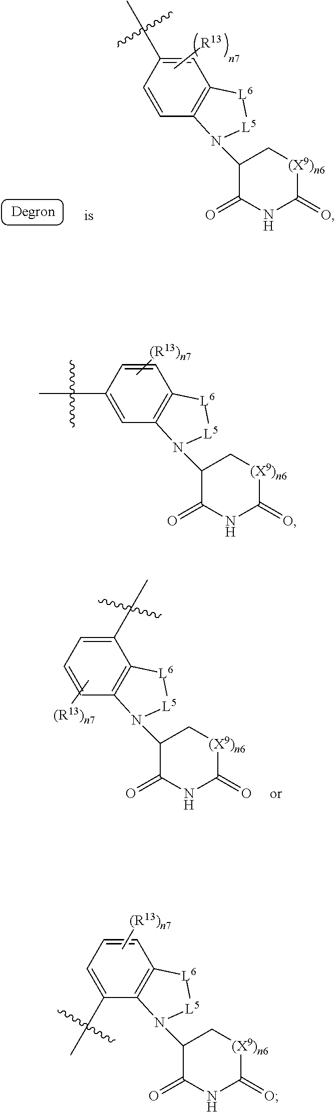
Wherein L⁵ and L⁶ are independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;
X⁹ is —CR^{a}R^{b}—;
R^{a} and R are each independently selected from hydrogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl and 5- to 12-membered heteroaryl is optionally substituted with at least one substituent halogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or
R^{a} and R^{b} together with the carbon atoms to which they are attached, form a 3-, 4-, 5-, 6-, 7-, 8-membered ring, said ring comprising 0-3 heteroatoms independently selected from nitrogen, oxygen or sulfur; said ring is optionally substituted with at least one substituent halogen, hydroxy, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
each R¹³ is independently selected from hydrogen, halogen, CN, —C₁-C₈alkyl, or —C₁-C₈alkoxy;
n₆ is 0 or 1; and
n₇ is 0, 1 or 2.

40. The compound of any one of claims 1-39, wherein 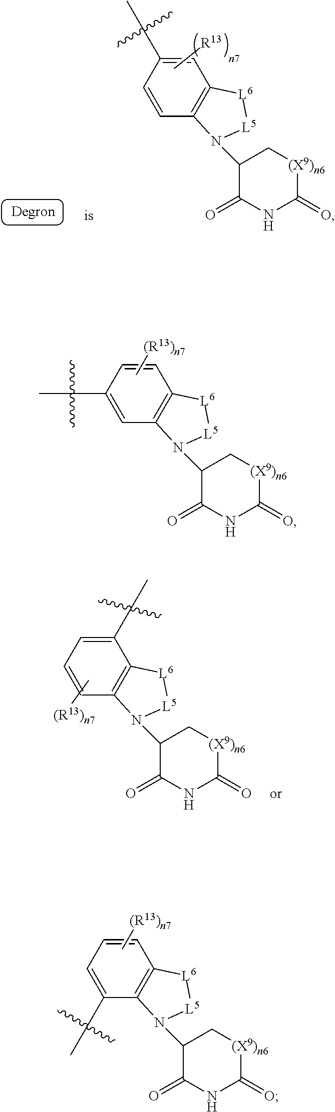
Wherein L⁵ and L⁶ is independently selected from a single bond, 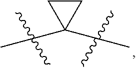 [SMILES: *C1(*)CC1]
—O—, —NH—, —NMe-, —N(CH₂CH₃)—, —CH₂—, —CHF—, —CF₂—, —C(CH₃)₂— or —CO— (preferably L⁶ is —CO— or —CH₂ and L⁶ is 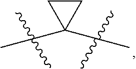 [SMILES: *C1(*)CC1]
—O—, —NH—, —NMe-, —N(CH₂CH₃)—, —CH₂—, —CHF—, —CF₂—, —C(CH₃)₂— or —CO—);
X⁹ is CH₂;
each R¹³ is independently selected from hydrogen, halogen, CN, —C₁-C₈alkyl, or —C₁-C₈alkoxy;
n₆ is 0 or 1; and
n₇ is 0, 1 or 2.

41. The compound of any one of claims 1-40, wherein 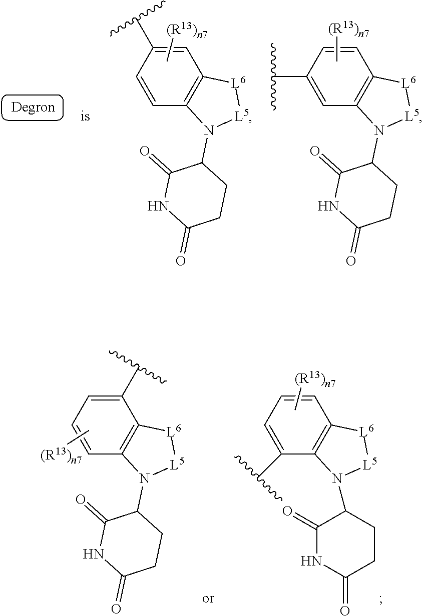
Wherein L⁵ and L⁶ are each independently selected from 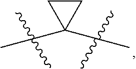 [SMILES: *C1(*)CC1]
—O—, —NH—, —NMe-, —N(CH₂CH₃)—, 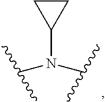 [SMILES: *N(*)C1CC1]
—CH₂—, —CHF—, —CF₂—, —C(CH₃)₂— or —CO—;
each R¹³ is independently selected from hydrogen, F, Cl, Br, I, CN, -Me, -Et, —C₃H₇, —C₄H₉, —OMe, —OEt, —OC₃H₇ or —OC₄H₉;
n₇ is 0, 1 or 2.

42. The compound of any one of claims 1-41, wherein 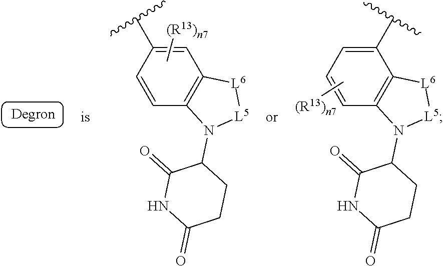
Wherein L⁶ is selected from 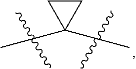 [SMILES: *C1(*)CC1]
—O—, —NMe-, —N(CH₂CH₃)—, —CH₂—, —CHF—, —CF₂— or —C(CH₃)₂—;
Wherein L⁵ is —CO—;
each R¹³ is independently selected from hydrogen, F, Cl, Br, I, CN, —C₁-C₈alkyl, or —C₁-C₈alkoxy;
n₇ is 0, 1 or 2.

43. The compound of any one of claims 1-42 wherein 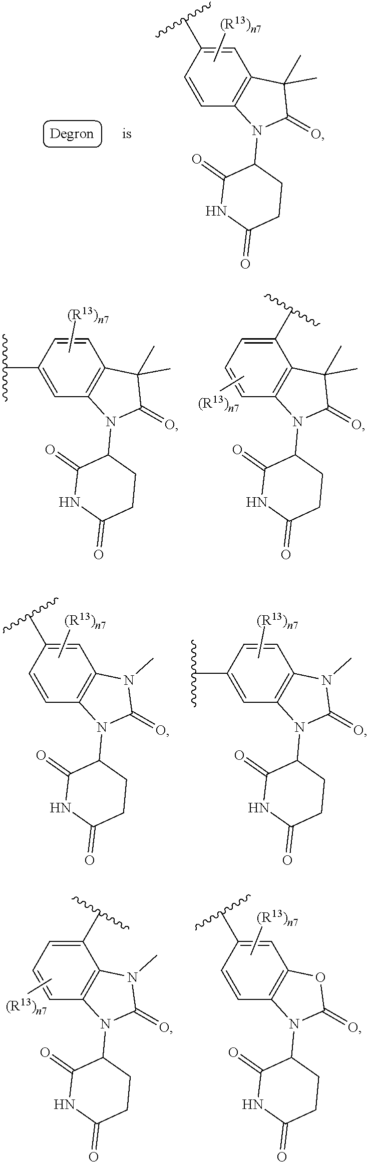 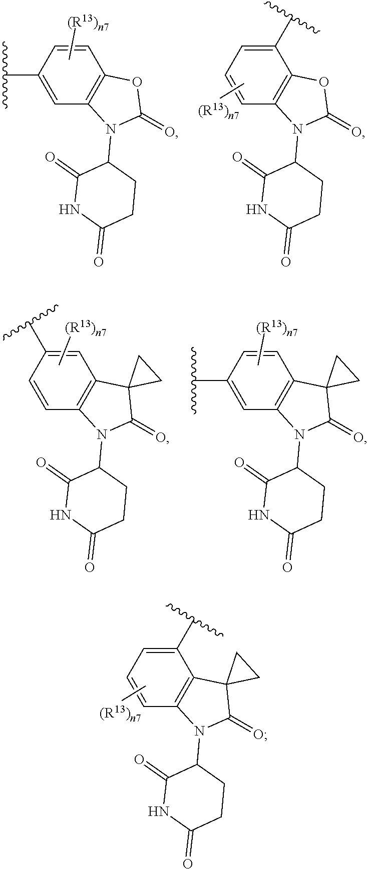
each R¹³ is independently selected from hydrogen, F, Cl, Br, I, CN, -Me, -Et, —C₃H₇, —C₄H₉, —OMe, —OEt, —OC₃H₇ or —OC₄H₉;
n₇ is 0, 1 or 2.

44. The compound of any one of claims 1-43, wherein 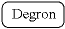
is selected from

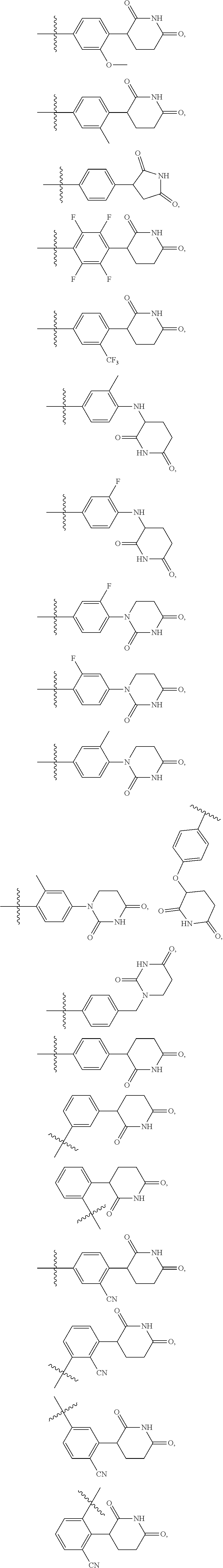

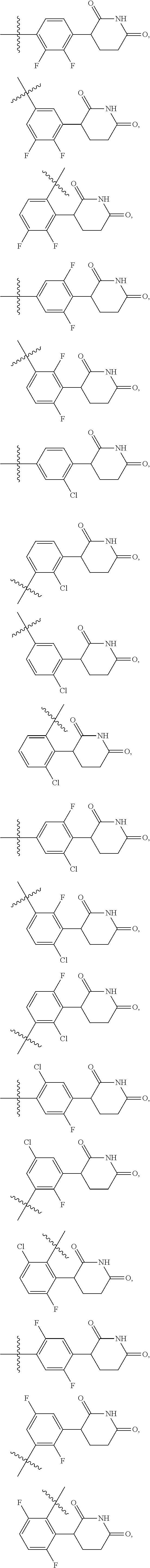

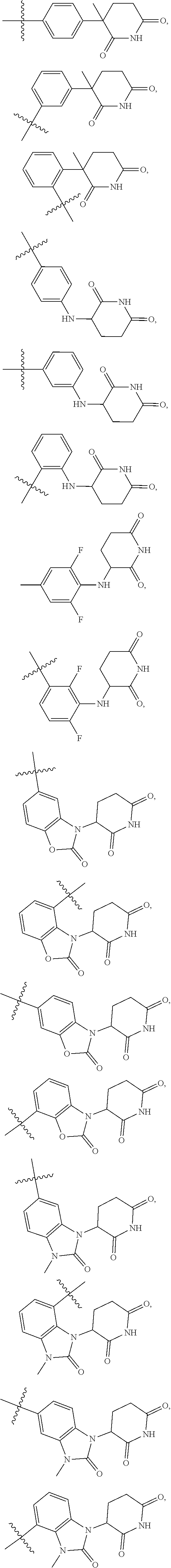

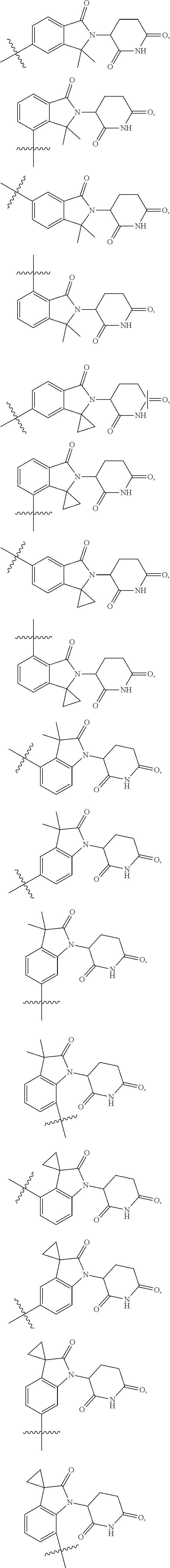

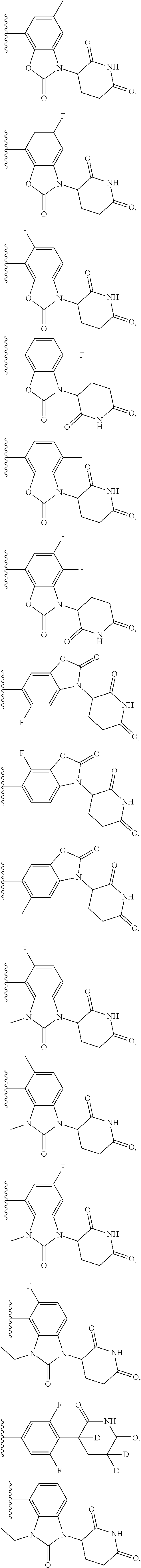

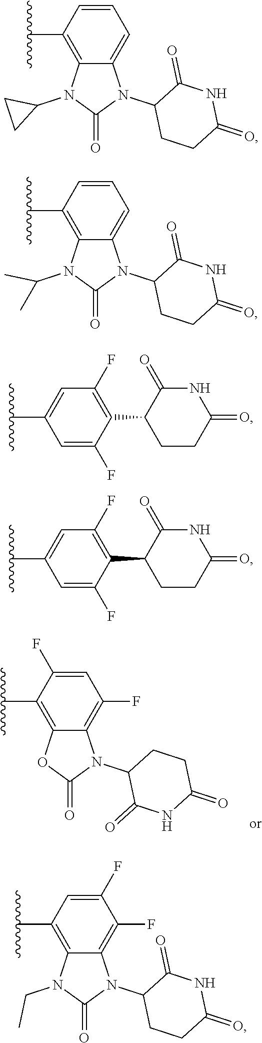

45. The compound of any one of claims 1-44, wherein Z¹, Z², Z³ and Z⁴ are each independently —CR^{z};
R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{Zc};
R^{Za} and R^{Zb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, —C₁-C₈alkoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

46. The compound of any one of claims 1-45, wherein R^{z}, at each occurrence, is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CH(OH)CH₃, or

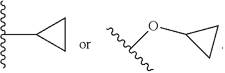

47. The compound of any one of claims 1-46 selected from Examples 23, 318, 413, 459, 460, 462, 473, 483, 484, 485, 486, 487, 488, 489, 491, 492, 493, 499, 501, 502, 503, 504, 505, 541, 549, 551, 558, 559, 563, 566, 570, 571, 572, 576, 577, 584, 589, 643, 711, 725, 727, 728, 729, 730, 731, 732, 733, 736, 737, 738, 739, 740, 741, 742, 744, 745, 746, 747, 748, 749, 750, 751, 752, 753, 754, 755 or 756.

48. A pharmaceutical composition comprising a compound of any one of claims 1-47 or a pharmaceutically acceptable salt, stereoisomer, tautomer or prodrug thereof, together with a pharmaceutically acceptable excipient.

49. A method of treating a disease that can be affected by EGFR modulation, comprises administrating a subject in need thereof an effective amount of a compound of any one of claims 1-47 or a pharmaceutically acceptable salt, stereoisomer, tautomer or prodrug thereof.

50. The method of claim 49, wherein the disease is selected from cancer, preferred pancreatic cancer, breast cancer, glioblastoma multiforme, head and neck cancer, or non-small cell lung cancer.

51. Use of a compound of any one of claims 1-47 or a pharmaceutically acceptable salt, stereoisomer, tautomer or prodrug thereof in the preparation of a medicament for treating a disease that can be affected by EGFR modulation.

52. The use of claim 51, wherein the disease is cancer, preferred pancreatic cancer, breast cancer, glioblastoma multiforme, head and neck cancer, or non-small cell lung cancer.
